SEQUENCE LISTING (3 sequences)

SEQ ID NO: 1 - protein, 9 aa, Artificial
  synthesized linear peptide
CFCDGRCDC

SEQ ID NO: 2 - protein, 5 aa, Artificial
  tumor homing peptide
CNGRC

SEQ ID NO: 3 - protein, 7 aa, Artificial
  microbe homing peptide
KLAKKLA

(19) United States
(12) Patent Application Publication
(10) Pub. No.: US 2007/0275911 A1
(43) Pub. Date: Nov. 29, 2007

(54) Targeted Drug-Formaldehyde Conjugates and Methods of Making and Using the Same

(76) Inventors: Tad H. Koch, Boulder, CO (US); Michael P. Coleman, Louisville, CO (US); Peter S. Cogan, Boulder, CO (US); Patrick J. Burke, Seattle, WA (US); Glen C. Post, Spokane, WA (US); David J. Burkhart, Spokane, WA (US); Andrew R. McKenzie, Belmont, MA (US); Katrina L. Jackson, Boulder, CO (US); Brian T. Kalet, Boulder, CO (US)

Correspondence Address: SHERIDAN ROSS PC, 1560 BROADWAY, SUITE 1200, DENVER, CO, 80202, US

(21) Appl. No.: 10/570,471

(22) PCT Filed: Sep. 7, 2004
(86) PCT No.: PCT/US04/29095

§ 371 Date: Apr. 23, 2007

Related U.S. Application Data
(60) Provisional application No. 60/500,608, filed on Sep. 5, 2003.

(51) Int. Cl. A61K 38/14; A61K 31/497; C07D 401/02; C07H 15/252; A61K 31/704

(52) U.S. Cl. 514/34; 514/187; 514/253.06; 514/312; 514/53; 536/55; 536/6.4; 544/363; 546/10; 546/156; 514/8

(57) ABSTRACT

The invention provides a prodrug platform technology for improving the therapeutic value of a variety of parent drug compounds by altering and improving drug characteristics such as aqueous solubility, hydrolytic stability, therapeutic index, toxicity profile, pharmacolcinetics and selectivity while allowing the potential for synthetic elaboration. The prodrug platform is particularly well suited for targeting therapeutic drugs, including anti-tumor drugs and antibiotics, to specific receptors on target cells (e.g., cancer cells and bacteria). The platform is a technology for providing an improved, preactivated form of a therapeutic drug, and for optionally targeting such drug to target cells or biological molecules. The invention is broadly applicable to many different therapeutic drugs, as well as to a variety of diseases and conditions, including a variety of forms of cancer and bacterial infections.

### FIELD OF THE INVENTION

The invention lies in the field of pharmaceutical compositions and specifically N-Mannich base prodrug conjugates.

### BACKGROUND OF THE INVENTION

The prodrug approach to modifying pharmaceuticals in order to overcome one or more undesirable property of the parent drug has been studied and applied to many compounds in clinical use today. The prodrugs formed are often intended to modify the absorption, metabolism, excretion, toxicity or activity of the parent compound in a desirable way. Additionally, prodrug modifications have been made to some compounds with the goal of creating a drug that is selectively activated or deactivated in a target tissue to increase the specificity of the intended drug effects while decreasing the unintended side effects associated with the parent compound. Thus, the prodrug approach is often looked to as a means of increasing the therapeutic index of a drug instead of trying to develop entirely new therapeutic compounds having more desirable pharmacokinetic and adverse effect profiles.

The prodrug approach has been applied to some of the most successful antibiotics and chemotherapeutic compounds that are designed to be toxic to some living cells and simultaneously non-toxic or much less toxic to other populations of living cells. For example, antibiotics, and particularly the anthracycline antibiotics including doxorubicin (Adriamycin™), have proven to be some of the most clinically useful antineoplastic agents. Considered a broad spectrum drug, doxorubicin (DOX) has been extensively employed in the treatment of Hodgkin's disease, non-Hodgkin's lymphomas, acute leukemias, sarcomas, and solid tumors of the lung, liver, breast, and ovary. Extensive investigations into the mechanism of action have failed to produce derivatives of superior therapeutic value. While hundreds of modifications to the anthraquinone core, the side chain, and the sugar moiety have been explored, very few have displayed even modest improvement with respect to the therapeutic index. Although several derivatives have been found to exhibit greater cytotoxicity than the clinically used anthracyclines, a concomitant increase in systemic toxicity is also commonly observed. Thus, anthracycline prodrugs have been studied with the general aim of improving the biodistribution of the drug and to diminish its systemic toxicity. To this end, several prodrugs of doxorubicin, which serve to carry the drug as an inactive species have been prepared and evaluated in recent years.

Some of the most promising work has focused on the development of prodrugs of doxorubicin which exploit part of the cytotoxic mechanism. Recent reports from several laboratories have suggested that the oxidative stress known to be induced by doxorubicin can lead to the generation of various aldehydes, as well as other reactive intermediates, which may serve to modify both the structure and activity of the parent drug. Of considerable interest is the production of formaldehyde, which has been demonstrated both in vitro and in living cells. Substantial evidence suggests that formaldehyde is generated by the anthracycline antibiotics in forming quasi-stable covalent adducts with DNA. These drug-DNA adducts have been directly observed by mass spectrometry, NMR, and X-ray crystallography and are inferred from the varying rates of release of doxorubicin from the nuclei of tumor cells, as well as from double stranded DNA in cell free systems. Further, the formaldehyde-releasing prodrugs, pivaloylmethyl butyrate and hexamethylenetetramine, enhance the cytotoxicity of doxorubicin.

To capitalize on this novel mode of action, a series of prodrugs has been developed that deliver formaldehyde along with the anthracycline compound to the cancer cell. This first generation of drug-formaldehyde conjugates was synthesized by the reaction of doxorubicin, daunorubicin, or epidoxorubicin with formaldehyde in acidic aqueous buffer. The prodrugs produced were found to be dimeric, consisting of two anthracycline molecules bonded together with three molecules of formaldehyde. The prodrugs were named doxoform, daunoform and epidoxoform respectively and are described in U.S. Pat. No. 6,677,309. These prodrugs were found to yield superior cytotoxins relative to the parent drugs upon hydrolysis to the respective formaldehyde-anthracycline Schiff bases, proposed to be active metabolites of the anthracyclines. In general, the formaldehyde conjugates are much more toxic than the corresponding anthracyclines and are equally toxic to both sensitive and resistant human tumor cells with doxoform showing the highest toxicity of the three prodrugs. While doxoform proved to be too toxic for mouse experiments, epidoxoform proved to be more effective for treating a mouse mammary tumor than its clinical predecessor, epidoxorubicin.

Unfortunately, these prodrugs were also characterized by hydrolytic instability and poor aqueous solubility, and, in the case of doxoform, high systemic toxicity. The low water solubility of these compounds is thought to result from high molecular symmetry and the absence of charged groups. They are also expected to demonstrate relatively indiscriminant pharmacokinetics and, therefore, offer less than optimal improvements with respect to the therapeutic index of the parent anthracycline antibiotics.

In addition to the research described above with regard to the anthracyclines, prodrug derivatives of other anti-tumor drugs have also been extensively studied. For example, cisplatin has been among the most widely used agents in cancer chemotherapy. As a single agent or in combination therapy, cisplatin is effective in the treatment of a wide variety of human malignancies, including testicular, ovarian, bladder, head and neck, lung, and breast cancers. However, there are two inherent problems associated with the use of cisplatin as a chemotherapeutic agent. The largest is the cumulative toxicity of cisplatin resulting in nephrotoxicity, ototoxicity and peripheral neuropathy and the second is the development of resistance in cancer cells that have been exposed to cisplatin. In efforts to circumvent these problems, thousands of prodrug derivatives of cisplatin have been synthesized and evaluated. The only derivative with activity comparable to cisplatin, though less toxic, is the second-generation analogue, carboplatin.

In addition to antineoplastic and anthracycline antibiotics, other antibiotic drugs can be improved through the development of prodrug derivatives that have improved specificity for the infectious organism. For example, the fluoroquinolones, represented by norfloxacin, ciprofloxacin, sparfloxacin, gatifloxacin, levofloxacin, and moxifloxacin, are an important class of antibiotics with clinical activity against Gram positive and Gram negative bacteria as well as mycobacteria. The structure of these fluoroquinolones and their target of activity share some features with the clinically important antitumor drugs, doxorubicin and epidoxorubicin, which are classified as topoisomerase II poisons. It has therefore been suggested that the continuing problem of bacterial resistance to antibiotics could be addressed through the application of the prodrug approach to known antitumor and antibiotic compounds to produce new antibacterial drugs with greater toxicity and/or greater selectivity for infectious organisms.

The search for effective prodrug compounds based on a particular parent compound can be very time consuming and expensive. Typically, dozens or even hundreds of chemical modifications are made to the parent compound and these derivatives are tested in vivo to evaluate differences in pharmacokinetics, toxicity, selectivity or efficacy. But very few prodrug approaches have been identified that are consistently useful when applied to a wide variety of drug compounds. Therefore, there is a need in the pharmaceutical arts for a prodrug system that is applicable to many classes of drugs, including antineoplastic and antibiotic drugs, that can enhance the clinical properties of these compounds through improved aqueous solubility, hydrolytic stability, selectivity, therapeutic index or efficacy without restricting the potential for synthetic elaboration.

### SUMMARY OF THE INVENTION

The present invention provides a prodrug platform technology for improving the therapeutic properties of a variety of drugs by addressing the above-described need for drugs having improved aqueous solubility, hydrolytic stability, pharmacokinetics, efficacy, toxicity and specificity with the potential for further synthetic elaboration. The present invention also provides a prodrug platform technology for targeting therapeutic drugs, including, but not limited to, anti-tumor drugs and antibiotics, to specific receptors on target cells (e.g., cancer cells and bacteria). More specifically, a technology for providing an improved, preactivated form of a therapeutic drug, and for targeting such drug to target cells is described. The invention has broad applicability to many different therapeutic drugs, as well as to a variety of diseases and conditions, including a variety of forms of cancer and bacterial infections.

The prodrug compounds of the present invention are described by the general formula: 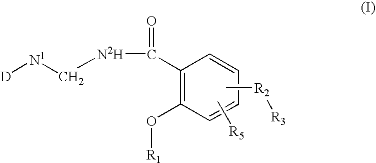
or a pharmaceutically acceptable salt thereof. In formula (I), D is a drug moiety that contains at least one primary or secondary amine designated N¹. In the instance in which the drug contains a secondary amine, the amine may be part of a branched or straight chain alkyl group or a cyclic secondary amine in which the nitrogen atom is a member of an alkyl ring structure. Thus, N¹ in formula (I) above is a nitrogen atom that is part of a primary or secondary amine that is contained within the structure of a drug molecule designated “D.” In this sense, N¹ is naturally a part of the drug molecule D and is donated by the drug molecule D to participate in prodrug system which is attached to D through N¹. Thus, the drug molecule, D, must contain a primary or secondary amine to be eligible for incorporation into the prodrug system of the present invention. If the drug molecule, D, contains a primary or secondary amine, the prodrug system of the present invention can be linked to the drug through the amine nitrogen that is then designated N¹ of formula (I).

R₁ in formula (I) is H or —CH₂—O—C(O)R₄ where R₄ is either H or a linear or branched alkyl, alkenyl, alkynyl, aryl, alkoxy, aryloxy, arylalkoxy, heteroaryl, arylalkyl, heteroarylalkyl, cycloalkyl, cycloalkylalkyl, polycycloalkyl, polycycloalkylalkyl, cycloalkenyl, cycloheteroalkyl, heteroaryloxy, cycloalkenylalkyl, polycycloalkenyl, polycycloalkenylalkyl, heteroarylcarbonyl, amino, alkyl-amino, arylamino, heteroarylamino, cycloalkyloxy, or cycloalkylamino moiety. It should be understood that the hydroxy/alkoxy group of R₁ must appear adjacent or ortho to the carbonyl carbon on the benzene ring to properly trigger release of the drug moiety, D, from the prodrug construct as explained in detail below.

The “tether” moiety, represented by R₂ in formula (I), may optionally be absent or, if present, is either a bond or an alkyl, alkenyl, alkynyl, allenyl, aryl, alkoxy, aryloxy, polyalkyloxy, arylalkoxy, heteroaryl, arylalkyl, heteroarylalkyl, cycloalkyl, cycloalkylalkyl, polycycloalkyl, polycycloalkylalkyl, cycloalkenyl, cycloheteroalkyl, heteroaryloxy, cycloalkenylalkyl, polycycloalkenyl, polycycloalkenylalkyl, heteroarylcarbonyl, amino, alkyl-amino, arylamino, heteroarylamino, cycloalkyloxy, or cycloalkylamino moiety. It should be appreciated that the R₂ group may be attached to any of the benzene ring carbons ortho, meta or para to the carbonyl group. Preferably, the R₂ group is attached to the benzene ring meta to the carbonyl group and para to the hydroxy or alkoxy R¹ group.

Like R₂, R₃ in formula (I) may be absent. If present, R₃ is a targeting compound that is capable of selectively binding to a specific target site in a mammal selected from the group consisting of a cell, a tissue, a bodily fluid, a receptor, a ligand and a cell surface molecule.

R₅ may be included to modify the timing of the trigger release of the drug compound from the prodrug conjugate of the present invention. An electron withdrawing substituent at R₅, such as cyano, acyl, nitro, alkoxycarbonyl or aminocarbonyl will make the trigger fire more quickly while an electron donating substituent such as hydroxyl, alkoxyl, acyloxy, or amido will make the trigger fire more slowly. Thus, the effect is predicted from how the R₅ substituent affects the acidity of the hydroxyl group of the salicylamide. Substituents that make the hydroxyl more acidic would accelerate the trigger firing and substituents that make the hydroxyl less acidic would slow the trigger firing. Thus, R₅ can be H, cyano, acyl, nitro, alkoxycarbonyl, aminocarbonyl, hydroxyl, alkoxyl, acyloxy, or amido. Similar to R₂, the R₅ group may be attached to any of the benzene ring carbons ortho, meta or para to the hydroxy/alkoxy substituent containing R₄. Preferably, R₅ is attached to the benzene ring ortho to the hydroxy/alkoxy substituent containing R₄.

In one embodiment of the present invention, the prodrug compounds described by formula (I) may be incorporated in a pharmaceutical composition that contains a therapeutically effective amount of a compound defined by formula (I) and one or more pharmaceutically acceptable excipients including carriers, binders, glidiants, buffers, and the like.

Preferably, the compounds of formula (I) include linear or branched alkyl, alkoxy, alkenyl, alkynyl, aryl, or heteroaryl group having between 1 and 20 carbons (C1-C20) at the R₄ position. Additionally, the moiety at the R₂ position of formula (I) is preferably a linear alkyl, alkenyl, alkynyl, allenyl, or polyalkyloxy entity having between 4 and 20 carbon atoms (C4-C20). Chemical entities that are particularly suitable at position R₂ in the prodrug compounds of the present invention defined by formula (I) include —CH₂OCH₂C}CCH₂—; —CH₂OCH₂—C≡C—; C≡C—CH₂—; —CH₂(OCH₂CH₂)ₙ— wherein n is an integer between 1 and 20; —CH═N—(OCH₂CH₂)ₙ—N(CH₃)CH₂CH₂— wherein n is 1, 2 or 3; —CH═N—OCH₂C(O)NHCH₂CH₂OCH₂CH₂—; —CH═N—OCH₂C≡C—CH₂—; —CH═N—OCH₂C—C≡C—C≡C—CH₂—; —CH≡NOCH₂CH₂OCH₂CH₂—; —CH═N—OCH₂C(O)—; and N,N′-disubstituted piperazines.

The “targeting compound” represented by R₃ in formula (I) can be a moiety that binds specifically to receptors overexpressed in cancer cells, thereby guiding the prodrug compound to cancer cells where the drug, D, may selectively exert a toxic effect. Alternatively, the targeting compound at R₃ may be a moiety that binds specifically to endothelial cells undergoing angiogenesis thereby delivering a drug to kill or suppress the growth of new vascular growth supporting tumor growth. As another example, R₃ may be a moiety that binds specifically to structures unique to bacterial cells, thereby guiding the prodrug complex specifically to bacterial cells where the drug, D, may exert an antibiotic effect.

The compounds defined by formula (I) include N-(2-hydroxybenzamidomethyl)-doxorubicin); N-(5-{4-[3-(4-Cyano-3-trifluoromethyl-phenyl)-5,5-dimethyl-2,4-dioxo-imidazolidin-1-yl]-but-2-ynyloxymethyl)-2-hydroxy-benzamidomethyl)-doxorubicin; and, E/Z-N-(2-Hydroxy-5- {[2-(2-{2-[(2-{4-[1-(4-hydroxy-phenyl)-2-phenyl-but-1-enyl]-phenoxy}-ethyl)-methyl-amino]-ethoxy}-ethoxy)-ethoxyimino]-methyl}-benzamidomethyl)-doxorubicin. The compounds defined by formula (I) include prodrug complexes represented by the chemical structures A-O shown in FIGS. 2-5.

Another embodiment of the present invention is a method of treating cancer in a mammal by administering a therapeutically effective amount of one of the prodrug compounds defined by formula (I) to a mammal. For example, administration of the prodrug compound N-(2-hydroxybenzamidomethyl)-doxorubicin) may be particularly effective in treating cancers such as Hodgkin's disease, non-Hodgkin's lymphoma, and acute leukemia. Additionally, administration of the prodrug compound N-(2-hydroxybenzamidomethyl)-doxorubicin) may be particularly effective in treating solid tumors in tissues such as lung, liver, breast, and ovary. Further, administration of the prodrug compound N-(5-{4-[3-(4-Cyano-3-trifluoromethyl-phenyl)-5,5-dimethyl-2,4-dioxo-imidazolidin-1-yl]-but-2-ynyloxymethyl)-2-hydroxy-benzamidomethyl)-doxorubicin may be particularly effective in treating prostate cancer. Using the prodrug compound E/Z-N-(2-Hydroxy-5-{[2-(2-{2-[(2-{4-[1-(4-hydroxy-phenyl)-2-phenyl-but-1-enyl]-phenoxy}-ethyl)-methyl-amino]-ethoxy}-ethoxy)-ethoxyimino]-methyl}-benzamidomethyl)-doxorubicin may be particularly effective in treating breast cancer.

Another embodiment of the present invention is a method of inhibiting or causing the regression of angiogenesis in a mammal by administering a therapeutically effective amount of a prodrug compound defined by formula (I). For example, any one of the prodrug compounds bicyclic DOXSF-RGD-4C, acyclic DOXSF-RGD-4C, cyclic-(N-Me-VRGDf-NH)DOXSF, anilinocyanoquinoline-cisplatinSF, anilinocyanoquinoline-DOXSF, cyclic-DOX-NGR, acyclic-DOX-NGR or a combination of these prodrug compounds may be particularly effective for the inhibition or regression of angiogenesis.

Another embodiment of the present invention is a method of cross-linking DNA in a cell by contacting the cell with a prodrug compound defined by formula (I). In this method, the cell in which the DNA is cross-linked by drug adducts may be located in a mammal, an organ or a tissue culture when the cell is contacted with the prodrug compound. Additionally, the cell may be any type of cell such as a mammalian cell, a bacterial cell, a cancer cell, an endothelial cell.

One embodiment of the present invention is a method of preventing or treating an infection in an organism by administering a therapeutically effective amount of a prodrug compound defined by formula (I) to an organism. In this embodiment, the infectious agent may be a gram positive or gram negative bacteria or a mycobacteria. Prodrug compounds that may be particularly useful in this method include vancociproform, ciprosaliform, moxisaliform, and ciprosaliform-KLAKKLA.

One embodiment of the present invention is a method of making a prodrug compound of the present invention by contacting salicylamide with formaldehyde in the presence of a drug moiety that has at least one primary or secondary amine to form an N-Mannich base. The N-Mannich base is then covalently-bound to a targeting compound capable of selectively binding to a specific target site in a mammal such as a cell, a tissue, a bodily fluid, a receptor, a ligand or a cell surface molecule. In a related embodiment of the present invention, a prodrug compound is produced by contacting a salicylamide analog with formaldehyde in the presence of a drug moiety that has at least one primary or secondary amine to form an N-Mannich base which is covalently bound to a targeting compound capable of selectively binding to a specific target site in a mammal such as a cell, a tissue, a bodily fluid, a receptor, a ligand and a cell surface molecule. Particularly suitable salicylamide analogs for use in the methods of the present invention are defined by the formula:

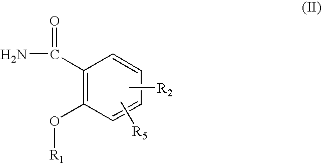

In formula (II), R₁ is H or —CH₂—O—C(O)R₄. R₄ is a linear or branched alkyl, alkenyl, alkynyl, aryl, alkoxy, polyalkyloxy, aryloxy, arylalkoxy, heteroaryl, arylalkyl, heteroarylalkyl, cycloalkyl, cycloalkylalkyl, polycycloalkyl, polycycloalkylalkyl, cycloalkenyl, cycloheteroalkyl, heteroaryloxy, cycloalkenylalkyl, polycycloalkenyl, polycycloalkenylalkyl, heteroarylcarbonyl, amino, alkyl-amino, arylamino, heteroarylamino, cycloalkyloxy, or cycloalkylamino moiety. R₂ is a compound such as —CH₂OCH₂C≡CCH₂—; —CH₂OCH₂—C≡C—C≡C—CH₂—; —CH═N—OCH₂C═C—CH₂—; —CH₂(OCH₂CH₂)ₙ— where n is an integer between 1 and 20; —CH═N—(OCH₂CH₂)ₙ—N(CH₃)CH₂CH₂— where n is 1, 2 or 3; —CH═N—OCH₂C(O)NHCH₂CH₂OCH₂CH₂—; —CH═N—OCH₂C—C≡C—C≡C—CH₂—; CH≡N—OCH₂C(O)—; —CH═NOCH₂CH₂OCH₂CH₂—; or an N,N′-disubstituted piperazine. Preferably, the salicylamide analogs used in the methods of the present invention are compounds of the formula:

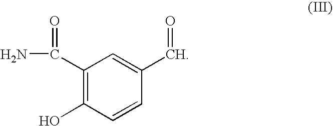

### BRIEF DESCRIPTION OF THE DRAWINGS

FIG. 1 shows the structures of the clinically relevant anthracyclines doxorubicin, daunorubicin, and epidoxorubicin.

FIG. 2 shows compounds A-F which are examples of useful prodrug pharmaceutical compounds of the present invention.

FIG. 3 shows compounds G-K which are examples of useful prodrug pharmaceutical compounds of the present invention.

FIG. 4 shows compounds L-N which are examples of useful prodrug pharmaceutical compounds of the present invention.

FIG. 5 shows compound O which is an example of a useful prodrug pharmaceutical compound of the present invention.

FIG. 6 is a schematic design illustrating a targeted prodrug-formaldehyde conjugate of the present invention distributing to a cell and the subsequent release of the drug-formaldehyde conjugate inside the cell.

FIG. 7 illustrates a proposed mechanism for hydrolysis of simple anthracycline-formaldehyde N-Mannich bases.

FIG. 8 illustrates a proposed mechanism for hydrolysis of doxsaliform and daunsaliform showing the participation of the phenolic hydroxyl group as a proton donor.

FIG. 9 is a schematic diagram of a targeted prodrug aldehyde conjugate activated by enzymatic reduction of a quinone.

FIG. 10 shows the structures of the commonly used chemotherapeutic agents cisplatin and carboplatin.

FIG. 11 is a diagram of the reaction of salicylamide with formaldehyde followed by doxorubicin HCl used to synthesize a prodrug of the present invention. As described in Example 1, the reaction conditions were: a.) 3 equiv CH₂O, DMF, 55° C., 15 min, and b.) 55° C., 15 min.

FIG. 12 is a diagram of the acetoxymethylation of salicylamide used to modify the prodrug constructs of the present invention. As described in Example 1, the reaction conditions were: a.) K₂CO₃, and b.) Chloromethyl acetate, KI.

FIG. 13 shows the scheme used to synthesize an androgen receptor targeting group with a series of ethylene glycol tethers of the present invention. As described in Example 2, the reaction conditions were:, a) K₂CO₃, DMF, 65° C.; b) NaIO₄, NaHCO₃/H₂O pH 8.0; c₁) B₁₀H₁₄, diethylene glycol, c₂) B₁₀H₁₄, triethyleneglycol, c₃) B₁₀H₁₄, 2-butyne-1,4-diol; d) 2,2-dimethoxypropane, p-TsOH, acetone; e) MsCl, TEA, THF; f) LiBr (10 eq); g) sodium salt of 2f, 55° C.; and h) p-TsOH, MeOH/H₂O, reflux.

FIG. 14 shows the scheme used to synthesize two prodrug derivatives of the present invention incorporating piperazine into the tether. As described in Example 2, the reaction conditions were a) B₁₀H₁₄, 2-bromoethanol; b) NaH, DME; c) 1,4-dibromobutane or bis(2-bromoethyl) ether, 60° C.; d) piperazine, THF, reflux; and e) 9, TEA, THF, reflux.

FIG. 15 shows the structures of various non-steroidal antiandrogens (2,3) AR targeting molecules (4,10) and the highly toxic prodrug, doxorubicin-formaldehyde, doxoform which were tested as described in Example 3.

FIG. 16 shows the synthetic scheme of E/Z-desmethyl-4-hydroxytamoxifen. As described in Example 4, the reaction conditions and reagents were: (a) NaH, MOM-Cl (95%); (b) 1) n-BuLi, KOtBu, TMEDA, 2) 1-78° C. to RT (97%); (c) 6 M HCl (93%); (d) (n-Bu)₄NHSO₄, NaOH, 1,2-dibromoethane (90%), (e) BBr₃ (57%); (f) MeNH₂, 60° C., sealed tube (91%).

FIG. 17 shows the synthetic scheme of targeting tether intermediates of the present invention. As described in Example 4, the reaction reagents and conditions were: (a) triethylamine, DMF (7a, 69%; 7b, 72%: 7c 66%); (b) DIPEA, THF, sealed tube, 60° C. (+NaI, 7c) (8a, 68%; 8b, 55%; 8c, 61%); (c) hydrazine, EtOH, 60° C. (9a, 71%; 9b, 67%; 9c, 74%).

FIG. 18 shows the synthetic scheme for oximation of 5-formlysalicylamide and DOX-5-formylsaliform. As described in Example 4, the reaction reagents and conditions were: (a) 9a-9c, EtOH (10a, 81%; 10b, 72%: 10c 88%); (b) 9a-9c, TFA, EtOH, H₂O, (11a-c −50%).

FIG. 19 shows the scheme for synthesis of the acyclic-RGD-4C-DOXSF prodrug constructs of the present invention.

FIG. 20 shows the scheme for synthesis of the cyclic-(N-Me-VRGDf-NH)-DOXSF prodrug constructs of the present invention.

FIG. 21 shows the scheme for synthesis of the acyclic- and cyclic-CNGRC-linker-ONH₂ prodrug constructs of the present invention.

FIG. 22 shows the scheme for synthesis of the cyclic-dox-NGR (cyclic-CNGRC-dox) and potential therapeutic byproducts of the present invention.

FIG. 23 shows the design and synthesis of the vancociproform (vancomycin targeting group tethered to ciprofloxacin via the salicylamide trigger release group) of the present invention.

FIG. 24 shows the synthesis of the ciprosaliform prodrug conjugate of the present invention and release of ciproform via the salicylamide trigger.

FIG. 25 shows the synthetic scheme for KLAKKLA peptide targeted ciprosaliform of the present invention.

FIG. 26 shows a proposed synthesis scheme for anilinocyanoquinoline-linker-ONH₂ of the present invention.

FIG. 27 shows a proposed synthesis scheme for the dox-tether-anilinocyanoquinoline of the present invention for targeting a doxorubicin-formaldehyde conjugate to the TK domain of EGFR.

FIG. 28 shows the design and proposed synthesis of a cisplatin derivative-formaldehyde conjugate tethered to an anilinocyanoquinoline of the present invention for targeting to EGFR-TK domain.

FIG. 29 shows a proposed mechanism of action of a cisplatin derivative-formaldehyde conjugate tethered to an anilinocyanoquinoline of the present invention.

FIG. 30 shows the design and proposed synthesis of a second cisplatin derivative-formaldehyde conjugate tethered to an anilinocyanoquinoline of the present invention for targeting to EGFR-TK domain. The cisplatin-formaldehyde conjugate (structure 16) released upon hydrolysis of the salicylamide trigger is also shown.

### DETAILED DESCRIPTION OF THE INVENTION

The following definitions apply to the terms as used throughout this specification, unless otherwise limited in specific instances.

The phrase “causing the regression of” as used in the present application refers to reducing and/or eliminating pathogenic states such as infection or neoplasia.

Unless otherwise indicated, the term “alkyl” as employed herein alone or as part of another group includes both straight and branched chain hydrocarbons, containing 1 to 40 carbons, preferably 1 to 20 carbons, more preferably 1 to 12 carbons, in the normal chain, such as methyl, ethyl, propyl, isopropyl, butyl, t-butyl, isobutyl, pentyl, hexyl, isohexyl, heptyl, 4,4-dimethylpentyl, octyl, 2,2,4-trimethylpentyl, nonyl, decyl, undecyl, dodecyl, the various branched chain isomers thereof, and the like as well as such groups including 1 to 4 substituents such as halo, for example F, Br, Cl or I or CF₃, alkoxy, aryl, aryloxy, aryl(aryl) or diaryl, arylalkyl, arylalkyloxy, alkenyl, cycloalkyl, cycloalkylalkyl, cycloalkylalkyloxy, amino, hydroxy, acyl, heteroaryl, heteroaryloxy, heteroarylalkyl, heteroarylalkoxy, aryloxyalkyl, aryloxyaryl, alkylamido, alkanoylamino, arylcarbonylamino, nitro, cyano, thiol, haloalkyl, trihaloalkyl and/or alkylthio.

Unless otherwise indicated, the term “cycloalkyl” as employed herein alone or as part of another group includes saturated or partially unsaturated (containing 1 or 2 double bonds) cyclic hydrocarbon groups containing 1 to 3 rings, including monocyclicalkyl, bicyclicalkyl and tricyclicalkyl, containing a total of 3 to 20 carbons forming the rings, preferably 4 to 12 carbons, forming the ring and which may be fused to 1 or 2 aromatic rings as described for aryl, which include cyclopropyl, cyclobutyl, cyclopentyl, cyclohexyl, cycloheptyl, cyclooctyl, cyclodecyl and cyclododecyl, cyclohexenyl, any of which groups may be optionally substituted with 1 to 4 substituents such as halogen, alkyl, alkoxy, hydroxy, aryl, aryloxy, arylalkyl, cycloalkyl, alkylamido, alkanoylamino, oxo, acyl, arylcarbonylamino, amino, nitro, cyano, thiol and/or alkylthio.

The term “cycloalkenyl” as employed herein alone or as part of another group refers to cyclic hydrocarbons containing 5 to 20 carbons, preferably 6 to 12 carbons and 1 or 2 double bonds. Exemplary cycloalkenyl groups include cyclopentenyl, cyclohexenyl, cycloheptenyl, cyclooctenyl, cyclohexadienyl, and cycloheptadienyl, which may be optionally substituted as defined for cycloalkyl.

The term “polycycloalkyl” as employed herein alone or as part of another group refers to a bridged multicyclic group containing 5 to 20 carbons and containing 0 to 3 bridges, preferably 6 to 12 carbons and 1 or 2 bridges. Exemplary polycycloalkyl groups include [3.3.0]-bicyclooctanyl, adamantanyl, [2.2.1]-bicycloheptanyl, [2.2.2]-bicyclooctanyl and the like and may be optionally substituted as defined for cycloalkyl.

The term “polycycloalkenyl” as employed herein alone or as part of another group refers to a bridged multicyclic group containing 5 to 20 carbons and containing 0 to 3 bridges and containing 1 or 2 double bonds, preferably 6 to 12 carbons and 1 or 2 bridges. Exemplary polycycloalkyl groups include [3.3.0]-bicyclooctenyl, [2.2.1]-bicycloheptenyl, [2.2.2]-bicyclooctenyl and the like and may be optionally substituted as defined for cycloalkyl.

The term “aryl” as employed herein alone or as part of another group refers to monocyclic and bicyclic aromatic groups containing 6 to 10 carbons in the ring portion (such as phenyl or naphthyl) and may optionally include one to three additional rings fused to Ar (such as aryl, cycloalkyl, heteroaryl or cycloheteroalkyl rings) and may be optionally substituted through available carbon atoms with 1, 2, 3 or 4 groups selected from hydrogen, halo, haloalkyl, alkyl, haloalkyl, alkoxy, haloalkoxy, alkenyl, trifluoromethyl, trifluoromethoxy, alkynyl, cycloalkylalkyl, cycloheteroalkyl, cycloheteroalkylalkyl, aryl, heteroaryl, arylalkyl, aryloxy, aryloxyalkyl, arylalkoxy, arylthio, arylazo, heteroarylalkyl, heteroarylalkenyl, heteroarylheteroaryl, heteroaryloxy, hydroxy, nitro, cyano, amino, substituted amino wherein the amino includes 1 or 2 substituents (which are alkyl, aryl or any of the other aryl compounds mentioned in the definitions), thiol, alkylthio, arylthio, heteroarylthio, arylthioalkyl, alkoxyarylthio, alkylcarbonyl, arylcarbonyl, alkylaminocarbonyl, arylaminocarbonyl, alkoxycarbonyl, aminocarbonyl, alkylcarbonyloxy, arylcarbonyloxy, alkylcarbonylamino, cylcarbonylamino, arylsulfinyl, arylsulfinylalkyl, arylsulfonylamino or trylsulfonaminocarbonyl.

The term “aralkyl” or “aryl-alkyl” as used herein alone or as part of another group refers to alkyl groups as discussed above having an aryl substituent, such as benzyl or phenethyl, or naphthylpropyl, or an aryl as defined above.

The term “alkoxy”, “aryloxy” or “aralkoxy” as employed herein alone or as part of another group includes any of the above alkyl, aralkyl or aryl groups linked to an oxygen atom.

The term “polyalkyloxy” as used herein includes diethylene glycol, dipropylene glycol, polyethylene glycols, polypropylene glycols and glycol derivatives.

The term “amino” as employed herein alone or as part of another group may optionally be substituted with one or two substituents such as alkyl and/or aryl.

The term “alkylthio”, “arylthio” or “aralkylthio” as employed herein alone or as part of another group includes any of the above alkyl, aralkyl or aryl groups linked to a sulfur atom.

The term “alkylamino”, “arylamino”, or “arylalkylamino” as employed herein alone or as part of another group includes any of the above alkyl, aryl or arylalkyl groups linked to a nitrogen atom.

The term “acyl” as employed herein by itself or part of another group as defined herein, refers to an organic radical linked to a carbonyl group, examples of acyl groups include alkanoyl, alkenoyl, aroyl, aralkanoyl, heteroaroyl, cycloalkanoyl and the like.

The term “alkanoyl” as used herein alone or as part of another group refers to alkyl linked to a carbonyl group. Unless otherwise indicated, the term “lower alkenyl” or “alkenyl” as used herein by itself or as part of another group refers to straight or branched chain radicals of 2 to 20 carbons, preferably 3 to 12 carbons, and more preferably 1 to 8 carbons in the normal chain, which include one to six double bonds in the normal chain, such as vinyl, 2-propenyl, 3-butenyl, 2-butenyl, 4-pentenyl, 3-pentenyl, 2-hexenyl, 3-hexenyl, 2-heptenyl, 3-heptenyl, 4-heptenyl, 3-octenyl, 3-nonenyl, 4-decenyl, 3-undecenyl, 4-dodecenyl, 4,8,12-tetradecatrienyl, and the like, and which may be optionally substituted with 1 to 4 substituents, namely, halogen, haloalkyl, alkyl, alkoxy, alkenyl, alkynyl, aryl, arylalkyl, cycloalkyl, amino, hydroxy, heteroaryl, cycloheteroalkyl, alkanoylamino, alkylamido, arylcarbonylamino, nitro, cyano, thiol and/or alkylthio.

Unless otherwise indicated, the term “alkynyl” as used herein by itself or as part of another group refers to straight or branched chain radicals of 2 to 20 carbons, preferably 2 to 12 carbons and more preferably 2 to 8 carbons in the normal chain, which include one triple bond in the normal chain, such as 2-propynyl, 3-butynyl, 2-butynyl, 4-pentynyl, 3-pentynyl, 2-hexynyl, 3-hexynyl, 2-heptynyl, 3-heptynyl, 4-heptynyl, 3-octynyl, 3-nonynyl, 4-decynyl,3-undecynyl, 4-dodecynyl and the like, and which may be optionally substituted with 1 to 4 substituents, namely, halogen, haloalkyl, alkyl, alkoxy, alkenyl, alkynyl, aryl, arylalkyl, cycloalkyl, amino, heteroaryl, cycloheteroalkyl, hydroxy, alkanoylamino, alkylamido, arylcarbonylamino, nitro, cyano, thiol, and/or alkylthio.

The term “alkylene” as employed herein alone or as part of another group (which also encompasses “alkyl” as part of another group such as arylalkyl or heteroarylalkyl) refers to alkyl groups as defined above having single bonds for attachment to other groups at two different carbon atoms and may optionally be substituted as defined above for “alkyl”. The definition of alkylene applies to an alkyl group which links one function to another, such as an arylalkyl substituent.

The terms “alkenylene” and “alkynylene” as employed herein alone or as part of another group (which also encompass “alkenyl” or “alkynyl” as part of another group such as arylalkenyl or arylalkynyl), refer to alkenyl groups as defined above and alkynyl groups as defined above, respectively, having single bonds for attachment at two different carbon atoms.

The term “allene” as used herein alone or as part of another group includes hydrocarbon chains having two double bonds from one carbon atom to two others (e.g. RC═C═CR′), and derivatives formed by substitution, such as propadiene.

Suitable alkylene, alkenylene or alkynylene groups (which may include alkylene, alkenylene or alkynylene groups) as defined herein, may optionally include 1, 2, or 3 alkyl, alkoxy, aryl, heteroaryl, cycloheteroalkyl, alkenyl, alkynyl, oxo, aryloxy, hydroxy, halogen substituents and in addition, may have one of the carbon atoms in the chain replaced with an oxygen atom, N—H, N-alkyl or N-aryl.

The term “halogen” or “halo” as used herein alone or as part of another group refers to chlorine, bromine, fluorine, and iodine as well as CF₃, with chlorine or fluorine being preferred.

The term “cycloheteroalkyl” as used herein alone or as part of another group refers to a 5-, 6- or 7-membered saturated or partially unsaturated ring which includes 1 to 2 hetero atoms such as nitrogen, oxygen and/or sulfur, linked through a carbon atom or a heteroatom, where possible, optionally via a linker. The above groups may include 1 to 3 substituent groups as defined above. In addition, any of the above rings can be fused to 1 or 2 cycloalkyl, aryl, heteroaryl or cycloheteroalkyl rings.

The term “heteroaryl” as used herein alone or as part of another group refers to a 5- or 6-membered ring or as part of another group includes 1, 2, 3 or 4 hetero atoms such as nitrogen, oxygen or sulfur, and such rings fused to an aryl, cycloalkyl, heteroaryl or cycloheteroalkyl ring (e.g. benzothiophenyl, indolyl), linked through a carbon atom or a heteroatom, where possible, and includes all possible N-oxide derivatives.

The heteroaryl groups including the above groups may optionally include 1 to 4 substituents such as any of the substituents listed for aryl. In addition, any of the above rings can be fused to a cycloalkyl, aryl, heteroaryl or cycloheteroalkyl ring.

The term “cycloheteroalkylalkyl” as used herein alone or as part of another group refers to cycloheteroalkyl groups as defined above linked through a C atom or heteroatom to a CH₂ chain.

The term “heteroarylalkyl” or “heteroarylalkenyl” as used herein alone or as part of another group refers to a heteroaryl group as defined above linked through a C atom or heteroatom to a (CH₂) chain, alkylene or alkenylene as defined above.

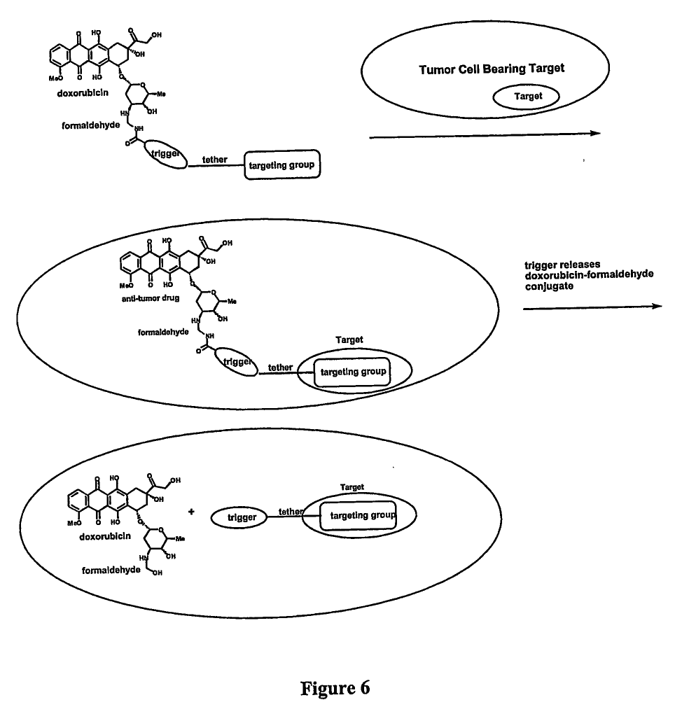

The present invention relates to the design, synthesis and evaluation of a platform prodrug design that can alter the pharmacokinetics, specificity and therapeutic index of many anti-tumor and antibiotic drugs, and can be extended to many other drugs. Referring to FIG. 6, the general concept of the prodrug platform is illustrated using the anthracycline antibiotic doxorubicin, which is bonded to an aldehyde, such as formaldehyde, and the resulting drug-aldehyde conjugate is protected with a chemical trigger which can be optionally tethered to a targeting group. The targeting group will direct the prodrug construct to a receptor or ligand expressed by a target cell (e.g., a tumor cell in the case of doxorubicin), and the trigger will keep the drug stable and inactive for a specific period of time. With a time constant suitable for therapeutic efficacy in vivo (e.g., about 1-24 hours, and preferably, between about 1 and about 4 hours, and more preferably, between about 1 and 2 hours, and more preferably, between about 50 and 70 minutes), the trigger will release the drug bonded to aldehyde from the targeting group to produce an active drug metabolite, such as the doxorubicin-formaldehyde conjugate in FIG. 6.

The prodrug technology of the present invention is provided by preparing an aldehyde-N-Mannich base using a drug-aldehyde conjugate. In a preferred embodiment, the aldehyde-N-Mannich base can then be tethered to a desired targeting molecule or peptide, if desired, for targeted delivery of the prodrug to a particular cell, ligand or receptor.

The term “drug-aldehyde conjugate” as used herein refers to a compound formed by a reaction of an aldehyde with a specific drug (e.g., an anthracycline or an antibiotic) and specifically includes monomeric, dimeric and multimeric drug-aldehyde conjugates. Various drug-aldehyde conjugates are described in detail in U.S. Pat. No. 6,677,309, particularly anthracycline-aldehyde conjugates. The aldehyde used to form the drug-aldehyde conjugate is preferably formaldehyde.

The N-Mannich base construct used to protect and trigger the drug-aldehyde conjugates of the present invention is a well characterized moiety resulting from the condensation of a primary or secondary amine with the electron deficient nitrogen atom of an appropriate functional group (i.e. amide, sulfonamide, imide, urea) via a single carbon atom bridge. The source of the carbon bridge can be either formaldehyde or, less commonly, any one of a number of substituted aldehydes of varying complexity. The N-Mannich base is formed using the aldehyde of the drug-aldehyde conjugate as the source of the single carbon atom bridge, and any amide, sulfonamide, carbamate or urea. Preferably, the N-Mannich base useful in the present invention is formed using an amide which produces an N-Mannich base that has a half-life of hydrolysis or decomposition to the Schiff base active metabolite under physiological conditions of between about 1 and 4 hours, and more preferably, between about 1 and about 2 hours. In a most preferred embodiment, the N-Mannich base useful in the present invention is formed using the amide, salicylamide (2-hydroxybenzamide), or a derivative thereof that provides a suitable half-life of hydrolysis or decomposition as discussed above.

According to the present invention, the “trigger” portion of the prodrug is provided by the functional group (e.g., the amide) used to form the N-Mannich base.

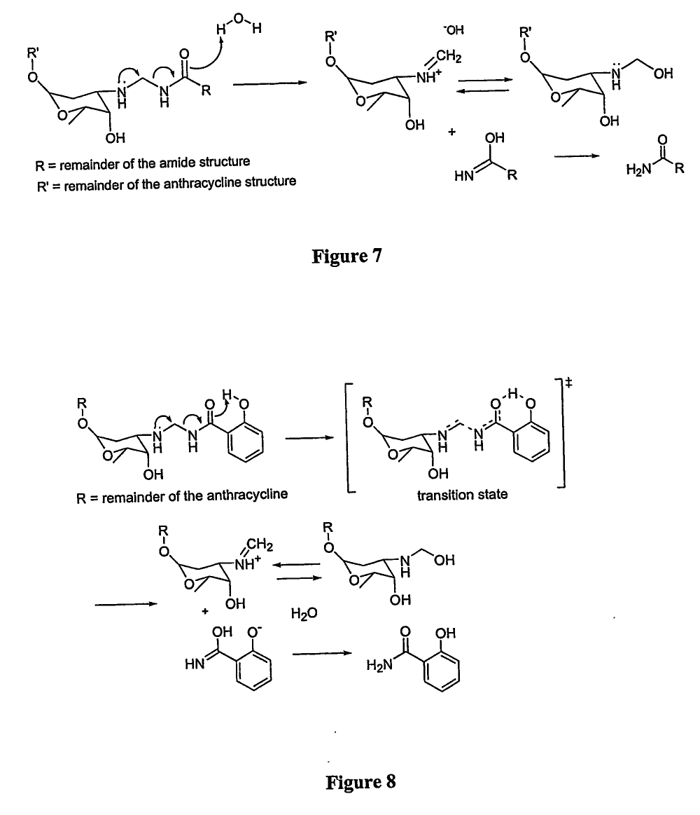

Under appropriate conditions, the N-Mannich base will hydrolyze or decompose (or a combination thereof) as a function of time described by first order kinetics. The rate of hydrolysis parallels the relative acidity of the amide employed. The aqueous stability of the N-Mannich base construct has been explored by both Bundgaard and Loudon (Bundgaard et al., Int. J. Pharm. 9:7-16 (1981); Loudon et al., J. Am. Chem. Soc. 103:4508-4515(1981)). The proposed mechanism of hydrolysis is shown in FIG. 7. In this mechanism, efficient hydrolysis depends upon the free electron pair of the amine and is accelerated by protonation of the carbonyl oxygen by a proximal water molecule followed by tautomerization of the liberated imidic acid to the stable amide. Importantly, this decomposition results in the retention of formaldehyde by the amino group of the drug, which serves to liberate the desired Schiff base active metabolite as an intermediate to full hydrolysis.

The analogous decomposition of an N-Mannich base prodrug of the present invention formed with the anthracycline, doxorubicin (doxsaliform) is illustrated in FIG. 8. The key difference in this reaction scheme is the ability of the phenolic group of salicylamide to protonate the carbonyl oxygen, in lieu of water, via a favorable hexagonal intramolecular transition state. This accounts for the relative instability of the salicylamide derived N-Mannich bases. It also indicates that the decomposition of doxsaliform is not an exclusively hydrolytic event and is expected to occur in non-aqueous solution. Indeed, the decomposition of doxsaliform occurs, albeit more slowly, in organic solvents as well as during storage as a dry solid. This unique method of decomposition acts as a “trigger release” and serves to inherently deliver the cytotoxin (or other therapeutic drug) in a manner that cannot be exclusively described as hydrolytic although, in aqueous solution, the two terms are used interchangeably.

Therefore, the trigger portion of the prodrug is the moiety of the N-Mannich base that stabilizes the drug-aldehyde conjugate as an inactive prodrug, but under appropriate conditions (e.g., physiological conditions or conditions under which the hydrolysis or decomposition reaction occurs), allows for the release of the metabolically active drug-formaldehyde conjugate. The mechanism of trigger release observed for the N-Mannich bases, including doxsaliform, can be exploited to stabilize the prodrug both in solution and during storage. With reference to the anthracycline, doxorubicin, the free electron pair on the 3′-amino group of doxorubicin is required for efficient release of the trigger. A similar scenario is created with the preparation of other N-Mannich bases of drug-aldehyde conjugages of the invention. Protonation of this nitrogen is, therefore, expected to stabilize the prodrug. Alternatively, acyloxymethylation of the phenolic moiety of salicylamide (see the discussion above with regard to the role of the phenolic moiety in the decomposition of salicylamide) also stabilizes the prodrug. In accordance with the proposed mechanism of trigger release, replacing the phenolic proton of salicylamide with an enzymatically cleavable protecting group, for example, will stabilize simple N-Mannich bases such as those resulting from benzamide or acetamide. Rapidly removed protecting groups may serve to improve the stability of the prodrug of the invention for handling in the laboratory as well as during formulation of the prodrug for administration in vivo.

Therefore, one embodiment of the present invention relates to an N-Mannich base of a drug-aldehyde conjugate as described and exemplified herein, wherein the N-Mannich base is formed using an amide, and preferably, salicylamide or equivalent derivative thereof, as the donor of the electron-deficient nitrogen atom. While the nitrogen atom is donated by the drug, it participates in the formation of the N-Mannich base prodrug construct of the present invention. Thus, the drug must contain a primary or secondary amine to be eligible for formation as a prodrug construct according to the present invention. The electron-deficient nitrogen can be present in the drug as a primary or secondary amine and the secondary amine may be part of a branched or straight alkyl chain or present as a cyclic secondary amine.

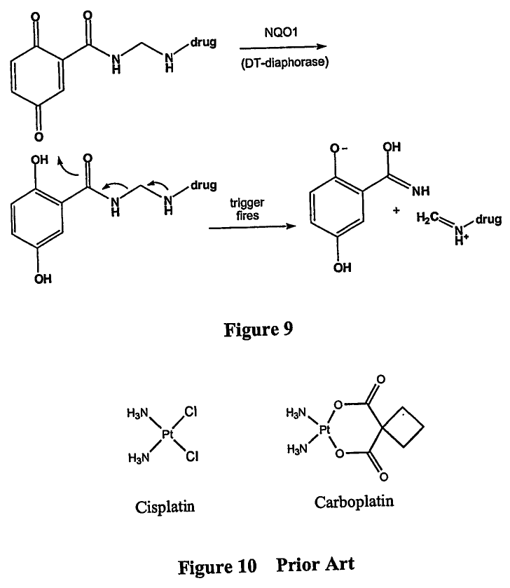

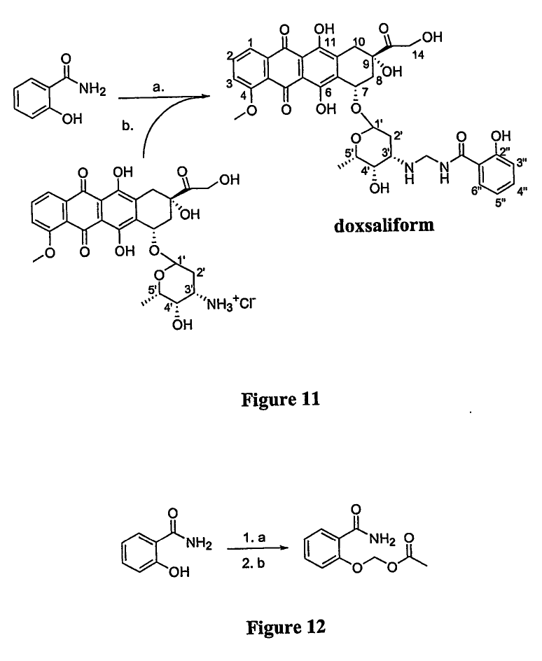

In a further embodiment, the N-Mannich base is stabilized (e.g., for storage) by any suitable method, including, but not limited to, protonation of the nitrogen in a 3′-amino group of the drug in the drug-aldehyde conjugate, acyloxymethylation of the phenolic moiety of the amide used to produce the N-Mannich base, or replacement of the phenolic moiety of the amide used to produce the N-Mannich base with an enzymatically cleavable protective group. According to the present invention, the enzymatically cleavable protective group can include, but is not limited to, an ester or acyloxymethyl ether of the salicylamide. For example, an enzymatically-activated trigger is the benzoquinone carboxamide shown in FIG. 9. Reduction of the quinone by the enzyme NAD(P)H:quinone oxidoreductase 1 (NQO1) provides a hydroxy functional group adjacent to the carboxyamide which facilitates the trigger firing to release the drug-formaldehyde conjugate. NQO1 is over expressed in many tumor cells (Siegal and Ross, Free Rad. Biol. Med. 29:246-253 (2000)), allowing this trigger to potentially release the drug-aldehyde conjugate in the vicinity of the tumor.

In one embodiment, the N-Mannich base is stabilized by attachment of a targeting moiety to the N-Mannich base. Preferably, the N-Mannich base of the drug-aldehyde conjugate of the invention, when the protection is released and under physiological conditions, has a half-life of at least about 30 minutes, and more preferably at least about 35 minutes, and more preferably at least about 40 minutes, and more preferably at least about 45 minutes, and more preferably at least about 50 minutes, and more preferably at least about 55 minutes, and more preferably at least about 60 minutes, and more preferably at least about 65 minutes, and more preferably at least about 70 minutes, and more preferably at least about 75 minutes, and more preferably at least about 80 minutes, and more preferably at least about 85 minutes, and more preferably at least about 90 minutes, and more preferably at least about 95 minutes, and more preferably at least about 100 minutes, and so on, up to at least about 240 minutes, including any interval in whole integers, between about 30 minutes and about 240 minutes (i.e., 30, 31, 32, 33, 34 . . . 238, 239, 240 minutes).

Although N-Mannich bases of drug-aldehyde conjugates according to the present invention can be used in this form without further modification, in one embodiment of the invention, it is desirable to tether the prodrug to a targeting moiety, in order to decrease systemic toxicity and enhance the efficacy of the drug at a desired site, and preferably at lower doses than are required when using the parent drug, for example. Therefore, in one embodiment of the invention, as described above, the drug-aldehyde conjugate comprising the trigger is attached via a tether to a targeting moiety. The “tether” can be any suitable chemical or peptide linkage between the salicylamide trigger (or derivative or similar moiety) and the desired targeting moiety. The entire construct of an N-Mannich base of a drug-aldehyde conjugate tethered to a targeting moiety is referred to herein as a “targeted drug-aldehyde conjugate” or a “targeted drug-aldehyde prodrug.” A tether must link the targeting moiety to the drug-aldehyde conjugate via the salicylamide trigger (or other trigger, as used) without causing a detectable negative steric or electronic interaction between the targeting moiety and its target. For example, the tether can include, but is not limited to, an ether group, polyalkyloxys, derivatized ethylene glycols, N,N′-disubstituted-piperazines, butyne-1,4-diol, 2,4-hexadiyne-1,6-diol, alkanes, polyethers, polyesters, polyamides, or peptides. Depending on the targeting moiety to be attached, suitable tethering moieties will be apparent to those of skill in the art based on this disclosure.

The targeting moiety used with the prodrug described herein can be virtually any targeting moiety that is desired for selectively delivering a prodrug of the invention to a specific cell type, receptor or ligand. Multiple targeting molecules and peptides are well known in the art and are in use for delivery of other therapeutic molecules to a site. Any of such targeting moieties are encompassed for use in the present invention. A “target site” refers to any site in vivo or in vitro to which one desires to deliver a composition or drug, and can include a cell, a tissue, a bodily fluid, or more specific sites, such as a receptor, a ligand, or cell surface molecule other than a receptor, for example. Suitable targeting compounds include any compounds capable of selectively (i.e., specifically) binding another molecule at a particular site. Examples of such compounds include a variety of synthetic molecules, steroidal compounds, non-steroidal compounds, glycoproteins, peptides, and proteins (antibodies, antigens, receptors and receptor ligands). In one embodiment, the targeting compound or moiety targets a cell surface molecule or intracellular molecule expressed by a tumor cell. Such a target can be overexpressed by tumor cells relative to non-tumor cells, or exclusively (or substantially exclusively) expressed by tumor cells. In one embodiment, a suitable target is any component of the cell wall or structure of a pathogenic microorganism (e.g., a bacterial cell wall component). In another embodiment, a suitable target is a particular cell surface molecule that distinguishes one cell or tissue type from another. In one embodiment, a targeting moiety that targets a particular receptor, for example, does not activate the receptor. Some examples of suitable targeting moieties, many of which are exemplified herein, include, but are not limited to: homing peptides (e.g., those isolated from phage display), non-steroidal hormones or derivatives thereof (e.g., non-steroidal antiestrogens, non-steroidal anti-androgens), other receptor or cell surface molecule ligands or derivatives, agonists or antagonists thereof, or antibiotics. Some specific examples of suitable targeting moieties include, but are not limited to, E/Z-4-hydroxytamoxifen, NGR peptides, acyclic-RGD-4C (CDCRGDCFC), cyclic-RGD-4C, or cyclic-(RGDf-N(Me)V) targeting peptide, cyanonilutamide, the peptide (KLAKKLA)₂, vancomycin, and anilinocyanoquinoline.

Therapeutic drugs useful in the present invention include, but are not limited to, any therapeutic drug that can be conjugated to an aldehyde and form an N-Mannich base thereof when reacted with an appropriate functional group (e.g., an amide, and preferably salicylamide or a derivative thereof), wherein the N-Mannich base has a a half-life of hydrolysis or decomposition to the Schiff base active metabolite under physiological conditions of between about 1 and 4 hours. In a preferred embodiment, the drug exerts biological activity via a mechanism that involves the use of an aldehyde to form covalent and/or non-covalent interactions with DNA to serve to virtually crosslink the DNA. The term virtual cross-link refers to a nucleic acid (mitochondrial, nuclear or synthetic DNA or RNA) in which the nucleic acid has at least a portion of double-strandedness and in which one strand is covalently bound to a drug by a methylene (derived from an aldehyde) on an amino group of the drug and the other strand of the nucleic acid is hydrogen bonded to the drug. Therefore, the term “virtual cross-link” is distinguished from “cross-link” wherein both strands of the nucleic acid are covalently bound to the drug.

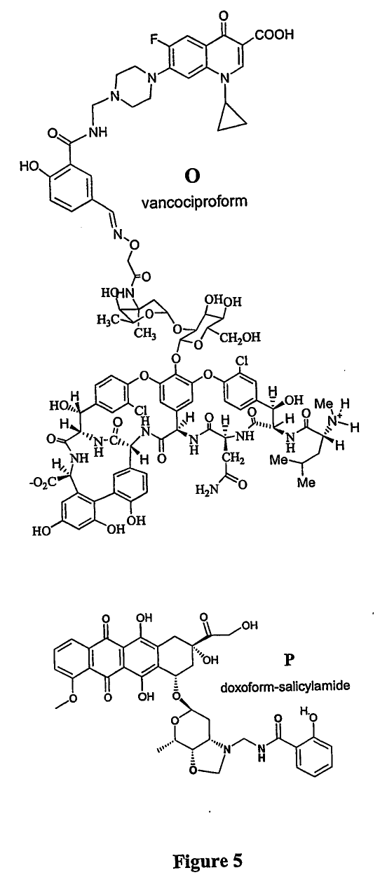

Drugs suitable for use in the present invention include many anti-cancer (anti-tumor) drugs and antibiotics, as well as multiple derivatives thereof. Any amino or 1,2-dihetero-substituted drug is particularly useful in the present invention. A “1,2-diheterosubstituted drug” refers to a drug with two heteroatoms on adjacent atoms. Preferably, the 1,2-hetero-substituted drug contains an amino moiety and an alcohol moiety on adjacent carbons of the drug. In one embodiment, the heteroatoms are located at the 3′ and 4′ carbons of a ring of the drug which is conjugated to the aldehyde component. The aldehyde component of the drug-aldehyde conjugate can react with the amino and alcohol moieties to form the conjugate. The main advantage of having a 1,2-diheterosubstituted drug is that it can carry a second molecule of formaldehyde in the form of a five-membered ring structure. For example, in the embodiment of the present invention in which the prodrug construct is formed with the drug doxorubicin, the 1,2-diheterosubstituted drug forms the oxazolidine ring of Doxoform. The structure of the corresponding prodrug construct containing the oxazolidine ring bound to the salicylamide trigger is shown as structure P, FIG. 5.

Some preferred drugs are those which, in addition to the 1,2-heteroatom substitution, have the following general structural components: (1) a nucleic acid intercalating region; and (2) a nucleic acid binding region (e.g., a “ring” or “arm” which is free to rotate out of the plane of the intercalating region. For example, the linear four-ring portion of an anthracycline is a nucleic acid intercalating region, and the sugar of the anthracycline is a nucleic acid binding region. Other linear, especially tetracyclic, ring systems with some, especially structural three, aromatic rings and a non-aromatic ring at the end are preferred. Drugs containing anthracene structures as the nucleic acid intercalating region are also preferred.

According to the present invention, a derivative is any variant of a given “parent” or “lead” compound which has structural and/or functional characteristics in common with the parent compound. Typically, the derivative differs from the parent or lead compound by one or more modifications of at least one functional group in the compound resulting in a compound that has one or more improved or different properties as compared to the parent compound. Derivatives of the various anti-tumor drugs and antibiotics encompassed by the present invention are well known in the art. Derivatives, including agonists and antagonists of a given lead compound, that are products of drug design can be produced using various methods known in the art. Various methods of drug design, useful to design mimetics or other therapeutic compounds useful in the present invention are disclosed in Maulik et al., 1997, Molecular Biotechnology: Therapeutic Applications and Strategies, Wiley-Liss, Inc., which is incorporated herein by reference in its entirety. A derivative of a given compound can be obtained, for example, from molecular diversity strategies (a combination of related strategies allowing the rapid construction of large, chemically diverse molecule libraries), libraries of natural or synthetic compounds, in particular from chemical or combinatorial libraries (i.e., libraries of compounds that differ in sequence or size but that have the similar building blocks) or by rational, directed or random drug design. See for example, Maulik et al., ibid.

Accordingly, anti-cancer drugs useful in the present invention include any anthracycline drug or derivative thereof, including, but not limited to, naturally occurring, semi-synthetic and synthetic anthracyclines. Several families of anthracyclines are included within the class of anthracycline drugs, any members of which are well suited for use in the prodrug system of the present invention. Such families include, but are not limited to, the daunorubicin family, the aclacinomycin family, the duocarmycin family and the nogalamycin family. Several thousand anthracycline derivatives are known in the art and are encompassed by the invention. Anthracycline drugs and derivatives thereof are described in detail in PCT Publication WO 98/46598, and are all incorporated herein by reference.

Another anti-cancer drug useful for incorporation into the prodrug system of the present invention is any cisplatin drug or congener thereof. Thousands of derivatives of cisplatin have been synthesized and evaluated. The only derivative with activity comparable to cisplatin, though less toxic, is the second-generation analogue, carboplatin.

The structures of cisplatin and caboplatin are shown in FIG. 10. The principal mechanism of action of both cisplatin and carboplatin is DNA alkylation. By forming interstrand or intrastrand covalent bonds with two guanine nucleotides of DNA, these drugs can effectively impede DNA replication. Additionally, cisplatin can crosslink proteins to DNA. Therefore, included in the invention are any drugs based on cisplatin or a derivative thereof. Modification of cisplatin and its derivatives using the prodrug system of the present invention may allow the use of ciplatin analogs which are currently unsuitable for therapeutic use (e.g., enloplatin), by producing prodrugs with effective targeting means that have therapeutic utility and efficacy with reduced systemic toxicity.

Other anti-cancer drugs that can be used in the present invention include, daorubicin, epidoxorubicin, idarubicin, mitoxanthrone, mitomycin C, derivatives of nitrogen mustards (chloroambucil), bleomycin and nucleoside analogues such as gemcitabine, fludarabine, and cytarabine.

Also included in the invention are antibiotics. Particularly preferred antibiotics for use in the present invention are any of the fluoroquinolones, including, but not limited to, norfloxacin, ciprofloxacin, sparfloxacin, gatifloxacin, levofloxacin, and moxifloxacin. Fluoroquinolones are an important class of antibiotics with clinical activity against Gram positive and Gram negative bacteria as well as mycobacteria. Any of the above-identified fluoroquinolones or derivatives thereof are suitable for conjugation in the prodrug system of the present invention. Other antibiotics that can be used in the present invention include, aminoglycoside, oxazolidone, beta-lactam, and glycopeptide antibiotics.

The present invention is not limited to anti-cancer drugs and antibiotics. For example, antiviral drugs are also candidates. Of particular relevance are the nucleoside analogues such as acyclovir, ganciclovir, dideoxycytidine, 3-thiocytidine, Viread (tenofovir disoproxil fumarate) and Hepsera (adefovir dipivoxil). Other drugs that would be suitable for use in the conjugates, compositions and methods of the invention will be apparent to those of skill in the art in light of the present disclosure of the invention.

One embodiment of the present invention relates to a method to produce any of the N-Mannich bases of drug-aldehyde conjugates as described herein, or any of the targeted drug-aldehyde conjugates which are described herein. Suitable methods for production of N-Mannich bases of a drug-aldehyde conjugates are described in detail in the Examples, but generally include adding an aldehyde to an appropriate functional group (i.e. amide, sulfonamide, imide, urea) followed by adding a drug to the reaction to form an N-Mannich base of a drug-aldehyde conjugate as described herein. Preparation of the targeted drug-aldehyde conjugates further includes a step of synthesizing or otherwise producing a targeting moiety attached to a tether and reacting the tethered targeting moiety with the N-Mannich base drug-aldehyde conjugate to link the targeting moiety to the trigger portion (e.g., salicylamide) of the N-Mannich base drug-aldehyde conjugate via the tether. In one embodiment, the tether is linked to the N-Mannich base using an oximation reaction, which does not require the use of protecting groups for the final assembly. Multiple specific methods for production of a variety of N-Mannich bases of drug-aldehyde conjugates and targeted drug-aldehyde conjugates are described in detail in the Examples.

Other embodiments of the present invention relate to the use of the targeted and non-targeted drug-aldehyde conjugates described herein to treat or ameliorate at least one symptom of a disease or condition in which delivery of the drug would be expected to be beneficial. According to the present invention, the drugs of the present invention can be used to treat any disease or condition for which the parent drug (e.g., the drug upon which the novel prodrug of the invention is based) can be used, or for which the parent drug is desired to be used (and may not be currently suitable due to problems with toxicity, specificity, etc.). For example, a variety of anti-tumor drugs are contemplated by the present invention to be useful for treating tumors (cancer) in a patient. Similarly, antibiotic derivatives described herein will be useful for treating bacterial infections and symptoms thereof.

Accordingly, in one embodiment, a therapeutic method of the present invention preferably provides a therapeutic benefit to a patient upon administration, alone or in conjunction with one or more additional therapeutic treatments, such that the patient is protected from a disease that is amenable to treatment by the given drug. As used herein, the phrase “protected from a disease” refers to reducing the symptoms of the disease; reducing the occurrence of the disease, and/or reducing the severity of the disease. Protecting a patient can refer to the ability of a therapeutic composition of the present invention, when administered to a patient, to prevent a disease from occurring and/or to cure or to treat the disease by alleviating disease symptoms, signs or causes. As such, to protect a patient from a disease includes both preventing disease occurrence (prophylactic treatment) and treating a patient that has a disease or that is experiencing initial symptoms or later stage symptoms of a disease (therapeutic treatment). The term, “disease” refers to any deviation from the normal health of a patient and includes a state when disease symptoms are present, as well as conditions in which a deviation (e.g., infection, gene mutation, genetic defect, etc.) has occurred, but symptoms are not yet manifested (e.g., a precancerous condition).

More specifically, a therapeutic composition as described herein, when administered to a patient by the method of the present invention, preferably produces a result which can include alleviation of the disease (e.g., reduction of at least one symptom or clinical manifestation of the disease), elimination of the disease, reduction of a tumor or lesion associated with the disease, elimination of a tumor or lesion associated with the disease, prevention or alleviation of a secondary disease resulting from the occurrence of a primary disease, or prevention of the disease.

According to the present invention, an effective administration protocol (i.e., administering a therapeutic composition in an effective manner) comprises suitable dose parameters and modes of administration that result in the desired effect in the patient (e.g., reduction of at least one symptom associated with the disease or condition), preferably so that the patient is protected from the disease (e.g., by disease prevention or by alleviating one or more symptoms of ongoing disease). Effective dose parameters can be determined using methods standard in the art for a particular disease. Such methods include, for example, determination of survival rates, side effects (i.e., toxicity) and progression or regression of disease.

In accordance with the present invention, a suitable single therapeutic dose is a dose that results in the desired result in a patient, or in the amelioration of at least one symptom of a condition in the patient, when administered one or more times over a suitable time period. Doses can vary depending upon the disease being treated. For example, in the treatment of cancer, a suitable single dose can be dependent upon whether the cancer being treated is a primary tumor or a metastatic form of cancer. One of skill in the art can readily determine appropriate single dose sizes for a given patient based on the size of a patient and the route of administration. In one embodiment, a preferred single dose of a drug of the present invention typically comprises between about 0.01 microgram/kilogram and about 10 milligram/kilogram body weight of an animal.

Another preferred single dose of a drug comprises between about 1 microgram/kilogram and about 10 milligram/kilogram body weight of an animal. Another preferred single dose of an agent comprises between about 0.1 microgram/kilogram and about 10 microgram/kilogram body weight of an animal.

In one aspect of the invention, a suitable single dose of a therapeutic composition of the present invention is an amount that, when administered by any route of administration, regulates at least one symptom of the disease or condition to be treated in the patient, as compared to a patient which has not been administered with the therapeutic composition of the present invention (i.e., a pre-determine control patient or measurement), as compared to the patient prior to administration of the composition, or as compared to a standard established for the particular disease, patient type and composition. A suitable single dose of a therapeutic composition to regulate a cancer or tumor, for example, is an amount that is sufficient to reduce, stop the growth of, cause the regression of, and preferably eliminate, the tumor following administration of the composition into the tissue of the patient that has cancer. A suitable single dose of a therapeutic composition to regulate an infectious disease, for example, is an amount that is sufficient to reduce the population of, and preferably eliminate, the infectious organism or to reduce or ameliorate a symptom of the infection, following contact of the drug composition with the tissue of the patient that is infected with the organism.

A therapeutic composition of the present invention is administered to a patient in a manner effective to deliver the composition to a cell, a tissue, and/or systemically to the patient, whereby the desired result is achieved as a result of the administration of the composition. Preferably, the composition is delivered to a specific site (i.e., a targeted site) in the patient. Suitable administration protocols include any in vivo or ex vivo administration protocol. The preferred routes of administration will be apparent to those of skill in the art, depending on the type of condition to be prevented or treated; and/or the target cell/tissue. For the prodrugs of the present invention, preferred methods of in vivo administration include, but are not limited to, intravenous administration, intraperitoneal administration, intramuscular administration, intranodal administration, intracoronary administration, intraarterial administration (e.g., into a carotid artery), subcutaneous administration, transdermal delivery, intratracheal administration, subcutaneous administration, intraarticular administration, intraventricular administration, inhalation (e.g., aerosol), intracranial, intraspinal, intraocular, intranasal, oral, bronchial, rectal, topical, vaginal, urethral, pulmonary administration, impregnation of a catheter, and direct injection into a tissue. Combinations of routes of delivery can be used and in some instances, may enhance the therapeutic effects of the composition.

Ex vivo administration refers to performing part of the regulatory step outside of the patient, such as administering a composition of the present invention to a population of cells removed from a patient under conditions such that the composition contacts and/or enters the cell, and returning the cells to the patient. Ex vivo methods are particularly suitable when the target cell type can easily be removed from and returned to the patient.

Many of the above-described routes of administration, including intravenous, intraperitoneal, intradermal, and intramuscular administrations can be performed using methods standard in the art. Aerosol (inhalation) delivery can also be performed using methods standard in the art (see, for example, Stribling et al., Proc. Natl. Acad. Sci. USA 189:11277-11281, 1992, which is incorporated herein by reference in its entirety). Oral delivery can be performed by complexing a therapeutic composition of the present invention to a carrier capable of withstanding degradation by digestive enzymes in the gut of an animal. Examples of such carriers, include plastic capsules or tablets, such as those known in the art.

One method of local administration is by direct injection. Direct injection techniques are particularly useful for administering a composition to a cell or tissue that is accessible by surgery, and particularly, on or near the surface of the body. Administration of a composition locally within the area of a target cell refers to injecting the composition centimeters and preferably, millimeters from the target cell or tissue.

One embodiment of the invention relates to a therapeutic composition comprising at least one N-Mannich base of a drug-aldehyde conjugate of the invention and/or at least one targeted drug-aldehyde conjugate of the invention, formulated with a pharmaceutically acceptable carrier. According to the present invention, a “pharmaceutically acceptable carrier” includes pharmaceutically acceptable excipients and/or pharmaceutically acceptable delivery vehicles, which are suitable for use in administration of the composition to a suitable in vitro, ex vivo or in vivo site. A suitable in vitro, in vivo or ex vivo site has been discussed above. Preferred pharmaceutically acceptable carriers are capable of assisting in maintaining a drug of the invention in a form that, upon arrival of the drug at the cell target in a culture or in patient, drug is capable of interacting with its target (e.g., a receptor, ligand or other cell surface molecule).

Suitable excipients of the present invention include excipients or formularies that transport or help transport, but do not specifically target a composition to a cell (also referred to herein as non-targeting carriers). Examples of pharmaceutically acceptable excipients include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity. Besides those representative dosage forms described above, pharmaceutically acceptable excipients and carries are generally known to those skilled in the art and are thus included in the instant invention. Such excipients and carriers are described, for example, in “Remingtons Pharmaceutical Sciences” Mack Pub. Co., New Jersey (1991), which is incorporated herein by reference.

One type of pharmaceutically acceptable carrier includes a controlled release formulation that is capable of slowly releasing a composition of the present invention into a patient or culture. As used herein, a controlled release formulation comprises a drug of the present invention in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Other carriers of the present invention include liquids that, upon administration to a patient, form a solid or a gel in situ. Preferred carriers are also biodegradable (i.e., bioerodible).

Various aspects of the present invention are described in detail in the following reports, each attached as an individual Example. The Examples are provided for the purpose of illustration and are not intended to limit the scope of the present invention.

### EXAMPLES

### Example 1

Design, synthesis, and preliminary evaluation of a prodrug of doxorubicin active metabolite: the formaldehyde-N-Mannich base of doxorubicin with salicyclamide (doxsaliform).

For over three decades, the anthracycline antibiotic doxorubicin has proven to be one of the clinically most useful antineoplastic agents. Considered a broad spectrum drug, doxorubicin (DOX) has been extensively employed in the treatment of Hodgkin's disease, non-Hodgkin's lymphomas, acute leukemias, sarcomas, and solid tumors of the lung, liver, breast, and ovary. Extensive investigations into the mechanism of action have failed to produce derivatives of superior therapeutic value. While hundreds of modifications to the anthraquinone core, the side chain, and the sugar moiety have been explored, very few have displayed even modest improvement with respect to the therapeutic index. Although several derivatives have been found to exhibit greater cytotoxicity than the clinically used anthracyclines, a concomitant increase in systemic toxicity is also commonly observed.

One approach to the challenge of developing new anthracyclines with an improved therapeutic index is the use of prodrug delivery systems. While prodrugs are often designed to improve solubility or absorption across physiological membranes, anthracycline prodrugs generally aim to improve the biodistribution of the drug and to diminish its systemic toxicity. To this end, several prodrugs of doxorubicin, which serve to carry the drug as an inactive species, have been prepared and evaluated in recent years.

Ongoing work has focused on the development of prodrugs of doxorubicin which exploit part of the cytotoxic mechanism. Recent reports from several laboratories have suggested that the long established induction of oxidative stress by doxorubicin can lead to the generation of various aldehydes, as well as other reactive intermediates, which may serve to modify both the structure and activity of the parent drug. Of considerable interest is the production of formaldehyde, which has been demonstrated both in vitro and in living cells. Substantial evidence suggests that formaldehyde is employed by the clinically relevant anthracyclines to generate quasi-stable covalent adducts with DNA.

These drug-DNA adducts have been directly observed by mass spectrometry, NMR, and X-ray crystallography and are inferred from the varying rates of release of doxorubicin from the nuclei of tumor cells, as well as from double stranded DNA in cell free systems.

Further, the formaldehyde-releasing prodrugs, pivaloylmethyl butyrate and hexamethylenetetramine, enhance the cytotoxicity of doxorubicin.

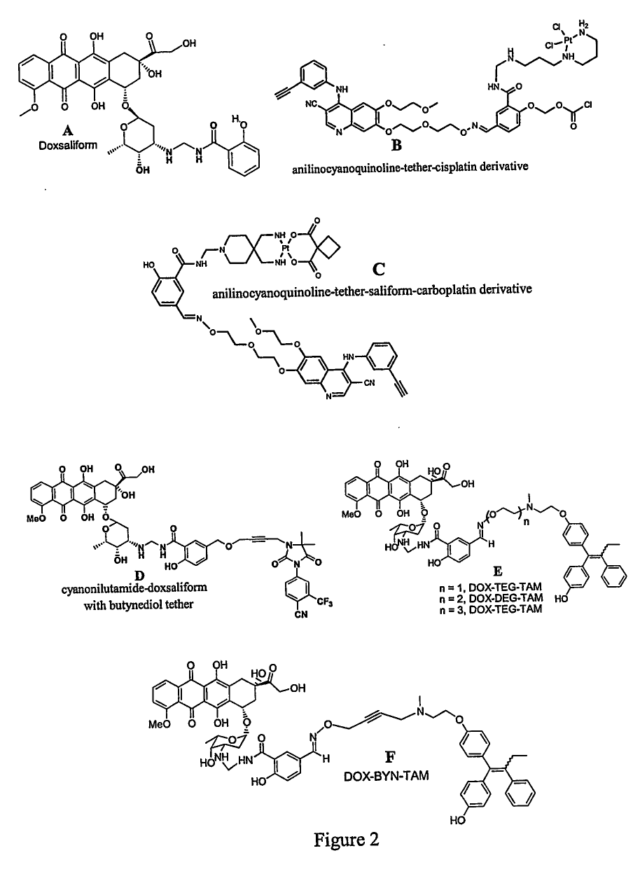

This example describes the rational design, synthesis, and preliminary evaluation of a second generation prodrug of the doxorubicin active metabolite, formaldehyde-N-Mannich base of doxorubicin with salicyclamide (doxsaliform, Structure A, FIG. 2). This prodrug construct has improved aqueous solubility, hydrolytic stability, and potential for synthetic elaboration.

¹H-NMR spectra were acquired with a 500 MHz spectrometer. Mass spectral data were acquired on a mass spectrometer by electron impact (EI) using a perfluorokerosene internal standard for [M+] data or liquid SIMS (LSIMS) ionization with a polyethylene glycol internal standard for [MH+] data. Mass spectral data for doxsaliform were obtained using a mass spectrometer with an electrospray ionization source (MH+) and were collected at the mass spectrometry and proteomics laboratory at The Ohio State University (Columbus, Ohio). Hydrolysis experiments were conducted in a constant temperature recirculation bath. UV-vis spectrometry was performed with a diode array spectrophotometer and workstation. HPLC analyses were performed with a liquid chromatograph equipped with a diode array UV-vis detector and workstation; chromatographies were performed with a 5 μm reverse phase C₁₈ microbore column, 2.1 mm i.d. ×100 mm, eluting at 0.5 mL/min, monitoring at 260, 310, and 480 nm. Acceptable analytical resolution was achieved with gradients of acetonitrile and triethylammonium acetate (Et₃NHOAc; TEAA), prepared as 20 mM triethylamine adjusted to pH 6.0 with acetic acid. The method employed for all analytical chromatography was as follows: A=CH₃CN, B=pH 6.0 buffer; A:B, 25:75 to 32:68 at 2 min, isocratic until 5 min, 40:60 at 5.1 min, isocratic until 7 min, 42:58 at 7.1 min, isocratic until 9 min, 25:75 at 10 min. Chloromethyl acetate was prepared according to the method of Iyer and co-workers (Iyer, R. P.; Yu, D.; Ho, N.; Agrawal, S. Synthetic. Commun. 1995, 25, 2739-2749.), and 2-(acetoxymethyloxy)-benzamide was prepared as described by Bundgaard and co-workers (Bundgaard, H.; Klixbull, U.; Falch, E. Int. J. Pharm. 1986, 29, 19-28.).

MCF-7 cells were obtained from American Type Culture Collection (Rockville, Md.). MCF-7/ADR cells were a gift from Dr. William W. Wells (Michigan State University; East Lansing, Mich.). PC-3 cells were a gift from Dr. Andrew Kraft (University of Colorado Health Science Center) and Dr. Kerry Burnstein (University of Miami, Fla.). All cell lines were maintained in vitro by serial culture in RPMI 1640 media supplemented with 10% fetal bovine serum, L-glutamine (2 mM), HEPES buffer (10 mM), penicillin (100 units/mL), and streptomycin (100 μg/mL). Cells were maintained at 37° C. in a humidified atmosphere of 5% CO₂ and 95% air.

Method for preparation of the N-Mannich base N-(2-Hydroxybenzamido-methyl)-doxorubicin: To a stirring solution of 20 mg of salicylamide (0.15 mmol) in 2.0 mL of DMF was added 10 μL of a 37% formalin solution (0.13 mmol). The reaction was stirred in a screw top vial for 15 min at 55° C., at which time 20 mg (0.034 mmol) of doxorubicin hydrochloride was added to form a red suspension that was stirred at 55° C. After 15 min, a clear red solution had formed and the reaction was removed from the heat. Transfer of the solution to a 250 mL round bottom flask, followed by rotary evaporation at 55° C. and 50 μm Hg gave a red film which was readily dissolved in 20 mL of methanol containing 30% pH 1 water (1% TFA). After 10 min at room temperature, the methanol was removed by rotary evaporation at 30° C. and the resulting aqueous solution was diluted to 50 mL with saturated brine, transferred to a separatory funnel, and washed 2× with 50 mL of chloroform. The aqueous solution was then diluted to 150 mL with 500 mM sodium phosphate buffer adjusted to pH 5.5. The desired N-Mannich base product was extracted into 50 mL of chloroform and collected. The solvent was then rotary evaporated at 30° C. to yield a red film. The washed product was then dissolved in 3 mL of chloroform and introduced to a silica gel flash column (2 cm×30 cm) that had been packed in 100% chloroform. Contaminants were eluted with 100% chloroform followed by 97.5% chloroform/2.5% methanol. The desired product was then collected in 95% chloroform/5% methanol. Addition of 1 mL of glacial acetic acid served to stabilize the N-Mannich base during subsequent rotary evaporation at 30° C. The solvent free product was dissolved in 1 mL of chloroform and precipitated by addition of 5 mL of hexanes. Centrifugation followed by decanting of the supernatant and drying under vacuum yielded 18.4 mg (72%) of N-(2-hydroxyrnethylbenzamido)-doxorubicin as a red solid: ¹H NMR (500 MHz, CDCl₃) Free base δ 1.39 (3H, d, J=6 Hz, 5′-Me), 1.79-1.85 (2H, m, 2′), 2.10 (1H, dd, J=15, 4 Hz, 8), 2.39 (1H, d, J=15 Hz, 8), 2.88 (1H, d, J=19 Hz, 10), 3.12-3.18 (1H, m, 3′), 3.20 (1H, d, J=19 Hz, 10), 3.70 (1H, s, 4′), 4.04 (3H, s, 4-OMe), 4.10 (1H, q, J=7 Hz, 5′), 4.27 (1H, dd, J=14, 5 Hz, NCH₂N), 4.44 (1H, dd, J=14, 5 Hz, NCH₂N), 4.64 (1H, bs, 9-OH), 4.74 (2H, s, 14), 5.21 (1H, s, 7), 5.47 (1H, s, 1′), 6.53 (1H, t, J=8 Hz, 5″), 6.65 (1H, d, J=8 Hz, 3″), 6.90 (1H, bt, J=5 Hz, NH), 7.10-7.16 (2H, m, 4″/6″), 7.35 (1H, d, J=8 Hz, 3), 7.77 (1H, t, J=8 Hz, 2), 7.94 (1H, d, J=8 Hz, 1), 11.95 (1H, bs, 2″OH), 13.02 (1H, bs, 6/11OH), 13.82 (1H, bs, 6/11OH); m/z 715.2111 [M+Na] (calculated for 715.2115).

Method for preparation of the N-Mannich base N-[(2-Acetoxymethyloxy)-benzamidomethyl]-doxorubicin: 2-(Acetoxymethyloxy)-benzamide (20 mg, 0.096 mmol) was reacted with formalin (10 μL, 0.13 mmol) followed by doxorubicin hydrochloride (20 mg, 0.034 mmol) to yield 21 mg (81%) of N-[2-(acetoxymethyloxy)-benzamidomethyl]-doxombicin as a red solid using the general procedure described above. The product was characterized from the following spectral data: ¹H NMR (500 MHz, CDCl₃) δ 1.40 (3H, d, J=7 Hz, 5′-Me), 1.63 (1H, dd, J=13, 5 Hz, 2′), 1.82 (1H, td, J=13, 4 Hz, 2′), 2.02 (3H, s, AcO), 2.14 (1H, dd, J=15, 4 Hz, 8), 2.38 (1H, d, J=15 Hz, 8), 2.99 (1H, d, J=19 Hz, 10), 3.07-3.12 (1H, m, 3′), 3.20 (1H, d, J=19 Hz, 10), 3.73 (1H, s, 4′), 4.03 (1H, q, J=7 Hz, 5′), 4.08 (3H, s, 4-Me), 4.40 (1H, dd, J=13, 5 Hz, NCH₂N), 4.38 (1H, dd, J=13, 5 Hz, NCH₂N), 4.70 (2H, s, 14), 4.79 (1H, bs, 90H), 5.30 (1H, d, J=1 Hz, 7), 5.53 (1H, d, J=4 Hz, 1′), 5.64 (1H, d, J=7 Hz, OCH₂O), 5.77 (1H, d, J=7 Hz, OCH₂O), 7.04 (1H, d, J=8 Hz, 3″), 7.09 (1H, dt, J=8, 1 Hz, 5″), 7.38 (1H, m, 4″), 7.39 (1H, d, J=8 Hz, 3), 7.78 (1H, t, J=8 Hz, 2), 7.95 (1H, bt, J=5 Hz, NH), 8.01 (1H, d, J=8 Hz, 1), 8.04 (1H, d, J=8 Hz, 6″), 13.18 (1H, bs, 6/11OH), 13.90 (1H, bs, 6/11OH); m/z 765.2508 [MH+] (calculated for 765.2507).

The hydrolysis of the N-Mannich base prodrugs was studied by preparing a 1.0 mM solution of the appropriate N-Mannich base in 1 mL of DMSO which had been dried over 3 Å molecular sieves for 48 h. This solution was added to 9 mL of pH 7.4 RPMI 1640 cell culture media maintained at 37° C. in a constant temperature water bath. Aliquots were removed at 15 min intervals and analyzed by BPLC, monitoring at 480 nm. The area under the curve for the N-Mannich base was determined at each time point and was used to establish the kinetics of decomposition, using regression software. Hydrolysis of AOM-doxsaliform was carried out in pH 8.0 100 mM sodium phosphate buffer at 37° C. AOM-doxsaliform was dissolved in 48.0 μL DMSO and this solution was diluted into phosphate buffer to give 4.8 mL of an 11.0 mM solution containing 1% DMSO. To this solution was added 6 μL of pig liver esterase (0.8 units/μL; Sigma; Milwaukee, Wis.) to achieve a final concentration of 1.0 units/mL. Aliquots (400 μL) were taken at 15 min intervals and were added to 1.0 mL ethanol to precipitate the protein. Brief centrifugation served to pellet the insoluble fraction. The drug solution was then transferred to a round bottom flask and concentrated by brief rotary-evaporation at 20° C. Reverse phase HPLC was used to analyze the extent of hydrolysis.

To evaluate the cytotoxicity of doxsaliform, cells were dissociated with trypsin EDTA, counted, and suspended in growth media to a concentration of 5×10³ cells/mL. This cell suspension was dispensed in 200 μL aliquots into 96-well tissue culture plates. Plates were then incubated for 24 h at 37° C. in a humidified atmosphere of 5% CO₂ and 95% air. The medium was replaced with 180 μL of growth medium prior to addition of the prodrug. Doxsaliform was dissolved in DMSO containing 1% glacial acetic acid at concentrations ranging from 1 mM to 10 mM and sterile-filtered through a 0.45 μm nylon centrifuge filter. The concentration was then corrected by measuring the solution absorbance at 480 nm (ε=11 500 L/(mol·cm). Serial dilutions (1:10) were made in sterile DMSO to yield seven solutions of decreasing drug concentration at 100× the respective working concentrations. The resulting solutions were individually diluted 1:10 in RPMI 1640; 20 μL of the resulting 10× solution was immediately added to the appropriate lane of cells. Additionally, two lanes were treated with 20 μL growth medium containing 10% sterile DMSO and one lane was treated with 200 μL of 1.5 M Tris buffer. The cells were incubated at 37° C. for 4 h, at which time the drug solutions were replaced with 200 μL of fresh growth medium. The cells were then incubated for 5-6 days at 37° C. in a humidified atmosphere of 5% CO₂ and 95% air. The extent of colony formation was determined using a crystal violet staining assay.

Results: The N-Mannich base construct is a well characterized moiety resulting from the condensation of a primary or secondary amine with the electron deficient nitrogen atom of an appropriate functional group (i.e. amide, sulfonamide, imide, urea) via a single carbon atom bridge. The source of the carbon bridge can be either formaldehyde or, less commonly, any one of a number of substituted aldehydes of varying complexity. Reaction of doxorubicin with an appropriate primary amide and 1-3 equivalents of formaldehyde in warm N,N-dimethylformamide generated the N-Mannich base in yields ranging from 60% to 85%.

Several amides were studied to identify prodrugs that were both sufficiently stable and active against growing tumor cells. Reaction of doxorubicin and formaldehyde with simple amides such as acetamide and benzamide, or various derivatives of these core structures, led to respective mixtures of two primary products, both of which were isolated and characterized. The major product obtained from each starting material was found to be the desired N-Mannich base, which was readily isolated by flash chromatography. The second product, in all cases, was found to rapidly hydrolyze, in less than 10 min at pH 7.4 (as well as under more acidic conditions) and 25° C., to yield the desired N-Mannich base. ¹H-NMR and mass spectral studies identified this product as the oxazolidine derivative of the N-Mannich base, with the 5-membered oxazolidine ring being formed by the reaction of formaldehyde with the 4′ hydroxyl and the 3′ amino groups of doxorubicin.

While addition of a large excess of formaldehyde (5-6 equiv.) to the reaction mixture was found to lead to the consumption of more starting material, this also served to change the ratio of the oxazolidine derivative to the uncyclized product. The major product under these conditions was often found to be the oxazolidine derivative, which was readily hydrolyzed to the desired uncyclized product. Unfortunately, the presence of excess formaldehyde also led to the generation of additional products under the reaction conditions. The dimeric compound doxoform was commonly the most abundant of these unwanted products and acted as a competing pathway for the consumption of both doxorubicin and formaldehyde.

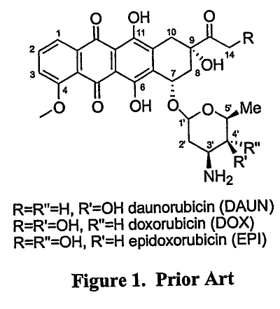

Reaction of doxorubicin with salicylamide (2-hydroxybenzamide) in the presence of 1-3 equiv. of formaldehyde led to a similar product distribution as was seen with the other amides employed. Several modifications to the synthetic protocol were investigated to optimize the production of the desired product. Referring to FIG. 1, it was found that only by briefly reacting salicylamide with formaldehyde before the addition of doxorubicin could the yield of the N-Mannich base be greatly improved. This method produced the N-Mannich base, nominally referred to here as doxsaliform, almost exclusively, with little of the oxazolidine or other unwanted side products.

While the generation of a transient species resulting from the reaction of salicylamide with formaldehyde was initially postulated as a rationale for the improved product ratio, NMR experiments indicated that this was unlikely. Formalin, an aqueous/methanolic solution of formaldehyde was used for all of the N-Mannich base reactions. Proton NMR indicates that there is initially no free formaldehyde in the formalin solution but rather that the aldehyde exists as a variety of acetals, hemiacetals, hydrates, and oligomers. Brief heating of 10 μL of formalin in 1.0 mL of DMF serves to liberate free formaldehyde into solution (as indicated by the appearance of a sharp singlet at δ 9.6 ppm), and it is this species which apparently facilitates production of the N-Mannich base.

Hydrolysis of the N-Mannich bases, prepared from either doxorubicin or daunorubicin and the respective amides, at 37° C. in pH 7.4 triethylammonium acetate (TEAA) buffer (20 mM) containing 10-30% MeOH was monitored by reverse phase HPLC. The disappearance of the prodrugs as a function of time is described by simple first order kinetics. As indicated in Table 1, the rate of hydrolysis paralleled the relative acidity of the amide employed. A primary concern for the products arising from the simple amides surveyed was that they would prove to be too stable for use as therapeutic agents resulting in stable N-Mannich bases would be subject to extensive metabolism and excretion before release of the active drug. Identification of a less robust construct that could be used to rapidly deliver the active Schiff base metabolite of doxorubicin to a growing tumor was undertaken.

TABLE 1
Half-life for hydrolysis of anthracycline-formaldehyde
N-Mannich bases as a function of amide structure.^{a}

| N-Mannich Base | Hydrolysis Medium | Half-Life | |
|---|---|---|---|
| DOX-Acetamide | 20 mM pH 7.4 TEAA | >50 | h |
| DOX-Benzamide | 20 mM pH 7.4 TEAA | >50 | h |
| DOX-Lactamide | 20 mM pH 7.4 TEAA | 45 | h |
| DAUN-4-Nitrobenzamide | 20 mM pH 7.4 TEAA | 25 | h |
| DOX-Fluoroacetamide | 20 mM pH 7.4 TEAA | 7 | h |
| DAUN-Salicylamide | 20 mM pH 7.4 TEAA | 73 | min |
| DOX-Salicylamide | PH 7.3 RPMI 1640 | 57 | min |

^{a}N-Mannich bases consisted of doxorubicin (DOX) or daunorubicin (DAUN) as the amine nitrogen donor and the respective amides as indicated and were synthesized using the general procedure given int he experimental section. All hydrolysis experiments were conducted at 37° C., and rate constants were determined from reverse phase HPLC measurement of AUC (area under the curve) values for the respectivee N-Mannich bases and liberated parent anthracyclines as a function of time.

Studies with the dimeric anthracycline-formaldehyde conjugates doxoform and epidoxoform have indicated that the half-life of hydrolysis to the Schiff base active metabolite plays a crucial role in determining the usefulness of the prodrug. While doxoform has a half-life at 37° C. and pH 7.4 of approximately 10 min, epidoxoform is much more robust with a half-life of 2 h. The marked variance in stability can be attributed to structural differences resulting from unique bonding between formaldehyde and the amino sugar moieties of the respective drugs. Although doxoform is more potent than epidoxoform when tested against a variety of cultured tumor-derived cell lines, it also is poorly tolerated by mice at doses as low as 1 mg/kg body weight. Epidoxoform, however, has shown improved efficacy in mice, relative to doxorubicin, and is well tolerated at doses up to 150% of the maximum tolerated dose of doxorubicin. The nature of the toxicity attributed to doxoform has not yet been determined, however, in light of the results obtained for the more stable epidoxoform, the rapid hydrolysis of the prodrug to the active metabolite is highly suspect as a contributing factor. A burst release of the intensely potent formaldehyde Schiff base of doxorubicin, which occurs before sufficient time is allowed for distribution of the prodrug to a growing tumor, may lead to insurmountable systemic toxicity. Based on these observations, an N-Mannich base prodrug with a half-life of 1-2 h was sought to yield a prodrug of sufficient stability to facilitate synthesis and purification and to allow for complete distribution of the cytotoxin in treated mice followed by relatively rapid release of the active drug.

N-Mannich bases derived from salicylamide have been demonstrated to deviate greatly from the stability predicted by the electronic character of the amide. While electron donating substituents on an aromatic amide are generally expected to increase the stability of the respective N-Mannich base, the presence of a hydroxyl group ortho to the amide moiety serves to greatly destabilize the salicylamide derived product. The half-life of hydrolysis of the daunorubicin N-Mannich base of salicylamide (daunsaliform) was found to be 73 min at 37° C. in pH 7.4 buffer. This fell perfectly within our somewhat arbitrary target time frame for drug release. The salicylamide N-Mannich base of doxorubicin (doxsaliform) was also prepared. Hydrolysis of this product was followed in pH 7.3 RPMI 1640 cell culture medium containing 10% DMSO to reflect, in part, the conditions under which cytotoxicity experiments would be carried out. The half-life of doxsaliform hydrolysis was found to be 57 min under these conditions, which made it the prime candidate for further analysis. The source of this observed difference in the half-life of daunsaliform and doxsaliform hydrolysis may reflect general acid-base catalysis arising from the varying salt concentrations and compositions of the buffers used to determine the stability of the respective prodrugs.

The aqueous stability of the N-Mannich base construct has been explored previously. The proposed mechanism of hydrolysis is shown in FIG. 7. This mechanism suggests that efficient hydrolysis depends on the free electron pair of the amine and is accelerated by protonation of the carbonyl oxygen by a proximal water molecule, which is followed by tautomerization of the liberated imidic acid to the stable amide. The most important result of this manner of decomposition is the retention of formaldehyde by the amino group, which serves to liberate the desired Schiff base active metabolite as an intermediate to full hydrolysis.

FIG. 8 illustrates the analogous decomposition of doxsaliform. The key difference is the ability of the phenolic group of salicylamide to protonate the carbonyl oxygen, in lieu of water, via a favorable hexagonal intramolecular transition state. This accounts for the relative instability of the salicylamide derived N-Mannich bases. It also indicates that the decomposition of doxsaliform is not an exclusively hydrolytic event and is expected to occur in non-aqueous solution. Indeed, the decomposition of doxsaliform has been observed, albeit more slowly, in organic solvents as well as during storage as a dry solid. This unique method of decomposition is a “trigger release” as it serves to inherently deliver the cytotoxin in a manner that cannot be exclusively described as hydrolytic; although, in aqueous solution the two terms are used interchangeably.

The mechanism of trigger release observed for the N-Mannich bases, including doxsaliform, can be exploited to stabilize the prodrug both in solution and during storage. As noted above, the free electron pair on the 3′-amino group of doxorubicin is required for efficient release of the trigger. Protonation of this nitrogen is, therefore, expected to stabilize the prodrug. This has been observed for doxsaliform, as the half-life in pH 2.0 trifluoroacetic acid (0.1% in water) at 37° C. was found to be 17.5 h. A sufficiently acidic solution is required to render this stabilizing effect, as formation of the N-Mannich base serves to decrease the pKa of the component amine; the amino moiety of doxsaliform has an estimated pKa of 3.0-3.5. Likewise, lyopholyzed salts of doxsaliform have been found to be stable at −20° C. for periods of several months, while the free base decomposes to an appreciable degree (˜20%) after 3 weeks.

A second method for retarding the release of doxorubicin from doxsaliform is the acyloxymethylation of the phenolic moiety of salicylamide illustrated in FIG. 12. In accordance with the proposed mechanism of trigger release, replacing the phenolic proton of salicylamide with an enzymatically cleavable protecting group is expected to instill the stability of simple N-Mannich bases such as those resulting from benzamide or acetamide. Initial attempts to simply acetylate the phenolic moiety of salicylamide had failed due to a well documented O to N acyl migration, which results in a stable imide product. Use of the acetoxymethylene (AOM) or butyryloxymethylene (BOM) groups, however, leads to phenol protected products that are stable, both as solids and in aqueous solution, for extended periods of time. At 37° C. in pH 7.3 RPMI 1640 cell culture medium, the half-life of release of doxorubicin from the acetoxymethylene-protected derivative is comparable to that of the simple amides tested; no hydrolysis was evident after 3 h of observation under these conditions. The utility of these compounds, however, is realized in the presence of non-specific esterases which rapidly cleave off the protecting group. Incubation of AOM-doxsaliform (11.0 mM) in pH 8.0 phosphate buffer in the presence of pig liver esterase (1.0 unit esterase/mL) is characterized by initial and rapid removal of the acetoxymethylene protecting group and concomitant hydrolysis of the newly deprotected doxsaliform to liberate doxorubicin-formaldehyde conjugate. The time for deprotection of half of the AOM-doxsaliform under these conditions has been estimated at 15 to 20 min. A similar time frame (16 min) for removal of half of the acetoxymethylene protecting group from salicylamide in 80% human plasma has been reported, as well as a time period of 5 min for removal of half of the butyryloxymethylene group. These rapidly removed protecting groups may serve to improve the stability of doxsaliform for handling in the laboratory as well as during formulation of the prodrug for administration in future in vivo mouse experiments.

The biological activities of several of the N-Mannich base derivatives were determined by their in vitro cytotoxicity against MCF-7 and MCF-7/ADR human breast cancer derived cell lines. While MCF-7 cells are sensitive to doxorubicin, the MCF-7/ADR cell line is an MCF-7 derivative which is characterized by its marked resistance to doxorubicin. IC₅₀ values were determined for prodrug exposure times of 3 or 24 h against both cell lines. The majority of the tested compounds, with the exception of doxsaliform, were found to be less active than doxorubicin against both cell lines after 3 h treatment. The lack of activity is attributed to the limited hydrolysis of the N-Mannich base products over the 3 h treatment time. Exposure to the N-Mannich bases for 24 h, however, led to IC₅₀ values that were generally comparable to that of the parent drug. Although this equipotency observed after 24 h indicates that, given substantial time, the release of doxorubicin results in efficient cell killing, the time frame required for sufficient release of the active drug from the more robust prodrugs is expected to allow for significant loss of the cytotoxin in vivo to elimination pathways.

Table 2 shows the results of 4 h treatment of MCF-7 and MCF-7/ADR breast and PC-3 prostate cancer cells with doxsaliform. This time frame was chosen in an attempt to demonstrate the efficiency of the formaldehyde-mediated toxicity of the prodrug. Long treatment times (>20 h) have been shown to partially nullify the cytotoxic advantage of the dimeric prodrug doxoform, presumably by allowing for the induction of oxidative stress and production of excessive formaldehyde by unmodified doxombicin. Conversely, short exposure periods (<3 h) to doxoform have been shown to elicit the most pronounced differences in efficacy between the prodrug and parent doxorubicin. However, these experiments capitalize on the rapid hydrolysis of doxoform to the N-(hydroxymethyl)-doxorubicin metabolite. Doxsaliform, being more stable, requires more time for release of the trigger and delivery of the active drug. Therefore, a time frame was chosen which allows for relatively rapid removal of the drugs from the treated cells, so as to elicit a measurable difference between doxombicin and the prodrug, while allowing ample time for prodrug trigger release and delivery of the active cytotoxin. Doxsaliform experiences approximately 4 half-lives of hydrolysis over the 4 h treatment time which serves to deliver greater than 95% of the administered dose as the active metabolite.

TABLE 2
IC₅₀ values for cancer cell growth inhibition
by doxsaliform compared with doxorubicin.

| Cell Type | Doxorubicin (nM) | Doxsaliform (nM) | DOX/doxsaliform^{a} |
|---|---|---|---|
| MCF-7 | 300 | 80 | 4 |
| MCF-7/ADR | 8000 | 800 | 10 |
| PC-3 | 300 | 80 | 4 |

^{a}The DOX/doxsaliform ratio indicates the fold increase in cytotoxicity in comparable 4 h drug treatment assays.

The results in Table 2 indicate that doxsaliform does indeed exhibit superior cytoxicity relative to doxorubicin against all three cell lines tested. It may be argued that the improved efficacy is the result of altered absorption or cellular distribution of the prodrug, but the inferior cytotoxicity of the more stable, yet chemically similar, N-Mannich bases tested indicates that the rate of release of the active Schiff base species dictates potency. In addition, previous studies have shown that administration of 1.5 equiv of formaldehyde with doxorubicin does not lead to improved potency relative to doxorubicin alone. This would indicate that it is the release of doxorubicin in the immediate proximity of formaldehyde, or, more accurately, the release or formation of the Schiff base active metabolite which is responsible for the improved potency of the prodrugs, doxoform and doxsaliform.

Although doxoform and doxsaliform are proposed to exploit formaldehyde in an identical manner to deliver the active Schiff base species upon partial hydrolysis, there is a marked difference in the potency of the two compounds. While doxoform has been shown to be 250 and 10,000 times as potent as doxorubicin against cultured sensitive MCF-7 and resistant MCF-7/ADR cells respectively, doxsaliform is only 4 times as active against PC-3 and MCF-7 cells and 10 times as active against the resistant MCF-7/ADR cell line (Table 2). Preliminary fluorescence microscopy studies have indicated that varying intracellular distribution of the prodrugs may be responsible for the observed difference in potency. Doxoform appears to accumulate in the nucleus, or immediately adjacent to the nucleus in what may be the Golgi apparatus, in both sensitive and resistant cells. Conversely, the N-Mannich base prodrugs are found to be more disperse, accumulating in multiple cytosolic focal points in sensitive cells, with little accumulation observed in resistant MCF-7/ADR cells. This indicates that the N-Mannich bases may be substrates for the gp120 multidrug resistance pump which is overexpressed in MCF-7/ADR cells (MDR1). Despite the need for further studies to unambiguously identify the nature of the final points of deposition for the prodrugs, it is obvious that doxoform and doxsaliform are characterized by unique patterns of intracellular distribution. Future work will focus on targeting doxsaliform to the nucleus of cancerous cells via tumor specific receptors so as to deliver the active Schiff base species to its proposed ultimate site of action, nuclear DNA.

### Example 2

Rational Design and Synthesis of Androgen Receptor Targeted Non-Steroidal Anti-Androgen Ligands for the Tumor Specific Delivery of a Doxorubicin-Formaldehyde Conjugate

Another approach to achieving anti-tumor specificity with concomitant reduction of systemic toxicity is the selective delivery of cytotoxins. While the targeting of cytotoxic agents to tumors via a carrier molecule is relatively new to the clinic, much pre-clinical work has been carried out in this promising field. Cytotoxins as varied as nitrogen mustards, nitroso-ureas, anthracyclines, taxanes, mitomycin C, membrane acting peptides, and assorted antibiotics have all been employed in the search for tumor selective therapeutics. Although these selective cytotoxins rely upon the expression of specific protein targets and are, therefore, prone to resistance mechanisms such as mutation or changes in expression of the target, they have several advantages over related non-toxic ligands. While the efficacy of molecules which interfere with the action of a specific cellular protein depend on expression of the target in every cell of a tumor, targeting compounds which release a non-specific cytotoxin can potentially act upon tissue surrounding the target expressing cell. Accumulation of the cytotoxin within the tumor is the goal, as opposed to direct action of the ligand on a cellular receptor. Ligands which act to merely deliver a cytotoxin may even be expected to exploit established resistance mechanisms such as over-expression of the targeted receptor.

The androgen receptor (AR) has been identified in a wide array of human tumors in both male and female patients. Carcinomas of the breast, ovary, esophagus, lung, and prostate have all been shown to express the androgen receptor. The AR exists primarily as a cytosolic receptor in complex with several heat-shock proteins (hsp7O, hsp9O, and hsp56-59). Ligand binding leads to dissociation of the heat-shock proteins, homodimerization, and translocation into the nucleus where the dimeric receptor recognizes hormone responsive elements and various components of the transcription machinery. The receptor is often over-expressed in hormone refractory prostate cancer and is also known to acquire mutations which lead to promiscuous binding of various non-androgen ligands. This example describes the synthesis of a series of non-steroidal anti-androgens which may be used to deliver a doxorubicin-formaldehyde conjugate to AR expressing tumors.

A variety of both steroidal and non-steroidal ligands for the AR have been described, providing many potential options to exploit as AR targeting molecules. The non-steroidal antiandrogens (NSAs) bear little resemblance to the endogenous steroids they antagonize. Most notably, they are smaller and are characterized by functional groups which lead to a considerably more polarizable surface area relative to the steroidal ligands. Although the clinically employed NSAs exhibit decreased AR binding affinity relative to DHT, binding can be readily improved through facile modifications of the core structures. Due to these aspects, as well as the general ease of synthesis, the modification of NSAs, through the introduction of varying tethers for the attachment of the salicylamide trigger, was used.

Nilutamide is one of a small group of clinically employed antiandrogens.

Discovered in 1979, nilutamide is classified as a pure anti-androgen. Unlike the most commonly employed clinical anti-androgen, flutamide, which acts as a partial agonist and actually promotes growth of AR expressing cells at higher concentrations, nilutamide shows no growth enhancing characteristics. Of considerable interest is the observation that the 3′ nitrogen of the 1-cyano derivative of nilutamide can be modified with a wide variety of substituents which lead to improved binding over the parent drug. The binding pocket of the AR apparently not only tolerates, but positively interacts with substituents such as primary alcohols of varying lengths, double and triple bonds, and aromatic ring systems.^{44,45,48} While the direct attachment of doxorubicin to nilutamide may not yield a viable ligand for the androgen receptor, the accommodating nature of the AR ligand binding domain is expected to allow for the development of a suitable tether by which nilutamide may be linked to salicylamide. A construct of this type not only allows for the concomitant delivery of doxorubicin and formaldehyde via preparation of an N-Mannich base with the tethered salicylamide, but also renders a generic targeting group which may be used to deliver a variety of other compounds to AR expressing cells.

Prompted by the superior AR binding affinity of a nilutamide alcohol, relative to nilutamide and hydroxyflutamide, the active metabolite of flutamide, the synthesis of a series of ethylene glycol derived tethers was undertaken. Polyethylene glycols are commonly used excipients for drug delivery. They are well tolerated and relatively stable to metabolic enzymes. Tethers consisting of diethylene glycol and triethylene glycol were explored based on their varying lengths and steric similarities to the hydroxybutane arm. The straight chain ethers were expected to occupy the same cleft of the androgen receptor ligand binding domain (AR-LBD) in which the hydroxybutyl chain resides. The ethylene glycols were also expected to offer superior aqueous solubility relative to simple homologous alkyl tethers. The ethylene glycol dimer and trimer were both employed in an effort to identify a tether of sufficient length to preclude interference of ligand binding by the salicylamide and anthracycline portions of the final drug.

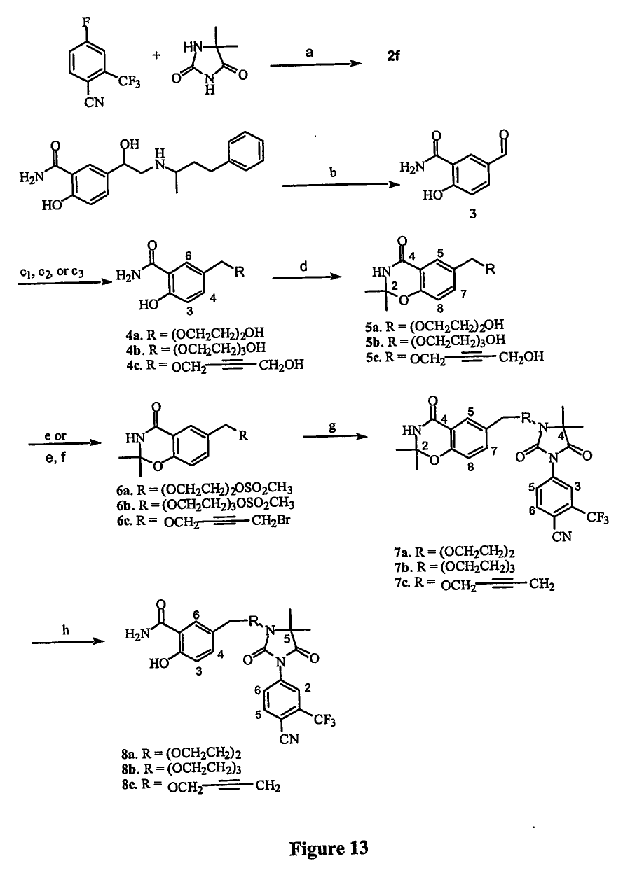

Synthesis of the targeting group with diethylene and triethylene glycol tethers was conducted as shown in FIG. 13. The 1-cyano derivative of nilutamide 2f was prepared in one step and 60% yield from 4-fluoro-2-(trifluoromethyl)-benzonitrile and 5,5-dimethylhydantoin in the presence of potassium carbonate. The oxidation of Labetalol with sodium periodate was accomplished using a modified literature procedure to give 5-formylsalicylamide 3 in 70% yield. Introduction of the tethers, to generate the alcohols 4a and 4b, was then carried out in good yields (up to 91%) via decaborane mediated reductive etherification using the respective ethylene glycol as solvent. Protection of the amide and phenolic moieties of 4a and 4b to give the dimethylbenzoxazines 5a and 5b was achieved in up to 88% yield by reflux in acetone and 2,2-dimethoxypropane, containing a catalytic amount of p-toluenesulfonic acid. The primary alcohol of each of the benzoxazine protected intermediates was then mesylated in 88-92% yield by treatment with triethylamine in the presence of pyridinum methanesulfonate, formed in situ, to give compounds 6a and 6b. Coupling of 2f and tether bearing salycilamide portions of the targeting group was accomplished by deprotonation of 2f with sodium hydride followed by addition of either 6a or 6b. The resulting benzoxazine protected targeting groups 7a and 7b were then deprotected by reflux in methanol containing 20% water, in the presence of a catalytic amount of p-toluenesulfonic acid, to yield the desired compounds 8a and 8b.

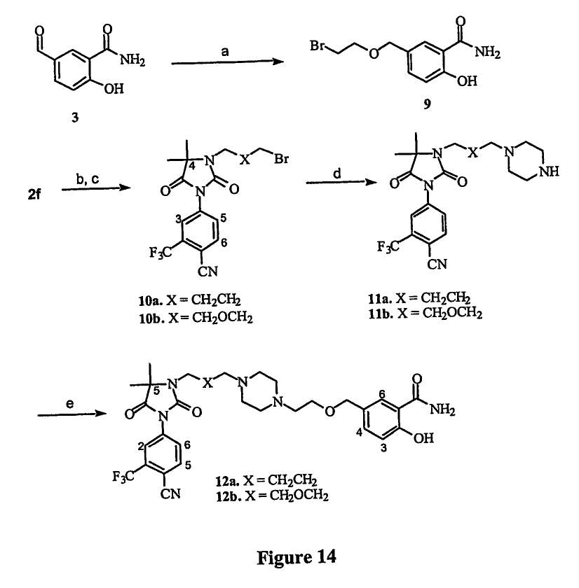

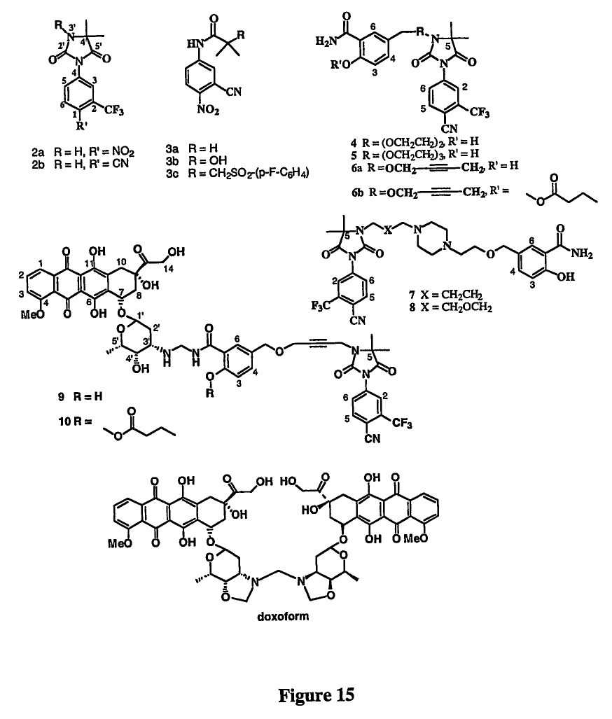

A second set of constructs was devised in order to introduce a solubilizing functionality and a source of rigidity into the tether. The heterocyclic diamine piperazine was chosen in an effort to address both concerns. The introduction of two ionizable amines into the tether should afford additional solubility relative to the uncharged ethylene glycols. Also, the conformational constraints imposed by the six membered piperazine ring should serve to inhibit intramolecular associations of the drug. The syntheses of two derivatives incorporating piperazine into the tether are presented in FIG. 14. Deprotonation of 2f with sodium hydride in DMF followed by addition of an excess of either 1,4-dibromobutane or bis(2-bromoethyl) ether yielded the brominated compounds 10a and 10b in 85% and 82% yields, respectively. Subsequent displacement of the bromide leaving group with excess piperazine in tetrahydrofuran gave the diamino derivatives 11a and 11b. Finally, the target compounds 12a and 12b were prepared by refluxing 11a or 11b in THF with the 2-bromoethoxy ether 9, which was prepared by the same route as the ethylene glycol derived benzylic ethers.

Although the four described compounds, 8a, 8b, 11a, and 11b, were expected to be sufficient to allow for preliminary evaluation of our targeting strategy, a final candidate was pursued in an attempt to capitalize on noted attributes of previously characterized AR binding molecules. A series of testosterone-geldanamycin conjugates which show a wide range of efficacy, dependant solely upon the length of an alkynyl tether employed to join the two drugs have been described. It has been demonstrated that a β-propargylic group at the 17-position of testosterone is necessary for biological activity in the tested series. Presumably, the triple bond serves to stringently direct the tether's protrusion from the binding pocket. The relevance of this requirement for tether rigidity in testosterone-geldanamycin conjugates to NSA derivative binding was not immediately clear. There is no direct evidence to suggest that the tethers of these conjugates reside in the same cleft in the AR binding pocket as do the 3′ substituents of the series 2b-e. However, much indirect evidence supports this assertion.

FIG. 13 shows the stepwise synthesis of the targeting group incorporating 2-butyne-1,4-diol in place of the ethylene glycol tethers. Reductive etherification of 3 with decaborane in the presence of molten 2-butyne-1,4-diol yields the corresponding benzylic ether 4c. After removal of excess butynediol by repeated extraction, the crude product was dissolved in acetone where it was refluxed with 2,2-dimethoxypropane and a catalytic amount of p-toluenesulfonic acid to yield 70% of the benzoxazine protected intermediate 5c after two steps. Attempts to mesylate the alcohol, as was done with the ethylene glycol derivatives, gave a mixture of products consisting of primarily the desired, yet unstable, mesylate and the corresponding chlorinated product in varying ratios depending on the conditions used and the reaction time. The chlorinated product apparently results from displacement of the successfully installed methanesulfonate ester by the chloride ion liberated from consumed methanesulfonyl chloride. In an attempt to improve upon the yield and selectivity achieved in the introduction of a leaving group to the propargylic position of the tether, the mesylation reaction was repeated in the presence of 10 equiv of LiBr. This served to completely brominate the terminus of the tether, in 87% yield, which rendered 6c as a superior substrate for subsequent reaction with the 2f anion. Displacement of the bromide with the sodium salt of 2f gave the protected product 7c in 82% yield. Finally, removal of the benzoxazine protecting group was carried out in 80% yield to give the desired compound 8c.

The androgen receptor (AR) was obtained from PC3 cells (donated by Dr. Kerry Burnstein, University of Miami; Miami, Fla.) which had been stably transfected with the human androgen receptor cDNA (PC3/AR). PC3/AR cells have been thoroughly characterized and have been shown to express the AR at ˜596 fmol/mg total cellular protein, which is comparable to the expression of a mutant AR in the established LNCaP cell line (˜816 fmol/mg). PC3/AR cells were grown to near confluence, sonicated, and centrifuged to consistently yield 5.0 mL of a lysate containing approximately 1.9 mg/mL total cellular protein. Division of the collected lysate into 100 μL fractions yielded approximately 113 fmol AR per aliquot (˜1.1 nM). Crude lysate was used as the binding reaction medium in order to account for undesirable yet specific ligand-protein interactions. While purified AR can be used for the binding assay, we felt it was necessary to identify any unwanted binding events which supercede the affinity of the targeting compounds for the AR.

The competitive binding assays were run for 30 min incubation periods to demonstrate the interaction of the nonsteroidal antiandrogens with AR during a relevant time frame for targeting. Tritiated Miboloerone (³H-MIB) was chosen as the radioligand on account of its availability and extensive use in this capacity in the literature. All assays were run at 4° C., to avoid proteolytic degradation of the receptor, using a modified protocol which employs hydroxyapatite to sequester and wash the protein fraction of the assay solution. Hydroxyapatite was supplied as an insoluble calcium phosphate coated agarose gel, which served to efficiently remove proteins from solution. The gel was then collected via filtration and washed extensively to remove background radioactivity due to nonspecific interactions with ³H-MIB. Scintillation counting of the dry, washed gel and filter was then employed to quantify total binding of 1.0 nM ³H-MIB in the presence of various concentrations of the test compounds. These numbers were then compared to controls for nonspecific binding and unchallenged total binding. Results are shown in Table 3. All assays were performed in duplicate and scintillation counting was repeated three times to insure reproducibility of the data.

TABLE 3
IC₅₀ and relative binding affinity (RBA) values of test ligands.^{a}

| Test Compound | IC50 (nM) | RBA |
|---|---|---|
| nilutamide | 9 | 100 |
| 2f | 6 | 150 |
| 8a | 77 | 12 |
| 8b | 332 | 3 |
| 8c | 49 | 18 |
| 12a | >1000 | <1 |
| 12b | 346 | 3 |
| 13 | 90 | 10 |
| flutamide | 154 | 6 |
| salicylamide | >>1000 | <<1 |

^{a}IC₅₀ and relative binding affinity values determined from competitive binding for the human AR of the various test ligands against 1.0 nM ³H-Mibolerone in PC3/AR cell lysate at 4° C.

There was an initial concern that the small differences in specific and nonspecific binding would not be accurately quantified by scintillation counting. To address this issue, a positive control using unlabled Mibolerone as the test ligand was performed. The cold Mibolerone was found to compete off 50% of the radioligand at a concentration of approximately 2.0 nM, suggesting that the developed method is a valid measure of competitive binding. Likewise, a negative control experiment was conducted using salicylamide, which is expected to show no specific binding to the AR. Table 3 shows that salicylamide was, in fact, ineffective at competing for AR binding in the presence of ³H-Mibolerone. Further control experiments using the cytosolic fraction of PC3/neo cells, which do not express the androgen receptor, also showed no specific binding of ³H-Mibolerone, suggesting that the differences measured in PC3/AR lysate are real and AR specific.

Relative binding affinities (RBA) for the ten analyzed compounds are listed in Table 3. The clinically employed nilutamide was found to inhibit ³H-Mibolerone with an apparent IC₅₀ of 10 nM and has been assigned an arbitrary RBA of 100. The RBAs of the other test compounds are expressed as fractions of nilutamide binding, based on their respective IC₅₀ values. The RBA of 2f (150%) suggests that the use of this molecule as the core of our targeting constructs was quite appropriate. The majority of the compounds tested exhibit RBA values between 1% and 20% of that observed for nilutamide, indicating that the introduction of our tethers has a detrimental effect on binding. However, the IC₅₀ value of the best of the targeting groups, 8c at 49 nM, is still on the same order of magnitude as the unmodified 2f.

The triethylene glycol derivative 8b, having the longest tether of the tested compounds, displayed only 3% of the binding affinity of nilutamide. This surprising finding is likely the result of the excessive flexibility of the triethylene glycol tether which is proposed to facilitate folding of the molecule and subsequent intramolecular interactions which preclude efficient receptor binding.⁵³ Also of interest is the poor ability of the piperazine analogs 11a and 11b to effectively displace ³H-Mibolerone binding in the tested concentration range. The added steric demands of the piperazine ring or the presence of a cationic amine in the tether may account for this lack of activity.

The diethylene glycol and butynediol derivatives, 8a and 8c respectively, exhibited the best RBA values, although they were only 12% and 18% as efficient as nilutamide, respectively, in competing for AR binding against ³H-Mibolerone. Due to the short length of the tethers in these compounds, it is possible that the salicylamide moiety of each is responsible for beneficial interactions which improve the binding affinity. Finally, the alkynyl tether of 8c apparently serves to maintain rigidity and direct the salicylamide portion of the molecule out from the binding pocket, much as it is proposed to do for Danishefsky's geldanamycin conjugates.²⁰

Having identified compound 8c as a potential targeting molecule, we proceeded to prepare the N-Mannich base which results from the condensation of 8c with doxorubicin and formaldehyde. The N-Mannich base was isolated in 60% yield and was found to compete with H³-Mibolerone for AR binding with an affinity (IC50=90 nM) which was comparable to that of unmodified 8c (Table 3). In order to address the concern of hydrolysis of 13 in the binding reaction, a solution of the targeted prodrug was prepared in the reaction buffer and incubated at 4° C. for 30 min. Reverse phase HPLC analysis indicated no appreciable hydrolysis of the N-Mannich base under these conditions, suggesting that the specific binding observed was attributed to 13 and not liberated 8c. These results suggest that doxorubicin-formaldehyde conjugates, and perhaps various other cytotoxins, may be efficiently targeted to AR expressing cells via attachment to non-steroidal antiandrogens by a suitable tether. Future work will explore the AR interaction of these constructs in whole cells and the efficacy of the targeted N-Mannich base in a prostate tumor expressing mouse model.

Several experiments were carried out in attempts to quantitate the cytotoxicity of 13, relative to doxorubicin and untargeted doxsaliform, against PC3/AR and PC3/neo cells. Experiments were run in cell culture media supplemented with either fetal bovine serum (FBS) or dextran-charcoal stripped calf serum in an attempt to account for the presence of testosterone in the unadulterated FBS. Unfortunately, an underlying problem prevented the accurate analysis of the effect of prodrug targeting. Extended treatment of cells with varying concentrations of the two prodrugs leads to release of the doxorubicin-formaldehyde conjugate by 13 and by untargeted doxsaliform, both inside and outside the cells, irrespective of receptor binding. Thus both prodrugs serve to bathe the cells in the doxorubicin-formaldehyde conjugate via hydrolysis over the course of the exposure period. Prodrug treatment times >3 h were required for extensive hydrolysis of the N-Mannich base, but over this time period, both 13 and doxsaliform release the same amount of the doxorubicin-formaldehyde conjugate. An in vivo system is expected to allow for accumulation of the targeted prodrug in AR expressing cells, where hydrolysis of the N-Mannich base will lead to localized delivery of the active drug. This should greatly contrast the deposition of the untargeted doxsaliform, which is expected to experience no preferential distribution. Simply stated, the targeted prodrug was designed to exploit a dynamic system of circulation, accumulation due to receptor binding, and release of the cytotoxin from an inactive conjugate, while cell culture only offers a static model for determining cytotoxicity. While these IC₅₀ studies indicated that the potency of the targeted drug was not diminished relative to doxorubicin or the untargeted N-Mannich base prodrug, the effect of AR binding and subsequent release of the doxorubicin-formaldehyde conjugate could not be ascertained through cell culture experiments. However, preliminary fluorescence microscopy has shown that both 8c and 13 do, in fact, bind to the AR in live cultured cells (work in progress). Based on the AR binding affinity of 13, as determined here in cell lysate as well as in whole cells (data not shown), we are currently developing a mouse model, employing orthotopicly implanted prostate tumors, which will serve as a dynamic test system for assessment of the efficacy of 13.

Experimental: Melting points were determined in open capillary tubes with a capillary melting point apparatus and are uncorrected. ¹H-NMR spectra were acquired with a 500 MHz spectrometer. Unambiguous NMR assignments for the protons of the 2f, salicylamide, and doxorubicin portions of the synthesized compounds are designated by “nil”, “sal”, or “dox” respectively. Mass spectral data were acquired on a mass spectrometer by electron impact (EI) using a perfluorokerosene internal standard for [M+] data or liquid SIMS (LSIMS) ionization with a polyethylene glycol (PEG) internal standard for [MH+] data. Mass spectral data for compound 13 were collected by Dr.

Chris Hadad (Ohio State University; Columbus, Ohio) with a Fourier Transform mass spectrometer. HPLC analyses were performed with a liquid chromatograph equipped with a diode array UV-vis detector and workstation; chromatographies were performed with a 5 μm reverse phase C₁₈ microbore column, 2.1 mm i.d.×100 mm, eluting at 0.5 mL/min, monitoring at 260 and 310 nm. Acceptable analytical resolution was achieved with gradients of acetonitrile and triethylammonium acetate (Et₃NHOAc; TEAA), prepared as 20 mM triethylamine adjusted to pH 6.0 with acetic acid. The method employed for all analytical chromatography was as follows: A=CH₃CN, B=pH 6.0 buffer; A:B, 0:100 to 70:30 at 10 min, isocratic until 12 min, 0:100 at 15 min. For preparative HPLC, a 5 μm spherical particle C₁₈ semi-preparative column was employed, 10 mm×25 cm with a 10 mm×5 cm guard column, eluting at 3.0 mL/min, monitoring at 260 and 310 nm. Adequate preparative separation was achieved using the following method: A=CH₃CN, B=20 mM triethylamine adjusted to pH 3.5 or 4.0 as indicated with glacial acetic acid (TEAA buffer); A:B, 0:100 to 70:30 at 20 min, isocratic until 30 min, 0:100 at 35 min. Water was distilled and purified with a Millipore Q-UF Plus™ purification system to 18 Mohm-cm. The flash silica gel used had a particle size: 32-63 μm and pore size: 60 Å.

PC3/AR and PC3/neo cells were a gift from Dr. Kerry L. Burnstein (University of Miami, Fla.). Both cell lines were maintained in vitro by serial culture in RPMI 1640 media supplemented with either 10% fetal bovine serum or 10% dextran-charcoal stripped (dilipidated) calf serum, L-glutamine (2 mM), HEPES buffer (10 mM), penicillin (100 units/mL), and streptomycin (100 μg/mL). Cells were maintained at 37° C. in a humidified atmosphere of 5% CO₂ and 95% air.

Syntheses: 4-(4,4-Dimethyl-2,5-dioxo-imidazolidin-1-yl)-2-trifluoromethylbenzonitrile (2f). To a stirring solution of 1.00 g (4.78 mmol) of 4-fluoro-2-(trifluoromethyl)-benzonitrile in 15.0 mL of DMF was added 3.10 g (23.9 mmol) of 5,5-dimethylhydantoin and 0.990 g (7.17 mmol) of K₂CO₃. The resulting suspension was stirred under an argon atmosphere at 55° C. for 16 h and then at 45° C. for 48 h. The reaction mixture was diluted to 300 mL with ethyl acetate, vacuum filtered and rotary evaporated at 40° C. followed by 50° C. and 50 μm Hg to yield a bright yellow paste. The paste was dissolved in 25% hexanes/75% ethyl acetate and eluted from a silica gel flash column (35 cm×3 cm) with 50% hexanes/50% ethyl acetate. The collected product was rotary evaporated at 40° C. to give a white solid which was recrystallized from ethyl acetate/hexanes to give 0.780 g (55%) of 2f as a white crystalline solid (mp 208-210° C.): ¹H NMR (500 MHz, (CD₃)₂CO) δ 1.53 (6H, s, 4-(CH₃)₂), 7.81 (1H, bs, NH), 8.13 (1H, dd, J=8, 2 Hz, 5), 8.20 (1H, d, J=8 Hz, 6), 8.25 (1H, d, J=2 Hz, 3); m/z 297.0723 [M+] (calculated for 297.0725); anal. (C₁₃H₁₀F₃N₃O₂) C, H, N.

5-Formyl-2-hydroxybenzamide (3). A 600 mL stirring aqueous solution of 2.00 g (5.48 mmol) of Labetalol hydrochloride in a 1.0 L round bottom flask was neutralized with 4 mL of saturated NaHCO₃. The reaction flask was then fitted with a dropping funnel containing 1.17 g (5.48 mmol) of sodium periodate in 50 mL of Millipore H₂O. Drop-wise addition of the periodate solution over 15 min at room temperature gave a pale pink solution which was stirred for an additional 20 min. The solution was acidified with 3.0 mL of concentrated aqueous HCl and stirred vigorously until a white precipitate was formed (approximately 2 min). The resulting suspension was stored for 12 h at 4° C., to facilitate precipitation, at which time it was filtered. The collected solid was recrystallized from 80 mL of boiling Millipore H₂O and allowed to sit for 12 h at 4° C. Vacuum filtration gave 0.634 g (70%) of 3 as white to pale golden needles (mp 204-206° C.): ¹H NMR (500 MHz, (CD₃)₂CO) δ 7.06 (1H, d, J=8 Hz, 3), 7.47 (1H, bs, NH), 7.98 (1H, dd, J=8, 2 Hz, 4), 8.33 (1H, bs, NH), 8.40 (1H, d, J=2 Hz, 6), 9.84 (1H, s, HCO), 13.87 (1H, s, 2-OH); m/z 165.0421 [M+] (calculated for 165.0426).

2-Hydroxy-5-[2-(2-hydroxy-ethoxy)-ethoxymethyl]-benzamide (4a). A solution of 200 mg (1.21 mmol) of 3 was prepared in 10 mL of diethylene glycol heated under an argon atmosphere to 70° C. in a mineral oil bath. After dissolution was complete, the solution was removed from the oil bath and allowed to cool for 5 min at which time 74 mg (0.61 mmol) of decaborane was added. Strong effervescence was observed over 5 min but then subsides The reaction was then placed back in the oil bath and was stirred at 70° C. for 5 h. The solvent was removed by rotary-evaporation at 60° C. and 50 μm Hg. After the bulk of the solvent was removed, the remaining oil was transferred to a separatory funnel with 300 mL of ethyl acetate. This solution was washed 4× with 50 mL portions of saturated brine and the organic layer was collected, dried over anhydrous magnesium sulfate, and rotary evaporated at 40° C. to a pale yellow oil. The desired product was then collected from a silica gel flash column (35 cm×3 cm diameter), eluted with 10% hexanes/90% ethyl acetate. Removal of the solvent by rotary-evaporation at 40° C. gave 291 mg (91%) of 4a as a clear, colorless oil: ¹H NMR (500 MHz, (CD₃)₂CO δ 3.49-3.54 (2H, m, OCH₂CH₂OH), 3.55-3.59 (2H, m, 1/2(OCH₂CH₂O)), 3.59-3.66 (4H, m, CH₂OH, 1/2(OCH₂CH₂O)), 3.89 (1H, bs, CH₂OH), 4.44 (2H, s, Bn), 6.86 (1H, d, J=9 Hz, 3), 7.19 (1H, bs, NH), 7.4 (1H, dd, J=9, 2 Hz, 4), 7.81 (1H, d, J=2 Hz, 6), 8.01 (1H, bs, NH), 12.9 (1H, bs, 2-OH); m/z 255.1106 [M+] (calculated for 255.1107).

6-[2-(2-Hydroxy-ethoxy)-ethoxymethyl]-2,2-dimethyl-2,3-dihydro-benzo[e][1,3]oxazin-4-one (5a). A sample of 200 mg (0.78 mmol) of 4a was dissolved in 20 mL of acetone and 10 mL of 2,2-dimethoxypropane. A catalytic amount of p-toluenesulfonic acid was added and the resulting solution was refluxed under an argon atmosphere at 80° C. for 1.5 h. The solvent was then removed by rotary-evaporation at 40° C. The resulting brown residue was transferred to a separatory funnel in 250 mL of ethyl acetate and was washed 3× with 50 mL portions of saturated brine containing 5% K₂CO₃. The organic layer was collected, dried over anhydrous magnesium sulfate, and rotary evaporated at 40° C. to give a yellow oil. The washed product was then eluted from a silica gel flash column (35 cm×3 cm) in 5% hexanes/95% ethyl acetate. Removal of solvent yielded 204 mg (88%) of pure Sa as a clear, colorless oil: ¹H NMR (500 MHz, (CD₃)₂CO) δ 1.61 (6H, s, 2-(CH₃)₂), 3.50-3.53 (2H, m, OCHCH₂OH), 3.59-3.65 (6H, m, CH₂OH, OCH₂CH₂O), 3.73 (1H, t, J=6 Hz, CH₂OH), 4.52 (2H, s, Bn), 6.92 (1H, d, J==9 Hz, 8), 7.47 (1H, dd, J=9, 2 Hz, 7), 7.82 (1H, bs, NH), 7.85 (1H, d, J=2 Hz, 5); m/z 295.1412 [M+] (calculated for 295.1420).

6-{2-12-(2-Hydroxy-ethoxy)-ethoxy]-ethoxymethyl}-2,2-dimethyl-2,3-dihydro-benzo[e][1,3]oxazin-4-one (5b). A solution of 200 mg (1.21 mmol) of 3 was prepared in 10 mL of triethylene glycol heated to 70° C. under an argon atmosphere in a mineral oil bath. After dissolution was complete, the solution was removed from the oil bath and allowed to cool for 5 min before 74 mg (0.60 mmol) of decaborane was added. Strong effervescence was observed over 5 min but then subsided. The reaction was then placed back in the oil bath and was stirred at 70° C. for 5 h. The solvent was then removed by heating to 125° C. in a Kügelrohr oven at 150 μm Hg for 2 h. After the bulk of the solvent was removed, the remaining viscous liquid was transferred to a separatory funnel with 100 mL of saturated brine. This solution was extracted into 4×200 mL portions of ethyl acetate, which were collected, pooled, dried over anhydrous magnesium sulfate, and rotary evaporated at 40° C. to a pale yellow oil. The desired, semi-pure product 4b was then collected from a silica gel flash column (35 cm×3 cm), eluting with 5% hexanes/95% ethyl acetate. This semi-pure product was dissolved in 20 mL of acetone and 10 mL of 2,2-dimethoxypropane. A catalytic amount of p-toluenesulfonic acid was added and the resulting solution was refluxed at 80° C. for 1.5 h. The solvent was then removed by rotary-evaporation at 40° C. The resulting brown residue was transferred to a separatory funnel in 300 mL of ethyl acetate and was washed 3× with 50 mL portions of saturated brine containing 5% K₂CO₃. The organic layer was collected, dried over magnesium sulfate, and rotary evaporated to a pale yellow oil at 40° C. The washed product was then eluted from a silica gel flash column (35 cm×3 cm) in 5% methanol/95% ethyl acetate. Removal of solvent yielded 231 mg (56% in two steps) of pure 5b as a clear, colorless oil: ¹H NMR (500 MHz, (CD₃)₂CO) δ 1.61 (6H, s, 2-(CH₃)₂), 3.50-3.53 (2H, m, OCH₂CH₂OH), 3.56-3.65 (10H, m, OCH₂CH₂OH, 2(OCH₂CH₂O)), 3.74-3.77 (1H, m, CH₂OH), 4.51 (2H, s, Bn), 6.92 (1H, d, J=8 Hz, 8), 7.48 (1H, dd, J=8, 2 Hz, 7), 7.84 (1H, d, J=2 Hz, 5), 7.99 (1H, bs, NH); m/z 339.1681 [M+] (calculated for 339.1682).

6-(4-Hydroxy-but-2-ynyloxymethyl)-2,2-dimethyl-2,3-dihydro-benzole[e][1,31-oxazin-4-one (5c). A solution of 200 mg (1.21 mmol) of 3 was prepared in 10 g of 1,4-butyne-2-diol by heating a mixture of the two solids at 70° C. in a mineral oil bath for 15 min. The resulting solution was removed from the oil bath and allowed to cool for 5 min before 74 mg (0.60 mmol) of decaborane was added. Strong effervescence was observed for approximately 5 min but then subsided. The reaction was then stirred under an argon atmosphere at 60° C. for 5 h and was subsequently diluted to 300 mL with ethyl acetate and transferred to a separatory funnel. After 8×40 mL washes with saturated brine, the organic layer was collected, dried over anhydrous magnesium sulfate, and rotary evaporated at 40° C. to give approximately 1.5 mL of a clear amber oil. The crude product was diluted to 10 mL in ethyl acetate and was eluted from a silica gel flash column (30 cm×2 cm) in 100% ethyl acetate. Rotary-evaporation of the solvent gave 4c as a semi-pure clear, pale yellow oil. The product from the flash column was dissolved in 20 mL of acetone and 10 mL of 2,2-dimethoxypropane. A catalytic amount of p-toluenesulfonic acid was added and the resulting solution was refluxed at 85° C. for 1.5 h. The solvent was removed by rotary-evaporation and the resulting oil was transferred to a separatory funnel in 300 mL of ethyl acetate. This solution was washed 3× with 40. mL portions of saturated brine containing 5% K₂CO₃ and the organic layer was collected, dried over anhydrous magnesium sulfate and rotary evaporated at 40° C. to give 233 mg (70% in two steps) of 5c as a clear, colorless oil: ¹H NMR (500 MHz, (CD₃)₂CO) δ 1.62 (6H, s, 2-(CH₃)₂), 3.45 (1H, bs, CH₂OH), 4.17 (2H, bm, CCH₂OH), 4.3 (2H, bm, OCH₂C), 4.53 (2H, s, Bn), 6.68 (1H, 2, J=8 Hz, 8), 7.43 (1H, dd, J=8, 2 Hz, 7), 7.90 (1H, d, J=2 Hz, 5), 8.21 (1H, bs, NH); m/z 275.1145 [M+] (calculated for 275.1158).

Methanesulfonic acid 2-12-(2,2-dimethyl4-oxo-3,4-dihydro-2H-benzole][1,3]oxazin-6-ylmethoxy)-ethoxy]-ethyl ester (6a). To a stirring solution of 200 mg (0.68 mmol) of 5a in 4 mL of THF was added 55 μl (0.68 mmol) of dry pyridine and 160 μl (2.1 mmol) of methanesulfonyl chloride. This solution was stirred under an argon atmosphere at room temperature for 30 min at which time 380 μl (2.8 mmol) of triethylamine was added. A white precipitate was formed immediately and the reaction was stirred for 2 h at room temperature. The reaction mixture was then diluted to 300 mL with ethyl acetate and transferred to a separatory funnel. After 3×40 mL washes with saturated brine containing 5% NaH₂PO₄, the organic layer was collected, dried over anhydrous magnesium sulfate, and rotary evaporated at 40° C. to a pale yellow oil. This crude oil was dissolved in 8 mL of ethyl acetate and introduced to a silica gel flash column (35 cm×3 cm) packed in 10% hexanes/90% ethyl acetate. Elution with the same, followed by removal of solvent by rotary-evaporation at 40° C., gave 233 mg (92%) of 6a as a clear, colorless oil: ¹H NMR (500 MHz, (CD₃)₂CO δ 1.62 (6H, s, 2-(CH₃)₂), 3.1 (3H, s, SO₂CH₃), 3.62-3.66 (2H, m, 1/2(OCH₂CH₂O)), 3.67-3.70 (2H, m, 1/2(OCH₂CH₂O)), 3.73-3.78 (2H, m, OCH₂CH₂OMs), 4.34-4.39 (2H, m, OCH₂CH₂OMs), 4.52 (2H, s, Bn), 6.92 (1H, d, J=9 Hz, 8), 7.48 (1H, dd, J=9, 2 Hz, 7), 7.74 (1H, bs, NH), 7.81 (1H, d, J=2 Hz, 5); m/z 373.1188 [M+] (calculated for 373.1195).

Methanesulfonic acid 2-{2-[2-(2,2-dimethyl-4-oxo-3,4-dihydro-2H-benzo[e][1,3]oxazin-6-ylmethoxy)-ethoxyl-ethoxy}-ethyl ester (6b). 6b was prepared as 6a in 90% yield: ¹H NMR (500 MHz, (CD₃)₂CO δ 1.61 (6H, s, 2-(CH₃)₂), 3.10 (3H, s, SO₂CH₃), 3.58-3.65 (8H, m, 2(OCH₂CH₂O)), 3.75 (2H, m, OCH₂CH₂OMs), 4.35 (2H, m, OCH₂CH₂OMs), 4.51 (2H, s, Bn), 6.92 (1H, d, J=9 Hz, 8), 7.48 (1H, dd, J=9, 2 Hz, 7), 7.83 (1H, d, J=2 Hz, 5), 7.92 (1H, bs, NH); m/z: 417.1452 [M⁺] (calculated for 417.1452).

6-(4-Bromo-but-2-ynyloxymethyl)-2,2-dimethyl-2,3-dihydro-benzo[e][1,3]oxazin-4-one (6c). To a stirring solution of 150 mg (0.55 mmol) of 5c in 2.0 mL of THF was added 45 μl (0.55 mmol) of pyridine and 170 μl (2.2 mmol) of methanesulfonyl chloride. After 30 min stirring under argon, 380 μl (2.7 mmol) of triethylamine was added and the resulting solution was allowed to stir for 1 h at room temperature, during which time a white precipitate gradually formed. To this stirring suspension was added 480 mg (5.5 mmol) of LiBr as a solution in 2 mL of THF. Once HPLC indicated completion of the reaction, the resulting suspension was diluted to 200 mL with ethyl acetate and was washed 3× with 40 mL portions of saturated brine containing 5% NaH₂PO₄. The organic layer was then collected, dried over anhydrous magnesium sulfate, and rotary evaporated at 40° C. to give a pale yellow oil. The washed product was eluted from a silica gel flash column (30 cm×2 cm) in 10% hexanes/90% ethyl acetate. Removal of solvent by rotary-evaporation at 40° C. yielded 162 mg (87%) of 6c as a clear, colorless oil: ¹H NMR (500 MHz, CDCl₃) δ 1.67 (6H, s, 2-(CH₃)₂), 3.95-3.98 (2H, m, OCH₂C), 4.21-4.25 (2H, m, CCH₂Br), 4.55 (2H, s, Bn), 6.21 (1H, d, J=8 Hz, 8), 7.47 (1H, dd, J=8, 2 Hz, 7), 7.89 (1H, d, J=2 Hz, 5), 8.08 (1H, s, NH); m/z 337.0314 [M+] (calculated for 337.0314).

5-(2-{2-[3-(4-Cyano-3-trifluoromethyl-phenyl)-5,5-dimethyl-2,4-dioxo-imidazolidin-1-yl]-ethoxy}-ethoxymethyl)-2-hydroxy-benzamide (8a). A solution of 100 mg (0.34 mmol) of 2f was prepared in 2.0 mL of DMF and to this was added 13.5 mg (0.34 mmol) of sodium hydride as a 60% emulsion in oil. The mixture was stirred under an argon atmosphere at room temperature for 3 h. The resulting yellow solution was then added to a stirring solution of 127 mg (0.34 mmol) of 6a in 2.0 mL of DMF and the reaction flask was heated to 60° C. in a mineral oil bath for 24 h. The product was precipitated by drop-wise addition of the reaction mixture to 100 mL of saturated aqueous NaH₂PO₃. The pale yellow precipitate was then extracted into 250 mL of ethyl acetate, which was collected and rotary evaporated at 40° C. to yield a yellow solution of crude product in DMF. Further rotary-evaporation at 50° C. and 100 μm Hg removed the DMF to give a viscous yellow oil. This residue was eluted from a silica gel flash column (30 cm×2 cm) in 10% hexanes/90% ethyl acetate to yield semi-pure 7a. The fractions containing 7a which were collected from the column were pooled and the solvent removed via rotary-evaporation at 40° C. to give a clear, colorless oil which was dissolved in 10 mL of 80% methanol/20% water. A catalytic amount of p-toluenesulfonic acid was added and the resulting solution was refluxed at 90° C. for 30 h. The methanol was removed and 50 mL of saturated brine was used to transfer the resulting emulsion to a separatory funnel where it was extracted into 250 mL of ethyl acetate and washed 2× with 40 mL portions of saturated brine containing 5% NaHCO₃. Collection of the organic layer, followed by drying over anhydrous magnesium sulfate and rotary-evaporation at 40° C., gave a pale yellow oil. Elution from a silica gel flash column (30 cm×2 cm) in 10% hexanes/90% ethyl acetate yielded 82 mg (42% in two steps) of 8a as a clear, colorless oil: ¹H NMR (500 MHz, (CDCl₃) δ 1.54 (6H, S, nil-5-(CH₃)₂), 3.54-3.59 (2H, m, OCH₂CH₂N), 3.61-3.65 (2H, m, 1/2(OCH₂CH₂O)), 3.66-3.70 (2H, m, 1/2(OCH₂CH₂O)), 3.74-3.79 (2H, m, OCH₂CH₂N), 4.46 (2H, s, Bn), 5.91 (1H, bs, NH), 6.62 (1H, bs, NH), 6.95 (1H, d, J=8 Hz, sal-3), 7.33 (1H, dd, J=8, 2 Hz, sal-4), 7.41 (1H, d, J=2 Hz, sal-6), 7.88 (1H, d, J=9 Hz, nil-S), 7.92 (1H, dd, J=9, 2 Hz, nil-6), 8.09 (1H, d, J=2 Hz, nil-2), 12.22 (1H, bs, 2-OH); m/z 534.1711 [M+] (calculated for 534.1726); anal. (C₂₅H₂₅F₃N₄O₆) C, H, N.

5-[2-(2-{2-[3-(4-Cyano-3-trifluoromethyl-phenyl)-5,5-dimethyl-2,4-dioxo-imidazolidin-1-yl]-ethoxy}-ethoxy)-ethoxymethyl]-2-hydroxy-benzamide (8b). 8b was prepared as 8a in 58% yield: ¹H NMR (500 MHz, (CD₃)₂CO) δ 1.57 (6H, s, nil-5-(CH₃)₂), 3.58-3.61 (4H, m, OCH₂CH₂O), 3.61-3.67 (6H, m, OCH₂CH₂ N, OCH₂CH₂O), 3.73-3.76 (2H, m, OCH₂CH₂N), 4.46 (2H, s, Bn), 6.90 (1H, d, J=8 Hz, sal-3), 7.19 (1H, bs, NH), 7.45 (1H, dd, J=8, 2 Hz, sal-4), 7.81 (1H, d, J=2 Hz, sal-6), 7.97 (1H, bs, NH), 8.17 (1H, dd, J=9, 2 Hz, nil-6), 8.22 (1H, d, J=9 Hz, nil-5), 8.29 (1H, d, J=2 Hz, nil-2), 12.95 (1H, s, 2-OH); m/z 578.2000 [M+] (calculated for 578.1988); anal. (C₂₇H₂₉F₃N₄O₇) C, H, N.

4-{3-[4-(2,2-Dimethyl-4-oxo-3,4-dihydro-2H-benzo[e][1,3]oxazin-6-ylmethoxy)-but-2-ynyl]-4,4-dimethyl-2,5-dioxo-imidazolidin-1-yl}-2-trifluoromethylbenzonitrile (7c). A solution of 130 mg (0.44 mmoles) of 2f was prepared in 2.0 mL of DMF and to this was added 18 mg (0.45 mmol) of sodium hydride as a 60% emulsion in oil. The mixture was stirred under an argon atmosphere at room temperature for 3 h. The resulting yellow solution was then added to a stirring solution of 150 mg (0.44 mmol) of 6c in 2.0 mL of DMF. The reaction was then stirred for 6 h at room temperature under Argon. The product was precipitated by drop-wise addition of the reaction mixture to 100 mL of saturated aqueous NaH₂PO₃. The pale yellow precipitate was then extracted into 300 mL of ethyl acetate which was collected and rotary evaporated at 40° C. to yield a yellow solution of crude product in DMF. Further rotary-evaporation at 50° C. and 100 μm Hg removed the DMF to give a viscous yellow oil. This residue was eluted from a silica gel flash column (30 cm×2 cm) in 10% hexanes/90% ethyl acetate. Removal of solvent by rotary-evaporation gave 200 mg (82%) of pure 7c as a pale yellow oil: ¹H NMR (500 MHz, CDCl₃) δ 1.64 (12H, s, sal-2-(CH₃)₂, nil-4-(CH₃)2), 4.16-4.19 (2H, m, OCH₂C), 4.29-4.33 (2H, m, CCH₂N), 4.53 (2H, s, Bn), 6.91 (1H, d, J=8 Hz, sal-8), 7.44 (1H, dd, J=8, 2 Hz, sal-7), 7.53 (1H, bs, NH), 7.87 (1H, d, sal-5), 7.21 (1H, d, J=8 Hz, nil-6), 8.00 (1H, dd, J=8, 2 Hz, nil-5), 8.15 (1H, d, J=2 Hz, nil-3); m/z 554.1757 [M+] (calculated for 554.1777).

5-{4-[3-(4-Cyano-3-trifluoromethyl-phenyl)-5,5-dimethyl-2,4-dioxo-imidazolidin-1-yl]-but-2-ynyloxymethyl}-2-hydroxy-benzamide (8c). A solution of 200 mg 7c (0.36 mmol) was prepared in 10 mL of 20% water/80% MeOH and a catalytic amount of p-toluenesulfonic acid was added. The reaction was refluxed under an argon atmosphere for 24 h at 90° C. The solvent was then removed by rotary-evaporation at 40° C. and the residue was transferred to a separatory funnel in 200 mL of ethyl acetate. This solution was washed 2× with 40 mL portions of saturated brine containing 5% NaHCO₃. Th organic layer was then collected, dried over anhydrous magnesium sulfate, and rotary evaporated at 40° C. to give a pale yellow oil. Elution from a silica gel flash column (30 cm×2 cm) in 5% hexanes/95% ethyl acetate followed by rotary-evaporation yielded a product of approximately 95% purity. Further purification by preparatory HPLC, eluting with 20 mM pH 4.0 TEAA buffer, yielded 148 mg (80%) of 8c as a clear colorless oil: ¹H NMR (500 MHz, CDCl₃) δ 1.63 (6H, s, nil-S-(CH₃)₂), 4.15 (2H, s, OCH₂C), 4.29 (2H, s, CCH₂N), 4.50 (2H, s, Bn), 5.90 (1H, bs, NH), 6.62 (1H, bs, NH), 6.96 (1H, d, J=9 Hz, sal-3), 7.37 (1H, dd, J=9, 2 Hz, sal-4), 7.46 (1H, d, J=2 Hz, sal-6), 7.92 (1H, d, J=8 Hz, nil-5), 7.97 (1H, dd, J=8, 2 Hz, nil-6), 8.11 (1H, d, J=2 Hz, nil-2), 12.30 (1H, s, 2-OH); m/z 514.1469 [M+] (calculated for 514.1464); anal. (C₂₅H₂₁F₃N₄O₅) C, H, N.

5-(2-Bromo-ethoxymethyl)-2-hydroxybenzamide (9). A solution of 150 mg (0.91 mmol) of 3 was prepared in 12 mL of 2-bromoethanol by heating a stirring mixture of the two to 55° C. under an argon atmosphere. The solution was then allowed to cool for 5 min at room temperature before 56 mg (0.46 mmol) of decaborane was added. Excessive evolution of H₂ was observed for 5 min, after which time the reaction was again heated to 55° C. After stirring for 4 h, the solvent was removed by rotary-evaporation at 40° C. and 100 μm Hg. The residue was dissolved in 8 mL of ethyl acetate and introduced to a silica gel flash column (30 cm×2 cm) packed in 25% hexanes/75% ethyl acetate. Elution with the same solvent system yielded 175 mg (70%) of 9 as a clear, colorless oil: ¹H NMR (500 MHz, (CD₃)₂CO) δ 3.55 (2H, t, J=6 Hz, OCH₂CH₂Br), 3.76 (2H, t, J=6 Hz, OCH₂CH₂Br), 4.47 (2H, s, Bn), 6.89 (1H, d, J=8 Hz, 3), 7.19 (1H, bs, NH), 7.44 (1H, dd, J=8, 2 Hz, 4), 7.81 (1H, d, J=2 Hz, 6), 7.97 (1H, bs, NH), 12.93 (1H, s, 2-OH); m/z 273.007 [M+] (calculated for 273.001).

4-[3-(4-Bromo-butyl)-4,4, dimethyl-2,5-dioxo-imidazolidin-1-yl]-2-trifluoromethyl-benzonitrile (10a). To a stirring solution of 306 mg (1.0 mmol) of 2f in 3.0 mL of DMF was added 49 mg (1.2 mmol) of sodium hydride (60% in oil). The resulting suspension was stirred at room temperature for 1.5 h at which time evolution of H₂ had ceased and a yellow solution persisted. To this solution was added 1.0 mL of 1,4-dibromobutane and the resulting reaction mixture was heated under an argon atmosphere to 60° C. for 0.5 hr. At this time, the reaction mixture was added drop-wise to 100 mL of saturated brine containing 5% NaH₂PO₄. A pale yellow precipitate was formed which was extracted into 250 mL of ethyl acetate. The organic layer was washed 2× with saturated brine, collected, and rotary evaporated at 40° C. to a yellow solution in DMF. Further rotary-evaporation at 50° C. and 100 μm Hg removed the DMF to yield a yellow oil. This crude product was dissolved in 10 mL of 50% hexanes/50% ethyl acetate and introduced to a silica gel flash column (30 cm×2 cm) packed in 75% hexanes/25% ethyl acetate. Elution with 75% hexanes/25% ethyl acetate followed by removal of solvent by rotary-evaporation at 40° C. gave 378 mg (85%) of 10a as a clear, colorless oil: ¹H NMR (500 MHz, CDCl₃) δ 1.54 (6H, s, 4-(CH₃)₂), 1.82-1.98 (2H, m, CH₂CH₂N), 1.82-1.98 (2H, m, BrCH₂CH₂), 3.36-3.42 (2H, m, CH₂CH₂N), 3.43-3.49 (2H, m, BrCH₂CH₂), 7.90 (1H, d, J=8 Hz, 6), 7.99 (1H, dd, J=8, 2 Hz, 5), 8.14 (1H, d, J=2 Hz, 3); m/z 431.0450 [M+] (calculated for 431.0456).

4-{3-[2-(2-Bromo-ethoxy)-ethyl]-4,4-dimethyl-2,5-dioxo-imidazoldin-1-yl}-2-trifluoromethyl-benzonitrile (10b). 10b was prepared as 10a in 82% yield: ¹H NMR (500 MHz, CDCl₃) δ 1.55 (6H, s, 4-(CH₃)₂), 3.47 (2H, m, OCH₂CH₂N), 3.56 (2H, m, OCH₂CH₂N), 3.76, (2H, m, BrCH₂CH₂O), 3.81 (2H, m, BrCH₂CH₂O), 7.91 (1H, d, J=9 Hz, 6), 8.00 (1H, dd, J=9, 2 Hz, 5), 8.14 (1H, d, J=2 Hz, 3); m/z 447.0405 [M+] (calculated for 447.0405).

4-[4,4-Dimethyl-2,5-dioxo-3-(4-piperazin-1-yl-butyl)-imidazolidin-1-yl]-2-trifluoromethyl-benzonitrile (11a). To a stirring solution of 365 mg (0.84 mmol) of 10a in 1.0 mL of THF was added 4.0 mL of THF containing 500 mg (5.8 mmol) of piperazine. The resulting solution was heated to 40° C. under an argon atmosphere for 2.5 h. At this time, the solvent was removed by rotary-evaporation at 40° C. and the residue was transferred to a separatory funnel in 100 mL of saturated brine containing 5% NaH₂PO₄. The aqueous solution was washed 3×with 50 mL portions of ethyl acetate and was then neutralized with NaHCO₃. The desired product was then extracted into 300 mL of ethyl acetate, collected, dried over anhydrous magnesium sulfate, and rotary evaporated at 40° C. to yield 160 mg (43%) of lla as a pale yellow oil: ¹H NMR (500 MHz, CDCl₃) δ 1.50 (6H, s, 4-(CH₃)₂), 1.48-1.58 (2H, m, (CH₂)₂NCH₂CH₂), 1.65-1.75 (2H, m, CH₂CH₂NCO), 2.33-2.39 (2H, m, (CH₂)₂NCH₂CH₂), 2.45 (4H, bs, (CH₂)₂NCH₂), 2.93 (4H, bs, NH(CH₂)₂), 3.30-3.38 (2H, m, CH₂NCO), 4.18 (1H, bs, NH), 7.88 (1H, d, J=8 Hz, 6), 7.97 (1H, dd, J=8, 2 Hz, 5), 8.11 (1H, d, J=2 Hz, 3); m/z 631.2847 [MH+] (calculated for 631.2856).

4-{4,4-Dimethyl-2,5-dioxo-3-[2-(2-piperazin-1-yl-ethoxy)-ethoxyl-imidazolidin-1-yl}-2-trifluoromethyl-benzonitrile (11b). 11b was prepared as 11a in 59% yield: ¹H NMR (500 MHz, CDCl₃) δ 1.48 (6H, s, 4-(CH₃)₂), 2.45-2.60 (4H, bm, (CH₂)₂NCH₂), 2.51-2.56 (2H, m, (CH₂)₂NCH₂), 2.86-2.98 (4H, bm, HN(CH₂)₂), 3.46-3.53 (2H, m, CH₂OCH₂), 3.55 (2H, t, J=6 Hz, CH₂NCO), 3.61-3.66 (2H, m, CH₂OCH₂), 5.20 (1H, bs, NH), 7.87 (1H, d, J=8 Hz, 6), 7.95 (1H, dd, J=8, 2 Hz, 5), 8.09 (1H, d, J=2 Hz, 3); m/z 452.1897 [M−H] (calculated for 452.1909).

5-[2-(4-{4-[3-(4-Cyano-3-trifluoromethyl-phenyl)-5,5-dimethyl-2,4-dioxo-imidazolidin-1-yl]-butyl}-piperazin-1-yl)-ethoxymethyl]-2-hydroxy-benzamide (12a). To a stirring solution of 120 mg (0.27 mmol) of 11a and 80 mg (0.29 mmol) of 9 in 2.0 mL of THF was added 100 μl (0.72 mmol) of triethylamine. The resulting solution was refluxed under an argon atmosphere for 20 h, at which time the solvent was removed by rotary-evaporation at 40° C. The residue was dissolved in 250 mL of ethyl acetate and transferred to a separatory funnel where it was washed 3×with 40 mL portions of saturated brine containing 5% NaH₂PO₄. The organic layer was collected, dried over anhydrous magnesium sulfate, and rotary evaporated free of solvent at 40° C. to give a pale brown oil. This residue was dissolved in 10 mL of ethyl acetate and introduced to a silica gel flash column (20 cm×2 cm) packed in the 100% ethyl acetate. Elution with 78% ethyl acetate/20% methanol/2% triethylamine followed by removal of the solvent by rotary-evaporation at 40° C. gave a pale golden oil. Further purification by preparatory HPLC using 20 mM pH 4.0 TEAA buffer was required and yielded 121 mg (71%) of 12a as a clear, colorless oil: ¹H NMR (500 MHz, CDCl₃) 1.49-1.60 (2H, m, (CH₂)₂NCH₂CH₂), δ 1.52 (6H, s, 5-(CH₃)₂), 1.67-1.76 (2H, m, CH₂CH₂NCO), 2.37 (2H, t, J=7 Hz, (CH₂)₂NCH₂CH₂), 2.49 (8H, bs, N(CH₂CH)₂N), 2.61 (2H, t, J=6 Hz, BnOCH₂CH₂N) 3.36 (2H, t, J=8 Hz, CH₂NCO), 3.56 (2H, t, J=6 Hz, BnOCH₂),4.42 (2H, s, Bn), 6.23 (1H, bs, NH), 6.87 (1H, bs, NH), 6.91 (1H, d, J=8 Hz, sal-3), 7.32 (1H, d, J=8 Hz, sal-4), 7.48 (1H, s, sal-6), 7.90 (1H, d, J=9 Hz, nil-5), 7.99 (1H, dd, J=9, 2 Hz, nil-6), 8.14 (1H, d, J=2 Hz, nil-2); m/z 631.2833 [MH+] (calculated for 631.2856).

5-{2-[4-(2-{2-[3-(4-Cyano-3-trifluoromethyl-phenyl)-5,5-dimethyl-2,4-imidazolidin-1-yl]-ethoxy}-ethyl-piperazine-1-yl]-ethoxymethyl{-2-hydroxy-benzamide (12b). 12b was prepared as 12a in 83% yield: ¹H NMR (500 MHz, CDCl₃) δ 1.53 (6H, s, 5—(CH₃)₂), 2.45-2.68 (8H, bm, N(CH₂CH₂)₂N), 2.54-2.61 (2H, m, (CH₂)₂NCH₂CH₂O), 2.63-2.90 (2H, m, BnOCH₂CH₂N), 3.50-3.63 (6H, m, BnOCH₂, CH₂OCH₂CH₂NCO), 3.65-3.71 (2H, m, OCH₂CH₂NCO), 4.43 (2H, s, Bn), 6.13 (1H, bs, NH), 6.92 (1H, d, J=8 Hz, sal-3), 7.31 (1H, dd, J=8, 2 Hz, sal-4), 7.57 (1H, d, J=2 Hz, sal-6), 7.90 (1H, d, J=8 Hz, nil-5), 8.00 (1H, dd, J=8, 2 Hz, nil-6), 8.14 (1H, d, J=2 Hz, nil-2); m/z 647.2816 [MH+] (calculated for 647.2805).

N-(5-{4-[3-(4-Cyano-3-trifluoromethyl-phenyl)-5,5-dimethyl-2,4-dioxo-imidazolidin-1-yl]-but-2-ynyloxymethyl)-2-hydroxy-benzamidomethyl)-doxorubicin (13). To a stirring solution of 20 mg of 8c (0.04 mmol) in 2.0 mL of DMF was added 10 μL of a 37% formalin solution (0.13 mmol). The reaction was stirred in a screw top vial for 15 min at 55° C., at which time 20 mg (0.03 mmol) of doxorubicin hydrochloride was added to form a red suspension which was stirred at 55° C. After 15 min, a clear red solution had formed and the reaction was removed from the heat. Transfer of the solution to a 250 mL round bottom flask, followed by rotary evaporation of the solvent at 55° C. and 50 μm Hg gave a red film which was readily dissolved in 20 mL of methanol containing 30% of 20 mM pH 2.9 1% TFA. After 10 min at room temperature, the methanol was removed by rotary evaporation at 30° C. and the resulting aqueous suspension was diluted to 100 mL with saturated brine and transferred to a separatory funnel. Extraction into 50 mL of chloroform followed by addition of 1 mL of glacial acetic acid and rotary evaporation at 30° C. gave a red film. The product was then dissolved in 1-2 mL of methanol and filtered through a 0.45 μm Spin-X centrifuge filter. Purification was achieved by preparative HPLC using a pH 3.5 TEAA buffer as the aqueous eluent. Pure material was collected into a test tube (100 mm×10 mm) containing 0.5 mL of 1.0 M HCl. Acetonitrile was removed by rotary evaporation at 30° C. to yield an aqueous suspension of the pure product which was diluted to 50 mL with saturated brine and transferred to a separatory funnel. Extraction into 50 mL of chloroform followed by addition of 1 mL of glacial acetic acid and rotary evaporation at 30° C. gave 23 mg (60%) of 13 as the acetate salt: ¹H NMR (500 MHz, (CD₃)₂CO) δ 1.32 (3H, d, J=6 Hz, dox-5′-CH₃), 1.62 (6H, s, 5-(CH₃)₂), 2.15-2.30 (3H m, 2(dox-2′), dox-8), 2.42 (1H, d, J=14 Hz, dox-8), 2.93 (1H, d, J=18 Hz, dox-10), 3.12 (1H, J=18 Hz, dox-10), 3.85-4.0 (1H, bm, dox-3′), 4.06 (3H, s, dox-4-OCH₃), 4.12 (3H, s, dox-9OH, CCH₂NCO), 4.38 (5H, s, dox-5′, Bn, BnOCH₂C), 4.62-4.78 (2H, m, dox-14), 4.91 (1H, bs, NCH₂N), 5.03 (1H, bs, NCH₂N), 5.21 (1H, s, dox-7), 5.56 (1H, s, dox-1′), 6.71 (1H, d, J=8 Hz, sal-3), 7.29 (1H, d, J=8 Hz, sal-4), 7.63 (1H, d, J=8 Hz, dox-3), 7.74 (1H, bs, sal-6), 7.90 (1H, t, J=8 Hz, dox-2), 7.96 (1H, d, J=8 Hz, dox-1), 8.14 (1H, d, J=8 Hz, nil-5), 8.20 (1H, d, J=9 Hz, nil-6), 8.25 (1H, s, nil-2), 10.32 (1H, bs, NH), 11.82 (1H, bs, sal-2-OH), 13.26 (1H, s, dox-6/11-OH), 14.18 (1H, s, dox-6111-OH); m/z 1092.3101 [MNa+] (calculated for 1092.3097).

Radioligand Competition AR Binding Assay: PC3/AR or PC3/neo cells were grown in RPMI 1640 medium to approximately 80% confluency in five Nunc T-175 flasks. Growth medium in each flask was then replaced with 50 mL of phenol red-free RPMI 1640 supplemented with 10% dextran-coated charcoal-stripped FBS, and the cells were grown for an additional 18-22 h. Two hours prior to harvesting, the growth medium was again replaced with fresh phenol red-free, charcoal-stripped RPMI. The cells were then washed with 10 mL of Hank's balanced salt solution and dissociated with trypsin. Trypsin was quenched with phenol red-free, charcoal stripped RPMI and the combined cells from each flask were centrifuged in a 50 mL conical tube at 100×g for 5 min. The cells were then resuspended in 50 mL of phenol red-free, charcoal-stripped RPMI and counted at this concentration. Centrifugation at 100×g gave approximately 1 mL of cells which were resuspended in 5 mL of 4° C. lysis buffer (10 mM Tris, 1.5 mM EDTA, 0.5 mM DTT, 10 mM NaMoO₄, 1.0 mM PMSF, 10% v/v glycerol) supplemented immediately before use with Complete-mini protease inhibitor cocktail. Cells were lysed via sonication at 4° C. with a microtip, set at maximum power, for 10 cycles of 6 s on and 24 s off. The cytosolic fraction of the lysate was isolated by ultracentrifugation at 4° C. and 225,000×g for 45 min. The centrifuged samples were dispensed into 100 μL aliquots and stored at -78° C. until used. Total protein was quantified either in fresh or frozen aliquots by the Sigma BSA micro protein determination method according to the prescribed protocol.

Aliquots of cell lysate were used fresh or thawed at 4° C. Stock solutions of 100× working concentration of the test ligands, ³H-Mibolerone and unlabled Mibolerone were prepared in DMSO and subsequently diluted to 10× in lysis buffer. Concentrations of test compounds were determined spectrophotometricly in DMSO by either absorbance at 310 nm for salicylamide containing molecules (□₃₁₀=3580 L/(mol×cm); as determined from a Beer-Lambert plot described by varying concentrations of 8a), 264 nm for 2f (□₂₆₄=13000 L/(mol×cm)), or 276 nm for nilutamide (□₂₇₆=4620 L/(mol×cm)). Aliquots of cell lysate were complemented with 10 μL of 10× ligand solutions and 10 μL of the 10× ³H-Mibolerone solution to yield concentrations of 1, 10, 100, and 1000 nM test compound and 1 nM ³H-Mibolerone. Each reaction was prepared in duplicate to yield 8 total test assays. Duplicate positive controls, consisting of 10 μL of lysis buffer in place of a test ligand (total radioligand binding), and negative controls, consisting of 1000 nM unlabled Mibolerone (nonspecific binding), each in the presence of 1 nM ³H-Mibolerone, were prepared. The reactions were gently mixed and briefly centrifuged before incubating at 4° C. for 30 min. After incubation was complete, 100 μL of each reaction was introduced to 400 μL of ice cold hydroxyapatite (HA), as a 60% suspension in pH 7.4 Tris buffer, on a 0.45 μm nylon filter in a Spin-X centrifuge tube. Upon addition of the reaction solution, the tubes were closed, briefly vortexed, and allowed to incubate on ice for 12 min with vortexing every 3-5 min. The HA suspensions were then centrifuged at 1200×g for 10 min. The filtrate was discarded and the dry pellet was resuspended in 400 μL of pH 7.3 20 mM Tris wash buffer containing 0.1% Triton-X100. Following seven rounds of resuspension and subsequent centrifugation, the final filtrate was discarded and the dry pellet was centrifuged for an additional 15 min. The pellet and filter bucket for each sample were then transferred to 20 mL scintillation vials and 4 mL of scintillation cocktail was added to each. Vortexing for 30 s thoroughly mixed the pellet with the scintillation liquid before counting. Each sample was counted for 5 repetitions of 3 min counts. This counting protocol was then repeated two additional times to assure precision. Specific binding for each test concentration was determined by subtracting the nonspecific binding control from the total binding determined for each concentration. Comparison to the specific binding for the positive control, in which no competing ligand was incubated with the ³H-Mibolerone, yielded the percent of ³H-Mibolerone displaced by a given concentration of test ligand. The IC₅₀ values for each test ligand were calculated by Logit-log (pseudo-Hill) analysis.

### Example 3

Studies of Targeting and Intracellular Trafficking of an Anti-Androgen-Doxorubicin-Formaldehyde Conjugate in PC-3 Prostate Cancer Cells Bearing Androgen Receptor-GFP Chimera

While immunohistochemical staining of various normal tissues indicates only low level expression outside of the reproductive tract, the androgen receptor has been identified in a wide array of human tumors in both male and female patients. Carcinomas of the breast, ovary, esophagus, lung and prostate have all been shown to express the AR at various levels. The expression or overexpression of AR in the majority of human prostate tumors also suggests that it may be required for growth in prostate cancer (CaP).

The AR exists primarily as a cytosolic receptor in complex with several heat-shock proteins (hsp70, hsp90, and hsp56-59). Ligand binding leads to dissociation of the heat-shock proteins, homodimerization, and translocation into the nucleus where the dimeric receptor recognizes hormone responsive elements and various components of the transcription machinery. The receptor is often over-expressed in hormone refractory prostate cancer and is also known to acquire mutations that lead to promiscuous binding of various non-androgen ligands. Several groups have successfully ligated the cDNA of the androgen receptor to that of a modified green fluorescent protein (GFP) in a construct which encodes the chimeric AR-GFP product. In the absence of ligand, AR-GFP has been shown to localize in the cytoplasm of transfected cells. However, upon binding of dihydrotestosterone (DHT), or other appropriate agonists, the receptor is observed to translocate into the nucleus. Antagonists, on the other hand, vary in their ability to cause migration of the receptor into the nucleus of treated cells. While some do effect a change in cellular localization of the fluorescent receptor, others serve to prevent the nuclear translocation through inhibition of DHT binding. The easily qualified response to receptor binding has been successfully used to ascertain the effect of various agonists and antagonists on cellular localization of the AR. This example describes the intracellular response of the AR-GFP receptor in PC3 cells upon exposure to a series of AR targeted derivatives of salicylamide, the amide moiety of the N-Mannich base doxsaliform. Also described is the action of the doxorubicin N-Marnich base product of the present invention formed from the most effective targeting compound of the tested series.

FIG. 15 shows the structures of the different AR ligands tested and Table 4 shows the IC₅₀ and relative binding affinities of these compounds. After establishing that the targeting groups were capable of binding specifically to the AR with reasonable affinity, the ability of the binding event to result in nuclear delivery of the constructs was investigated. PC3 cells were, therefore, grown in 6 well plates and transiently transfected with a plasmid containing the AR-GFP construct obtained from Dr. Arun Roy (UTHSC; San Antonio, Tex.). After 18 h incubation, the cell culture media was removed and replaced with RPMI 1640 supplemented with 10% fetal bovine serum (FBS) which had been stripped of steroids (and other components) with dextran coated charcoal. Growth in the stripped media for 18 h allowed for predominantly cytosolic localization of the AR-GFP receptor and also served to remove steroids which can potentially interfere with the binding of the test compounds. The transfected cells were then treated with various targeting groups and controls in the presence or absence of Mibolerone. Mibolerone causes nuclear translocation of the AR-GFP receptor in approximately 30 min at concentrations as low as 1.0 nM. Digital imaging of the cells allows for facile analysis of the activity of the various ligands.

TABLE 4
IC₅₀ and relative binding affinity values determined
from competitive binding for the human AR of the various
test ligands against 1.0 nM ³H-Mibolerone in
PC3/AR cell lysate at 4° C.

| Test Compound | IC₅₀ | | RBA |
|---|---|---|---|
| 2a | 9 | nM | 100 |
| 2b | 6 | nM | 150 |
| 4 | 77 | nM | 13 |
| 5 | 332 | nM | 3 |
| 6a | 49 | nM | 18 |
| 7 | >1000 | nM | <1 |
| 8 | 346 | nM | 3 |
| 9 | 90 | nM | 10 |
| 10 | 63 | nM | 14 |
| flutamide | 154 | nM | 6 |
| salicylamide | >>1000 | nM | <<1 |

Compound 2b was found to bind the AR and induce partial translocation as manifest by a clear morphological change and redistribution of fluorescence . It must be noted that the effect of 2b on cells is somewhat ambiguous as the morphological change and nearly homogenous distribution of fluorescence could be indicative of simple cytosolic redistribution of AR-GFP leading to nuclear masking. This masking effect, in which a non-fluorescing nucleus would be hidden by excess cytoplasmic GFP in the line of sight, was not observed in any other cells treated with inactive ligands. However, the absence of a clearly discemable nucleus in cells treated with 2b leaves open this possibility. In any event, it is not clear why the seemingly subtle substitution of the cyano group of 2b for the nitro moiety of nilutamide leads to a compound that is capable of initiating translocation. Conformational changes induced by ligand binding are known to be required for migration of the AR into the nucleus. The varying activities of structurally similar AR antagonists suggests that antiandrogenic activity is manifested at different stages of AR activation. While hydroxyflutamide 3b and the structurally similar antiandrogen bicalutamide 3c do induce the appropriate conformational changes to allow for nuclear translocation and, therefore, must block AR activity at some downstream event, nilutamide apparently acts simply by blocking steroid binding. The structural similarity of the nonsteroidal antiandrogens, however, suggests that small changes to the nilutamide core may be expected to impart the necessary receptor interactions to induce a conformational change that will lead to nuclear localization of the receptor.

Treatment of AR-GFP expressing cells with the targeting constructs 5, 7, or 8 does not instigate translocation. These ligands, which were the least effective at displacing ³H-Mibolerone in the receptor binding assay (Table 4), are also not capable of inhibiting the action of 1.0 nM Mibolerone on AR-GFP. Of interest is the result obtained from treatment of the cells with 4. While this compound was not able to instigate translocation of AR-GFP at concentrations up to 1.0 μM, it did serve to partially inhibit the activity of 1.0 nM Mibolerone on treated cells.

The most encouraging results obtained for any of the tested compounds came from 6a. Treatment of AR-GFP expressing cells with the butyne tethered product at a concentration of 1.0 nM successfully caused nuclear localization of the receptor. Although the binding efficiency of the antiandrogens is not directly related to their ability to initiate translocation, it was determined that, in the tested series, the compound which is most effective at competing for AR binding with ³H-Mibolerone is also capable of initiating migration of the AR-GFP receptor to the nucleus. These findings qualify 6a as a lead compound for further development as a delivery vehicle for the doxorubicin prodrug 1a.

Following the identification of a viable targeting group, evaluation of a targeted derivative 9 of the prodrug 1a via the AR binding assay was evaluated, just as the targeting groups had been evaluated. Since the AR-GFP translocation assay must be run at 37° C., and the N-Mannich base 9 readily hydrolyzes to regenerate 6a, the O-butyryloxymethylene protected 10 was prepared for use as a stable derivative.

Acyloxymethylation of the phenolic moiety of salicylamide leads to a stable N-Mannich base product upon reaction with doxorubicin. Preparation of this derivative allows for study of the intact prodrug without concern for the activity of 6a, which is released upon partial hydrolysis of 9 and can be expected to compete for AR binding. Competitive binding of both 9 and 10 has been confirmed using the cell free assay. Both compounds demonstrate binding affinities similar to that of 6a (Table 4), and both have been shown to be stable under the assay conditions (30 min incubation at 4° C.).

The targeted prodrug 9 was also evaluated in cytotoxicity experiments employing the androgen receptor expressing PC3/AR and control PC3/neo cell lines. PC3/AR and PC3/neo cells, provided by Dr. Kerry L. Burnstein, University of Miami, Miami, Fla., were treated for 3 min, 10 min and 20 min with either 500 nM doxorubicin or 500 nM 9. The short dosing periods were chosen to capitalize on any binding of the prodrug to the AR which would serve to concentrate it in the cells. Earlier experiments employing a 4 h treatment had shown no difference in effect between the targeted prodrug and doxorubicin due to the constant exposure of the cells to cytotoxin released from hydrolysis of the N-Mannich base. Rapid removal of doxorubicin may leave little drug in the cells, while binding of 9 to the AR would serve to retain the prodrug after removal of the treatment solution. No difference was observed, however, at any treatment time. Several factors may account for this, including relatively poor or excessively slow binding, insufficient cytotoxicity of doxorubicin, or equally extensive uptake of both the targeted and untargeted drug by cultured cells, independent of AR binding. To address the shortcomings of this construct in cultured cells, the cellular distribution of doxorubicin, doxsaliform, and the targeted prodrug was also investigated.

The fluorescence of doxorubicin can be monitored in order to determine the rate of uptake and intracellular distribution of the anthraquinone fluorophore. Curiously, the fluorescence of doxorubicin is partially quenched by the introduction of salicylamide in the N-Mannich base construct as in 1a and 9. However, modification of the phenolic moiety of salicylamide with the butyryloxymethylene protecting group serves to fully restore fluorescence in 10. These interesting observations allow for the tracking of both the targeted prodrug 10, as well as the intracellular distribution of doxorubicin, which fluoresces, once it is released from 9 in which fluorescence is greatly attenuated.

The O-acetyloxymethylene derivative of doxsaliform 1b was prepared to allow for comparison of the targeted and untargeted prodrugs. The initial distribution of 10 was predominantly cytosolic, with noticeable accumulation in several focal points throughout both PC3/AR and PC3/neo cells. Similar localization was observed for 1b upon initial treatment with a 500 nM solution of the prodrug. However, fluorescence from 1b was seen to accumulate, at least to some extent over time (>3 h), in the nuclei and in some perinuclear depots of treated cells. The origin of this nuclear fluorescence is yet uncertain since any hydrolysis of 1b (which is perhaps dependant on any intracellular esterase activity) releases doxorubicin, which shows its own pattern of distribution. Faint fluorescence in the nuclei of cells treated with 1b may be due to limited accumulation of 1b or complete accumulation of small amounts of liberated doxorubicin, which is seen to rapidly localize to the nucleus. It should be noted that similar 3 h treatment of the same cell lines with 1a, in which hydrolysis of the N-Mannich base is not retarded, leads to exclusive nuclear accumulation of fluorescence. Since the half-life of hydrolysis for 1a is approximately 57 min, this nuclear fluorescnce at 3 h is attributed entirely to liberated doxorubicin.

The variable intensity of fluorescence observed due to accumulation of free doxorubicin or the various prodrugs, as well as the inherent instability of the N-Mannich bases, makes continuous tracking of these constructs over time a difficult task. What is more useful is the comparison of the deposition of the targeted prodrugs 9 and 10 with the deposition of doxorubicin upon initial dosing and after sufficient time for release of the N-Mannich base trigger. After 3 h treatment with the active targeted prodrug 9, the fluorescence was primarily localized to the nucleus. The fluorescence is attributed to doxorubicin, which accumulates after hydrolysis of the prodrug over the 3 h treatment time. These results together with those obtained from following the fluorescence of 10, which remains primarily cytosolic over time, suggest that the prodrug 9 releases doxorubicin in the cytosol of treated cells, and not in the nucleus. In addition, the similar distribution of fluorescence observed in both AR expressing PC3/AR and non-expressing PC3/neo cells indicates that the bulk of the prodrug retained by the cells is not associated with the AR. This further supports the proposal that measurements of cytotoxicity in cell culture are not sufficient to determine the targeting ability of 9, since the prodrug readily accumulates in treated cells, regardless of AR content. Whether in vivo targeting of the prodrug can overcome this non-AR specific accumulation is yet to be determined.

Treatment of AR-GFP expressing PC3 cells with 1.0 μM 10 indicated that, unlike 6a, the full prodrug 10 did not instigate translocation into the nucleus. However, the presence of 1.0 μM 10 did serve to inhibit the action of 1.0 nM Mibolerone on the AR-GFP receptor. The exact cause for the loss of activity upon introduction of the doxorubicin N-Mannich base is not clear. It is possible that the tether portion of the targeting group is too short, allowing for interactions between doxorubicin and the receptor, which serve to preclude the necessary conformational change of the AR. It is possible that the introduction of the butyryloxymethylene protecting group to 6a is responsible for the loss of activity. This construct 6b, however, was found to act in much the same manner as 6a, causing nuclear translocation of the AR upon binding. Unfortunately, the assay can not be used to evaluate the unprotected construct 9, because of the inherent instability of the prodrug. It should be noted, however, that there is a clear distinction between the activity of an efficient AR binder like 10 and a lower affinity ligand such as 5. Compound 10 inhibits the action of Mibolerone, while 5 does not.

This data demonstrates that nonsteroidal antiandrogens modified with an appropriate tether retain reasonable binding affinity for the AR and initiate nuclear translocation of the receptor. It further shows that a prodrug of doxorubicin can be successfully targeted to cells via specific interaction with the AR.

Experimental Section: ¹H-NMR spectra were acquired with a 500 MHz spectrometer. Unambiguous NMR assignments for the protons of the nilutamide, salicylamide, and doxorubicin portions of 10 are designated by “nil”, “sal”, or “dox” respectively. Mass spectral data were acquired on a mass spectrometer by liquid SIMS (LSIMS) ionization with a polyethylene glycol (PEG) internal standard for [MH+] data. Mass spectral data [MNa+] for compound 10 were collected by Dr. Chris Hadad (Ohio State University; Columbus, Ohio) with a Fourier Transform mass spectrometer. UV-vis spectrometry was performed with a diode array spectrophotometer and workstation. Fluorescence microscopy was conducted with a stereo microscope equipped with an ebq 100 mercury lamp power source. Fluorescence of doxorubicin and derivatives was monitored at wavelengths above 590 nm, with excitation at 540±20 nm. DAPI fluorescence was observed at wavelengths above 425 nm with excitation at 360±20 nm. Green fluorescent protein was observed at wavelengths above 515 nm with excitation at 470±20 nm. HPLC analyses were performed with a liquid chromatograph equipped with a diode array UV-vis detector and workstation; chromatographies were performed with a 5 μm reverse phase C₁₈ microbore column, 2.1 mm i.d.×100 mm, eluting at 0.5 mL/min, monitoring at 260, 310, and 480 nm. Acceptable analytical resolution was achieved with gradients of acetonitrile and triethylammonium acetate (Et₃NHOAc; TEAA), prepared as 20 mM triethylamine adjusted to pH 6.0 with acetic acid. The method employed for all analytical chromatography was as follows: A=CH₃CN, B=pH 6.0 buffer; A:B, 0:100 to 70:30 at 10 min, isocratic until 12 min, 0:100 at 15 min. For preparative HPLC, a 5 μm spherical particle C₁₈ semi-preparative column was employed, 10 mm×25 cm with a 10 mm×5 cm guard column, eluting at 3.0 mL/min, monitoring at 260, 310, and 480 nm. Adequate preparative separation was achieved using the following method: A=CH₃CN, B=1% aqueous HCl; A:B, 50:50 to 55:45 at 20 min, isocratic until 25 min, 70:30 at 30 min, isocratic until 35 min, 50:50 at 40 min. Water was distilled and purified with a Millipore Q-UF Plus purification system to 18 Mohm-cm. Flash silica gel had a particle size of 32-63 μm and a pore size of 60 Å.

The pEGFP-C2 rcAR plasmid was a gift from Dr. Arun Roy (UTHSC; San Antonio, Tex.). All cell lines were maintained in vitro by serial culture in RPMI 1640 media supplemented with either 10% fetal bovine serum or dextran-charcoal stripped (delipidated) fetal calf serum as indicated, L-glutamine (2 mM), HEPES buffer (10 mM), penicillin (100 units/mL), and streptomycin (100 μg/mL). Cells were maintained at 37° C. in a humidified atmosphere of 5% CO₂ and 95% air.

Syntheses: 5-{4-[3-(4-Cyano-3-trifluoromethyl-phenyl)-5,5-dimethyl-2,4-dioxo-imidazolidin-[yl]-but-2-ynyloxymethyl}-2-butyryloxymethoxy-benzamide (6b). A mixture of 40 mg (0.078 mmol) of 6a and 21 mg (0.15 mmol) of potassium carbonate was stirred for 30 min at room temperature in 5 mL of acetone. In a separate flask, 16 mg (0.12 mmol) of chloromethyl butyrate and 22 mg (0.13 mmol) of potassium iodide were stirred in 5 mL of acetone at room temperature. The two mixtures were then combined and refluxed for 4 h. The reaction was stopped by cooling to room temperature and filtering through a glass frit. The collected liquid was rotary evaporated at 30° C. and the residue was dissolved in 100 mL of ethyl acetate. After 3× washes with 50 mL saturated brine, the organic layer was collected, dried over anhydrous magnesium sulfate, and concentrated by rotary evaporation at 40° C. The washed product was then dissolved in 3 mL of ethyl acetate and introduced to a silica gel flash column (2 cm×30 cm) packed in 50% hexanes/50% ethyl acetate. The desired product was eluted with 25% hexanes/75% ethyl acetate. Concentration by rotary evaporation at 30° C. yielded approximately 80% conversion. The semi-pure product was characterized by the following spectral properties and was used without further purification for the preparation of 10; ¹H NMR (500 MHz, CDCl₃) δ 0.95 (3H, t, J=7 Hz, Bu-4), 1.63-1.71 (2H, m, Bu-3), 1.67 (6H, s, 5-CH₃'s), 2.37 (2H, t, J=8 Hz, Bu-2), 4.20 (2H, s, tether-3), 4.34 (2H, (2H, s, tether-1), 5.91 (2H, s, OCH₂O), 6.06 (1H, bs, NH), 7.18 (1H, d, J=8 Hz, 3), 7.52 (1H, dd, J=8, 2 Hz, 4), 7.57 (1H, bs, NH), 7.95 (1H, d, J=8 Hz, 5), 8.03 (1H, dd, J=8, 2 Hz, 6), 8.18 (2H, s, 6/2); mass spectrum, m/z 615.2064 [MH+] (calculated for 615.2067).

N-(5-{4-[3-(4-Cyano-3-trifluoromethyl-phenyl)-5,5-dimethyl-2,4-dioxo-imidazolidin-1-yl]-but-2-ynyloxymethyl}-2-butyryloxymethoxybenzamidomethyl)-doxorubicin (10). To a stirring solution of 20 mg of 6b (0.033 mmol) in 2.0 mL of DMF was added 10 μL of a 37% formalin solution (0.13 mmol). The reaction was stirred in a screw top vial for 15 min at 55° C., at which time 20 mg (0.034 mmol) of doxorubicin hydrochloride was added to form a red suspension which was stirred at 55° C. After 15 min, a clear red solution had formed and the reaction was removed from the heat. Transfer of the solution to a 250 mL round bottom flask, followed by rotary evaporation at 55° C. and 50 μm Hg gave a red film which was readily dissolved in 20 mL of methanol containing 30% of 20 mM pH 2.9 1% TFA. After 10 min at room temperature, the methanol was removed by rotary evaporation at 30° C. and the resulting aqueous suspension was diluted to 100 mL with saturated brine and transferred to a separatory funnel. Extraction into 50 mL of chloroform followed by rotary evaporation at 30° C. gave a red film. The product was then dissolved in 1-2 mL of methanol and filtered through a 0.45 μm Spin-X centrifuge filter. Purification was achieved by preparative HPLC using a pH 3.5 TEAA buffer as the aqueous eluent. Pure material was collected into a test tube (100 mm×10 mm) containing 0.5 mL of 1.0 M HCl . Acetonitrile was removed by rotary evaporation at 30° C. to yield an aqueous suspension of the pure product which was diluted to 50 mL with saturated brine and transferred to a separatory funnel. Extraction into 50 mL of chloroform followed by rotary evaporation at 30° C. gave 30 mg (76%) of 10 as the free base; ¹H NMR (500 MHz, (CDCl₃) δ 0.85 (3H, t, J=7 Hz, OOCCH₂CH₂CH₃), 1.40 (3H, d, J=7 Hz, dox-5′-CH₃), 1.49-1.61 (2H, m, OOCCH₂CH₂CH₃), 1.60-1.68 (1H, m, dox-2′), 1.63 (6H, s, nil-5-(CH₃)₂), 1.84 (1H, td, J=13, 4 Hz, dox-2′), 2.15 (1H, dd, J=4, 15 Hz, dox-8), 2.20-2.30 (2H, m, OOCCH₂CH₂CH₃), 2.33-2.39 (1H, dt, J=15, 2, 15 Hz, dox-8), 3.03 (1H, bs, dox-14-OH), 3.04 (1H, d, J=19 Hz, dox-10), 3.06-3.13 (1H, bm, dox-3′), 3.22 (1H, dd, J=2, 19 Hz, dox-10), 3.75 (1H, bs, dox-4′), 4.02 (1H, q, J=6 Hz, dox-5′), 4.09 (3H, s, dox-4-OCH₃), 4.15 (2H, t, J=2 Hz, CCH₂NCO), 4.30 (2H, t, J=2 Hz, BnOCH₂C), 4.35 (2H, d, J=6 Hz, NCH₂N), 4.51 (2H, s, bn CH₂), 4.67 (1H, d, J=21 Hz, 14), 4.69 (1H, d, J=21 Hz, 14), 4.81 (1H, s, 9-OH), 5.35 (1H, m, dox-7), 5.56 (1H, d, J=4 Hz, dox-1′), 5.68 (1H, d, J=7 Hz, OCH₂O), 5.78 (1H, d, J=7 Hz, OCH₂O), 7.08 (1H, d, J=8 Hz, sal-3), 7.41 (1H, dd, J=1, 8 Hz, dox-3), 7.42 (1H, dd, J=2, 8 Hz, sal-4), 7.80 (1H, t, J=8 Hz, dox-2), 7.93 (1H, d, J=9 Hz, nil-5), 8.01 (1H, dd, J=2, 9 Hz, nil-6), 8.03 (1H, dd, J=1, 8 Hz, dox-1), 8.05 (1H, d, J=2, sal-6), 8.15 (1H, d, J=2 Hz, nil-2), 7.96-8.06 (1H, bm, NH), 13.27 (1H, s, dox-6/11-OH), 13.97 (1H, s, dox-6/11-OH); mass spectrum, m/z 1192.3665 [MNa+] (calculated for 1192.3621).

AR-GFP Localization by Fluorescence Microscopy: PC3 cells were dissociated with trypsin EDTA, counted, and suspended in growth media to a concentration of 3.5×10⁴ cells/mL. This cell suspension was dispensed in 2 mL aliquots into 6-well tissue culture plates. Plates were then incubated for 12 h at 37° C. in a humidified atmosphere of 5% CO₂ and 95% air. A transfection cocktail was prepared by adding 8 μL of FUGENE 6 transfection reagent to sterile sample tubes containing 100 μL of serum free, phenol red-free RPMI 1640 medium for each well to be transfected. To each solution was added 2 μL of a 800 μg/mL solution of the pEGFP-C2 rcAR plasmid in Millipore water. After gentle mixing, the transfection cocktail was allowed to incubate at room temperature for 40 min. At this time, 100 μL of transfection cocktail was added to each well of 12 h old cells. The cells were then incubated for 24 h at 37° C. in a humidified atmosphere of 5% C0₂ and 95% air. The transfection medium was then removed and the cells were washed with 1 mL of FBS free, phenol red-free RPMI 1640 growth medium. Following the wash, 1 mL of phenol red-free RPMI 1640 medium supplemented with dextran-coated charcoal-stripped FBS was added to each well and the cells were incubated for an additional 24 h. The growth medium was again replaced with 1 mL of phenol red-free RPMI supplemented with dextran-coated charcoal-stripped FBS. Candidate AR-GFP expressing cells in each well were identified and marked before an appropriate concentrations of the test compounds were added in 10 μL of sterile DMSO. The treated cells were then incubated for the necessary time at 37° C. before marked AR-GFP expressing cells were observed for drug activity. Nuclear staining with DAPI was carried out by 15 min treatment with 1 mL of a 1% gluteraldahyde solution, followed by 15 min treatment with 1 mL of 0.2 μg/mL DAPI in phenol red-free RPMI 1640. The DAPI solution was then replaced with 1 mL of phenol red-free RPMI 1640 and fluorescence over 425 nm was observed at 400× with excitation at 360±20 nm.

Doxorubicin Localization by Fluorescence Microscopy: Cells were dissociated with trypsin EDTA, counted, and suspended in growth media to a concentration of 3.5×10⁴ cells/mL. This cell suspension was dispensed in 2 mL aliquots into 6-well tissue culture plates. Plates were then incubated for 36 h at 37° C. in a humidified atmosphere of 5% CO₂ and 95% air. The medium was replaced with 1 mL of phenol red-free RPMI 1640 growth medium supplemented with 10% dextran-coated charcoal stripped FBS prior to addition of the test compound. The appropriate compound was dissolved in DMSO and the concentration was adjusted to 50-200 μM by measuring the solution absorbance at 480 nm. Addition of 10 μL of the appropriate doxorubicin or prodrug solution was followed by incubation at 37° C. as indicated. The drug solution in individual wells was removed at the appropriate time and the cells were washed with 1 mL of the phenol red-free growth medium. The washed cells were then supplemented with 1 mL of phenol-red free growth medium for imaging.

Radioligand Competition AR Binding Assay: PC3/AR or PC3/neo cells were grown in RPMI 1640 medium to approximately 80% confluency in five Nunc T-175 flasks. Growth medium in each flask was then replaced with 50 mL of phenol red-free RPMI 1640 supplemented with 10% dextran-coated charcoal-stripped FBS, and the cells were grown for an additional 18-22 h. Two hours prior to harvesting, the growth medium was again replaced with fresh phenol red-free, charcoal-stripped RPMI. The cells were then washed with 10 mL of Hank's balanced salt solution and dissociated with trypsin. Trypsin was quenched with phenol red-free, charcoal stripped RPMI and the combined cells from each flask were centrifuged in a 50 mL conical tube at 100×g for 5 min. The cells were then resuspended in 50 mL of phenol red-free, charcoal-stripped RPMI and counted at this concentration. Centrifugation at 100×g gave approximately 1 mL of cells which were resuspended in 5 mL of 4° C. lysis buffer (10 mM Tris, 1.5 mM EDTA, 0.5 mM DTT, 10 mM NaMoO₄, 1.0 mM PMSF, 10% v/v glycerol) supplemented immediately before use with Complete-mini protease inhibitor cocktail. Cells were lysed via sonication at 4° C. with a microtip, set at maximum power, for 10 cycles of 6 s on and 24 s off. The cytosolic fraction of the lysate was isolated by ultracentrifugation at 4° C. and 225,000×g for 45 min. The centrifuged samples were dispensed into 100 μL aliquots and stored at −78° C. until used. Total protein was quantified either in fresh or frozen aliquots by the Sigma BSA micro protein determination method according to the prescribed protocol.

Aliquots of cell lysate were used fresh or thawed at 4° C. Stock solutions of 100× working concentration of the test ligands, ³H-Mibolerone and unlabled Mibolerone were prepared in DMSO and subsequently diluted to 10× in lysis buffer. Concentrations of test compounds were determined spectrophotometricly in DMSO by either absorbance at 310 nm for salicylamide containing molecules as determined from a Beer-Lambert plot described by varying concentrations of salicylamide, 264 nm for 2b, or 276 nm for 2a. Aliquots of cell lysate were complemented with 10 μL of 10× ligand solutions and 10 μL of the 10×³H-Mibolerone solution to yield concentrations of 1, 10, 100, and 1000 nM test compound and 1 nM ³H-Mibolerone. Each reaction was prepared in duplicate to yield 8 total test assays. Duplicate positive controls, consisting of 10 μL of lysis buffer in place of a test ligand (total radioligand binding), and negative controls, consisting of 1000 nM unlabled Mibolerone (nonspecific binding), each in the presence of 1 nM ³H-Mibolerone, were prepared. The reactions were gently mixed and briefly centrifuged before incubating at 4° C. for 30 min. After incubation was complete, 100 μL of each reaction was introduced to 400 μL of ice cold hydroxyapatite (HA), as a 60% suspension in pH 7.4 Tris buffer, on a 0.45 μm nylon filter in a Spin-X centrifuge tube. Upon addition of the reaction solution, the tubes were closed, briefly vortexed, and allowed to incubate on ice for 12 min with vortexing every 3-5 min. The HA suspensions were then centrifuged at 1200×g for 10 min. The filtrate was discarded and the dry pellet was resuspended in 400 μL of pH 7.3 20 mM Tris wash buffer containing 0.1% Triton-X100. Following seven rounds of resuspension and subsequent centrifugation, the final filtrate was discarded and the dry pellet was centrifuged for an additional 15 min. The pellet and filter bucket for each sample were then transferred to 20 ML scintillation vials and 4 mL of scintillation cocktail was added to each. Vortexing for 30 s thoroughly mixed the pellet with the scintillation liquid before counting. Each sample was counted for 5 repetitions of 3 min counts. This counting protocol was then repeated two additional times to assure precision. Specific binding for each test concentration was determined by subtracting the nonspecific binding control from the total binding determined for each concentration. Comparison to the specific binding for the positive control, in which no competing ligand was incubated with the ³H-Mibolerone, yielded the percent of ³H-Mibolerone displaced by a given concentration of test ligand. The IC₅₀ values for each test ligand were calculated by Logit-log (pseudo-Hill) analysis.

Cytotoxicity: In an attempt to determine targeting of 9 in PC3/AR and PC3/neo cells, cells were dissociated with trypsin EDTA, counted, and suspended in fully supplemented growth media to a concentration of 2.5×103 cells/mL. This cell suspension was dispensed in 200 μL aliquots into 96 well plates and was incubated for 36 h at 37° C. in a humidifed atmosphere of 5% CO₂ and 95% air. After 36 h growth, the medium was replaced with 180 μL phenol red-free RPMI 1640 supplemented with 10% dextran-coated charcoal-stripped FBS and the cells were allowed to grow an additional 24 h. Solutions of 9 and doxorubicin were prepared in DMSO at a 100× working concentration of 50 μM as determined by the 480 nm absorbance of the solution. After sterile filtering, the DMSO solutions were diluted 1:10 in phenol red-free, charcoal stripped RPMI medium; 20 μL of the appropriate 10× drug solution was immediately added to three lanes of both PC3/AR and PC3/neo cells. Additionally, 2 lanes were treated with 20 μL of stripped medium containing 10% DMSO and 1 lane was treated with 200 μL of 1.5 M Tris in millipore water. After 5, 10, and 20 min, the drug solution was removed from one lane of treated cells and replaced with 100 μL of phenol red-free, charcoal stripped RPMI medium. Media in the control lanes was replaced after 20 min. The cells were incubated for 12 h at 37° C., at which time 200 μL of fully supplemented RPMI 1640 growth medium was added to each well, without removal of the stripped medium. Cells were allowed to grow for 6 days at 37° C. in a humidified atmosphere of 5% CO₂ and 95% air.

The extent of colony formation was determined by use of a crystal violet staining assay. Cells were fixed with 200 μL of 1% gluteraldehyde in Hank's balanced salt solution. The cells were then stained with 100 μL of 0.1% crystal violet in Millipore water for 30 min. Following removal of the crystal violet solution, plates were submerged in distilled water and shaken vigorously to remove the excess water. After several hours drying time, 200 μL of 70% ethanol was added to each well to solubilize the dye. The plates were stored at 4° C. for 4 h as the dye was extracted from the cells. The optical density of each well was then measured on an ELISA plate reader at 588 nm. Relative colony size was established by comparison of the drug-treated lanes to the control lanes.

### Example 4

Design, synthesis, and biological evaluation of doxorubicin-formaldehyde conjugates targeted to breast cancer cells.

Estrogen receptors, which are commonly overexpressed in breast tumor cells, have long been exploited as therapeutic targets. Tamoxifen, a non-steroidal, antiestrogen has been used over the past three decades in the treatment of hormone responsive breast cancers. The estrogen receptor (now referred to as ERα) resides primarily in the nucleus; the binding of an agonist, such as estradiol, triggers the expression of multiple genes ultimately leading to cell proliferation. Upon binding estradiol, ER undergoes a conformational change, dissociates from heat shock proteins, homodimerizes, and binds estrogen response elements leading to transcription and cell proliferation. More recently, a new ER subtype, ERβ, has been identified. While ERα has been extensively studied, the physiological role of ERβ, particularly with respect to breast pathobiology, remains unclear.

This example describes the design, synthesis, and preliminary evaluation of a third generation of doxorubicin-formaldehyde conjugate that bears the doxsaliform moiety tethered to hydroxytamoxifen as a targeting group. The lead compound, DOX-TEG-TAM, Structure E of FIG. 2, consists of doxsaliform tethered to hydroxytamoxifen (TAM) via a triethylene glycol derivative (TEG).

Design: Several classes of molecules bind with high affinity to ER from which a targeting strategy could be developed. While an obvious choice would be to tether the doxorubicin-formaldehyde conjugate to estradiol, the native ER ligand, the presence of a growth stimulating hormone with a cytotoxin may not result in the most potent growth inhibitory conjugate. Alternatively, conjugation to an antiestrogen, such as tamoxifen, would deliver the cytotoxin to ER-overexpressing breast cancer cells without the concomitant growth stimulation.

The co-crystal structure of 4-hydroxytamoxifen (4-OHT), the active metabolite of tamoxifen, bound to the ligand binding domain of ERα reveals that one methyl group of the dimethylamino function of 4-OHT is exposed at the surface, perhaps providing an ideal location to tether a cytotoxic moiety. A further advantage to a targeting strategy based on tamoxifen, or 4-hydroxytamoxifen, is the binding interaction that triarylbutene antiestrogens have with antiestrogen binding sites (AEBS). Antiestrogen binding sites are cytosolic, membrane bound protein complexes that tightly bind tamoxifen and 4-OHT but exhibit virtually no affinity for estradiol. The structure and natural function of AEBS remains poorly understood; however, there is some evidence to suggest that AEBS overexpression plays a role in tamoxifen resistance. Additionally, AEBS are commonly expressed in hormone refractory, ER-negative breast cancer cell lines. Therefore, a targeting strategy that utilizes a ligand, such as 4-hydroxytamoxifen, that has high affinity to both ER and AEBS could serve to deliver a cytotoxin to a broader range of breast cancer cell types.

In the ER-targeted doxorubicin-formaldehyde conjugates of the present invention, formaldehyde is incorporated in the form of an N-Mannich base between the amide function of the salicylamide moiety and the amine of doxorubicin. A functionalized salicylamide is used as a trigger moiety to release the doxorubicin active metabolite. The trigger was tethered to the targeting group with derivatized ethylene glycol units, to confer enhanced water solubility. The tamoxifen active metabolite, 4-hydroxytamoxifen, was utilized as a targeting group based on the favorable attributes described above. An equimolar mixture of E and Z geometric isomers of the targeting group was utilized as previous work has demonstrated that para-hydroxy substituted triarylbutenes isomerize under cell culture conditions, compromising the interpretation of the activity of pure isomers. Furthermore, both geometric isomers of tamoxifen have been found to bind AEBS comparably.

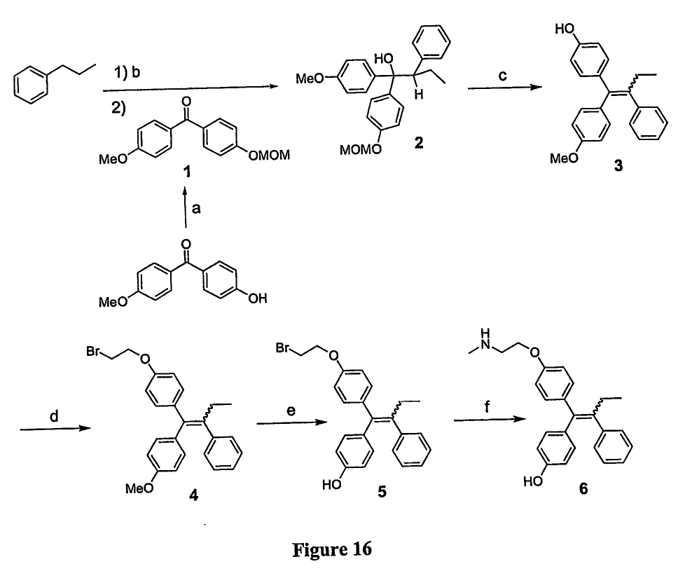

Chemistry: The overall synthetic strategy for 11a-c required the synthesis of desmethyl-4-hydroxytamoxifen 6, which could then be joined to various protected tethers, followed ultimately, by oximation with DOX-5-formylsaliform. The synthesis of desmethyl-4-hydroxytamoxifen was accomplished as shown in FIG. 16. The phenolic function in 4-hydroxy4′-methoxybenzophenone was first protected as the methoxymethyl (MOM) ether under standard conditions providing benzophenone 1 in good yield. The other commercially available starting material, n-propylbenzene, was metallated at the α position using Schlosser's base and then combined with 1 to provide carbinol 2 in 97% yield. Carbinol 2 was then dehydrated and MOM-deprotected in one step under strongly acidic conditions to yield triarylbutene 3 in good overall yield. The phenolic function of triarylbutene 3 was bromo-ethylated under phase transfer conditions to achieve a 90% yield of 4. Triarylbutene 4 was then demethylated with boron tribromide to provide free phenol 5 in serviceable yield (57%). Early attempts at the demethylation resulted in the facile loss of the bromoethyl group as well as the methyl ether, providing the triarylbutene bis-phenol as the unwanted major product. Running the reaction at higher dilution and closely following the reaction by HPLC circumvented this problem; once the reaction had proceeded to the point at which roughly 60% of the starting material was demethylated the reaction was quenched. The starting material 4 was then recycled, to improve the overall yield from 57% to 75%. Finally, the primary bromide 5 was aminated with methylamine in 91% yield to complete the synthesis of the targeting group, E/Z-desmethyl-4-hydroxytamoxifen 6.

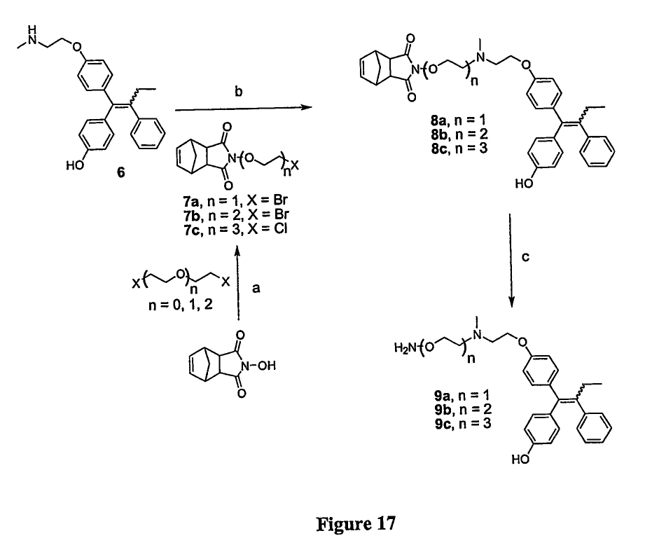

The targeting/tether intermediates, 9a-c, were synthesized as illustrated in FIG. 17. Commercially available N-hydroxynorbornyl dicarboximide, utilized as a protected amino-oxy ether function, was O-alkylated under mildly basic conditions with bis-halo derivatives of ethylene glycols to provide protected tethers 7a-c in 66-72% yield. The protected tethers, 7a-c, were joined to the targeting group (6) in the presence of Hunig's base to achieve the protected targeting/tether intermediates 8a-c in serviceable yields. Finally, the norbornyl protecting group was removed via hydrazinolysis, exposing the amino-oxy ether functionality (9a-c) in 67-74% yield.

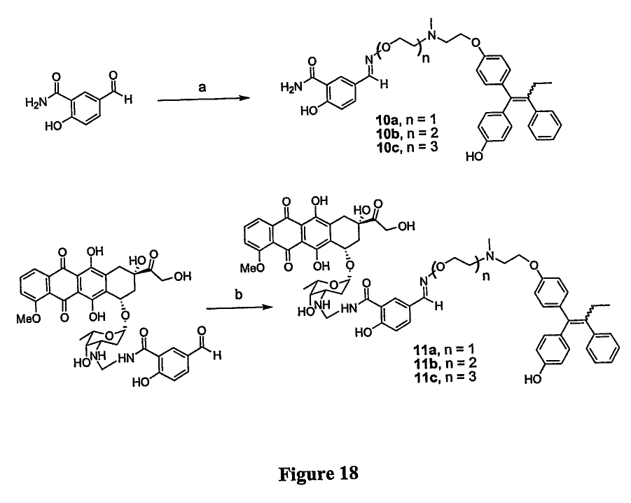

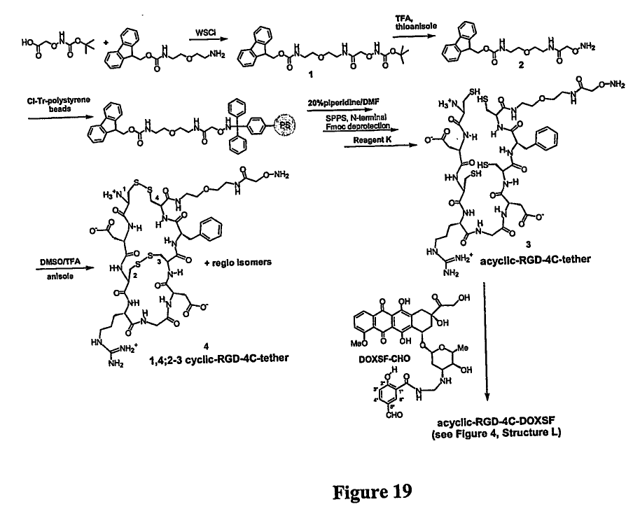

The synthesis was completed as shown in FIG. 18. First, the amino-oxy ether targeting/tether intermediates 9a-c were joined with the triggering molecule, 5-formylsalicylamide⁴⁵ to provide 10a-c in 72-88% yield. HPLC indicated the formation of one isomer about the oxime double bond, which presumably is the less sterically demanding anti product. The trigger/targeting molecules termed SAL-EG-TAM (10a), SAL-DEG-TAM (10b), and SAL-TEG-TAM (10c) were synthesized to evaluate the presence (11a-c) and absence (10a-c) of doxorubicin on the ER relative binding affinity of derivatized hydroxytamoxifen targeting group.

Second, the complete drug was prepared by joining, via oximation, DOX-5-formylsalicylamide to 9a-c. The reaction was performed in a 1:1 mixture of 95% ethanol and 0.5% aqueous trifluoroacetic acid to stabilize the base-labile N-Mannich linkage. The targeted formaldehyde conjugates, 11a-c, were isolated by preparative HPLC in 50% yield. The targeted formaldehyde conjugates, termed DOX-EG-TAM (11a), DOX-DEG-TAM (11b), and DOX-TEG-TAM (11c) to denote the length of the tether in ethylene glycol units, were fully characterized by COSY and HSQC 2D-NMR, and ESI-HRMS.

Results: Hydrolysis and stability. A standard solution of DOX-TEG-TAM (11c) in dimethylsulfoxide (DMSO) containing 1% v/v acetic acid (AcOH) was diluted 1:100 in two different buffers: lysis buffer (pH 7.4, 10 mM Tris, 1.5 mM EDTA, 10 mM Na₂MoO₄) used for binding experiments or TE buffer (pH 7.6, 10 MM Tris, 1 mM EDTA). Samples incubated at 37° C. and 4° C. were monitored by HPLC to detect the loss of intact targeted drug and the formation of doxorubicin. The hydrolysis data was fit using first-order reaction kinetics to provide first-order rate constants; the hydrolysis half-life was then calculated using t_{1/2}=ln2/k. The half-life for hydrolysis of 11c was found to be 76 min (pH 7.4) and 58 min (pH 7.6) at 37° C.; while at 4° C. the half-life was 180 h (pH 7.4) and 119 h (pH 7.6).

Estrogen receptor binding: The estrogen receptor source for the competitive binding experiments was an MCF-7 cell lysate. The crude cell lysate was utilized as a binding medium to account for other specific protein-ligand interactions that may occur under physiological conditions as well. The lysate was co-incubated with 1 nM tritium-labeled estradiol (³H-E2) and various radio-inert competitors at various concentrations for 18 h at 4° C. Following incubation free, unbound steroids were stripped from solution with 1% dextran-coated charcoal (DCC) buffered suspension; bound ³H-E2 in solution was then quantified via scintillation counting. Non-specific ³H-E2 binding was determined with 2000-fold diethylstilbestrol, an ER-competitive ligand, which competes off all ER-bound ³H-E2. All assays were performed in at least duplicate and scintillation counting was performed in triplicate to ensure reproducibility. The IC₅₀ for each competitor is defined as the concentration required to inhibit 50% of the ³H-E2 binding. The relative binding affinity (RBA) is, by definition, the ratio (as a percentage) of the molar concentrations of a reference competitive compound and a test compound required to decrease the proportion of specifically bound ³H-E2 by 50%. The RBA^{OHT} is the relative binding affinity of a competitor relative to E/Z-4-OHT.

To ensure that the developed assay would provide meaningful data, a control experiment in which cold E2 was used as the competitor was performed in duplicate. In both cases 1.5 nM cold E2 was found to reduce the bound ³H-E2 by 50%, indicating that the developed method is a valid measure of competitive binding. As a further control tamoxifen, which weakly binds ER, was utilized as a competitor. As expected tamoxifen, exhibited a weak interaction with ER with an IC₅₀ of 2000 nM.

First, the effect of tethering to 4-hydroxytamoxifen on ER binding was measured. A 1:1 mixture of E and Z geometric isomers of 4-hydroxytamoxifen, 12, gave an IC₅₀ of 5 nM, which was assigned an RBAOHT of 100. In the absence of the sterically demanding doxorubicin moiety (10a-c), the shortest tether, derived from ethylene glycol (10a), exhibits an RBA^{OHT} of 1.7; while 10b and 10c possess similar RBAOHT's of 3.3. While in the presence of the doxorubicin moiety, the formaldehyde conjugates, 11a-c, were found to have IC₅₀'s with values from 200-300 nM. The formaldehyde conjugate with the longest tether, DOX-TEG-TAM (11c) was found to possess slightly better binding characteristics (RBA^{OHT}=2.5) relative to 11a and 11b (RBA^{OHT}=1.7).

It is interesting to note that in the case of the triethylene glycol-derived tether, for example, that the addition of doxorubicin only decreases the RBA^{OHT} from 3.3 (10c) to 2.5 (11c). This suggests that it is the presence of the (poly)ethylene glycol tether unit or the triggering salicylamide moiety that are dominating the inhibition of the native antiestrogen binding interaction. However, the data indicate, albeit to a lesser extent, that ER binding is enhanced as the tether length increases. While the binding affinity of E/Z-4-OHT is clearly compromised by the steric demands of the tether/trigger/DOX moiety, the observed binding affinities of 11a-c may be sufficient to elicit a targeting response. It is encouraging that all three targeted conjugates possess substantially better binding characteristics than tamoxifen.

Breast cancer cell growth inhibition: The ER-targeted DOX-formaldehyde conjugates, 11a-c, were evaluated against four breast cancer cell lines that differ in terms of estrogen receptor and multidrug resistance expression. MCF-7 cells are human breast adenocarcinoma cells that express estrogen receptor at a level of 195,000 sites/cell. MCF-7/Adr cells are an ER-negative, doxorubicin-resistant variant of the parent MCF-7 that express the multidrug resistance (MDR) phenotype. MDA-MB-231 and MDA-MB-435 are ER-negative human breast adenocarcinoma and ductal carcinoma cells, respectively. All cytotoxins were formulated as 100× solutions in DMSO/1% AcOH and delivered to cells as 1% DMSO (0.01% AcOH) in cell culture medium. In all experiments cell treatment lasted 4 h.

In all four cell lines the targeted formaldehyde conjugates, 11a-c, were more cytotoxic than doxorubicin. In the case of doxorubicin-sensitive MCF-7 cells, the most active targeted conjugates, 11b and 11c, were 6-10 fold more cytotoxic than doxorubicin. In the case of ER-negative, multidrug resistant MCF-7/Adr cells the targeted formaldehyde conjugates were 40-fold (11a) and 140-fold (11b and 11c) more active than doxorubicin. In the case of ER-negative, drug-sensitive breast cancer cells, MDA-MB-231 and MDA-MB-435, all three targeted formaldehyde conjugates (11a-c) were 8-10 fold and 2-4 fold more cytotoxic, respectively.

There are several relevant controls necessary to interpret the growth inhibition data. When a 1:1 mixture of E and Z isomers of 4-hydroxytamoxifen (12) was administered to the cells, only ER-overexpressing MCF-7 cell growth was appreciably inhibited with an IC₅₀ of 300 nM. In the ER-negative cell lines (MCF-7/Adr, MDA-MB-231, and MDA-MB435) the IC₅₀ was 20,000-40,000 nM, likely the result of non-specific toxicity. Interestingly, when DOX and E/Z-4-OHT were co-administered as an equimolar mixture a synergistic effect was observed with a 2-5 fold increase in cytotoxicity relative to doxorubicin for all four cell lines. It is intriguing that the synergism would be observed in all four cell line regardless of ER or MDR expression.

Perhaps the most relevant control is the comparison of the targeted formaldehyde conjugates (11a-c) with the untargeted doxorubicin-formaldehyde equivalent, doxsaliform. Doxsaliform was prepared as the N-Mannich base as previously described. Doxsaliform was found to be equally cytotoxic (MDA-MB-435) or slightly (2-3 fold) less cytotoxic (MCF-7 and MDA-MB-231) than 11a-c in three of the four breast cancer cell lines tested. However, an interesting result is observed in the case of the multidrug resistant MCF-7/Adr cells; the targeted formaldehyde conjugates are 8-fold (11a) and 28-fold (11b, 11c) more cytotoxic than untargeted doxsaliform. This result is even more interesting in the context of the fact that MCF-7/Adr cells are ER-negative. One possible explanation for this observation is that the p-170 glycoprotein drug-efflux pump, that is overexpressed as part of the multidrug resistance phenotype, is rapidly pumping out doxorubicin and untargeted doxsaliform, while the targeted formaldehyde conjugates (11a-c) are entering the cell and experiencing a binding interaction with AEBS that serves to sequester the molecule, preventing drug-efflux pump mediated excretion. Indeed, increased lipophilicity, endowed by the presence of the triarylbutene moiety, should make the targeted formaldehyde conjugates poor substrates for the p-170 glycoprotein. In summary, the targeted formaldehyde conjugates, 11a-c, were more cytotoxic relative to doxorubicin and untargeted doxsaliform in both ER-positive (MCF-7) and ER-negative (MCF-7/Adr, MDA-MB-231) breast cancer cell lines.

Based the ER binding affinity and in vitro growth inhibition data, 11c was selected as the lead compound. This doxorubicin-formaldehyde conjugate, derived from the longest tether, is characterized by the most favorable in vitro data. Preliminary mouse in vivo formulation experiments demonstrate that the acute toxicity of 11c is substantially less than that of doxoform.

This data confirms the synthesis of a new class of targeted doxorubicin-formaldehyde conjugates that exhibit favorable in vitro characteristics in the treatment of sensitive and resistant breast cancer relative to both the clinical drug, doxorubicin, and an untargeted doxorubicin-formaldehyde conjugate, doxsaliform. Tethering from the N-methyl group of 4-hydroxytamoxifen does not eliminate the native ER binding affinity of the antiestrogen.

Experimental Section: General Remarks: Thin-layer chromatography (TLC) was performed on pre-coated aluminum sheets of silica gel 60 F₂₅₄. Flash column chromatography was performed on silica gel with particle size 32-63 μm and 60 Å pore size. Analytical HPLC was performed on a chromatograph equipped with a diode array UV-vis detector. Analytical HPLC injections were performed on a 5 μm reverse phase octadecylsilyl (ODS) microbore column, 2.1 mm ID×100 mm, eluting at 0.5 mL/min while monitoring at 256 and 310 nm. Analytical separation was achieved using Method #1. Method #1: flow rate, 0.5 mL/min; eluents, A=CH₃CN, B=pH 7.4 20 mM triethylammonium acetate; gradient, 5:95 A:B at 0 min to 50:50 A:B at 7 min, to 85:15 A:B at 12 min, isocratic to 17 min, back to 5:95 A:B at 20 min. Preparative HPLC was performed on a hybrid chromatograph consisting of a gradient pumping system and a diode array UV-vis detector. Preparative HPLC purification of the targeted doxorubicin-formaldehyde conjugates was performed on a C8 9.4 mm×25 cm semi-preparative column. Preparative purification was achieved using Method #2. Method #2: flow rate, 2.5 mL/min; eluents, A=CH₃CN, B=0.1% trifluoroacetic acid in purified water; gradient, 2:98 A:B at 0 min to 62:38 A:B at 22 min, isocratic to 28 min, back to 2:98 A:B at 30 min. As a consequence of the hydrolytic instability of the targeted compounds, the degree of purity was established by two independent HPLC methods. The first system (Method #2) was the HPLC configuration described above for the preparative purification of 11a-c. The second HPLC method for establishing purity, denoted Method #3, was performed on the system described above using an 8 4.6mm×15 cm analytical column. Method #3: flow rate, 1.0 mL/min; eluents, A=CH₃CN, B=0.1% trifluoroacetic acid in purified water; gradient, 2:98 A:B at 0 min to 70:30 A:B at 24 min, isocratic to 28 min, back to 2:98 A:B at 30 min. In all cases the E and Z isomers were reported as two peaks, typically overlapping at half peak height. The HPLC method for purification of DOX-5-formylsaliform, denoted Method #4, was performed on the hybrid chromatograph described above (Methods #1 and #2) with a 10 mm C8-guard column. Method #4: flow rate, 2.0 mL/min; eluents, A=CH₃CN, B=0.1% trifluoroacetic acid in purified water; gradient, 2:98 at 0 min to 35:65 at 16 min, 35:65 at 16 min to 85:15 at 20 min, back to 2:98 at 22 min. Molecular ions for all intermediates and final structures were determined using ESI-MS performed with a Fourier Transform mass spectrometer at Ohio State University (Prof. Christopher Hadad). ¹H-NMR spectra were acquired with a 500 MHz spectrometer, with the exception of the doxorubicin-formaldehyde conjugates that were analyzed on a 400 MHz spectrometer with a 3 mm micro-probe. NMR assignments for the protons in the tamoxifen, salicylamide, and doxorubicin portions of the final structures, 10a-c and 11a-c, are denoted “TAM”, “SAL”, and “DOX.”

MCF-7 and MDA-MB-231 cells were obtained from American Type Culture Collection (Rockville, Md.). MCF-7/Adr doxorubicin resistant cells were a gift from W. W. Wells (Michigan State University, East Lansing, Mich.). MDA-MB-435 cells were generously provided by Dr. Renata Pasqualini (MD Anderson Cancer Center, Houston, Tex.). MCF-7, MCF-7/Adr, and MDA-MB3-231 cells were maintained in vitro by serial culture in RPMI 1640 medium supplemented with 10% fetal bovine serum (Gemini Bioproducts, Calbassas, Calif.), L-glutamine (2 mM), HEPES buffer (10 mM), penicillin (100 units/mL), and streptomycin (100 μg/mL). MDA-MB-435 cells were maintained in vitro by serial culture in DMEM medium supplemented with 5% fetal bovine serum, L-glutamine (2 mM), sodium pyruvate (1 mM), and non-essential amino acids and vitamins for minimum essential media. Cells were maintained at 37° C. in a humidified atmosphere of 5% CO₂ and 95% air.

Synthesis: 4-(Methoxymethoxy)-4′-methoxybenzophenone (1). Sodium hydride (352 mg, 8.8 mmol) as a 60% dispersion in mineral oil was dissolved in 7 mL dry dimethylformamide (DMF) and cooled to 0° C. under argon (Ar). In a separate vial 4-hydroxy-4′-methoxybenzophenone (1 g, 4.4 mmol) was dissolved in 4 mL of dry DMF and added to the reaction flask containing the NaH/DMF mixture. The reaction mixture was stirred at 0° C. under Ar for 1 h. Chloromethyl methyl ether (0.67 mL, 8.8 mmol) was added and the reaction mixture allowed to warm to RT. After 2 h TLC revealed complete consumption of the benzophenone starting material. The reaction mixture was diluted with 120 mL methylene chloride (CH₂Cl₂), washed 2× with 50 mL 1 M sodium carbonate, dried (Na₂SO₄), concentrated in vacuo to give the crude product as an oil. The product was flash chromatographed on silica gel (4:1 hexanes:ethyl acetate to 3:2 hexanes:ethyl acetate) to yield 1.14 g (95%) of desired product as a clear, oily residue. TLC (SiO₂, 3:2 hexanes:ethyl acetate): R_{f}=0.55. ¹H NMR (500 MHz, CDCl₃) δ 3.49 (3H, s, MOM-OMe), 3.87 (3H, s, -OMe), 5.24 (2H, s, MOM-CH₂—), 6.95 (2H, d, J=8.9 Hz, 2′), 7.09 (2H, d, J=8.7 Hz, 2), 7.76 (2H, d, J=8.9 Hz, 3′), and 7.79 (2H, d, J=8.7 Hz, 3). HRMS [M+Na]+calculated 295.0941, found 295.0941.

1-(4-Methoxymethoxy-phenyl)-1-(4-methoxy-phenyl)-2-phenyl-butan-1-ol (2). To a solution of 23 mL of dry hexanes in an oven-dried 3-neck, 500 mL round bottom flask was added 25.1 mL of potassium tert-butoxide (25.1 mmol) as a 1.0 M solution in tetrahydrofuran (THF) and n-propylbenzene (3.5 mL, 25.1 mmol). The reaction mixture was stirred at RT under Ar. Using an oven-dried, Ar-purged syringe n-butyllithium (15.7 mL, 25.1 mmol) as a 1.6 M solution in hexanes was added, followed by tetramethylethylenediamine (7.6 mL, 50.2 mmol). The reaction mixture was stirred at RT under Ar for 30 min at which point it was cooled to −78° C. In a separate flask electrophile 1 (1.14 g, 4.19 mmol) was dissolved in 50 mL dry THF and added dropwise over 30 min. Following the addition of the electrophile, the reaction was allowed to warm to RT over 4 h, at which time TLC revealed complete consumption of the electrophile 1. The reaction was quenched through the addition of 50 mL of saturated ammonium chloride followed by 100 mL of distilled water. The aqueous phase was extracted 3× with 100 mL CH₂Cl₂. The combined organic layers were dried (Na₂SO₄) and concentrated in vacuo, providing a colorless, oily residue. The crude product was purified via flash chromatography on silica gel (4:1 hexanes:ethyl acetate) providing 1.6 g (97%) of carbinol 2 as an equimolar mixture of diastereomers. TLC (SiO₂, 3:1 hexanes:ethyl acetate): R_{f}=0.38. ¹H NMR (500 MHz, CDCl₃) δ 0.76 (3H, t, J=7.5 Hz, 4), 1.83 (2H, m, J=9.8, 7.5, 4.7 Hz, 3), 3.42 (1.5H, s, MOM-OMe diast. 1), 3.51 (1.5H, s, MOM-OMe diast. 2), 3.56 (1H, dd, J=9.8, 4.7 Hz, 3), 3.70 (1.5H, s, -OMe diast. 2), 3.82 (1.5H, s, -OMe diast. 1), 5.08 (1H, s, MOM-CH₂— diast. 1), 5.20 (1H, s, MOM-CH₂— diast. 1), 6.66 (1H, d, J=8.8 Hz, MeO-Ph 2 diast. 1), 6.80 (1H, d, J=8.8 Hz, MeO-Ph 2 diast. 2), 6.91 (1H, d, J=8.6 Hz, MOM-Ph 2 diast. 1), 7.04 (1H, d, J=8.8 Hz, MOM-Ph 2 diast. 2), 7.10 (2H, m, phenyl-ortho), 7.14 (1H, d, J=8.8 Hz, MeO-Ph 3 diast. 1), 7.15 (1H, d, J=8.8 Hz, MeO-Ph 3 diast. 2), 7.15 (3H, m, phenyl-meta, para), 7.46 (1H, d, J=8.6 Hz, MOM-Ph 3 diast. 1), 7.48 (1H, d, J=8.8 Hz, MOM-Ph 3 diast.2). HRMS [M+Na]⁺ calculated 415.1880, found 415.1886.

E/Z-1-(4-Hydroxyphenyl)-1-(4-methoxyphenyl)-2-phenyl-butene (3). To a solution of carbinol 2 (989 mg, 2.5 mmol) in 19 mL CH₂Cl₂ was added 19 mL 95% ethanol and 19 mL 6 M hydrochloric acid. The reaction mixture was stirred vigorously and heated under reflux overnight. After 18-24 h, 120 mL of sodium carbonate was added and the aqueous layer was extracted 4× with 100 mL CH₂Cl₂. The combined organic layers were dried (Na₂SO₄) and concentrated in vacuo, providing the crude reaction product. The crude product was purified via flash chromatography on silica gel (85% hexanes:15% ethyl acetate) providing 772 mg (93%) of triarylbutene 3 as an equimolar mixture of E and Z stereoisomers as a light yellow oil. TLC (SiO₂, 3:1 hexanes:ethyl acetate): R_{f}=0.27. ¹H NMR (500 MHz, CDCl₃) δ 0.94 (1.5H, t, J=7.5 Hz, 4 diast. 1), 0.95 (1.5H, t, J=7.5 Hz, 4 diast. 2), 2.50 (2H, q, J=7.5 Hz, 3), 3.70 (1.5H, s, -OMe diast. 1), 3.85 (1.5H, s, -OMe diast. 2), 6.48 (1H, d, J 8.8 Hz, HO-Ph 3 diast. 2), 6.57 (1H, d, J 8.8 Hz, HO-Ph 3 diast. 1), 6.75 (1H, d, J=8.8 Hz, MeO-Ph 3 diast. 2), 6.79 (1H, d, J=9.0 Hz, HO-Ph 2 diast. 2), 6.82 (1H, d, J=8.6 Hz, MeO-Ph 3 diast. 1), 6.90 (1H, d, J=8.8 Hz, HO-Ph 2 diast. 1), 7.12 (4H, d, J=8.6 Hz, MeO-Ph 2 and phenyl-ortho), 7.18 (3H, d, J=8.6 Hz, phenyl-meta,para). HRMS [M+Na]⁺ calculated 353.1512, found 353.1505.

E/Z1-[4-(2-Bromo-ethoxy)-phenyl]-1-(4-methoxy-phenyl)-2-phenylbut-1-ene (4). To a stirred solution of 3 (676 mg, 2.05 mmol) dissolved in 16.7 mL 1,2-dibromoethane was added 18.9 mL of 1 M sodium hydroxide and tetrabutylammonium hydrogensulfate (646 mg, 1.85 mmol). The biphasic reaction mixture was stirred vigorously at RT overnight. After 18 h, TLC revealed complete consumption of the starting material 3. The reaction was worked up via the addition of 100 mL CH₂Cl₂ and 100 mL sodium bicarbonate. The aqueous layer was washed 1× with 100 mL CH₂Cl₂; the combined organic layers were dried with Na₂SO₄ and concentrated in vacuo to yield the crude product as a light yellow oil. The material was purified via flash chromatography on silica gel (85% hexanes:15% ethyl acetate) providing 807 mg (90%) of triarylbutene 4 as an equimolar mixture of diastereomers. TLC (SiO₂, 4:1 hexanes:ethyl acetate): R_{f}=0.53. ¹H NMR (500 MHz, CDCl₃) δ 0.98 (3H, dt, J=7.5 Hz, 4), 2.53 (2H, dq, J=7.5 Hz, 3), 3.57 (1H, t, J=6.4 Hz, Br—CH₂— diast. 1), 3.68 (1H, t, J=6.4 Hz, Br—CH₂— diast. 2), 3.71 (1.5H, s, -OMe diast. 1), 3.86 (1.5H, s, -OMe diast. 2), 4.17 (1H, t, J=6.4 Hz, —O—CH₂— diast. 2), 4.34 (1H, t, J=6.4 Hz, —O—CH₂— diast. 1), 6.59 (2H, d, J=8.8 Hz, Br-EtO-Ph 3 both diast.), 6.82 (2H, dd, J=8.8, 9.0 Hz, MeO-Ph 3 both diast.), 6.93 (2H, dd, J=8.8 Hz, Br-EtO-Ph 2 both diast.), 7.15 (3H, m, phenyl-meta, para), 7.20 (4H, m, J=8.8 Hz, MeO-Ph 2 and phenyl-ortho). HRMS [M+Na]⁺ calculated 459.0930, found 459.0935.

E/Z1-[4-(2-Bromo-ethoxy)-phenyl]-1-(4-hydroxyphenyl)-2-phenyl-butene (5). To a stirred solution of triarylbutene 4 (689 mg, 1.58 mmol) in 130 mL of CH₂Cl₂ was added boron tribromide (1.58 mmol) as a 1 M solution in CH₂Cl₂. The reaction mixture was stirred under an inert atmosphere of Ar. The reaction was quenched at 4.5 h when analytical HPLC revealed about 60% formation of desired demethylated product; further reaction resulted in loss of the bromo-ethyl functional group. The reaction was quenched via the addition of 200 mL 2 M NaCl. The aqueous phase was extracted 2× with 100 mL CH₂Cl₂; the combined organic layers were washed 1× with 100 mL distilled water. The organic phase was concentrated in vacuo to yield the crude product as a light yellow oil. The material was purified via flash chromatography on silica gel (95% hexanes:5% ethyl acetate to 80% hexanes:20% ethyl acetate) providing 383 mg (57%) of phenol 5 as an equimolar mixture of diastereomers. The remaining starting material was then recycled to achieve a two-reaction yield of 75%. TLC (SiO₂, 4:1 hexanes:ethyl acetate): R_{f}=0.23. ¹H NMR (500 MHz, CDCl₃) δ 0.97 (3H, dt, J=7.5 Hz, 4), 2.52 (2H, dq, J=7.5 Hz, 3), 3.57 (1H, t, J=6.4 Hz, Br—CH₂— diast. 1), 3.68 (1H, t, J=6.4 Hz, Br—CH₂— diast. 2), 4.17 (1H, t, J=6.4 Hz, —O—CH₂— diast. 2), 4.34 (1H, t, J=6.4 Hz, —O—CH₂— diast. 1), 6.49 (1H, d, J=8.6 Hz, HO-Ph 3 diast. 2), 6.59 (1H, d, J=9.0 Hz, HO-Ph 3 diast. 1), 6.76 (1H, d, J=8.8 Hz, RO-Ph 3 diast.2), 6.83 (2H, dd, J=8.8, 9.0 Hz, HO-Ph 2 both diast.) 6.93 (1H, d, J=8.8 Hz, RO-Ph 3 diast. 1), 7.14 (4H, m, RO-Ph 2 and phenyl-ortho), 7.20 (3H, m, phenyl-meta, para). HRMS [M+Na]⁺ calculated 445.0774, found 445.0760.

E/Z1-(4-Hydroxyphenyl)-1-[4-(2-methylamino-ethoxy)-phenyl]-2-phenyl-butene (6). Bromide 5 (344 mg, 0.81 mmol) was dissolved in 8.1 mL of a 2 M solution of methylamine (16.2 mmol) in THF. The reaction was stirred in a sealed tube for 48 h at 60° C. After 48 h analytical HPLC revealed complete consumption of starting material. Reaction workup was accomplished via the addition of 100 mL CH₂Cl₂. The organic phase was washed 1× with 50 mL of a pH˜10 Na₂CO₃:NaHCO₃ aqueous buffer. The aqueous phase was then extracted 4× with 50 mL CH₂Cl₂. The combined organics were then dried (Na₂SO₄) and concentrated in vacuo to yield a yellow, oily reaction product.

The material was purified via flash chromatography on silica gel (90% chloroform:10% methanol) providing 276 mg (91%) of E/Z-desmethylhydroxytamoxifen 6 as an equimolar mixture of diastereomers. TLC (SiO₂, 4:1 chloroform:methanol): R_{f}=0.21. ¹H NMR (500 MHz, CDCl₃) δ 0.93 (3H, dt, J=7.5 Hz, 4), 2.49 (1.5H, s, N-Me diast. 1), 2.51 (2H, dq, J=7.5 Hz, 3), 2.55 (1.5H, s, N-Me diast. 2), 2.94 (1H, t, J=5.1 Hz, —N—CH₂— diast. 1), 3.04 (1H, t, J=5.1 Hz, —N—CH₂-diast. 2), 3.95 (1H, t, J=5.1 Hz, —O—CH₂— diast. 1), 4.11 (1H, t, J=5.1 Hz, —O—CH₂— diast. 2), 6.43 (1H, d, J=8.6 Hz, HO-Ph 3 diast. 2), 6.49 (1H, d, J=8.8 Hz, HO-Ph 3 diast. 1), 6.67 (1H, d, J=8.6 Hz, RO-Ph 3 diast. 2), 6.75 (2H, dd, J=8.4, 8.8 Hz, HO-Ph 2 both diast.) 6.83 (1H, d, J=8.6 Hz, RO-Ph 3 diast. 1), 7.03 (1H, d, J=8.6 Hz, RO-Ph 2 diast. 2), 7.11 (3H, m, RO-Ph 2 diast. 1 and phenyl-ortho), 7.16 (3H, m, phenyl-meta, para). HRMS [M+H]⁺ calculated 374.2115, found 374.2113.

4-(2-Bromo-ethoxy)4aza-tricyclo[5.2.1.0^{2,6}]dec-8-ene-3,5-dione (7a). To a solution of N-hydroxynorbornyl dicarboximide (1 g, 5.6 mmol) in 6.8 mL dry DMF was added triethylamine (2 mL, 14.0 mmol) and 1,2-dibromoethane (2.4 mL, 28.0 mmol). The reaction mixture was stirred at room temperature overnight. At 18 h TLC revealed consumption of starting material. The reaction workup was achieved via the addition of 200 mL CH₂Cl₂ and 150 mL 1 M NaHCO₃; the phases were separated and the aqueous phase was extracted 2× with 100 mL CH₂Cl₂. The combined organic layers were washed 1× with 100 mL 3 M NaCl, dried (Na₂SO₄), and concentrated in vacuo. The material was purified via flash chromatography on silica gel (60% hexanes:40% ethyl acetate) providing 1.105 g (69%) 7a as a white solid. TLC (SiO₂, 4:1 ethyl acetate:hexanes): R_{f}=0.61. ¹H NMR (500 MHz, CDCl₃) δ 1.50 (1H, dbs, 10b), 1.76 (1H, dt, J=1.8 Hz, 10a), 3.20 (2H, m, 1, 7), 3.42 (2H, m, 2, 6), 3.47 (2H, m, Br—CH₂—), 4.20 (2H, m, —O—CH₂—), 6.15 (2H, t, J=1.9 Hz, 8, 9). HRMS [M+Na]⁺ calculated 307.9893, found 307.9890.

4-[2-(2-Bromo-ethoxy)-ethoxy]4-aza-tricyclo[5.2.1.0^{2,6}]dec-8-ene-3,5-dione (7b). To a solution of N-hydroxynorbornyl dicarboximide (0.5 g, 2.8 mmol) in 2.8 mL dry DMF was added triethylamine (1 mL, 7.0 mmol) and 2-bromoethyl ether (1.8 mL, 14.0 mmol). The reaction and workup were performed exactly as described for 7a above. The material was purified via flash chromatography on silica gel (60% hexanes:40% ethyl acetate) providing 669 mg (72%) 7b as a clear, colorless oil. TLC (SiO₂, ethyl acetate): R_{f}=0.63. ¹H NMR (500 MHz, CDCl₃) δ 1.48 (1H, dbs, 10b), 1.73 (1H, dt, J=1.8 Hz, 10a), 3.18 (2H, m, 1, 7), 3.39 (2H, m, 2, 6), 3.45 (2H, m, Br—CH₂ ), 3.73 (2H, m, —N—O—CH₂—CH₂), 3.80 (2H, m, —N—O—CH₂—CH₂—), 4.10 (2H, m, —O—CH₂—CH₂—Br), 6.12 (2H, t, J=1.9 Hz, 8, 9). HRMS [M+Na]⁺ calculated 352.0155, found 352.0163.

4-{2-[2-(2-Chloro-ethoxy)-ethoxyl-ethoxy}-4-aza-tricyclo[5.2.1.0^{2,6}]dec-8-ene-3,5-dione (7c). To a solution of N-hydroxynorbomyl dicarboximide (0.5 g, 2.8 mmol) in 2.4 mL dry DMF was added triethylamine (1 mL, 7.0 mmol) and 1,2-bis(2-chloroethoxy)-ethane (2.2 mL, 14.0 mmol). The reaction mixture was stirred at 60° C. overnight. At 18 h TLC revealed consumption of starting material. The reaction workup was performed exactly as described for 7a above. The material was purified via flash chromatography on silica gel (50% hexanes:50% ethyl acetate) providing 614 mg (66%) 7c as a clear, colorless oil. TLC (SiO₂, ethyl acetate): R_{f}=0.59. ¹H NMR (500 MHz, CDCl₃) δ 1.47 (1H, dbs, 10b), 1.71 (1H, dt, J=1.8 Hz, 10a), 3.16 (2H, m, 1, 7), 3.37 (2H, m, 2, 6), 3.61 (6H, m, —O—CH₂—CH₂—O—CH₂—CH₂—Cl), 3.70 (4H, m, —N—O—CH₂—CH₂—), 4.08 (2H, m, —O—CH₂—CH₂—Cl), 6.10 (2H, t, J=1.9 Hz, 8, 9). HRMS [M+Na]⁺ calculated 352.0922, found 352.0924.

E/Z-4-{2-[(2-{4-[1-(4-Hydroxy-phenyl)-2-phenyl-but-1-enyl]-phenoxy}-ethyl)-methyl-amino]-ethoxy}-4-aza-tricyclo[5.2.1.0^{2,6}]dec-8-ene-3,5-dione (8a). A solution of 7a (99.3 mg, 0.35 mmol) in dry THF (1.5 mL) was added to 6 (86.3 mg, 0.23 mmol). Diisopropylethylamine (60 μL, 0.35 mmol) was added and the reaction was transferred to a sealed tube and heated to 60° C. for 24 h. Analytical HPLC revealed that the reaction had reached equilibrium with over 90% of the starting material consumed. The reaction workup consisted of dilution into 50 mL of ethyl acetate followed by washing the organic phase 1× with 50 mL of a pH˜10 Na₂CO₃:NaHCO₃ aqueous buffer. The aqueous phase was extracted 3× with 25 mL ethyl acetate; the combined organic layers were dried (Na₂SO₄) and concentrated in vacuo. The crude material was purified via flash chromatography on silica gel (90% ethyl acetate: 10% hexanes) providing 90.4 mg (68%) 8a as a clear, colorless oil. TLC (SiO₂, 4:1 ethyl acetate:hexanes): R_{f}=0.55. ¹H NMR (500 MHz, CDCl₃) δ 0.92 (3H, dt, J=7.3 Hz, TAM 4), 1.48 (1H, m, 10b), 1.74 (1H, m, 10a), 2.46 (5H, m, TAM 3, N-Me), 2.89 (3H, m, —CH₂—N—CH₂—CH₂—O—Ar, —CH₂—N—CH₂-CH₂—O—Ar diast. 1), 2.99 (1H, t, J=5.3 Hz, —CH₂—N—CH₂—O—CH₂—O—Ar diast. 2), 3.16 (2H, m, 1, 7), 3.40 (2H, m, 2, 6), 3.94 (1H, t, J=5.5 Hz, —N—O—CH₂— diast. 2), 4.11 (3H, m, Ar—O—CH₂—, —N—O—CH₂— diast. 1), 6.11 (2H, dt, J=2.0 Hz, 8, 9), 6.45 (2H, dd, J=8.8, 8.6 Hz, RO-Ph 2), 6.71 (2H, dd, J=8.8 Hz, HO-Ph 3), 6.80 (2H, dd, J=8.8, 8.6 Hz, RO-Ph 3), 7.10 (7H, m, HO-Ph 2, phenyl). HRMS [M+Na]⁺ calculated 601.2673, found 601.2692.

E/Z-4-{2-{2-[(2-{4-[1-(4-Hydroxy-phenyl)-2-phenyl-but-1-enyl]-phenoxy}-ethyl)-methyl-amino]-ethoxy}-ethoxy)-4-aza-tricyclo[5.2.1.0^{2,6}]dec-8-ene3,5-dione (8b). A solution of 7b (223 mg, 0.6 mmol) in dry THF (4.0 mL) was added to 6 (297 mg, 0.9 mmol). Diisopropylethylamine (157 μL, 0.9 mmol) was added. The reaction and workup were performed exactly as described for 8a above. The crude material was purified via flash chromatography on silica gel (100:0 to 98:2 ethyl acetate:hexanes) providing 205 mg (55%) 8b as a light yellow oil. TLC (SiO₂, 4:1 chloroform:methanol): R_{f}=0.54. ¹H NMR (500 MHz, CDCl₃) δ 0.92 (3H, dt, J=7.3 Hz, TAM 4), 1.45 (1H, m, 10b), 1.73 (1H, m, 10a), 2.46 (5H, m, TAM 3, N-Me), 2.78 (1H, t, J=5.6 Hz, —CH₂—N—CH₂—CH₂—O—Ar diast. 1), 2.85 (1H, t, J=5.6 Hz, —CH₂—N—CH₂—CH₂—O—Ar diast. 2), 2.90 (1H, t, J=5.6 Hz, —CH₂—N—CH₂—CH₂—O—Ar diast. 1), 3.00 (1H, t, J=5.6 Hz, —CH₂—N—CH₂—CH₂—O—Ar diast. 2), 3.15 (2H, m, 1, 7), 3.38 (2H, m, 2, 6), 3.65 (4H, m, —N—O—CH₂—CH₂—O—CH₂—CH₂—N—), 3.95 (1H, t, J=5.6 Hz, —N—O—CH₂— diast.2), 4.08 (3H, m, Ar—O—CH₂—, —N—O—CH₂— diast. 1), 6.12 (2H, m, 8. 9), 6.42 (1H, d, J=8.7 Hz, RO-Ph 2 diast. 2), 6.46 (1H, d, J=8.7 Hz, RO-Ph 2 diast. 1), 6.67 (1H, d, J=8.7 Hz, HO-Ph 3 diast. 1), 6.72 (1H, d, J=8.9 Hz, HO-Ph 3 diast. 2), 6.78 (2H, m, RO-Ph 3), 7.10 (7H, m, HO-Ph 2, phenyl). HRMS [M+H]⁺ calculated 623.3116, found 623.3133.

E/Z-4-[2-(2-{2-[(2-{4-[1-(4-Hydroxy-phenyl)-2-phenyl-but-1-enyl]-phenoxy}-ethyl)-methyl-amino]-ethoxy}-ethoxy)-ethoxy]-4-aza-tricyclo[5.2.1.0^{2,6}]dec-8-ene-3,5-dione (8c). A solution of 7c (190 mg, 0.57 mmol) in dry THF (2.5 mL) was added to 6 (143 mg, 0.38 mmol). Diisopropylethylamine (100 μL, 0.57 mmol) and sodium iodide (114 mg, 0.76 mmol) were added and the reaction mixture was transferred to a sealed tube and heated to 60° C. for 24 h. Analytical HPLC revealed that the reaction had proceeded to 90% consumption of starting material. The reaction workup was performed exactly as describe above for 8a. The crude material was purified via flash chromatography on silica gel (98% ethyl acetate:2% methanol to 92% ethyl acetate:8% methanol) providing 154 mg (61%) 8c as a light yellow oil. TLC (SiO₂, 4:1 ethyl acetate:methanol): Rf=0.14. ¹H NMR (500 MHz, CDCl₃) δ 0.91 (3H, dt, J=7.3 Hz, TAM 4), 1.47 (1H, m, 10b), 1.73 (1H, m, 10a), 2.42 (3H, ds, N-Me), 2.47 (2H, q, J=7.3 Hz, TAM 3), 2.75 (1H, t, J=5.7 Hz, —CH₂—N—CH₂—CH₂—O—Ar diast. 1), 2.81 (1H, t, J=5.7 Hz, —CH₂—N—CH₂—CH₂—O—Ar diast. 2), 2.86 (1H, t, J=5.5 Hz, —CH₂—N—CH₂—CH₂—O—Ar diast. 1), 2.96 (1H, t, J=5.5 Hz, —CH₂—N—CH₂—CH₂—O—Ar diast. 2), 3.15 (2H, m, 1, 7), 3.39 (2H, m, 2, 6), 3.63 (8H, m, —N—O—CH₂—CH₂—O—CH₂—CH₂—O—CH₂—CH₂—N—), 3.94 (1H, t, J=5.7 Hz, —N—O—CH₂— diast. 2), 4.10 (3H, m, Ar—O—CH₂—, —N—O—CH₂— diast. 1), 6.12 (2H, m, 8, 9), 6.41 (1H, d, J=9.0 Hz, RO-Ph 2 diast. 2), 6.47 (1H, d, J=8.8 Hz, RO-Ph diast. 1), 6.68 (2H, dd, J=8.8, 9.0 Hz, HO-Ph 3), 6.79 (2H, dd, J=8.8, 8.6 Hz, RO-Ph 3), 7.10 (7H, m, HO-Ph 2, phenyl). HRMS [M+Na]⁺ calculated 689.3197, found 689.3203.

E/Z-1-(4-{2-[(2-Aminooxy-ethyl)-methyl-amino]-ethoxy}-phenyl)-1-(4-hydroxyphenyl)-2-phenyl-butene (9a). To a solution 8a (22.7 mg, 39 μmol) dissolved in 0.5 mL of 95% ethanol was added hydrazine monohydrate (7 μL, 195 μmol). The reaction mixture was transferred to a sealed tube and heated to 60° C. After 2 h, TLC revealed complete consumption of starting material. The reaction mixture was transferred to a round-bottom flask and concentrated in vacuo. The crude product was purified via flash chromatography on silica gel (90% chloroform:10% methanol) providing 16.9 mg (71%) 9a as a clear, colorless oil. TLC (SiO₂, 9:1 chloroform:methanol): Rf=0.17. ¹H NMR (500 MHz, CD₃CN) δ 0.87 (3H, dt, J=7.3 Hz, 4), 2.25 (1.5H, s, N-Me diast. 1), 2.32 (1.5H, s, N-Me diast. 2), 2.42 (2H, dq, J=7.3 Hz, 3), 2.59 (1H, t, J=5.7 Hz, H₂N—O—CH₂—CH₂—N—CH₂— diast. 1), 2.65 (1H, t, J=5.7 Hz, H₂N—O—CH₂—CH₂—N—CH₂— diast. 2), 2.69 (1H, t, J=5.9 Hz, H₂N—O—CH₂—CH₂—N—CH₂— diast. 1), 2.79 (1H, t, J=5.9 Hz, H₂N—O—CH₂—CH₂—N—CH₂— diast. 2), 3.64 (1H, t, J=5.7 Hz, H₂N—O—CH₂— diast. 1), 3.69 (1H, t, J=5.7 Hz, H₂N—O—CH₂— diast. 2), 3.90 (1H, t, J=5.9 Hz, Ar—O—CH₂— diast.1), 4.06 (1H, t, J=5.9 Hz, Ar—O—CH₂— diast. 2), 6.45 (1H, d, J=8.6 Hz, HO-Ph 3 diast. 2), 6.54 (1H, d, J=9.0 Hz, HO-Ph 3 diast. 1), 6.70 (1H, d, J=8.6 Hz, RO-Ph 3 diast. 2), 6.77 (1H, d, J=8.8 Hz, RO-Ph 3 diast. 1), 6.79 (1H, d, J=8.6 Hz, HO-Ph 2 diast. 2), 6.88 (1H, d, J=8.8 Hz, HO-Ph 2 diast. 1), 7.05 (1H, d, J=8.6 Hz, RO-Ph 2 diast. 2), 7.13 (6H, m, RO-Ph 2 diast. 1, phenyl). HRMS [M+Na]⁺ calculated 455.2305, found 455.2275.

E/Z-1-[4-(2-{([2-(2-Aminooxy-ethoxy)-ethyl]-methyl-amino}-ethoxy)-phenyl]-1-(4-hydroxyphenyl)-2-phenyl-butene(9b). To a solution of 8b (45.7 mg, 73.4 μmol) dissolved in 1.0 mL of 95% ethanol was added hydrazine hydrate (11.5 μL, 367 μmol). The reaction was performed exactly as described for 9a above. The crude product was purified via flash chromatography on silica gel (98:2 to 92:8 chloroform:methanol) providing 23.5 mg (67%) 9b as a clear, colorless oil. TLC (SiO₂, 9:1 chloroform:methanol): R_{f}=0.12. ¹H NMR (500 MHz, CD₃CN) δ 0.87 (3H, dt, J=7.3 Hz, TAM 4), 2.26 (1.5H, s, N-Me diast. 1), 2.33 (1.5H, s, N-Me diast. 2), 2.42 (2H, dq, J=7.3 Hz, TAM 3), 2.58 (1H, t, J=5.8 Hz, Ar—O—CH₂—CH₂—N—CH₂— diast. 1), 2.64 (1H, t, J=5.8 Hz, Ar—O—CH₂—CH₂—N—CH₂— diast. 2), 2.71 (1H, t, J=5.8 Hz, Ar—O—CH₂—CH₂—N—CH₂— diast. 1), 2.81 (1H, t, J=5.8 Hz, Ar—O—CH₂—CH₂—N—CH₂— diast. 2), 3.52 (4H, m, —N—O—CH₂—CH₂—O—CH₂—), 3.66 (2H, m, —N—O—CH₂—), 3.90 (1H, t, 3=5.6 Hz, Ar—O—CH₂— diast. 2), 4.06 (1H, t, J=5.6 Hz, Ar—O—CH₂— diast. 1), 6.45 (1H, d, J=8.5 Hz, HO-Ph 3 diast. 2), 6.53 (1H, d, J=8.7 Hz, HO-Ph 3 diast. 1), 6.70 (1H, d, J=8.5 Hz, RO-Ph 3 diast. 2), 6.77 (1H, d, J=8.7 Hz, RO-Ph 3 diast. 1), 6.79 (1H, d, J=8.5 Hz, HO-Ph 2 diast. 2), 6.88 (1H, d, J=8.7 Hz, HO-Ph 2 diast. 1), 7.05 (1H, d, J=8.5 Hz, RO-Ph 2 diast. 2), 7.14 (6H, m, RO-Ph 2 diast. 1, phenyl). HRMS [M+Na]⁺ calculated 499.2567, found 499.2545.

E/Z-1-{4-[2-({2-[2-(2-Aminooxy-ethoxy)-ethoxyl-ethyl}-methyl-amino)-ethoxy]-phenyl}-1-(4-hydroxyphenyl)-2-phenyl-butene (9c). To a solution of 8c (135 mg, 203 μmol) dissolved in 2.6 mL of 95% ethanol was added hydrazine hydrate (31.8 μL, 1.02 mmol). The reaction was performed exactly as described for 9a above. The crude product was purified via flash chromatography on silica gel (98:2 to 90:10 chloroform:methanol) providing 78.4 mg (74%) 9c as a clear, colorless oil. TLC (SiO₂, 4:1 chloroform:methanol): R_{f}=0.16. ¹H NMR (500 MHz, CD₃CN) δ 0.87 (3H, dt, J=7.3 Hz, 4), 2.25 (1.5H, s, N-Me diast. 1), 2.32 (1.5 H, s, N-Me diast. 2), 2.42 (2H, dq, J=7.3 Hz, 3), 2.57 (1H, t, J=5.7 Hz, —O—CH₂—CH₂—N—CH₂—CH₂—O—Ar diast. 1), 2.64 (1H, t, J=5.9 Hz, —O—CH₂—CH₂—N—CH₂—CH₂—O—Ar diast. 2), 2.70 (1H, t, J=5.9 Hz, —O—CH₂—CH₂—N—CH₂—CH₂—O—Ar diast. 1) 2.80 (1H, t, J=5.7 Hz, —O—CH₂—CH₂—N—CH₂—CH₂—O—Ar diast. 2), 3.52 (8H, m, H₂N—O—CH₂—CH₂O—CH₂—CH₂—O—CH₂—CH₂—N—), 3.67 (2H, m, H₂N—O—CH₂—), 3.89 (1H, t, J=5.9 Hz, Ar—O—CH₂— diast. 1), 4.05 (1H, t, J=5.7 Hz, Ar—O—CH₂— diast. 2), 6.45 (1H, d, J=8.8 Hz, HO-Ph 3 diast. 2), 6.53 (1H, d, J=8.8 Hz, HO-Ph 3 diast. 1), 6.69 (1H, d, J=8.8 Hz, RO-Ph 3 diast. 2), 6.76 (1H, d, J=8.8 Hz, RO-Ph 3 diast. 2), 6.79 (1H, d, J=8.6 Hz, HO-Ph 2 diast. 2), 6.87 (1H, d, J=8.6 Hz, HO-Ph diast. 1), 7.04 (1H, d, J=8.6 Hz, RO-Ph 2 diast. 1), 7.14 (6H, m, RO-Ph 2 diast. 2, phenyl). HRMS [M+Na]⁺ calculated 543.2829, found 543.2796.

E/Z-2-Hydroxy-5-({2-1(2-{4-[1-(4-hydroxy-phenyl)-2-phenyl-but-1-enyl]-phenoxy}-ethyl)-methyl-amino]-ethoxyimino}-methyl)-benzamide (10a). To a solution of 9a (33 mg, 76.3 μmol) in 7.6 mL of 95% ethanol was added 5-formylsalicylamide (10 mg, 68.7 μmol). The reaction mixture was stirred vigorously overnight. Analytical HPLC revealed complete consumption of starting material after 18 h. The material was concentrated in vacuo and purified via flash chromatography on silica gel (100:0 to 95:5 chloroform:methanol) providing 32.2 mg (81%) 10a as a clear, colorless oil. TLC (SiO₂, 9:1 chloroform:methanol): R_{f}=0.23. ¹H NMR (500 MHz, CD₃CN) δ 0.86 (3H, dt, J=7.5 Hz, TAM 4), 2.30 (1.5H, s, N-Me diast. 1), 2.39 (3.5H, m, N-Me diast. 2, TAM 3), 2.74 (2H, m, ═N—O—CH₂—CH₂—N—), 2.82 (2H, m, Ar—O—CH₂—CH₂—N—), 3.90 (1H, t, J=5.9 Hz, ═N—O—CH₂— diast. 1), 4.06 (1H, t, J=5.9 Hz, ═N—O—CH₂— diast. 2), 4.17 (1H, t, J=5.7 Hz, Ar—O—CH₂— diast. 1), 4.22 (1H, t, J=5.7 Hz, Ar—O—CH₂— diast. 2), 6.44 (1H, d, J=8.6 Hz, HO-Ph 3 diast. 2), 6.52 (1H, d, J=8.8 Hz, HO-Ph 3 diast. 1), 6.68 (1H, d, J=8.8 Hz, RO-Ph 2 diast. 2), 6.73 (1H, d, J=8.8 Hz, RO-Ph 2 diast. 1), 6.79 (1H, d, J=8.6 Hz, HO-Ph 2 diast. 2), 6.86 (1H, d, J=8.8 Hz, HO-Ph 2 diast. 1), 6.92 (1H, dd, J=8.6, 2.2 Hz, SAL 3), 7.10 (7H, m, RO-Ph 3, phenyl), 7.66 (1H, m, SAL 4), 7.81 (1H, dd, J=7.3, 2.0 Hz, SAL 6), 8.00 (0.5H, s, oxime diast. 1), 8.02 (0.5H, s, oxime diast. 2). HRMS [M+H]⁺ calculated 580.2806, found 580.2837. Degree of purity: HPLC Method #2, retention times: 26.1 and 26.5 min, 97.3%; Method #3, retention times: 21.4 and 21.7 min, 95.4%.

E/Z-2-Hydroxy-5-[(2-{2-[(2-{4-[1-(4-hydroxy-phenyl)-2-phenyl-but-1-enyl]-phenoxy}-ethyl)-methyl-amino]-ethoxy}-ethoxyimino)-methyl]-benzamide (10b). To a solution of 9b (35 mg, 73 μmol) in 7.3 mL of 95% ethanol was added 5-formylsalicylamide (10.9 mg, 66 μmol). The reaction was performed exactly as described for 10a above. The crude product was purified via flash chromatography on silica gel (100:0 to 90:10 chloroform:methanol) providing 33.1 mg (72%) 10b as a clear, colorless oil. TLC (SiO₂, 9:1 chloroform:methanol): R_{f}=0.16. ¹H NMR (500 MHz, CD₃CN) δ 0.86 (3H, dt, J=7.5 Hz, TAM 4), 2.26 (1.5H, s, N-Me diast. 1), 2.33 (1.5H, s, N-Me diast. 2), 2.40 (2H, dq, J=7.5 Hz, TAM 3), 2.59 (1H, t, J=5.9 Hz, Ar—O—CH₂—CH₂—N—CH₂— diast. 1), 2.65 (1H, t, J=5.7 Hz, Ar—O—CH₂—CH₂—N—CH₂— diast. 2), 2.71 (1H, t, J=5.7 Hz, Ar—O—CH₂—CH₂—N—CH₂— diast. 1), 2.81 (1H, t, J=5.9 Hz, Ar—O—CH₂—CH₂—N—CH₂— diast. 2), 3.52 (1H, t, J=5.9 Hz, ═N—O—CH₂—CH₂—O—CH₂— diast. 2), 3.58 (1H, t, J=5.7 Hz, ═N—O—CH₂—CH₂—O—CH₂— diast. 1), 3.68 (2H, m, ═N—O—CH₂—CH₂—O—), 3.89 (1H, t, J=5.7 Hz, Ar—O—CH₂— diast. 1), 4.05 (1H, t, J=5.9 Hz, Ar—O—CH₂— diast. 2), 4.20 (2H, m, ═N—O—CH₂—CH₂—O—), 6.44 (1H, d, J=8.6 Hz, HO-Ph 3 diast. 2), 6.52 (1H, d, J=8.8 Hz, HO-Ph 3 diast. 1), 6.68 (1H, d, J=8.6 Hz, RO-Ph 2 diast. 2), 6.74 (1H, d, J=8.8 Hz, RO-Ph 2 diast. 1), 6.79 (1H, d, J=8.6 Hz, HO-Ph 2 diast. 2), 6.86 (1H, d, J=8.8 Hz, HO-Ph 2 diast. 1), 6.92 (1H, d, J=8.8 Hz, SAL 3), 7.03 (1H, d, J=8.8 Hz, RO-Ph 3 diast. 1), 7.12 (6H, m, RO-Ph 3 diast, 2, phenyl), 7.66 (1H, m, SAL 4), 7.83 (1H, m, SAL 6), 8.02 (0.5H, s, oxime diast. 1), 8.04 (0.5H, s, oxime diast. 2). HRMS [M+H]⁺ calculated 624.3068, found 624.3023. Degree of purity: HPLC Method #2, retention times: 26.8 and 27.2 min, 99.7%; Method #3, retention times: 21.7 and 22.0 min, >99.7%.

E/Z-2-Hydroxy-5-{[2-(2-{2-[(2-{4-[1-(4-hydroxy-phenyl)-2-phenyl-but-1-enyl]-phenoxy}-ethyl)-methyl-amino]-ethoxy}-ethoxy)-ethoxyiminol-methyl}-benzamide (10c). To a solution of 9c (33 mg, 63 μmol) in 6.3 mL of 95% ethanol was added 5-formylsalicylamide (9.4 mg, 57 μmol). The reaction was performed exactly as described for 10a above. The crude product was purified via flash chromatography on silica gel (100:0 to 95:5 chloroform:methanol) providing 33.5 mg (88%) 10c as a clear, colorless oil. TLC (SiO₂, 9:1 chloroform:methanol): R_{f}=0.17. ¹H NMR (500 MHz, CD₃CN) δ 0.89 (3H, t, J=7.5 Hz, TAM 4), 2.28 (1.5H, s, N-Me diast. 1), 2.35 (1.5H, s, N-Me diast. 2), 2.43 (2H, dq, J=7.5 Hz, TAM 3), 2.60 (1H, t, J=5.9 Hz, Ar—O—CH₂—CH₂—N—CH₂— diast. 1), 2.66 (1H, t, J=5.7 Hz, Ar—O—CH₂—CH₂—N—CH₂— diast. 2), 2.73 (1H, t, J=5.7 Hz, Ar—O—CH₂—CH₂—N—CH₂— diast. 1), 2.83 (1H, t, J=5.7 Hz, Ar—O—CH₂—CH₂—N—CH₂— diast. 2), 3.56 (6H, m, ═N—O—CH₂—CH₂—O—CH₂—CH₂—O—CH₂—CH₂—N—), 3.72 (2H, m, ═N—O—CH₂—CH₂—), 3.92 (1H, t, J=5.7 Hz, Ar—O—CH₂— diast. 1), 4.08 (1H, t, J=5.7 Hz, Ar—O—CH₂— diast. 2), 4.23 (2H, m, ═N—O—CH₂—), 6.47 (1H, d, J=8.6 Hz, HO-Ph 3 diast. 2), 6.56 (1H, d, J=8.8 Hz, HO-Ph 3 diast. 1), 6.71 (1H, d, J=8.6 Hz, RO-Ph 2 diast. 2), 6.79 (1H, d, J=8.8 Hz, RO-Ph 2 diast. 1), 6.82 (1H, d, J=8.6 Hz, HO-Ph 2 diast. 2), 6.91 (1H, d, J=8.8 Hz, HO-Ph 2 diast. 1), 6.95 (1H, d, J=8.6 Hz, SAL 3), 7.07 (1H, d, J=8.6 Hz, RO-Ph 3 diast. 1), 7.16 (6H, m, RO-Ph 3 diast. 2, phenyl), 7.70 (1H, m, SAL 4), 7.87 (1H, m, SAL 6), 8.06 (0.5H, s, oxime diast. 1), 8.07 (0.5H, s, oxime diast. 2). HRMS [M+Na]⁺ calculated 690.3150, found 690.3096. Degree of purity: HPLC Method #2, retention times: 27.1 and 27.4 min, 99.4%; Method #3, retention times: 21.7 and 22.0 min, >99.7%.

E/Z-N-[2-Hydroxy-5({2-[(2-{4-[1-(4-hydroxy-phenyl)-2-phenyl-but-1-enyl]-phenoxy}-ethyl)-methyl-amino]-ethoxyimino}-methyl)-benzamide]-doxorubicin (11a). DOX-5-formylsaliform was synthesized based on the procedure previously described for doxsaliform.^{21,23} To a solution of 23 mg of 5-formylsalicylamide⁴⁵ dissolved in 2 mL of DMF was added 20.8 μL of formalin and 20 mg of doxorubicin hydrochloride. The reaction mixture was stirred at 55° C. for 45 min. Following reaction, the solvent was concentrated in vacuo and the material was purified by preparative HPLC using Method #4 and carried forward without further characterization. A solution of DOX-5-formylsaliform acetate salt (4.4 mg, 5.8 μmol) in a mixture of 3.0 mL 0.5% trifluoroacetic acid in water and 1.5 mL 95% ethanol was measured for DOX chromophore concentration spectrophotometrically at 480 nm (ε=11,500 l·mol/cm). Targeting/tether group 9a (3.0 mg, 7.0 μmol) was dissolved in 1.5 mL 95% ethanol and added to the reaction mixture. The reaction was stirred at room temperature for 2 h. The reaction mixture was then filtered with a 4 mm, 0.45 um HPLC syringe filter (Alltech Associates, Inc., Deerfield, Ill.) and purified via preparative HPLC. Following each injection the desired product peaks (both E and Z isomers) at t_{R}=24.5 and 24.8 min were collected into a round bottom flask and 200 μL glacial acetic acid was added. Following peak collections the material was concentrated in vacuo providing 3.4 mg of the acetate salt of 11a (50%) as a red solid. ¹H NMR (400 MHz, CD₃0D) 8 0.86 (3H, dt, J=7.4 Hz, TAM 4), 1.32 (3H, d, J=5.5 Hz, DOX 5′-Me), 2.17 (3H, m, DOX 8 and DOX 2′), 2.41 (3H, m, DOX 2′ and TAM 3), 2.98 (1.5H, s, TAM N-Me), 3.02 (2H, ab, DOX 10), 3.06 (1.5H, s, TAM N-Me), 3.66 (6H, bm, DOX 3′ and —CH₂—N—CH₂—), 3.98 (3H, s, DOX 4-OMe), 4.21 (1H, t, J=4.9 Hz, ═N—O—CH₂— diast. 1), 4.30 (1H, bm, DOX 5′), 4.37 (1H, t, J=4.9 Hz, ═N—O—CH₂— diast. 2), 4.42 (1H, t, J=4.7 Hz, Ar—O—CH₂— diast. 1), 4.47 (1H, bt, Ar—O—CH₂— diast. 2), 4.70 (4H, m, DOX 14 and —N—CH₂—N—), 4.87 (1H, under CD₃OH, DOX 4′), 5.13 (1H, bs, DOX 7), 5.47 (1H, bs, DOX 1′) 6.37 (1H, d, J=8.7 Hz, TAM HO-Ph 3 diast. 2), 6.57 (3H, m, SAL 3 and TAM HO-Ph 3 diast. 1, TAM RO-Ph 2 diast. 2), 6.73 (2H, m, TAM RO-Ph 2 diast. 1 and TAM HO-Ph 2 diast. 2), 6.94 (3H, m, TAM HO-Ph 2 diast. 1 and TAM RO-Ph 3), 7.08 (5H, m, TAM phenyl), 7.33 (1H, m, SAL 4), 7.52 (1H, d, J=8.5 Hz, DOX 3), 7.85 (4H, m, oxime, SAL 6, DOX 1 and DOX 2). HRMS [M+H]⁺ calculated 1135.4547, found 1135.4564. Degree of purity: HPLC Method #2, retention times: 24.5 and 24.8 min, 98.3%; Method #3, retention times: 20.5 and 20.8 min, 98.5%.

E/Z-N-(2-Hydroxy-5-[(2-{2-[(2-{4-[1-(4-hydroxy-phenyl)-2-phenyl-but-1-enyl]-phenoxy}-ethyl)-methyl-amino]-ethoxy}-ethoxyimino)-methyl]-benzamide)-doxorubicin (11b). The reaction and purification as described above for 11a were utilized only substituting targeting/tether group 9b. Purification provided the acetate salt of 11b (50%) as a red solid. ¹H NMR (400 MHz, CD₃OD) δ 0.86 (3H, dt, J=7.3 Hz, TAM 4), 1.33 (3H, d, J=6.4 Hz, DOX 5′-Me), 2.16 (3H, bm, DOX 8 and DOX 2′), 2.42 (3H, m, TAM 3 and DOX 2′), 2.94 (1.5H, s, TAM N-Me diast. 1), 3.01 (1.5H, s, TAM N-Me diast. 2), 3.04 (2H, ab, DOX 10), 3.50 (4H, bm, —CH₂—N—CH₂—), 3.80 (5H, bm, ═N—O—CH₂—CH₂—O—CH₂—CH₂—N— and DOX 3′), 3.97 (3H, s, DOX 4′-OMe), 4.18 (2H, m, ═N—O—CH₂—), 4.23 (1H, bt, TAM Ar—O—CH₂— diast. 1), 4.31 (1H, bq, DOX 5′), 4.34 (1H, bt, TAM Ar—O—CH₂— diast. 2), 4.70 (4H, ds, DOX 14 and —N—CH₂—N—), 4.94 (1H, under CD₃OH, DOX 4′), 5.13 (1H, bs, DOX 7), 5.48 (1H, bs, DOX 1′), 6.36 (1H, d, J=8.4 Hz, TAM HO-Ph 3 diast. 2), 6.57 (3H, m, SAL 3 and TAM HO-Ph 3 diast. 1, TAM RO-Ph 2 diast. 2), 6.73 (2H, m, TAM RO-Ph 2 diast. 1 and TAM HO-Ph 2 diast. 2), 6.95 (3H, m, TAM HO-Ph 2 diast. 1 and TAM RO-Ph 3), 7.07 (5H, m, TAM phenyl), 7.29 (1H, m, SAL 4), 7.52 (1H, d, J 8.5 Hz, DOX 3), 7.70 (1H, ds, oxime), 7.81 (2H, m, DOX 2 and SAL 6), 7.91 (1H, d, J=7.6 Hz, DOX 1). HRMS [M+H]⁺ calculated 1179.4809, found 1179.4709. Degree of purity: HPLC Method #2, retention times: 24.9 and 25.2 min, >99.5%; Method #3, retention times: 20.4 and 20.6 min, 97.8%.

E/Z-N-(2-Hydroxy-5-{[2-(2-{2-[(2-{4-[1-(4-hydroxy-phenyl)-2-phenyl-but-1-enyl]-phenoxy}-ethyl)-methyl-amino]-ethoxy}-ethoxy)-ethoxyimino]-methyl}-benzamide)-doxorubicin (11c). The reaction and purification as described above for 11a were utilized only substituting targeting/tether group 9c. Purification provided the acetate salt of 11c (50%) as a red solid. ¹H NMR (400 MHz, CD₃OD) δ 0.86 (3H, t, J=7.4 Hz, TAM 4), 1.34 (3H, d, J=6.5 Hz, DOX 5′-Me), 2.14 (2H, m, DOX 8), 2.30 (1H, m, DOX 2′), 2.41 (3H, m, DOX 2′ and TAM 3), 2.94 (1.5H, s, N-Me diast. 1), 3.0 (2H, ab, DOX 10), 3.02 (1.5H, s, N-Me diast. 2), 3.64 (9H, m, ═N—O—CH₂—CH₂—O—CH₂—CH₂—O—CH₂—CH₂—N—CH₂—CH₂—O—Ar and DOX 3′), 3.82 (4H, m, ═N—O—CH₂—CH₂—O—CH₂—),3.94 (3H, s, DOX 4′-OMe), 4.13 (2H, bm, ═N—O—CH₂—), 4.19 (1H, t, J=5.0 Hz, Ar—O—CH₂— diast. 1), 4.32 (1H, m, DOX 5′), 4.36 (1H, t, J=4.9 Hz, Ar—O—CH₂— diast. 2), 4.72 (4H, ds, DOX 14 and —N—CH₂—N—), 4.86 (1H, under CD₃O H, DOX 4′), 5.09 (1H, bs, DOX 7), 5.45 (1H, bs, DOX 1′), 6.37 (1H, d, J=8.6 Hz, TAM HO-Ph 3 diast. 2), 6.45 (1H, bm, SAL 3), 6.59 (2H, m, TAM HO-Ph 3 diast. 1 and TAM RO-Ph 2 diast. 2), 6.74 (TAM RO-Ph 2 diast. 1 and TAM HO-Ph 2 diast. 2), 6.95 (2H, m, TAM HO-Ph 2 diast. 1 and TAM RO-Ph 3 diast. 2), 7.07 (6H, m, TAM RO-Ph 3 diast. 1 and TAM phenyl), 7.23 (1H, bm, SAL 4), 7.47 (1H, d, J=8.3 Hz, DOX 3), 7.57 (1H, ds, oxime), 7.66 (1H, bs, SAL 6), 7.70 (1H, bm, DOX 2), 7.84 (1H, bd, DOX 1), HRMS [M+Na]⁺ calculated 1245.4890, found 1245.4966. Degree of purity: HPLC Method #2, retention times: 24.8 and 25.0 min, >99.7%; Method #3, retention times: 20.2 and 20.5 min, 98.8%.

E/Z-4-Hydroxytamoxifen (12). Bromide 5 (112 mg, 0.26 mmol) was dissolved in 2.6 mL of a THF solution containing a 2 M concentration of dimethylamine (5.2 mmol). The mixture was transferred to a sealed tube and heated to 60° C. After 43 h TLC revealed consumption of starting material 5. Reaction workup was accomplished via the addition of 30 mL CH₂Cl₂. The organic phase was extracted 1× with 50 mL of a pH˜10 Na₂CO₃:NaHCO₃ aqueous buffer. The aqueous phase was washed 4× with 15 mL CH₂Cl₂. The combined organics were then dried (Na₂SO₄) and concentrated in vacuo to yield a yellow, oily reaction product. The material was purified via chromatography on silica gel (95:5 to 90:10 chloroform:methanol) providing 31 mg (31%) of E/Z-4-hydroxytamoxifen 12. ¹H NMR (500 MHz, CDCl₃) established the structure as previously described⁴⁴ and indicates >99% purity.

Biological Evaluation: Hydrolysis and stability: The half-life for hydrolysis was determined for the lead compound, DOX-TEG-TAM (11c), at 4° C. and 37° C. The concentration of a stock solution of DOX-TEG-TAM in DMSO/1% AcOH was found to be 1.2 mM by vis absorption at 480 nm (ε=11,500 l·mol/cm). The DOX-TEG-TAM was diluted 1:100 in either pH 7.6 TE buffer (10 mM tris, 1 mM EDTA buffer) or pH 7.4 lysis buffer (10% v/v glycerol, 10 mM Tris, 1.5 mM EDTA, 10 mM Na₂MoO₄). A sample of each buffer was kept at 37° C. and 4° C. and monitored by HPLC using Method #2 (described above) to track the loss of DOX-TEG-TAM and the subsequent formation of doxorubicin. The area-under-the-curve (AUC) was used to calculate the percentage of intact material versus time. The hydrolysis data were fit to first-order kinetics using Regression (Blackwell Scientific Publishing, London) software. The reaction rate constants were 0.012±0.0007 min⁻¹ (pH 7.6) and 0.0092±0.0003 min⁻¹ (pH 7.4) at 37° C.; 0.0058±0.0005 h⁻¹ (pH 7.6) and 0.0038±0.0003 h⁻¹ (pH 7.5) at 4° C. The half-life for hydrolysis was then calculated from the rate constants using t_{1/2}=ln2/k.

Estrogen receptor binding assay: The relative binding affinity of each test compound was measured through competition assay with tritiated estradiol (³H-E2) through a procedure adapted from several sources.^{50,53} MCF-7 cells were utilized as the ERα source. Cells were cultured in six T-175 flasks to 80% confluence at which time the full RPMI media was replaced with phenol red-free RPMI media supplemented with 10% dextran-coated charcoal (DCC) stripped fetal calf serum (henceforth referred to as “stripped media”); the cells were cultured for an additional 24 h. Four hours prior to harvesting, the growth medium was replaced with fresh stripped media. To harvest, cells were washed with 10 mL of Hank's balanced salt solution and dissociated from the flasks with 2 mL trypsin. Trypsinization was quenched with 10 mL of fresh stripped media; cells from six T-175 flasks were combined and pelleted by centrifugation at 300 g for 5 min at 25° C. The supernatant was decanted, the cells were resuspended in 50 mL stripped media and enumerated with a hemacytometer. The cells were then pelleted again by centrifugation. The supernatant was decanted and the cells were suspended in pH 7.4 lysis buffer (10% v/v glycerol, 10 mM Tris, 1.5 mM EDTA, 0.5 mM dithiothreitol, 10 mM Na₂MoO₄, 1.0 mM phenylmethylsulfonyl fluoride, supplemented with Complete-Mini™ protease inhibitors) at 4° C. such that the cell density was 25 million cells per mL lysis buffer. Cells were lysed at 0° C. via sonication with a microtip set at maximum power for 10 cycles of 6 s on followed by 24 s off. The ER-enriched lysate was obtained by ultracentrifugation of the homogenate at 225,000 g for 45 min at 4° C. The supernatant was dispensed into 100 μL aliquots and stored at −70° C. The lysate protein density was measured with a Sigma Diagnostics Total Protein kit; for all experiments the protein density was between 3.1-4.0 mg/mL.

Competitive ligands were prepared as 120× stock solutions in DMSO containing 1% acetic acid. Competitor concentrations were determined for ligands containing the DOX chromophore by optical density at 480 nm (ε=11,500 l/mol·cm), while ligands containing only the salicylamide/triarylbutene chromophore were measured at 280 nm (ε=29,500 l/mol·cm). Typically four different concentrations of competitor were prepared as 120× solutions in DMSO containing 1% acetic acid. Tritiated estradiol was prepared as a 120 nM stock solution (120×) in DMSO containing 1% acetic acid. The 120× solutions were then diluted 1:10 in pH 7.6 TE (10 mM Tris, 1 mM EDTA) buffer to provide 12× solutions of ³H-E2 and competitor. Aliquots of cell lysate (100 μL) were thawed at 4° C.; 10 μL of 12× competitor was added, followed by 10 μL 12×³H-E2. Total binding was measured by addition of vehicle in the absence of competitor, while non-specific binding was determined by incubation of ³H-E2 in the presence of 2000× diethylstilbestrol. Reaction lysates were vortexed vigorously and stored at 4° C. for 18 h. Following incubation, unbound steroids were stripped from the lysate through the addition of 280 μL of DCC as a 1% w/v suspension in pH 7.6 TE buffer (10 mM tris, 1.0 mM EDTA). Following the addition of the DCC, the reaction lysates were vortexed and stored on ice for 15 min, with vortexing every 5 min. DCC was pelleted by centrifugation at 3000 g for 10 min at 4° C.; 300 μL of lysate supernatant was transferred to scintillation vials containing 4 mL of biodegradable scintillation cocktail. The vials were then vortexed vigorously and each sample was counted for 5 repetitions of 3 min counts. This counting protocol was then repeated to ensure reproducibility. Scintillation counting background was subtracted from all measurements. The relative binding affinity (RBA) for each test compound was calculated from the ratio of the molar concentrations of unlabeled E2 and the test compound required to decrease the proportion of specifically bound ³H-E2 by 50%. Scintillation counting was performed in triplicate and each competitor was assayed in at least duplicate. Error bars for each determination represent one standard deviation about the mean for scintillation counting statistics.

In Vitro cellular growth inhibition experiments: The IC₅₀ for the targeted formaldehyde conjugates and all control compounds were performed as previously described with minor modifications. All compounds were solubilized in dimethylsulfoxide containing 1% v/v acetic acid. The concentrations of all 100× DMSO/1% AcOH drug solutions was determined spectrophotometrically by absorbance at 480 nm (ε=11,500 l·mol/cm). Drug treatment lasted 4 h; and cells were cultured until the control wells had achieved 80% confluence (typically 4-5 days). For every experiment each drug level and controls were performed in hexuplicate; each experiment was performed in at least duplicate. Error bars represent one standard deviation about the mean for the six wells per lane measured for each drug concentration.

### Example 5

Antiestrogen Binding Site (AEBS) and Estrogen Receptor (ER) Mediate Uptake and Distribution of 4-Hydroxytamoxifen-targeted Doxorubicin-formaldehyde Conjugate in Breast Cancer Cells

In Example 4, the design, synthesis, and preliminary evaluation of a class of doxorubicin-formaldehyde conjugates targeted to the estrogen receptor (ER) and antiestrogen binding site (AEBS), proteins commonly present in large quantities in breast cancer cells was described. The targeting group was 4-hydroxytamoxifen (4-OHT), the active metabolite of the antiestrogen tamoxifen. At least two isoforms of the estrogen receptor have been identified, designated alpha and beta. They are both expressed in MCF-7 breast cancer cells, bind estradiol equally, and bind 4-OHT with comparable affinity. Photoaffmity labeling experiments indicate that AEBS in liver microsomes consists of at least four proteins, three of which have been identified as microsomal epoxide hydrolase, carboxyesterase (ES 10) and liver fatty acid binding protein (L-FABP), and all are involved in lipid metabolism. Recent experiments indicate that two of the proteins of AEBS in MCF-7 cells are 3β-hydroxysterol-Δ⁸-Δ⁷-isomerase and 3β-hydroxysterol-Δ⁷-reductase and that both are involved in cholesterol biosynthesis.

The formaldehyde function was incorporated in the form of an N-Mannich base joining the amide of a salicylamide moiety to the amine of doxorubicin (Structure E, FIG. 2). The salicylamide moiety was used as a time based chemical trigger to release the doxorubicin-formaldehyde conjugate, the presumed doxorubicin active metabolite, with a half-life for hydrolysis of about 60 min under physiological conditions. The salicylamide trigger was tethered via ethylene glycol units to 4-hydroxytamoxifen, the active metabolite of tamoxifen. The targeting group was selected based on its high binding affinity to ER and AEBS. An equimolar mixture of E and Z geometric isomers of 4-hydroxytamoxifen was utilized because previous work indicates that para-hydroxy-substituted triarylbutenes isomerize under cell culture conditions, compromising the interpretation of results with pure isomers.

Preliminary biological evaluation identified DOX-TEG-TAM (2c), the conjugate containing the triethylene glycol derived tether, as the lead compound. DOX-TEG-TAM was more cytotoxic than DOX (1) and untargeted control conjugate DOX-saliform (3, DOXSF) in all four breast cancer cell lines tested, regardless of ER and multidrug resistance (MDR) expression. The most dramatic enhancement in activity for DOX-TEG-TAM relative to DOX and DOXSF was observed in MCF-7/Adr cells, an ER-negative breast cancer cell line that expresses MDR. DOX-TEG-TAM was 140-fold and 28-fold more cytotoxic to MCF-7/Adr cells than DOX and DOXSF, respectively. MCF-7/Adr cells are a doxorubicin-resistant variant of MCF-7 cells. In addition to growth inhibition assays, the targeted conjugates' estrogen receptor binding affinity was investigated. DOX-TEG-TAM retained 2.5% of the estrogen receptor binding affinity relative to targeting group alone.

The dramatic enhancement in growth inhibition of the targeted doxorubicin-formaldehyde conjugates in MCF-7/Adr cells relative to doxorubicin and untargeted DOX-saliform cannot be explained in terms of targeting the estrogen receptor; MCF-7/Adr cells are ER-negative. These observations raise the possibility that targeting is occurring, at least in part, through binding interaction with AEBS. The AEBS targeting hypothesis is as follows: (1) the targeted conjugates passively diffuse across the cytoplasmic membrane, (2) the targeting group binds to cytosolic AEBS, (3) the AEBS serves to sequester the conjugate, preventing drug efflux by the p-glycoprotein drug efflux pump (expressed as part of the MDR phenotype in MCF-7/Adr cells), and (4) the trigger fires, releasing the doxorubicin active metabolite, which then intercalates and alkylates DNA, leading to cell death. Whereas, in the case of doxorubicin and DOXSF following diffusion across the cell membrane, the p-glycoprotein efflux pump would rapidly transport them out of the MCF-7/Adr cells.

The validity of the hypothesis was tested by a series of experiments to measure the uptake and retention of the lead compound, DOX-TEG-TAM, relative to doxorubicin and untargeted DOXSF. The cellular accumulation of drug was observed by tracking the presence of the anthraquinone fluorophore via flow cytometry. Enhanced accumulation of targeted conjugate relative to controls would suggest effective targeting. Furthermore, uptake of the targeted conjugate should be reduced in the presence of a competing ligand if targeting is mediated by an AEBS binding interaction.

The reliance on anthracycline fluorescence to quantify cell uptake is complicated by the effect of local environments on the anthracycline fluorophore. For example, drug fluorescence is enhanced in lipid membranes and partially quenched by drug-DNA intercalation. However, cellular doxorubicin fluorescence has been shown to increase in a time- and dose-dependent manner; as well, cell growth inhibition is directly correlated with doxorubicin fluorescence. Therefore, drug fluorescence provides a reliable indication of the relative degree of drug uptake and retention.

In addition to flow cytometry, fluorescence microscopy was utilized to assess the cellular distribution of doxorubicin fluorophore following treatment with targeted conjugate and untargeted controls. If the targeted conjugate experiences binding to extranuclear AEBS as part of the mechanism, the drug should appear cytosolic following short (5-60 min) treatment times, prior to trigger hydrolysis.

This example demonstrates that DOX-TEG-TAM is taken up and retained by AEBS-positive MCF-7, MCF-7/Adr, MDA-MB-231, and MDA-MB435 cancer cell lines to a greater extent than clinical DOX and untargeted DOXSF. Furthermore, DOX-TEG-TAM uptake in MDA-MB-435 cells is reduced in the presence of tamoxifen, as a competing ligand, in a dose dependent manner. DOX-TEG-TAM appears cytosolic by fluorescence microscopy after short treatment times (5-60 min) in contrast to DOX and DOXSF which both appear nuclear. DOX-TEG-TAM retains 63% of the AEBS binding affinity relative to the targeting group alone. DOX-TEG-TAM is also taken up by AEBS negative, ER-positive, Rtx-6 cells, but with these cells uptake is inhibited by the ER specific ligand estradiol. These data support a targeting mechanism mediated by AEBS as well as ER.

Uptake and release of DOX-TEG-TAM: The uptake of 500 nM DOX, DOXSF, and DOX-TEG-TAM following treatment for various times up to 1 h was assessed by flow cytometry. Uptake beyond that treatment time was not explored since the half-life for hydrolysis of the formaldehyde conjugates is 1 h. The release of drugs following a 500 nM treatment for 1 h was assessed at various times out to 6 h post-treatment. Resistant MCF-7/Adr cells were first assessed as they provide a means to evaluate the AEBS targeting hypothesis to explain the dramatic improvement in tumor cell growth inhibition observed for DOX-TEG-TAM relative to DOXSF and clinical DOX. The uptake in MCF-7/Adr cells following treatment for 20,40, and 60 min with 500 nM DOX, DOXSF, or DOX-TEG-TAM shows that DOX and DOXSF were taken up by the cells to the extent of one relative fluorescent unit (RFU); while DOX-TEG-TAM was taken up almost twice as much. Drug release for all three compounds was observed at 0.5, 1, 3, and 6 h following a 1 h treatment with 500 nM of each cytotoxin. In all three cases the drug fluorescence decreased dramatically up to 1 h post-treatment, as free drug was transported from the cell. At 6 h post-treatment DOX-TEG-TAM maintained a higher level of drug retention relative to DOX and DOXSF. The enhanced uptake and retention of DOX-TEG-TAM relative to DOX and DOXSF parallels the growth inhibition data of Example 4 and provides evidence in favor of the targeting hypothesis involving interaction with AEBS.

The uptake and release of DOX, DOXSF, and DOX-TEG-TAM in estrogen receptor-positive, drug sensitive MCF-7 cells shows that DOX-TEG-TAM was taken up to a greater extent (25 RFU) than both DOXSF (16 RFU) and DOX (5 RFU). DOX-TEG-TAM showed the highest level of doxorubicin fluorophore retention following drug release after 6 h. Again, the uptake and retention parallel the growth inhibition data for DOX, DOXSF, and DOX-TEG-TAM of Example 4.

Additionally, uptake and release data were obtained for DOX, DOXSF, and DOX-TEG-TAM in estrogen receptor-negative MDA-MB-435 and MDA-MB-231 cells. In both cell lines DOX-TEG-TAM was also taken up to a much greater extent than DOX and DOXSF. In MDA-MB-435 cells DOX-TEG-TAM was taken up 3-fold and 5-fold more than DOXSF and DOX, respectively. In MDA-MB-23 1 cells DOX-TEG-TAM was taken up 2-fold and 6-fold more than DOXSF and DOX, respectively. In both cell lines doxorubicin fluorophore, following I h treatment with 500 nM DOX-TEG-TAM, was retained to a greater extent than it was following treatment with 500 nM DOXSF or DOX.

The comparison of the extent of DOX-TEG-TAM uptake as a function of breast cancer cell type illustrates that MDA-MB-435 cells take up substantially more drug following a 1 h treatment with 500 nM DOX-TEG-TAM. MCF-7 and MDA-MB-231 cells take up about 25% relative to MDA-MB-435 cells; while MCF-7/Adr cells, consistent with the overexpression of the p-glycoprotein drug efflux pump, take up a relatively small amount of drug. The enhanced uptake of DOX-TEG-TAM in MDA-MB-435 cells relative to MCF-7 and MDA-MB-231 cells was not anticipated based on growth inhibition data, as the IC₅o's for all three cell lines following 4 h DOX-TEG-TAM treatment are quite similar (30-40 nM).

Competitive inhibition of drug uptake: If DOX-TEG-TAM targeting is AEBS mediated, the presence of a competitive ligand should inhibit uptake. MDA-MB-435 cells were utilized for competition experiments as they take up all three compounds to a greater extent than the other breast cancer cells evaluated. MDA-MB-435 cells were treated with 0.5 μM DOX-TEG-TAM, DOX or DOXSF for 1 h in the presence of various concentrations of tamoxifen. In the presence of 10 μM tamoxifen competitor, the uptake of DOX and DOXSF relative to DOX-TEG-TAM decreased by only 8% and 6%, respectively. While in the case of cells treated with DOX-TEG-TAM, uptake of targeted drug decreased dramatically in a dose-dependent manner. At 10 μM tamoxifen, the uptake was 47% of the uptake of DOX-TEG-TAM in the absence of competitor, supporting the hypothesis that DOX-TEG-TAM targeting is AEBS mediated.

Analysis of drug distribution by fluorescence microscopy: If drug targeting is mediated by extranuclear AEBS, the cellular distribution of doxorubicin fluorophore following treatment with DOX-TEG-TAM should be predominantly cytosolic following short treatment times (treatment time less than the half-life for hydrolysis). Furthermore, once the trigger has fired the DOX fluorophore should appear nuclear. DOX was used as a control since with DOX treatment, drug fluorescence typically appears nuclear as the clinical drug accumulates in nuclear DNA.

Fluorescence micrographs of MDA-MB-435 cells following treatment with 500 nM DOX or 500 nM DOX-TEG-TAM as a function of time showed that following treatment for 5 min, almost no fluorescence was observed for the DOX treated cells and very little was observed for DOX-TEG-TAM cells. However, after 20 min, DOX fluorophore was observed in the nucleus of the cells following treatment with DOX, while cytosolic fluorescence was observed for DOX-TEG-TAM treated cells. Following 40 min of treatment, the DOX treated cells were observed to have accumulated more nuclear fluorophore. However, in the cells treated with DOX-TEG-TAM for 40 min the fluorescence still appeared extra-nuclear, however, the fluorophore appeared to have localized at a cytosolic site. The observed localization of DOX-TEG-TAM fluorophore appeared even more dramatic following treatment for 1 h. Following DOX treatment for 1 h and 3 h, the fluorescence continued to appear exclusively nuclear. Following DOX-TEG-TAM treatment for 3 h, the fluorescence appeared predominantly nuclear. This is consistent with the observation that the half-life for hydrolysis of the trigger is about 60 min; after three half-lives the majority of the DOX-TEG-TAM should have hydrolyzed from the targeting group and the liberated doxorubicin active metabolite translocated to the nucleus, forming DNA virtual crosslinks. In an additional control experiment, untargeted DOXSF was found to mimic the localization pattern (exclusively nuclear) that was observed for DOX.

DOX-TEG-TAM binding affinity to AEBS: The binding affinity of DOX-TEG-TAM relative to E/Z-4-OHT was determined using MCF-7 cell lysate as an AEBS source. The lysate was incubated with ³H-tamoxifen and cold competitors; 1000 nM estradiol was added to saturate estrogen receptors present in the cell lysate. Following incubation, free, unbound tamoxifen was stripped from solution with 2% dextran-coated charcoal (DCC) buffered suspension; bound ³H-tamoxifen in solution was then quantified via scintillation counting. Three concentrations of ³H-tamoxifen total binding (no competitor) was subtracted from ³H-tamoxifen binding in the presence of competitor. Theoretically, a compound (competitor) with no AEBS affinity would result in no difference between total binding and competitor. At all three concentrations of ³H-tamoxifen, DOX-TEG-TAM binding affinity was less than that of the targeting group alone. DOX-TEG-TAM binding affinity was 70%, 72%, and 48% relative to the targeting group (5) at 0.05 nM, 0.5 nM, and 5 nM ³H-tamoxifen respectively. Taken as an average, DOX-TEG-TAM retains 63% ±13% of the total binding affinity of the targeting group, E/Z-4-OHT.

Effect of DOX-TEG-TAM on AEBS-negative Rtx-6 breast cancer cells: Rtx-6 cells, kindly provided by Dr. Marc Poirot (Toulouse, France), were evaluated as an AEBS-negative control cell line. Rtx-6 cells, a tamoxifen resistant breast cancer cell line, are a clonal variant of MCF-7 cells. The Rtx-6 cells were utilized to determine the effect of the absence of AEBS on the cytotoxicity and uptake of DOX, DOXSF, and DOX-TEG-TAM.

Rtx-6 cells, in logarithmic growth, were treated with DOX, DOXSF, or DOX-TEG-TAM to establish the concentration that inhibited 50% of cell growth (IC₅₀) following a 4 h treatment. The IC₅₀'s are compared in Table 6 with those previously determined for MCF-7, MCF-7/Adr, MDA-MB-231 and MDA-MB-435 cells. The concentrations inhibiting 50% of cell growth following 4 h treatment with DOX, DOXSP, and DOX-TEG-TAM were 200 nM, 60 nM, and 70 nM, respectively.

The uptake and release in Rtx-6 cells were also investigated. Rtx-6 cells were treated with DOX, DOXSF, or DOX-TEG-TAM as described above to determine both the uptake and the release of drug following a 1 h treatment. DOX-TEG-TAM was taken up to a greater extent than DOX (>3-fold) and DOXSF (>2-fold). The enhanced uptake relative to DOX and DOXSF could be attributed to the presence of ER and the lipophilicity of DOX-TEG-TAM. The uptake of DOX-TEG-TAM in Rtx-6 cells is perhaps best compared to the uptake in the parent MCF-7 cell line. DOX-TEG-TAM was taken up to a greater extent in MCF-7 cells (25 RFU) relative to Rtx-6 cells (14 RFU). One possible explanation for this difference is the presence of both AEBS and ER in MCF-7 cells versus only ER in Rtx-6 cells. Consistent with this explanation is the dose dependent inhibition of uptake of DOX-TEG-TAM by the ER specific ligand, estradiol. Estradiol at 20 nM inhibited the uptake by 70%. No further inhibition occurs at higher concentrations, possibly because of non-specific binding of DOX-TEG-TAM at hydrophobic sites in Rtx-6 cells. In contrast, 20 nM estradiol had no effect on the uptake of DOX-TEG-TAM by ER negative, MDA-MB-435 cells.

TABLE 6
Comparison of growth inhibition for various breast cancer
cell lines by targeted and untargeted drugs as a function
of ER, MDR, and AEBS expression. IC₅₀ values are
reported in nM and represent the concentration of drug
that inhibits 50% of the cell growth.^{a}

| | ER/AEBS/ | | | DOX-TEG- |
|---|---|---|---|---|
| Cell Line | MDR | DOX | DOXSF | TAM |
| MCF-7 | +/+/− | 200 ± 26 | 70 ± 5 | 40 ± 6 |
| MCF-7/Adr | −/+/+ | 10000 ± 1300 | 2000 ± 320 | 60 ± 9 |
| Rtx-6 | +/−/− | 200 ± 20 | 60 ± 6 | 70 ± 7 |
| MDA-MB-231 | −/+/− | 300 ± 33 | 80 ± 9 | 30 ± 5 |
| MDA-MB-435 | −/+/− | 150 ± 14 | 50 ± 9 | 40 ± 6 |

^{a}IC₅₀ values for MCF-7, MCF-7/Adr, MDA-MB-231, and MDA-MB-435 cells were reported earlier.¹⁷ IC₅₀ values for Rtx-6 cells were determined as described in the Experimental Section.

Detection of AEBS in MCF-7/Adr and MDA-MB-435 cell lines: An exhaustive search of the literature uncovered no indication of the presence or expression level of AEBS in MCF-7/Adr and MDA-MB-435 cell lines. There is evidence for the presence of AEBS in MCF-7 and MDA-MB-231 cells lines and the expression level has been reported 140,000 sites/cell and 82,000 sites/cell for MCF-7 (R+) and MDA-MB-231 (ER⁻), respectively. AEBS have been reported to be present in breast cancer cells independent of estrogen receptor expression, with levels typically higher in ER⁺ cells lines.

MCF-7/Adr and MDA-MB-435 cell lysates were prepared in the same manner as the MCF-7 lysate utilized for AEBS binding assays. MCF-7, MCF-7/Adr, and MDA-MB-435 cell lysates containing 5 nM ³H-tamoxifen were incubated in the presence and absence of 5000 nM cold tamoxifen. Additionally, cold estradiol (1000 nM) was added to every sample to saturate the estrogen receptor present in the low speed lysate. Total lysate binding was measured for each cell lysate in the absence of cold tamoxifen; while non-specific binding was measured in the presence of 1000-fold cold tamoxifen. The difference between total binding and non-specific binding is, by definition, the AEBS specific binding. The ratio of AEBS specific binding for MCF-7/Adr and MDA-MB-435 lysates relative to the AEBS specific binding for MCF-7 lysate was determined. MCF-7/Adr cell lysate contained about 53% of the AEBS present in the MCF-7 cell lysate. MDA-MB-435 cell lysate contained 126% of the AEBS present in the MCF-7 cell lysate.

In conclusion, the enhanced uptake measured by flow cytometry and fluorescence microscopy experiments support the hypothesis that DOX-TEG-TAM targeting is mediated through the antiestrogen binding site as well as the estrogen receptor. In addition to DOX-TEG-TAM retaining roughly 60% of the AEBS binding affinity of the targeting group for AEBS, DOX-TEG-TAM is taken up and retained to a significantly greater extent (up to 6-fold) than DOX and DOXSF. When MDA-MB-435 cells are treated with DOX or DOXSF for various times up to 3 h, anthracycline fluorescence appears nuclear; while DOX-TEG-TAM remains in the cytosol following treatment for times less (5-40 min) than the 60 min half-life for hydrolysis. From 40-60 min, DOX-TEG-TAM fluorophore appears to localize at a cytosolic site, and following release from the trigger/targeting group, the DOX fluorophore appears nuclear.

Perhaps the most compelling evidence for the role of AEBS and ER in the targeting mechanism comes from the competition experiments. The uptake of DOX-TEG-TAM by AEBS-positive, ER negative MDA-MB-435 cells was substantially reduced in the presence of tamoxifen, an AEBS ligand and by AEBS negative, ER-positive Rtx-6 cells, in the presence of estradiol, a specific ER ligand. The competitive inhibition was observed to be dose dependent, with DOX-TEG-TAM uptake reduced by over 50% in the presence of 20-fold tamoxifen or 0.02-fold estradiol, respectively, to a level similar to untargeted DOXSF. In summary, the data support the hypothesis that the DOX-TEG-TAM targeting mechanism involves an interaction with the antiestrogen binding site and estrogen receptor.
Experimental Section: The concentrations of test compounds were determined spectrophotometrically by UVNis absorption with a Diode Array spectrophotometer interfaced to an data system as described for each biological assay. Flow cytometric measurements were performed using a flow cytometer. Fluorescence microscopy was performed using a IRB fluorescence microscope with an ebq 100 mercury lamp power source equipped with a digital CCD camera system. Cell lysis was performed with a Ultrasonic processor fitted with a microtip.

MCF-7 and MDA-MB-231 cells were obtained from American Type Culture Collection (Rockville, Md.). MCF-7/Adr doxorubicin-resistant cells were a gift from Dr. William W. Wells (Michigan State University, East Lansing, Mich.). MDA-MB435 and Rtx-6 cells were generously provided by Dr. Renata Pasqualini (MD Anderson Cancer Center, Houston, Tex.) and Dr. Marc Poirot (Toulouse, France), repectively. MCF-7, MCF-7/Adr, and MDA-MB-23 1 cells were maintained in vitro by serial culture in RPMI 1640 medium supplemented with 10% fetal bovine serum (Gemini Bioproducts, Calbassas, Calif.), L-glutamine (2 mM), HEPES buffer (10 mM), penicillin (100 units/mL), and streptomycin (100 μg/mL). MDA-MB-435 cells were maintained in vitro by serial culture in DMEM medium supplemented with 5% fetal bovine serum, L-glutamine (2 mM), sodium pyruvate (1 mM), and non-essential amino acids and vitamins for minimum essential media. Rtx-6 cells were maintained in vitro by serial culture in RPMI 1640 medium supplemented with 5% FBS, L-glutamine (2 mM), HEPES buffer (10 mM), penicillin (100 units/mL), streptomycin (100 μg/mL) and 1.00 μM tamoxifen (Sigma, St. Louis, Mo.). Tamoxifen was excluded from the media when experiments were performed. Cells were maintained at 37° C. in a humidified atmosphere of 5% CO₂ and 95% air.

Uptake and release of DOX, DOXSF, and DOX-TEG-TAM: The uptake and release of DOX-TEG-TAM in breast cancer cells relative to DOX and untargeted DOXSF was performed as described with modifications. Breast cancer cells in log phase growth were dissociated with trypsin-EDTA, counted, resuspended in media at 1.5×10⁵ cells/mL, and plated into six well plates (450,000 cells/well) and allowed to adhere overnight. The cells were treated with 0.5 μM DOX, DOXSF, or DOX-TEG-TAM for various amounts of time (20 min, 40 min, and 60 min). Drug treatment was accomplished by addition of 30 μL of 100× drug solution (50 μM) to 3 mL of media which was mixed and immediately added to the cells. For each time point, the media was removed, cells were trypsinized, and trypsinization was quenched with 3 mL phenol red-free RPMI 1640 media (no serum) at 4° C. Cells were pelleted by centrifugation at 300 g for 5 min at 10° C. The supernatant was decanted and the cells were re-suspended in 1 mL serum- and phenol red-free RPMI 1640 media and placed on ice.

For drug retention samples, the cells were treated for 1 h with 0.5 μM DOX, DOXSF, or DOX-TEG-TAM. Following drug treatment, the media was removed and replaced with 3 mL fresh, 37° C., full (containing serum and phenol red indicator) cell media and the cells were incubated for various times (0.5 h, 1 h, 3 h, and 6 h). Following the allotted release times, the cells were prepared as described above. Drug treatment was performed such that all cell samples would be prepared within 2 h. Previous work has demonstrated that no loss of fluorescence is observed from cells stored on ice for up to 4 h.

The extent of drug uptake and retention was measured by flow cytometry. Cells were analyzed with excitation at 488 nm (15 mW Ar ion laser), with emission monitored between 570 and 600 nm. Instrument settings were optimized for each cell line and held constant for all experiments; 10,000 cells were analyzed for anthracycline fluorescence. The data are presented as the mean fluorescence for each condition with the background, drug-free cell fluorescence subtracted.

Tamoxifen competition experiments: MDA-MB-435 cells were treated with 0.5 μM DOX, DOXSF, or DOX-TEG-TAM in the absence and presence of the competitor, tamoxifen. Following drug treatment for 1 h, both anthracycline and tamoxifen were removed. The cell samples were prepared and analyzed by flow cytometry as described above.

Estradiol competition experiments: Rtx-6 cells were treated with 0.5 μM DOX-TEG-TAM in the absence and presence of the competitor, estradiol. Following drug treatment for 1 h, both anthracycline and estradiol were removed. The cell samples were prepared and analyzed by flow cytometry as described above.

Analysis of drug distribution by fluorescence microscopy: MDA-MB-435 cells in log phase growth were dissociated with trypsin-EDTA and counted. Cells were suspended in media at 8.3×10⁴ cells/mL and 3 mL of cell solution was aliquoted into 6 well plates and allowed to adhere overnight. The cells were treated with 0.5 μM DOX, DOXSF, or DOX-TEG-TAM for various amounts of time (5 min, 20 min, 40 min, 1 h, and 3 h). Drug treatment was accomplished by addition of 30 μL of 100× drug solution (50 μM) to 3 mL of media which was mixed and immediately added to the cells.

Following drug treatment, the media was removed and cells were washed once with 3 mL of serum- and phenol red-free RPMI 1640 at room temperature and 3 mL of the same media was then added back to each well. The cells were then immediately analyzed by fluorescence microscopy. Each condition was performed individually to minimize the amount of time between drug treatment and fluorescence detection.

Microscopic images of the cells were observed at a magnification of 40× and recorded with a IRB fluorescence microscope equipped with a digital CCD camera system. Cell images were observed with a shutter time of 0.05 s; fluorescence images were observed with a shutter time of 1.000 s. Drug fluorescence was observed at wavelengths above 590 nm with excitation between 515 and 560 nm.

Evaluation of DOX-TEG-TAM binding affinity to AEBS: The binding affinity of DOX-TEG-TAM (2c) relative to E/Z-4-OHT (5) was measured through competition assay with tritiated tamoxifen (³H-TAM) through a procedure adapted from several sources. MCF-7 cells were utilized as the AEBS source. Cells were cultured in six T-175 flasks to 90% confluence. To harvest, cells were washed with 10 mL of Hank's balanced salt solution and dissociated from the flasks with 2 mL of trypsin. Trypsinization was quenched with 10 mL of phenol red-free RPMI media supplemented with 10% dextran-coated charcoal (DCC)-stripped fetal calf serum (“stripped media”); cells from six T-175 flasks were combined and pelleted by centrifugation at 300 g for 5 min at 25° C. The supernatant was decanted, the cells were resuspended in 50 mL stripped media and enumerated with a hemacytometer. The cells were then pelleted again by centrifugation, as described above. The supernatant was decanted and the cells were suspended in pH 7.4 lysis buffer (10% v/v glycerol, 10 mM Tris, 1.5 mM EDTA, 0.5 mM dithiothreitol, 10 mM Na₂MoO₄, 1.0 mM phenylmethylsulfonyl fluoride, supplemented with Complete-Mini™ protease inhibitors) at 4° C. such that the cell density was 38 million cells per mL lysis buffer. Cells were lysed at 0° C. via sonication with a microtip set at maximum power for 10 cycles of 6 s on followed by 24 s off. The AEBS-enriched lysate was obtained by centrifugation of the homogenate at 12,000 g for 30 min at 4° C. The supernatant was dispensed into 100 μL aliquots and stored at -70° C. The lysate protein density was measured and for all experiments the stock lysate was diluted such that the protein density was 2.0 mg/mL.

Competitive ligands were prepared as 120× solutions (50 nM working concentrations) in DMSO containing 1% acetic acid. Competitor concentrations were determined for ligands containing the DOX chromophore by optical density at 480 nm (ε=11,500 l/mol·cm). Three different concentrations (5 nM, 0.5 nM, and 0.05 nM) of tritiated tamoxifen were prepared as 240× solutions in DMSO containing 1% acetic acid. Cold estradiol (1 μM) was added to every sample to saturate the estrogen receptor present in the low speed lysate; cold estradiol was prepared as a 240 μM (240×) stock solution to provide a 1 μM estradiol working concentration. Equal volumes of the 240× solutions of tritiated tamoxifen and cold estradiol were added together to provide 120× solutions of each ligand in DMSO containing 1% acetic acid. The 120× solutions were then diluted 1:10 in pH 7.6 TE (10 mM Tris, 1 mM EDTA) buffer to provide 12× solutions of ³H-TAM/estradiol and competitors. Aliquots of cell lysate (100 μL) were thawed at 4° C.; 10 μL of 12× competitor was added, followed by 10 μL 12×³H-TAM/estradiol at each of the three tritiated tamoxifen concentrations. Total binding was measured by addition of vehicle (DMSO containing 1% acetic acid) in the absence of competitor; ³H-TAM binding inhibition was measured by addition of E/Z-4-OHT or DOX-TEG-TAM. Reaction lysates were vortexed vigorously and stored at 4° C. for 18 h.

Following incubation, unbound aromatic organics were stripped from the lysate by addition of 280 μL of DCC as a 2% w/v suspension in pH 7.6 TE buffer (10 mM Tris, 1.0 mM EDTA). Following the addition of the DCC, the reaction lysates were vortexed and stored on ice for 15 min, with vortexing every 5 min. DCC was pelleted by centrifugation at 3000 g for 10 min at 4° C.; 300 μL of lysate supernatant was transferred to scintillation vials containing 4 mL of Econosafem biodegradable scintillation cocktail. The vials were then vortexed vigorously and each sample was counted for 5 repetitions of 3 min counts. This counting protocol was then repeated to ensure reproducibility. Scintillation counting background was subtracted from all measurements. The percentage of targeting group binding for DOX-TEG-TAM relative to targeting group alone was determined by comparison of the reduction of tritiated tamoxifen at each concentration Scintillation counting was performed in triplicate and each competitor was assayed in duplicate. Error bars represent one standard deviation for the percentage DOX-TEG-TAM binding relative to E/Z-4-OHT at three different tritiated tamoxifen concentrations.

Growth inhibition of Rtx-6 cells: The concentration inhibiting half the growth (IC₅₀) was determined as previously described with minor modifications. All compounds were solubilized in dimethylsulfoxide containing 1% v/v acetic acid. The concentrations of all 100× DMSO/1% AcOH drug solutions was determined spectrophotometrically by absorbance at 480 nm (ε=11,500 l/mol·cm). Drug treatment lasted 4 h; and cells were cultured until the control wells had achieved 80% confluence (typically 4-5 days). For every experiment each drug level and controls were performed six time. Error bars represent one standard deviation about the mean for the six wells per lane measured for each drug concentration.

Detection of AEBS in MCF-7/Adr and MDA-MB-435 cells: The presence of AEBS was measured through the binding of ³H-tamoxifen in MCF-7, MCF-7/Adr, and MDA-MB-435 cell lysates. MCF-7/Adr and MDA-MB-435 cell lysates were prepared as described above for the AEBS binding assay. All three cell lysates were diluted to achieve a uniform protein density of 2.0 mg/mL.

Binding ligands were prepared as 120× solutions in DMSO containing 1% acetic acid and delivered to cell lysates as described above. MCF-7, MCF-7/Adr, and MDA-MB-435 cell lysates containing 5 nM ³H-tamoxifen were incubated in the presence and absence of 5000 nM cold tamoxifen. Additionally, cold estradiol (1000 nM) was added to every sample to saturate the estrogen receptor present in the low speed lysate; cold estradiol was prepared as described above. Total lysate binding was measured for each cell lysate in the absence of cold tamoxifen; while non-specific lysate binding was measured in the presence of cold tamoxifen. The difference between total binding and non-specific binding is, by definition, the AEBS specific binding. Following incubation, the cell lysates were prepared for liquid scintillation counting as described above. The ratio of AEBS specific binding for MCF-7/Adr and MDA-MB-435 relative to the AEBS specific binding for MCF-7 is presented. Error bars represent one standard deviation from the mean of the scintillation counting statistics.

### Example 6

Doxorubicin-formaldehyde Conjugates Targeting α_{ν}β₃ Integrin Significant limitations for DOX treatment of cancer are drug resistance and chronic cardiotoxicity. One of the most promising methods to reduce the side effects of a cytotoxin like DOX is selective delivery to cancer cells and/or their associated angiogenesis. A protein complex that may be a good target for drug delivery is the α_{ν}β₃ integrin. α_{ν}β₃ is involved in many cell-matrix recognition and cell adhesion phenomena giving it an important role in angiogenesis and tumor metastasis. The α_{ν}β₃ integrin is overexpressed on the surface of tumor and endothelial cells responsible for angiogenesis, and its expression correlates with tumor progression in glioma, melanoma, breast, and ovarian cancer. α_{ν}β₃ exists in discrete activation states and activation can be induced with manganous ion. Activated α_{ν}β₃ supports breast cancer cell arrest during blood flow and strongly promotes breast cancer metastasis. In tumor-induced angiogenesis, invasive endothelial cells bind via this integrin to extracellular matrix components. The inhibition of this interaction induces apoptosis of the proliferative angiogenic vascular cells. These factors combined make α_{ν}β₃ an attractive target for antiangiogenic and antimetastatic therapies. A number of RGD peptide and peptide mimetics developed over the last decade exhibit excellent binding affinity and selectivity for α_{ν}β₃. The peptide cyclic-(N-Me-VRGDf) known as Cilengitide™ has proceeded as far as phase II clinical trials as a potent antagonist of α_{ν}β₃. Small RGD containing peptides have successfully been employed to deliver cytotoxins, MRI contrast agents, radionuclides, liposomes, and fluorescent agents to tumors which express α_{ν}β₃.

Ruoslahti and coworkers' report that a DOX-CDCRGDCFC (RGD-4C) conjugate that targets α_{ν}β₃ substantially inhibited tumor growth in mice relative to DOX with fewer side effects prompted further exploration. Scheeren and coworkers reported that DOX conjugated with RGD-4C via a plasmin cleavable tether inhibited HUVEC cell binding to plates coated with vitronectin with an IC50 of ˜150 nM and exhibited a cytotoxicity IC50 of 750 nM against the same cell line. The plasmin activated prodrug failed to inhibit tumor growth in vivo better than DOX alone but did exhibit less toxicity based on weight loss in a tumor bearing mouse model. This example describes the synthesis and biological evaluation of DOXSF conjugated to two different RGD containing peptides, RGD-4C and cyclic-(N-Me-VRGDf).

The conjugation of DOXSF to α_{ν}β₃ targeting peptides serves several purposes. The drug conjugate is a prodrug with little or no activity until the trigger (N-Mannich base hydrolysis) releases the cytotoxin from the peptide. RGD-4C and cyclic-(N-Me-VRGDf) have both been shown to accumulate in tumor relative to other tissue, with a peak accumulation point of approximately 40-60 min. Based on this delivery schedule a triggered release of DOX active metabolite with a half-life of 60 min should localize a good portion of the drug in tumor relative to other tissue. This design may reduce side effects such as cardiotoxicity and increase the amount of active drug in and around the tumor.

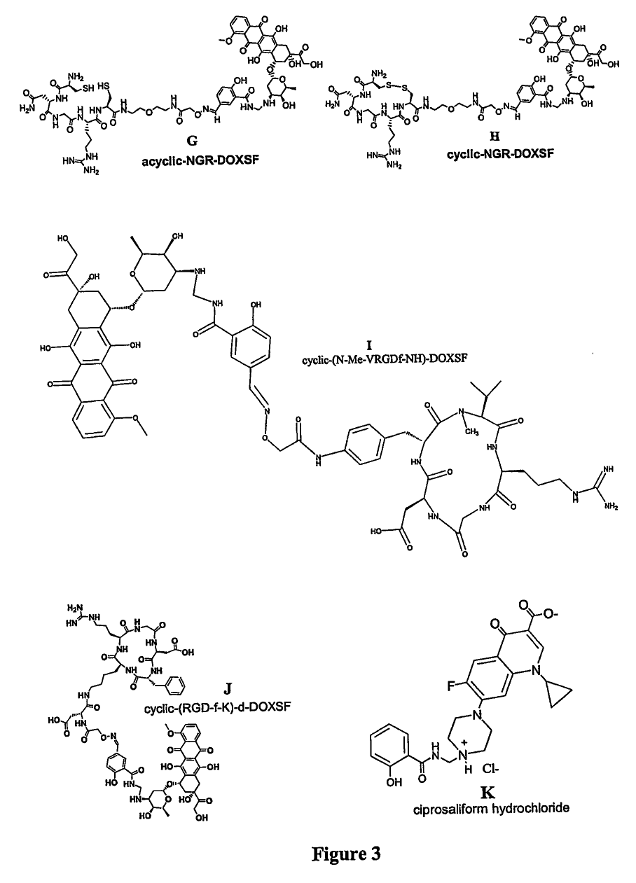

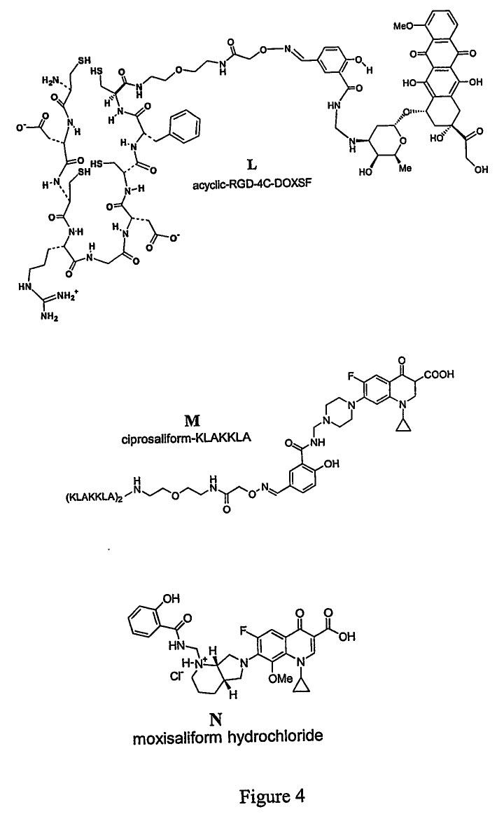

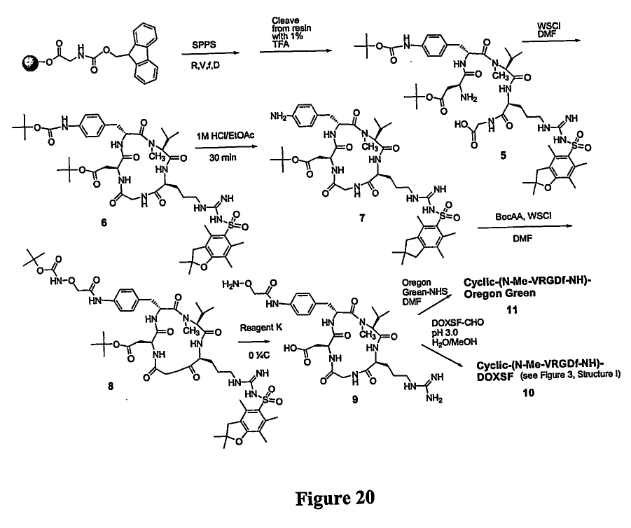

Synthesis: Design: DOXSF was conjugated to the RGD containing peptides RGD-4C and cyclic-(N-Me-VRGDf) via a short hydroxylamine ether tether which forms an oxime bond with a formyl group added at the 5′-position of the salicylamide of DOXSF. This oxime was found to be quite stable under a variety of aqueous conditions. The N-Mannich base that contains the formaldehyde equivalent necessary to produce the DOX active metabolite hydrolyzes with a half life of 60 min at physiological temperature and pH. Hydrolysis of the N-Mannich base is also the trigger that releases the DOX active metabolite from the targeting peptide. The synthesis of acyclic-RGD-4C-DOXSF (Structure L, FIG. 4) and cyclic-(N-Me-VRGDf-NH)-DOXSF (Structure I, FIG. 3) are detailed below and the synthetic schemes are shown in FIGS. 19 and 20.

Materials and instruments: All reactions were performed under inert atmosphere. For amino acids with sensitive side chains the following were used: Fmoc-Asp-tBu, Fmoc-Cys-trt, Fmoc-Arg-pbf, Fmoc-D-4-aminoPhe(Boc). Melting points were uncorrected. The 1H, COSY, HSQC, HMBC, and 13C NMR high-resolution spectra were obtained with a spectrometer. Electrospray mass spectra were measured with a Perkin Elmerm Sciex API III, equipped with an ion-spray source, at atmospheric pressure. Analytical HPLC was carried out on a system consisting of an auto injector and pumping system, fluorescence detector, diode array UV-Vis detector. A protein C-4 column (4.6×250 mm) was used for analytical HPLC with a flow rate of 0.5 mL/min and a gradient solvent system of 0.1% TFA/acetonitrile: 0 to 15 min, 98% to 40% aqueous; 15 to 20 min, 40% to 15% aqueous; 20 to 25 min, 15% to 98% aqueous; detection at 220, 254, 280, and 480 nm. For preparative HPLC, a C-4 column (22×250 mm) was used with the same solvent system on a semipreparative HPLC consisting of pumping system, UV-1 detector, and data system eluting at 15 mL/min. The gradient used for cyclic-(N-Me-VRDGf-NH)-tether was: 0 to 11.5 min, 98% to 80% aqueous; 11.5 to 15 min, 80% to 30% aqueous; 15 to 16 min, 30% to 55% aqueous; 16 to 20 min, 55% to 98% aqueous; detection at 254 nm. The gradient used for complete drug conjugates was: 0 to 30 min, 90% to 60% aqueous; 30 to 50 min, 60% to 90% aqueous; detection at 470 nm.

Synthesis of the hydroxylamine ether tether, (2-{2-[2-(2,2-dimethyl-propionylaminooxy)-acetylamino]-ethoxy}-ethyl)-carbamic acid 9H-fluoren-9-ylmethyl ester 1. Fmoc-2-(2-aminoethoxy)-ethylamine hydrochloride 1.30 g was weighed out and placed in a dry 250 mL round bottom flask under an argon atmosphere. Anhydrous dimethylformamide (10 mL) was added by syringe, followed by 2.0 mL of pyridine with stirring. (Boc-aminoxy)-acetic acid 1.04 g (2 equiv.) and WSCI 0.69 g (2 equiv.) were measured out and added in one portion to the solution of amine. The reaction was monitored by analytical HPLC and 0.33 equiv. of (Boc-aminoxy)-acetic acid and WSCI were added after 1 h to drive the reaction to completion. The reaction was then diluted with ethyl acetate (100 mL) and washed with dilute acetic acid (3×50 mL), followed by pH=10.0 sodium bicarbonate. The organic phase was dried with sodium sulfate and concentrated under vacuum to yield 1.78 g (99%) of clear solid product 1. 1H-NMR in chloroform-d 1.42 (s, 9H), 3.20 (m, 2H), 3.53 (m, 6H), 4.20 (t, J=6.8 Hz, 1H), 4.27 (s, 2H), 4.43 (d, J=6.8 Hz, 2H), 5.75 (br, 1H), 7.26 (t, J=7.6 Hz, 2H), 7.43 (dd, J=4.8, 7.2 Hz, 2H), 7.62 (d, J=7.2 Hz, 2H), 7.76 (d, J=7.6 Hz, 2H); ESI-MS m/z: 500, calculated for (M+H⁺) m/z 500.23.
Partial deprotection of 1 to {2-[2-(2-aminooxy-acetylamino)-ethoxy]-ethyl}-carbamic acid 9H-fluoren-9-ylmethyl ester 2 and loading of 2 onto resin. The Fmoc-2-(2-aminoethoxy)-ethyl-Boc-aminoxy-amide (1, 1.7 g) was dissolved in a solution containing 10 mL of trifluoroacetic acid and 1.1 mL of thioanisole at 0 oC. The solution was allowed to stir for 1 h at room temperature and then concentrated (<5 mL) and the product precipitated into cold diethyl ether (100 mL). The precipitate was then collected as the TFA salt by filtration and washed with ether (3×20 mL). 1H NMR in methanol-d4 showed complete removal of the Boc protecting group so the compound 2)was then loaded on trityl-chloride resin as follows. To a dry 250 mL round bottom flask was added 50 mL of dry methylene chloride, 2.2 mL anhydrous pyridine, and 2. After the amine went into solution with stirring, 1.1 g of trityl chloride resin was added in one portion and the mixture allowed to stir for 22 h. The resin was then collected by filtration, washed with 17:2:1 (v/v) methylene chloride:MeOH:DIEA (2×25 mL), and with methylene chloride (2×30 mL), followed by methanol (3×50 mL). The resin was then dried under vacuum and the loading was determined by treatment of an aliquot (5 mg) with 0.5 mL of 20% piperidine/DMF for 15 min and dilution to 50 mL with DNF followed by UV absorbance measurement at 301 nm. Resin loading ranged from 0.5 mmol/g to 0.84 mmol/g.

General procedure for the synthesis of linear peptides: The linear peptides were synthesized by the solid-phase method using Fmoc strategy, starting with the preloaded Fmoc tether from above. The peptides were prepared on a 0.25 mmol scale by single amino acid couplings using a 4-fold excess of Fmoc-amino acids and TBTU/HOBT activation on a peptide synthesizer. Fmoc groups were removed by sequential treatment (3×) with 20% piperidine/DMF. Acyclic RGD-4C was synthesized in the following order Cys-Phe-Cys-Asp-Gly-Arg-Cys-Asp-Cys (SEQ ID NO: 1) and final Fmoc deprotection of the peptide was performed while still on the resin. The linear peptide was cleaved from the resin and deprotected by a 3 h treatment with degassed reagent K The resin was then filtered and the mother liquor concentrated under vacuum (<5 mL) and the product precipitated drop-wise into cold ether (60 mL). The peptide was collected by filtration (#1 filter paper) and washed with ether (3×20 mL). The crude peptide was dried under vacuum overnight and analyzed by analytical HPLC and mass spectrometry. The analytical HPLC trace showed a single peak (r.t., 22.22 min), ESI-MS, m/z 1180.6, calculated for (M+H⁺) 1179.39
Synthesis of the acyclic-RGD-4C-DOXSF. To a 50 ML pear shaped flask containing 3 mg of DOXSF-CHO was added 2 mL 0.1% TFA and the solution was degassed with argon by bubbling for 5 min. Acyclic-RDG-4C-tether (3, 12 mg, 3 equiv.) was dissolved in 1.5 mL of degassed methanol and then added in one portion to the DOXSF-CHO solution by syringe. The reaction was allowed to stir at room temperature and was monitored by HPLC. After approximately 3 h the reaction was found to be complete by HPLC analysis (new peak found r.t.=17.14 min, 480 nm). The reaction was purified directly by preparative HPLC and all major peaks analyzed by mass spectrometry. Product showed a mass spectral ion at m/z 1883.2 (M+H⁺) (calculated 1883.6) and a base peak at m/z 942.2 ((M+2H⁺)/2). The yield of acyclic-RGD-4C-DOXSF was 1.2 mg of compound, pure by ESI-MS and analytical HPLC. Drug was then formulated with 3 equiv. of citric acid and 6 equiv. of lactose and stored at −80° C.

Synthesis of bicyclic-RGD-4C-tether 4. Acyclic RGD-4C-tether 3 (30 mg) was dissolved in a solution of 50 mL of TFA and 2.5 mL of dimethyl sulfoxide. Anisole (0.5 mL) was then added by syringe with stirring and the solution stirred for 1 h. The reaction was monitored by HPLC and stopped when complete (usually I h). The solution was then concentrated under high vacuum to yield a mixture (approximately 50:50) of bicyclic isomers. HPLC gave two peaks at r.t., 27.05 and 27.31 min for the two isomers; ESI-MS, m/z 1175.6, calculated for (M+H⁺) 1175.4 for both isomers,
Synthesis of protected acyclic-N-Me-VRGDf-NH2, 5: General Fmoc synthesis was performed as for acyclic-RGD-4C-tether, but TBTU/HOBt coupling was found to be inefficient for coupling to N-methyl valine. Peptide still on the resin was treated with 2 equiv. PyBroP, Fmoc-D-4-aminophe(Boc) and 4 equiv. DIEA in dry dichloromethane (5 mL per gram resin). The mixture was placed on a shaker for 16 h, washed with 3×10 mL of dichloromethane, and checked by the chloranil test for coupling completion. If not complete, coupling was repeated for 3 h. When coupling was finished, the resin was then treated with 3×10 mL of 20% piperidine in DMF for a period of 10 min to complete deprotection. Resin was then returned to the ABI synthesizer to complete the peptide synthesis using standard Fmoc synthesis protocol. Cleavage of the linear peptide was effected with 1% TFA in dichloromethane (3×10 mL) with shaking for 5 min each time.

The solution was concentrated under high vacuum to give the linear peptide with protecting groups intact in 98% yield as determined by analytical BPLC (one peak with r.t., 17.9 min); ESI-MS, m/z 1029.6, calculated for (M+H⁺) 1029.51.
Cyclization of protected N-Me-VRGDf-NH2 5 to yield 6: Linear peptide (386 mg) with all protecting groups intact was dissolved in 50 mL of EtOH and 1.2 equiv. of 10% aqueous HCl (v/v) was added to displace the TFA salt. When this step was omitted, trifluoracetylation of the peptide occurred during the cyclization reaction. The solution was concentrated under vacuum. Linear peptide was then dissolved in anhydrous DMF (125 mL) and 2 equiv. of WSCI were added in one portion. Reaction was monitored by HPLC and typically complete within 3 h.

The solution was concentrated under vacuum and the residue was dissolved in 50 mL of EtOAc and washed with 10% HCl (v/v) (2×50 mL). The organic phase was dried with sodium sulfate and concentrated under vacuum to give pure cyclic-(N-Me-VRGDf-NH2) with protecting groups intact 6. Analytical HPLC showed one peak (r.t., 17.2 min); ESI-MS m/z 1012.5, calculated for (M+H⁺) 1012.51.

Selective removal of Boc protecting group from D-4-amino-Phe of fully protected cyclic-(N-Me-VRGDf-NH2) 6 to yield 7: Fully protected cyclic-(N-Me-VRGDf-NH₂) (6, 319 mg) was dissolved in 10 mL of dry EtOAc, and 1 M anhydrous HCl in EtOAc (1.5 mL) was added while the mixture was maintained at 0° C. with an ice bath. Mixture is allowed to stir for 3 h at 0° C. and then concentrated under vacuum. Product was then lyophilized from water to give a clear solid. This method completely removed the Boc group from the D-4-amino-Phe, but a small amount of peptide also experienced hydrolysis of the Asp t-Bu protecting group to release the acid. This mixture was carried forward since the deprotected Asp was not deemed problematic. HPLC analysis shows two peaks (r.t., 12.2 and 13.9 min); ESI-MS for these two peaks m/z 856.4 and 912.6, respectively. Calculated for deprotection of both D-4-amino-Phe(Boc) and Asp(tBu) (M+H⁺) 856.39; calculated for deprotection of only D-4-amino-Phe(Boc) (M+H⁺) 912.46.

Addition of Boc-aminoxyacetic acid tether to partially protected cyclic-(N-Me-VRGDf-NH₂) 7. Clear solid (288 mg) from the above reaction was dissolved in 50 mL anhydrous DMF and 3 equiv. of Boc-aminooxyacetic acid were added, followed by 1.5 equiv. of WSCI. After stirring for 1.5 h, the reaction was complete based on analytical BPLC. The mixture was concentrated under vacuum, the residue dissolved in EtOAc (50 mL), and washed with water (2×20 mL) and then 10% HCl (v/v) (2×50 mL). The organic phase was separated, dried with sodium sulfate, and concentrated under vacuum. Two peaks were observed by HPLC (r.t., 12.7 and 14.4 min); ESI-MS for the two products m/z 1028.8 and 1084.5, respectively, calculated for (M+H⁺) 1028.47 and 1084.53.

Removal of all protecting groups from cyclic-(N-Me-VRGDf-NH)-tether to yield 9. Peptide from the above reaction was added to a dry 50 mL round bottom flask and cooled to 0° C. under an argon atmosphere. Reagent K (5 mL) was added and the solution allowed to stir for 3 h at room temperature. Solution was then added dropwise to 100 mL of anhydrous ether with vigorous stirring, that had been cooled with an ice bath. The white precipitate was collected by filtration and washed three times with ether (15 mL) and dried under vacuum. Pure product cyclic-(N-Me-VRGDf-NH)-tether (9, 171 mg) was obtained as determined by HPLC (r.t., 7.2 min); ESI-MS, m/z 677.2, calculated for (M+H⁺) 677.33. To assign the 1H NMR spectrum unequivocally, the following spectra were run; 1H NMR, COSY, HSQC, and HMBC all in D2O. 1H NMR in D2O: 0.47 (3H, d, J=6 Hz, CH3, Val), 0.80 (3H, d, J=6 Hz, CH3, Val), 1.48, (2H, m, CH2, Arg), 1.83 (2H, m, CH2, Arg), 1.85 (1H, m, CH, Val), 2.62 (1H, dd, J=17 and 6 Hz, CH2, D-Phe) 2.80 (3H, s, CH3, N-Me Val), 2.6-2.9 (3H, m, CH2, D-Phe, and Asp), 3.07-3.13 (2H, m, CH2, Arg), 3.45 (1H, d, J=14 Hz, Gly), 3.83 (1H, m, CH, Arg), 4.04 (1H, d, J=14 Hz, Gly), 4.23 (1H, d, J=11 Hz, CH, Val), 4.47 (1H, t, J=6 Hz, CH, D-Phe), 4.6-4.8 (under HOD peak, CH2, hydroxylamine ether tether), 5.09 (1H, t, J=7 Hz, CH, Asp), 7.18 (2H, d, J=8 Hz, CH, Phe), 7.29 (1H, d, J=8 Hz, CH, Phe).

Conjugation of cyclic-(N-Me-VRGDf-NH)-tether 9 to DOXSF to yield cyclic-(N-Me-VRGDf-NH)-DOXSF 10 (FIG. 20): DOXSF-CHO (4 mg) was dissolved in a 3:1 mixture (2 mL) of pH 2.0 water:EtOH (v/v), and 8 mg cyclic-(N-Me-VRGDf-NH)-tether 9 was added. The solution was stirred at room temperature for 5 h until one peak with absorbance at 480 nm was observed by analytical HPLC. The product was purified by preparative HPLC to yield 4.2 mg of pure cyclic-(N-Me-VRGDf-NH)-DOXSF 10. The conjugate was concentrated under vacuum at room temperature and stored as a red solid at −80° C. HPLC analysis showed one peak (r.t., 12.7 min); ESI-MS, m/z 1379.6, calculated for (M+H⁺) 1379.54. To assign the 1H NMR spectrum the following spectra were obtained in DMF-d7; 1H NMR, COSY, HSQC, HMBC, ROESY. 1H NMR; 0.41 (3H, d, J=6 Hz, CH3, Val), 0.77 (3H, d, J=6 Hz, CH3, Val), 1.14 (3H, d, J=7 Hz, CH3, 5′), 1.45-1.51 (2H, m, CH2, Arg), 1.87-1.93 (2H, m, CH2, Arg), 1.93-1.95 (2H, m, CH2, 2′), 2.03-2.05 (1H, m, CH, Val), 2.12 (1H, dd, J=6 and 15 Hz, CH2, 8), 2.22 (1H, m, CH2, 8), 2.46 (1H, dd, J=6 and 17 Hz, CH2, D-Phe), 2.74 (3H, s, CH3, N-Me Val), 2.80 (2H, m, under DMF peak, CH2, 10), 2.9-2.99 (2H, m, CH2, Asp), 3.13-3.19 (3H, m, CH2 and CH, Arg, and 3′), 3.32 (1H, d, J=14 Hz, CH2, Gly), 3.74 (2H, m, CH, 9 and Arg), 3.95 (3H, s, CH3, 4, O-Me), 3.97 (1H, m, CH2, Gly), 4.20 (1H, q, J=7 Hz, CH, 5′), 4.34 (1H, d, J=11 Hz, CH, Val), 4.51 (1H, t, J=7 Hz, CH, D-Phe), 4.60 (4H, two singlets, CH2, 14, and hydroxylamine ether tether), 4.73 (1H, d, J=13 Hz, CH2, N-Mannich base), 4.83 (1H, d, J=13 Hz, CH2, N-Mannich base), 4.92 (1H, dd, J=6 and 9 Hz, CH, Asp), 5.0 (1H, bs, CH, 7), 5.34 (1H, bs, CH, 1′), 6.92 (1H, d, J=9 Hz, CH, 3″), 7.04 (2H, d, J=9 Hz, CH, D-Phe), 7.50 (2H, d, J=9 Hz, CH, D-Phe), 7.55 (1H, dd, J=3 and 9 Hz, CH, 4″), 7.61 (1H, dd, J=2 and 7 Hz, CH, 3), 7.85 (1H, under DMF peak, CH, 2), 7.91 (1H, under DMF peak, CH, 1), 8.03 (1H, d, J=3 Hz, CH, 6″), 8.14 (1H, s, NH, 4-Phe).

Conjugation of cyclic-(Me-VRGDf-NH)-tether 9 to Oregon Green to yield cyclic-(N-Me-VRGDf-NH)-Oregon Green 11: To a dry 25 mL round bottom flask was added 2 mg of 5′-Oregon Green 488-NHS, 1 equiv. of cyclic-(Me-VRGDf-NH)-tether (2.7 mg) and 5 mL of anhydrous DMF. The solution was allowed to stir for 5.5 h while monitored by HPLC. Solution was then concentrated under vacuum and the residue resuspended in MeOH for purification by preparative HPLC which yielded 1.1 mg (26%) of a solid yellow product. Analytical HPLC showed a single peak (r.t.=16.9 min); ESI-MS, m/z 1071.5, calculated for (M+H⁺) 1071.36.
Biological Evaluation: Cell culture: Human breast carcinoma cell line MDA-MB-435 was maintained in DMEM media supplemented with 10% FBS, penicillin (100 U/mL), streptomycin (0.1 mg/mL), L-glutamine (2 mM), sodium pyruvate (1 mM), non-essential amino acids and vitamins.

Purified proteins. Human vitronectin and bovine serum albumin (BSA; A-7030) were purchased from Sigma (St. Louis, Mo.). α_{ν}β₃-specific monoclonal antibody (mAb) LM609 was purchased from Chemicon™ (Temecula, Calif.).

Cell adhesion assay. Cell adhesion was determined by coating wells of 96 well plates with 100 μL 5 μg vitronectin/mL in Dulbecco's phosphate buffered saline (D-PBS) from 2.0 h to over night at room temperature. Wells were washed twice with deionized water and nonspecific binding sites were blocked with 200 μL heat inactivated (20 min at 60° C.) 1.0% BSA in D-PBS from 2.0 h to over night at 37° C. Wells were washed five times with deionized water and allowed to dry for 30 min at room temperature or stored at 4° C. for extended periods. Cells were harvested from a subconfluent T-175 tissue culture flask by rinsing with 35 mL D-PBS and incubating with 2 mL of 4 mM EDTA for 3 min at 37° C. The EDTA solution was neutralized by adding 48 mL of DMEM containing penicillin-streptomycin. Cells were washed once with 50 mL DMEM+penicillin-streptomycin and resuspended in DMEM+penicillin-streptomycin at a final concentration of 8.5×105 cells/mL. MnCl₂ was added resulting in a final concentration of 500 μM. Peptides or antibody were added prior to adding cells (100 μL) to 96 well plates. Cells were allowed to adhere for 90-100 min at 37° C. Non-adherent cells were removed by aspiration and washing twice with D-PBS containing 900 μM Ca2⁺ and 500 μM Mg2⁺. Adherent cells were quantified via measuring cellular metabolism of 3-(4,5-dimethylthiazol-2-yl)-2,5-diphenyl tetrazolium bromide at 37° C. For every experiment, each condition was performed in triplicate; experiments were performed at least twice. α_{ν}β₃ binding assessed by flow cytometry with cyclic-(N-Me-VRGDf-NH)-Oregon Green. Cells were harvested from five subconfluent T-175 cell culture flasks by rinsing with 35 mL D-PBS and incubating with 2 mL of 4 mM EDTA for 3 min at 37° C. The EDTA solution was neutralized by adding 40 mL D-PBS. Cells were washed once with 9 mL D-PBS and resuspended in D-PBS+0.5% BSA or D-PBS+0.5% BSA, 500 μM MnCl₂ and 500 μM MgCl₂. Various concentrations of cyclic-(N-Me-VRGDf-NH)-Oregon Green were added and allowed to incubate for 90 min at 37° C. Cells were washed twice, resuspended in 500 μL D-PBS+0.5% BSA and analyzed by flow cytometry. Cells were analyzed with excitation at 488 nm (Ar ion laser), with emission monitored between 510 and 550 nm. Ten thousand cells were analyzed per condition. The data are presented as the mean fluorescence for each condition with the background, drug-free cell fluorescence subtracted.

Uptake of DOX, DOXSF, and acyclic-RGD-4C-DOXSF: A flow cytometry method of measuring uptake of DOX, DOXSF, and acyclic-RGD-4C-DOXSF in breast cancer cells was performed as previously described with modifications. MDA-MB435 breast cancer cells in log phase growth were dissociated with trypsin-EDTA, counted, resuspended in media at 2×105 cells/mL, and plated into six-well plates (5.0×105 cells/well) and allowed to adhere overnight. Drug solutions of DOX, DOXSF, and ayclic-RGD-4C-DOXSF were prepared in DMSO with 1% acetic acid at 50 μM. Before treatment with drug, cell media was removed, cells were washed with HBSS (0.5 mL), and then fresh cell media, with or without 500 μM Mn²⁺, was placed into the wells (2 mL). Drug treatments of 0.5 μM were accomplished by the addition of 20 μL from the drug solutions to the desired wells and incubation for various amounts of time (20 min, 40 min, and 60 min). For each time point, the cell media was removed, cells were washed with HBSS, trypsinized, and trypsinization was quenched with 5 mL of cell media at 4° C. Cells were pelleted by centrifugation at 200 g for 5 min at 10° C. The supernatant was decanted, and the cells were resuspended in 5 mL of D-PBS at 4° C., repelleted, resuspended in 2 mL of D-PBS at 4° C., and placed on ice. Drug treatments were performed in such a manner that all cell treatment times would end at approximately the same time to ensure comparable measurements with the FACScan instrument. The amount of drug uptake was measured by flow cytometry. Cells were analyzed with excitation at 488 nm (15 mW Ar ion laser), with emision monitored between 570 and 600 nm. Instrument settings were optimized for the cell line and held constant for all experiments; 10,000 cells were analyzed for each sample's anthracycline fluorescence. The data are presented as the mean fluorescence for each condition divided by the background, drug-free mean fluorescence.

Growth inhibition assay: Cells were treated for 4 h then allowed to grow until control wells reached ˜80% confluence (4-5 days). Cells were quantified via measuring crystal violet staining or cellular metabolism of MTT. For every experiment, each condition was performed in hexuplicate; experiments were performed at least twice.

Synthesis: The two predominant bicyclic structures are formed by oxidation of the four thiols to two disulfide bridges. Upon formation of the disulfide bridges, RGD-4C became poorly water soluble over a range of pH. Based on the observed change in solubility upon formation of the disulfide bridges, it was hypothesized that acyclic-RGD 4C was the actual peptide that targeted phage to MDA-MB-435 tumors in mice. Linear RGD containing peptides are known to have a short circulation time in the blood stream due to the activity of proteases, but since targeted delivery to tumor is relatively rapid, this peptide drug conjugate was tested. The synthetic strategy for acyclic-RGD-4C-DOXSF outlined in FIG. 19, used an oximation reaction of a formyl group placed at the 5′-position of the salicylamide group of DOXSF and a hydroxylamine ether tether at the carboxyl terminus of the peptide. The oximation reaction was regioselective for the aryl aldehyde and produced a robust connection between the targeting group and the salicylamide trigger, time release group. Both acyclic-RGD-4C-tether and acyclic-RGD-4C-DOXSF have good water solubility.

An attractive alternative to RGD-4C is the cyclic peptide, cyclic-(N-Me-VRGDf), developed by Merck as a selective and potent α_{ν}β₃ antagonist. The X-ray crystal structure of cyclic-(N-Me-VRGDf) bound to ανβ3 shows the D-Phe and N-Me-Val directed toward solvent making these residues attractive attachment points for conjugation of cytotoxin, or other molecular probe. A short tether was attached to the 4-position of D-Phe since this would take advantage of the rigid nature of the aromatic ring, essentially creating a short linear tether and projecting the steric bulk of DOXSF toward solvent. Cyclic-(N-Me-VRGDf-NH)-tether with the hydroxylamine functional group at the terminus of the tether was synthesized from start to finish in high yield with no chromatography. Again, the targeting group was connected to DOXSF-CHO via the oximation reaction. The conjugate, cyclic-(N-Me-VRGDf-NH)-DOXSF, obtained in pure form after preparative HPLC, was stable for months while stored at −80° C. as the TFA salt of its N-Mannich base.

α_{ν}β₃ binding: bicyclic-RGD-4C-tether (as a 50:50 mixture of the 1-4;2-3 and 1-3;2-4 isomers), acyclic-RGD-4C-tether, and cyclic-(N-Me-VRGDf-NH)-tether were assayed for their ability to bind the α_{ν}β₃ integrin present on viable MDA-MB-435 cells using a vitronectin competition assay. Vitronectin is the endogenous ligand for the α_{ν}β₃ integrin. Conditions for inhibition of MDA-MD-435 cell adhesion to vitronectin, including the requirement of Mn²⁺, were established by using an α_{ν}β₃ specific monoclonal antibody (LM609). Targeting compounds or targeted-DOXSF were added to cell suspensions created by release of cells from cell culture flasks with EDTA as opposed to trypsin to preserve the integrity of α_{ν}β₃. Drug treated cells were then added to cell culture plates coated with bovine serum albumin (BSA)±vitronectin. Cells were allowed to adhere, wells were washed with D-PBS, and cells were quantified via measuring cellular metabolism of 3-(4,5-dimethylthiazol-2-yl)-2,5-diphenyl tetrazolium bromide (MTT). Nonspecific binding (cells bound to BSA coated wells) was subtracted from total binding (cells bound to BSA and vitronectin coated wells) in order to determine specific binding to vitronectin. The concentrations of compound required to inhibit binding of 50% of the cells to vitronectin (IC₅₀ values) are shown in Table 7. The acyclic-RGD-4C isomer was chosen over the bicyclic isomer for further experiments due to better water solubility and higher binding affinity for the α_{ν}β₃ integrin. Next, acyclic-RGD-4C-DOXSF and cyclic-(N-Me-VRGDf-NH)-DOXSF compounds were assayed for their ability to bind the α_{ν}β₃ integrin (Table 7). The binding affinities of both acyclic-RGD-4C-tether and cyclic-(N-Me-VRGDf-NH)-tether decreased by only one order of magnitude upon addition of DOXSF indicating that the tethering system does not preclude binding. IC₅₀ values for both acyclic-RGD-4C-DOXSF and cyclic-(N-Me-VRGDf-NH)-DOXSF in the vitronectin assay are significantly lower than those for the RGD-4C-DOX conjugate with the plasmin cleavable tether, pioneered by Scheeren and coworkers (10 and 5 nM vs. 150 nM).

TABLE 7
IC₅₀ values for inhibition of MDA-MB-435 cell binding to vitronectin
and cell growth as a function of targeting group or drug design.

| | IC₅₀ for inhibition | IC₅₀ for inhibition |
|---|---|---|
| | of cell | of cell growth (nM), |
| Compound | binding (nM) | treatment time |
| Bicyclic-RGD-4C-tether 4 | 10 ± 1 | |
| Acyclic-RGD-4C-tether 3 | 1 ± 0.2 | |
| Cyclic-(N-Me-VRGDf- | 0.5 ± 0.1 | |
| NH)-tether 9 | | |
| Cyclic-(N-Me-VRGDf- | 2 ± 0.4 | |
| NH)-Oregon Green 11 | | |
| Acyclic-RGD-4C-DOXSF | 10 ± 2 | (1000 ± 200), 20 min; |
| | | (50 ± 10), 4 h |
| Cyclic-(N-Me-VRGDf- | 5 ± 1 | (1000 ± 200), 20 min; |
| NH)-DOXSF 10 | | (250 ± 50), 1 h; |
| | | (90 ± 20), 4 h |
| DOXSF | >10⁴ | (50 ± 10), 4 h |
| DOX | >10⁴ | (800 ± 200), 20 min; |
| | | (300 ± 60), 1 h; |
| | | (120 ± 30), 4 h |

Cyclic-(N-Me-VRGDf-NH)-tether was also analyzed for binding to α_{ν}β₃ on MBA-MB-435 cells as a function of Mn²⁺ activation with cyclic-(N-Me-VRGDf-NH)-tether bound to Oregon Green fluorescent dye. Binding as a function of cyclic-(-Me-VRGDf-NH)-Oregon Green concentration in the presence and absence of Mn²⁺ was measured by flow cytometry. The experiment was performed with cells in suspension, released from the growth flask with EDTA. In the presence of Mn²⁺, binding of dye to cells increased with concentration of dye and plateaued at about 100 nM. In the absence of Mn²⁺ little binding of dye was observed even at 100 nM cyclic-(N-Me-VRGDf-NH)-Oregon Green, consistent with targeted dye binding to activated α_{ν}β₃. As reported in Table 7, the IC50 for targeted dye binding to cells is 2 nM, approximately midway between the values for cyclic-(N-Me-VRGDf-NH)-tether and cyclic-(N-Me-VRGDf-NH)-DOXSF.

Uptake of acyclic-RGD-4C-DOXSF: uptake of acyclic-RGD-4C-DOXSF by MDA-MB-435 cells was measured by flow cytometry after drug treatment for various periods of time in the presence and absence of additional Mn²⁺ beyond that present in FBS. Concentration of targeted drug relative to DOX and DOXSF in cells was determined from emission of the DOX fluorophore. After a 1 h drug treatment time, DOX was taken up 3-fold more than acyclic-RGD-4C-DOXSF, and uptake of acyclic-RGD-4C-DOXSF was independent of additional Mn²⁺. In Table 8, uptake of acyclic-RGD-4C-DOXSF is compared with uptake of DOX and DOXSF at two time points, 30 min and 4 h, and three drug concentrations, 100, 500 and 1000 nM, in the absence of additional Mn²⁺. At the 30 min time point approximately 30% of the time-release trigger of acyclic-RGD-4C-DOXSF or DOXSF had fired and at the 4 h time point more than 90% of the trigger had fired based upon the known half-life for the trigger. After treatment for 30 min with 500 nM drug, uptake of fluorophore from acyclic-RGD-4C-DOXSF was 60% of DOX and 20% of DOXSF. However, after treatment for 4 h with 500 nM drug, uptake of fluorophore from acyclic-RGD-4C-DOXSF and DOX was comparable and uptake of fluorophore from DOXSF was only 2-fold higher. These results suggest that acyclic-RGD-4C-DOXSF does not significantly penetrate the cell membrane and that the DOX fluorophore only enters after the trigger releases the DOX-formaldehyde conjugate.

TABLE 8
Uptake of acyclic-RGD-4C-DOXSF by MDA-MB-435 cells as a
function of dose and time in the absence of additional Mn²⁺
compared with uptake of DOX and DOXSF. Relative uptake was
measured by flow cytometry observing fluorescence from the DOX
fluorophore. Results are presented in relative fluorescence units (RFU).

| | RFU with | RFU with | RFU with |
|---|---|---|---|
| Drug treatment | 100 nM drug | 500 nM drug | 1000 nM drug |
| Acyclic-RGD-4C-DOXSF | 1.00 | 1.34 | 1.65 |
| (30 min) | | | |
| DOX (30 min) | 1.23 | 2.12 | 3.21 |
| DOXSF (30 min) | 1.88 | 5.55 | 9.23 |
| Acyclic-RGD-4C-DOXSF | 1.80 | 6.35 | 9.92 |
| (4 h) | | | |
| DOX (4 h) | 1.90 | 5.88 | 12.03 |
| DOXSF (4 h) | 3.23 | 13.25 | 24.51 |

Cancer cell growth inhibition: acyclic-RGD-4C-DOXSF and cyclic-(N-Me-VRGDf-NH)-DOXSF were also assayed for their ability to inhibit growth of MDA-MB-435 cells relative to DOX and DOXSF. Cells treated in cell culture plates and non-treated (-control) cells were allowed to grow to near confluency, and then quantified via measuring crystal violet staining or cellular metabolism of MTT. The concentrations of drug required to inhibit growth of cells by 50% (IC50 values) are shown in Table 7 as a function of drug treatment time. The data in Table 7 were obtained in the absence of additional Mn²⁺ because control experiments showed no effect from Mn²⁺ on cytotoxicity. Both acyclic RGD-4C-DOXSF and cyclic-(N-Me-VRGDf)-DOXSF are more cytotoxic than clinical DOX and comparable in cytotoxicity to DOXSF with a drug treatment time of 4 h. With shorter drug treatment times the cytotoxicities of targeted drugs and DOX are comparable. The slight decrease in cytotoxicity observed for cyclic-(N-Me-VRGDf-NH)-DOXSF relative to DOXSF is comparable to the loss relative to parent drug observed by Scheeren and coworkers. Earlier control experiments established that the miniscule amounts of formaldehyde that would be released even from complete hydrolysis of the conjugate would contribute nothing to the observed growth inhibition. Conjugation of cytotoxic drugs to triggers and targeting groups often causes a drop in cytotoxicity.

DOXSF prodrug-RGD conjugates were synthesized and evaluated for binding to α_{ν}β₃ in the vitronectin cell adhesion assay and for inhibition of MDA-MB-435 cancer cell growth. A prodrug with this design should bind α_{ν}β₃ and localize in/or near the tumor and vascular endothelial cells of the developing blood supply. Upon hydrolysis of the N-Mannich base, the conjugate would release the DOX active metabolite locally because of its short lifetime with respect to further hydrolysis to DOX (half-life, approximately 5 min). The advantage of delivering the DOX active metabolite is that it is more cytotoxic to both sensitive and resistant tumor cells.

RGD-4C as a targeting group was explored first because of significant activity in tumor bearing mice reported for RGD-4C-DOX conjugates with the peptide in its oxidized form. The structures for the conjugates, however, were not well defined by the synthetic strategy or from spectroscopic data. A later report established that 14;2-3-bicyclic-RGD-4C has an order of magnitude better affinity for α_{ν}β₃ than the other major regioisomer, l-3;2-4-bicyclic-RGD-4C. Oxidation of acyclic-RGD-4C-tether gave roughly a 50:50 mixture of the 1-4;2-3- and 1,3;2,4-bicyclic isomers. It was found that acyclic-RGD-4C-tether had better affinity for α_{νβ}₃ (Table 7) and much better aqueous solubility than a 50:50 mixture of the two regioisomers of bicyclic-RGD-4C-tether. The result that acyclic-RGD-4C bound with higher affinity than the mixture of bicyclic isomers is surprising since formation of the disulfide bridges make the structure more rigid. Based upon this result, acyclic-RGD-4C-tether was selected for conjugation to DOXSF. Acyclic-RGD-4C-DOXSF conjugate exhibited a decrease in affinity for α_{ν}β₃ relative to the peptide alone (10 nM vs 1 nM), but cytotoxicity against MDA-MB-435 cells (IC50=50 nM) was comparable to that of DOXSF. Comparison of uptake of DOX fluorophore by MDA-MB-435 cells treated with either acyclic-RGD-4C-DOXSF, DOX or DOXSF as a function of treatment time suggests that targeted drug doesn't penetrate the plasma membrane. Appearance of DOX fluorophore from targeted drug in cells requires release of the DOX-formaldehyde conjugate by the salicylamide trigger.

Because acyclic-RGD-4C-DOXSF has the potential for instability due to 4 sulfhydryl groups, a cyclic RGD peptide was created with the cycle created via a peptide linkage between the amino and carboxyl termini, cyclic-(N-Me-VRGDf). Although a variant, cyclic-(KRGDf), has been used to attach various molecules to the α_{ν}β₃ targeting peptide at the ε-amino group of the Lys, the linear D-Phe of cyclic-(N-Me-VRGDf) was used as an attachment point guided by the co-crystal structure of the ligand binding domain of α_{ν}β₃ bound to cyclic-(Me-VRGDf). Based on molecular modeling of our conjugate bound to α_{ν}β₃ this linear tether should permit attachment of a large molecule without a significant decrease in binding affinity to α_{ν}β₃ Indeed, cyclic-(N-Me-VRGDf-NH)-DOXSF exhibited an IC₅₀ in the vitronectin binding assay of 5 nM. Further, a conjugate of cyclic-(N-Me-VRGDf-NH) with Oregon Green showed dose and Mn²⁺ dependent binding to MDA-MB-435 cells by flow cytometry. The cancer cell growth inhibition by cyclic-(N-Me-VRGDf-NH)-DOXSF is better than DOX but reduced by a factor of two relative to DOXSF. A higher IC50 relative to DOXSF is attributed to a reduced rate of uptake because the targeted drugs don't appear to penetrate the plasma membrane, and for uptake, the salicylamide trigger must first release the DOX-formaldehyde conjugate.

The likely scenario for prodrug activity in vivo based upon these experiments would be binding to α_{ν}β₃ overexpressed by tumor and/or tumor vascular endothelial cells during circulation, followed by hydrolysis of the N-Mannich base releasing the DOX active metabolite extracellularly. The active metabolite should enter the cell more rapidly than free DOX due to its lack of charge, then induce apoptosis via the formation of covalent crosslinks in cellular DNA. Possibly, some conjugate could be internalized via receptor mediated or fluid phase endocytosis and hydrolyzed to the active metabolite intracellularly. Since the active metabolite is not cationic, as opposed to DOX, the P-170 drug efflux pump resistance mechanism would likely have less effect. Similarly, resistance mechanisms which suppress oxidative stress and the production of formaldehyde will have little impact since the active metabolite released by the trigger already has formaldehyde incorporated.

Both RGD-DOXSF conjugates have good affinity for α_{ν}β₃ and are more cytotoxic than clinical DOX. The salicylamide N-Mannich base trigger hydrolyzes with a half-life of 60 min, which is appropriate for the rate of targeted drug delivery to tumor. Both RGD-targeted drug designs show good water solubility and are promising candidates for in vivo testing in tumor bearing nude mice.

### Example 7

Use of the Platform Technology of the Present Invention for the Design, Synthesis and Evaluation of Doxorubicin-Formaldehyde Conjugate Targeted to Cancer Cells and their Associated Angiogenesis with NGR Peptides

Using in vivo phage display, the peptide CNGRC (NGR) (SEQ ID NO:2) was identified as a tumor homing peptide. In the homing peptide, it has been suggested that the Cys residues are oxidized to cystine and that the peptide in its most active form has a cyclic structure. A conjugate of the cytotoxic antitumor drug doxorubicin with the cyclic peptide prepared with poorly defined chemistry is significantly more active against MDA-MB-435 human breast tumors in nude mice. The peptide conjugate was proposed to target doxorubicin to aminopeptidase N, overexpressed by immature vascular endothelial cells associated with tumor angiogenesis.

CNGRC (SEQ ID NO:2) has been conjugated to tumor necrosis factor alpha (TNF-alpha) using recombinant technology. The conjugate, NGR-TNF, was 12-15 fold more efficient than murine TNF in decreasing the tumor burden in lymphoma and melanoma animal models. In a subsequent study it was concluded that the CNGRC (SEQ ID NO:2) targeting peptide of NGR-TNF had a cyclic structure resulting from oxidation of the Cys residues to cystine.

Doxorubicin was conjugated at the 14-hydroxyl group to cyclic-CNGRC via a succinate linker. The antitumor activity was tested in nude mice bearing human ovarian cancer xenografts (OVCAR-3). Weekly i.v. administration (3 mg/kg Dox-equiv., 3×) showed 40% growth delay which was not better than an equivalent treatment with untargeted doxorubicin.

This Example describes the design, synthesis, and preliminary evaluation of acyclic and cyclic-CNGRC peptides tethered to doxorubicin-formaldehyde conjugate via a cleavable group tethered to the NGR peptide. The cleavable group is the salicylamide N-Mannich base of doxorubicin-formaldehyde conjugate of the present invention. The complete drug is assembled by an oximation reaction of a formyl group at the 5-position of the salicylamide with a hydroxylamine ether functional group at the end of a tether bonded to the C-terminus of the peptide.

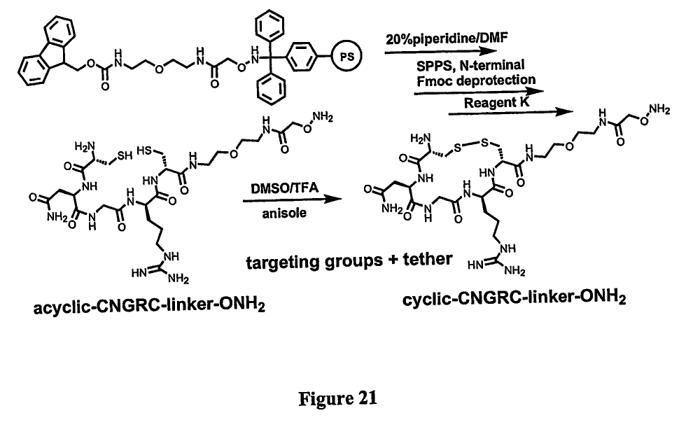

Design and Syntheses: Acyclic-CNGRC-tether was synthesized starting with Fmoc-protected tether bonded to polystyrene resin (FIG. 21) prepared as previously described. The amino acid residues were added stepwise using standard solid state Fmoc amino acid synthesis procedures. The peptide-bearing tether was removed from the resin and deprotected with reagent K to give acyclic-CNGRC-tether (acyclic-CNGRC-linker-ONH₂). Subsequent oxidation with dimethylsulfoxide (DMSO) catalyzed with trifluoroacetic acid (TFA) gave cyclic-CNGRC-tether (cyclic-CNGRC-linker-ONH₂) bonded to tether.

Acyclic- and cyclic-dox-NGR were assembled by reaction of the respective CNGRC-tether with 5-formyldoxsaliform at low pH as shown with cyclic-CNGRC-linker-ONH₂ in FIG. 21, and the products were purified by reverse phase HPLC. 5-Formyldoxsaliform was prepared by reaction of doxorubicin with 5-formylsalicylamide and formaldehyde as previously described.

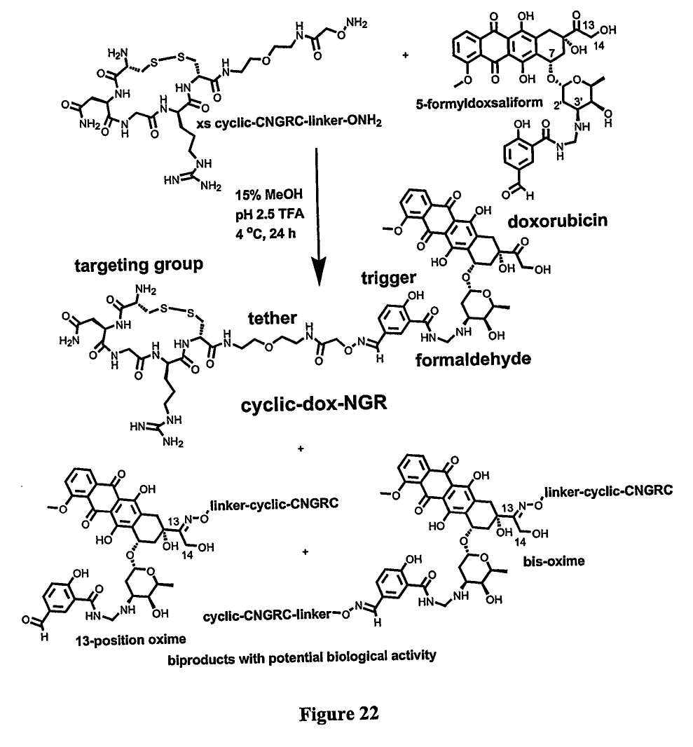

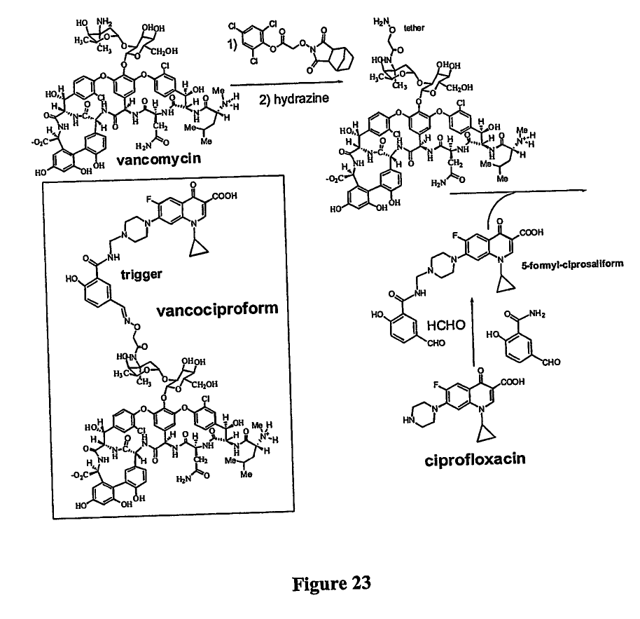

Byproducts of the oximation reaction with both acyclic- and cyclic-CNGRC-linker-ONH₂ (shown in FIG. 22 with cyclic-CNGRC-linker-ONH₂) resulted from oxime formation at the 13-position of doxorubicin and bis-oxime formation at the formyl substituent and the 13-positon of doxorubicin. These byproducts also have potential antitumor activity in targeted therapy and will be investigated as potential drugs.

In a mouse experiment with acyclic-dox-NGR, five nude mice were inoculated with MDA-MB-435 human breast cancer cells in the mammary fat pad and tumor allowed to progress to approximately 20 mm³ in volume. Mice were then treated i.v. weakly with 30 μg dox equivalent doses of dox-NGR or as a control, 30 μg of doxorubicin for 6 weeks, and tumor growth was measured weekly. After 8 weeks the experiment shows significant advantage to targeted drug over untargeted clinical doxorubicin at an equivalent dose.

Formulation: as reported earlier, the salicylamide trigger is stabilized by low pH through protonation of the 3′-amino group of the doxorubicin moiety. Clinical samples of doxorubicin are commonly formulated with lactose to increase the rate of dissolution in saline. Consequently, acyclic- and cyclic-dox-NGR were formulated with citric acid and lactose. Formulation was performed by addition of 3 equiv of citric acid and 8 equiv of lactose to partially concentrated solution of the purified drug from HPLC and centrifugal vacuum evaporation to dryness.
Experimental: UV-vis spectrometry was performed with a diode array spectrophotometer and workstation. Mass spectral data was collected with a Perkin Elmer™ Sciex API III, equipped with an ion-spray source and workstation, scanned at 0.2 amu resolution. 5-Formylsalicylamide was prepared as previously described above.
HPLC Elution Methods: the following HPLC elution gradients were employed with A=acetonitrile and B=0.15% TFA aqueous solution (pH=1.7) unless otherwise noted. Aqueous solutions for HPLC solvents were filter with a 0.45-micron nylon filter and acetonitrile for HPLC was filtered with a 0.22-micron nylon filter.

Analytical HPLC[1]: is a liquid chromatograph equipped with a diode array UW-vis detector and workstation; chromatographies were performed with a 5 μm reverse phase C₁₈ microbore column, (2.1 mm×100 mm), eluting at 0.5 mL/min: method HPLC I (480 nm, 280 nm, 230 nm), A:B, 2:98 at 0 min, isocratic until 1 min, 25:75 at 12 min, 70:30 at 20 min, isocratic until 23 min, 2:98 at 25 min; method HPLC II (480 nm, 280 nm, 230 nm), A:B, 2:98 at 0 min, isocratic until 1 min, 35:65 at 36 min, 85:15 at 40 min, isocratic until 43 min, 2:98 at 49 min, isocratic until 50 min.

Analytical HPLC[2]: is a pump, auto-injector, diode array detector with workstation; chromatographies were performed with a 5 μm spherical C18 column (2.1 mm×100 mm), eluting at 0.5 mL/min; method HPLC IV (480 nm, 280nm, 230 nm), A:B, 5:95 to 35:65 at 36 min, 70:30 at 40 min, isocratic until 36 min, 5:95 at 49 min, isocratic until 50 min.

Semi-preparative HPLC: is a liquid chromatograph equipped with a diode array UV-vis detector and workstation; chromatographies were performed with a high speed 10 mm×100 mm, 3 μm spherical, C-18, semi-preparative column (methods HPLC V and HPLC VI) or a 10 mm×350 mm, 5 μm spherical, C18, semi-preparative column (method HPLC VII), eluting at 2.5 mL/min: method HPLC V (230 nm, 220 nm), A:B, 0:100 at 0 min, isocratic until 3 min, 15:85 at 8 min, 85:15 at 10 min, isocratic until 12.5 min, 0:100 at 15 min, isocratic until 16 min; method HPLC VI (480 nm, 280 nm, 230 mn), A:B, 20:80 at 0 min, isocratic until 24.5 min, 30:70 at 25 min, isocratic until 26 min, 90:10 at 28 min, isocratic until 28.5 min, 20:80 at 31 min, isocratic until 32 min; method HPLC VII (480 nm, 280 nm, 230 nm), A:B, 2:98 at 0 min, isocratic until 6 min, 30:70 at 10 min, isocratic until 30 min, 90:10 at 32.5 min, isocratic until 37 min, 2:98 at 41 min, isocratic until 45 min.

Preparative HPLC: is a pump with an absorbance detector and workstation; chromatographies were performed with a reverse phase C₄ column (22×250 mm), eluting at 9.0 mL/min: method HPLC VIII (450 nm), A:B, 10:90 at 0 min, isocratic to 1 min, 15:85 at 20 min, 30:70 at 40 min, 70:30 at 45 min, 10:90 at 50 min, isocratic until 55 min.

Synthesis of NGR peptides: The linear peptides were synthesized by the solid-phase method using Fmoc chemistry, starting with the preloaded Fmoc tether prepared as previously described. The peptides were synthesized by single amino acid couplings using a 5-fold excess of Fmoc-amino acids and TBTU/HOBT activation on a peptide synthesizer on a 0.25 mmol scale. Fmoc groups were removed by sequential treatment (4X) with 20% piperidine/DMF. Acyclic NGR was synthesized with amino acid residues in the following order, Cys-Asn-Gly-Arg-Cys (SEQ ID NO: 2), and the final Fmoc deprotection of the peptide was performed while still on the resin. The resin was divided into two equal portions. One portion was saved in the freezer and the other portion cleaved from the resin and deprotected by 6 h treatment with 5 mL of degassed reagent K (82.5% TFA: 5% water: 5% phenol: 5% thioanisole: 2.5% ethylene dithiol). The resin was then removed by filtration and the filtrate concentrated to 0.5 mL by rotary evaporation. Two equal portions of the peptide were precipitated by drop-wise addition into cold ether (50 mL×2). Sample was centrifuged at 3000g for 20 min; the ether was decanted off and the product washed once with cold ether (50 mL×2). The crude peptide was then dried under vacuum for 20 min and stored at −80° C. until needed. The yield was 120 mg of crude product.

Purification of acyclic-NGR-linker-ONH₂: Crude peptide (20 mg) recovered after treatment with reagent K was dissolved in 1 mL of 0.15% TFA aqueous solution. Using method HPLC V and 50 μL injections, sample was purified with the desired product eluting at 6.9 min. A major impurity eluting at 10.5 min was determined to be trityl-protected peptide by mass spectrometry. Collected samples were combined and first, rotary evaporated to dryness at 45° C. and 0.6 mbar, then on a vacuum line at 0.05 mbar overnight to yield 10 mg (14 μmol) of acyclic-NGR-linker-ONH₂: ESI-MS, m/z 711.0 [M+1] (calculated, 711.3)

Cyclic-NGR-linker-ONH₂ from oxidation of acyclic-NGR-linker-ONH₂: Crude peptide (20 mg) recovered after treatment with reagent K was dissolved in 4.5 mL of TFA and transferred to 25 mL pear flask at 0° C. The solution was allowed to cool for 30 min with stirring; then, 0.5 mL of DMSO was added drop-wise. After another 30 min, 100μL of anisole was added drop-wise. The solution was kept at 0° C. for an additional 30 min, then allowed to stir at room temperature overnight (exposed to air). After solution had set for 24 h, mass spectrometry showed complete oxidation to desired product with some product further oxidizing at the hydroxylamine ether functional group to give oxidative cleavage to an alcohol functional group. The solution was then evaporated to dryness by rotary evaporation at 45° C. and 0.4 mbar, then put on a vacuum line at 0.05 mbar for 30 min. Crude product was dissolve in 1 mL 0.15% TFA aqueous solution and purified by method HPLC V with 100 μL injections. Desired product eluted at 6.4 min with major impurities at 9.9 min (anisole) and 10.5 min (trityl-protected peptide). Collected samples were combined and evacuated to dryness by rotary evaporation at 45° C. and 0.6 mbar, then on a vacuum line at 0.05 mbar overnight to yield 8.8 mg (12.4 μmol) of purified cyclic-NGR-linker-ONH₂: ESI-MS, m/z 709.2 [M+1] (calculated 709.3)

N-(5-Formyl-2-hydroxybenzamido-methyl)-doxorubicin: To a stirring solution of 3 mg (18.3 μmol) of 5-formylsalicylamide in 1.0 mL of DMF was added 10 μL (120 μmol) of a 37% formalin solution. The reaction was stirred in a sealed 25 mL pear flask for 25 min at 60° C., at which time 5.3 mg (9.8 μmol) of doxorubicin hydrochloride was added to form a red suspension that was stirred at 60° C. After 25 min, a clear red solution had formed and the reaction was removed from the heat. The solution was evacuated to dryness by rotary evaporation at 55° C. and 6 mbar to give a red film. Product was dissolved in 0.5 mL DMF and purified with method HPLC VIII with product eluting at 31.2 min and a minor product eluting at 34.0 min. Collected sample was transfer to a 100 mL round bottom flask and evacuated to dryness by rotary evaporation at 45° C. and 0.6 mbar. Product was then put on a vacuum line at 0.1 mbar for 30 min, and then dissolved in 2 mL of 0.05% TFA. The concentration was determined by UV-vis spectroscopy and purity by method HPLC I. The yield was 2.8 mg (3.9 μmol, 39.8%) of N-(5-formyl-2-hydroxybenzamido-methyl)-doxorubicin with 95.4% purity (4.6% doxorubicin): MS-ESI, m/z 721.2 [M+1] (calculated 721.2).

Acyclic-dox-NGR: N-(5-Formyl-2-hydroxybenzamido-methyl)-doxorubicin (2 mg, 2.8 μmol) was dissolved in 2 mL of 0.05% TFA (pH 2.5) and 350 μL of methanol. The solution was kept on ice while being degassed with argon for 30 min. Acyclic-NGR-linker-ONH₂ (10 mg, 14 μmol) was added and the solution kept at 0° C. overnight. Reaction was followed by method HPLC IV and allowed to proceed at room temperature while being monitored and at 0° C. at night. Reaction was allowed to progress to about 45% conversion (5 days) of desired product then purified by method HPLC VI with 25 μL injections. The HPLC trace showed three products and starting material. Desired product eluted at 26.5 min (45.9%), 2 minor products eluted at 13.3 min (10.4%) and 23.2 min (8.2%), and unreacted N-(5-formyl-2-hydroxybenzamido-methyl)-doxorubicin eluted at 27.9 min (24.3%). The two minor products were collected and determined to be the product of oximation at the 13-position carbonyl of doxorubicin (peak at 23.2 min) and the double oximation product (both the 13 position of doxorubicin and the 5-formyl group, peak at 13.3 min) by mass spectrometry. Collected samples of desired product were combined and concentrated down by rotary evaporation to approximately 10 mL.

Concentration was determined by UV-vis spectrometry assuming a molar absorbtivity of 11,000 M⁻¹cm⁻¹. The sample was divided into 100 μg aliquots in 1.7 mL Eppendorf tubes. Sample were formulated with 3 equiv. of citric acid and 8 equiv. of lactose then placed in a Speedvac at 0.2 mbar for 4 h. When vacuum reached 0.04 mbar, samples were removed and placed at -80° C. until needed. Purity of final product was determined by method HPLC I. The yield was 1.2 mg (0.85 μmol, 30%) of acyclic-dox-NGR at 96.7% purity (3.3% doxorubicin): ESI-MS, m/z 1413.4 [M+1] (calculated 1413.5).

Cyclic-dox-NGR: N-(5-Formyl-2-hydroxybenzamido-methyl)-doxorubicin (2.8 mg, 3.9 μmol) was dissolved in 2 mL of 0.05% TFA (pH 2.5). The solution was kept on ice while being degassed with argon for 30 min. Cyclic-NGR-linker-ONH₂ (8.8 mg, 12.4 μmol) was added and the solution kept at room temperature for 8 h. Reaction was followed with method HPLC II and allowed to run at room temperature while being monitored and stored at −80° C. overnight. Reaction was allowed to progress to about 60% conversion (˜10 h at room temperature) and then purified with method HPLC VII using 60 μL injections (purified 510 μL, 25.5% of total material). HPLC trace showed three products and starting material. Desired product eluted at 24.4 min (61.7%), 2 minor products eluted at 18.3 min (15.2%) and 23.2 min (5.3%), and unreacted N-(5-formyl-2-hydroxybenzamido-methyl)-doxorubicin eluted at 37.5 min (13.4%). The two minor products were collected and determined to be the product of oximation at the 13-position carbonyl of doxorubicin (peak at 23.2 min) and the double oximation product (both the 13 position of doxorubicin and the 5-formyl group, peak at 18.3 min) by mass spectrometry. Collected samples of desired product were combined and concentrated down by rotary evaporation to approximately 10 mL. Concentration was determined by UV-vis spectroscopy. The sample was divided into 100 μg aliquots in 1.7 mL Eppendorf tubes. Samples were formulated with 3 equiv of citric acid and 8 equiv of lactose then put on a Speedvac at 0.2 mbar for 4 h. When the vacuum reached 0.04 mbar, samples were removed and placed at -80° C. until needed. Purity of final product was determined by method HPLC IV. The yield was 0.8 mg (0.57 μmol, 57.3% yield from purified portion) of acyclic-dox-NGR at 100% purity: ESI-MS, m/z 1411.4 [M+1] (calculated 1411.5).

### Example 8

Antibiotic-Aldehyde Conjugates Tethered to a Targeting Moiety.

The fluoroquinolones, represented by norfloxacin, ciprofloxacin, sparfloxacin, gatifloxacin, levofloxacin, and moxifloxacin, are an important class of antibiotics with clinical activity against Gram positive and Gram negative bacteria as well as mycobacteria. Ciprofloxacin, has recently been a drug of choice for the treatment of Bacillus anthracis infection in humans. Targets are prokaryotic DNA topoisomerase II (gyrase) and topoisomerase IV leading to DNA strand breaks. The structure of the drug-DNA-enzyme complex is still unclear; models have been proposed based upon indirect evidence. Some data point to a direct interaction between the drug and DNA. In the absence of the enzyme, drug-DNA binding is weak, and slight preference for binding in the minor groove is reported. Some fluoroquinolones such as ciprofloxacin are more effective against topo IV, others such as sparfloxacin are more effective against topo II, and some such as moxifloxacin and gatifloxacin target topo II and IV equally. Mechanistic insights result from a crystal structure of a yeast topo II fragment and the effect of mutations in the enzymes on fluoroquinolone resistance. DNA strand breaks are proposed to result from the fluoroquinolone stabilization of the cleaved DNA enzyme complex. Exactly how the drug stabilizes the complex remains unknown although various models have been presented. Studies of drug DNA interactions in the absence of enzyme show weak binding with preference for the minor groove and the involvement of Mg²⁺ and the amino groups of G-bases.

The structure of the fluoroquinolones and their target of activity share some features with the clinically important antitumor drugs, doxorubicin (dox) and epidoxorubicin (epi), which are classified as topo II poisons. Recent studies indicate a pathway by which doxorubicin and epidoxorubicin become covalently bonded to DNA. The mechanism involves the iron complex of the drugs inducing oxidative stress to produce formaldehyde, followed by the drugs using formaldehyde to attach themselves to G-bases of DNA. At NGC sites in DNA, the combination of covalent and non-covalent drug interactions serve to virtually crosslink the DNA, leading to apoptotic as well as non-apoptotic cell death. Dox secures iron through disruption of the iron homeostasis mechanism. Resistance to dox results in part from overexpression of enzymes which neutralize oxidative stress, and overexpression of the drug efflux pump P-170 glycoprotein. Lower levels of formaldehyde have recently been observed in dox-treated resistant cancer cells than in sensitive cancer cells. Presumably, DNA-doxorubicin virtual crosslinks lead to topo II lesions and ultimately cell death.

To solve the continuing problem of bacterial resistance to antibiotics, new drugs for new targets can be discovered or old drugs can be improved. This Example describes the formation of prodrug conjugates with fluroquinolone antibiotics to improve known antibiotic drugs.

Resistance of Staphylococcus aureus to vancomycin is of current international concern. Vancomycin inhibits bacterial cell wall biosynthesis through interference with a transpeptidation reaction resulting in a weaker cell wall. Antibiotic activity occurs through vancomycin complexation with a dipeptide unit D-Ala-D-Ala at the site of transpeptidation. One strain of S. aureus which shows intermediate resistance (VISA) is Mu50, and resistance has been proposed to result from a thicker cell wall with decoy D-Ala-D-Ala binding sites which tie up the drug. The K_{d} for the vancomycin-peptide complex is micromolar and is even lower for the binding of a second vancomycin because of a cooperative interaction. On this basis and from the experience with the anthracycline antitumor drugs, vancomycin might be useful for targeting another drug, ciprofloxacin, to Mu50 using the strategy analogous to that shown previously herein for targeting doxorubicin. In the design, vancomycin replaces the peptapeptide CNGRC and ciprofloxacin replaces doxorubicin. In the literature, the formaldehyde-isatin N-Mannich base of ciprofloxacin has been synthesized and that it has antibiotic activity comparable to that of ciprofloxacin against a variety of Gram positive and Gram negative bacteria. Previous experience with N-Mannich bases, however, suggests that the isatin N-Mannich base would cleave only very slowly under physiological conditions to release ciprofloxacin, and thus, the salicylamide N-Mannich base was used. The design and synthesis of vancociproform (Structure O, FIG. 5) are shown in FIG. 23. The salicylamide trigger in vancociproform has a half-life under physiological conditions of 90 min.

The strategy of using vancomycin to target a fluoroquinolone to Gram positive bacteria will fail with bacterial strains in which the resistance mechanism involves a change in the D-Ala-D-Ala binding site to D-Ala-D-lactate at the site of transpeptidation through mutation and gene exchange. The vancomycin D-Ala-D-lactate K_{d} is larger by three orders of magnitude. This form of resistance has appeared in enterrococci bacteria. An alternative targeting strategy that does not use vancomycin is described later.

MIC data for vancomycin-targeted-ciprofloxacin were obtained at the University of Chicago Medical School with several strains of S. aureus including Mu50 and are shown in Table 9. Vancociproform is approximately 4-fold more active than vancomycin against Mu50.

TABLE 9
MIC Data (μM) for Resistant and Sensitive Strains of Staphylococcus
aureus. Data are reported in units of micromolar to facilitate
molecular comparison of antibiotic activity; VISA stands for vancomycin
intermediate resistant Staphylococcus aureus.

| | Strain Treatment time | | | | |
|---|---|---|---|---|---|
| | Vancociproform | Vancosalicylamide | Ciprosaliform | Vancomycin | Ciprofloxacin |
| | 24/48 hr | 24/48 hr | 24/48 hr | 24/48 hr | 24/48 hr |
| Mu 50 | 1/1 | 2/4 | 4/8 | 4/4 | 96/>96 |
| VISA | | | | | |
| Mu 3 | 1/2 | 4/4 | 4/8 | 0.5/1 | 96/>96 |
| Hetero VISA | | | | | |
| IL-A | 2/2 | 4/4 | 8/8 | 1/1 | >96/>96 |
| Hetero VISA | | | | | |
| IL-F | 1/1 | 4/4 | 8/8 | 3/3 | >96/>96 |
| VISA | | | | | |
| 8325 | 2/2 | 0.6/1 | 1/1 | 0.3/0.3 | 6/12 |
| susceptible | | | | | |

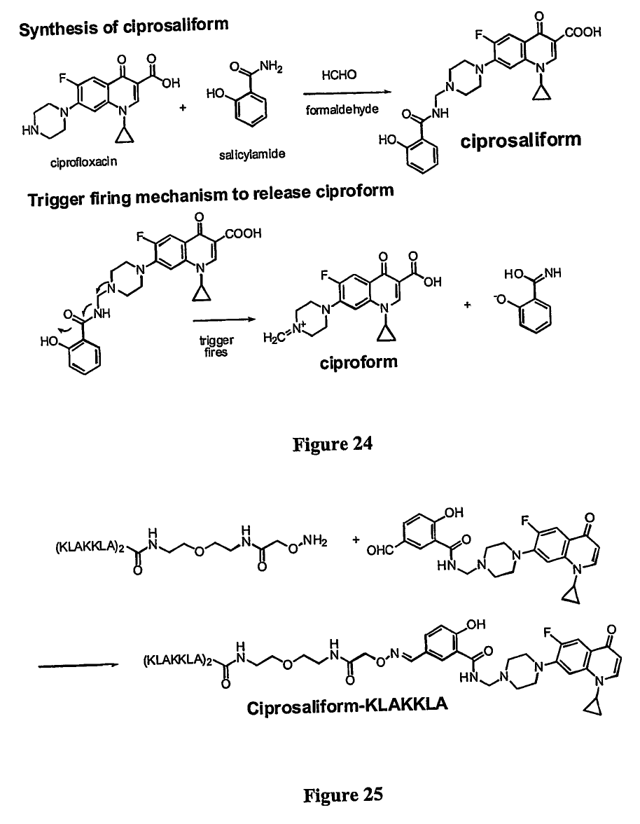

To ascertain the source of the biological activity of vancociproform, MIC data were also obtained for the fragments resulting from the trigger firing, vancosalicylamide and ciproform, as well as the clinical drug ciprofloxacin. Since ciproform is not stable, ciprosaliform (Structure K, FIG. 3) was synthesized which releases ciproform upon the firing of its salicylamide trigger. The synthesis of ciprosaliform and the mechanism for release of ciproform via the trigger mechanism are shown in FIG. 24. Surprisingly, ciprofloxacin was inactive against VISA and hetro VISA strains, and ciprosaliform was 10 to 20-fold more active against the VISA and hetero VISA strains and 6-fold more active against a susceptible strain shown in Table 9. Further, comparison of the data in Table 9 suggests that the vancomycin portion of vancociproform is serving to target ciproform to MuSO and possibly IL-F VISA strains.

The significantly increased antibiotic activity of ciprosaliform relative to ciprofloxacin and modest success in targeting ciprosaliform to Mu50 via vancosaliform prompted the exploration of the mechanism of action of ciprosaliform relative to ciprofloxacin and to explore other strategies for the delivery of ciprosaliform to bacteria.

A working hypothesis is that ciproform creates a more robust covalent interaction with DNA leading to topoisomerase DNA strand breaks even with mutated topoisomerase enzymes in resistant bacteria.

A second fluoroquinolone of interest for conjugation to formaldehyde is moxifloxacin because it targets both topoisomerase II and IV and is more active against resistant bacteria than is ciprofloxacin. The conjugate moxisaliform (Structure N, FIG. 4) may be formulated as the hydrochloride salt. In the structure of the hydrochloride salt, protonation is predicted to occur at the most basic nitrogen. This serves to stabilize the trigger mechanism and improve the solubility in water. The trigger is stabilized because the release mechanism requires a lone pair on the amino nitrogen of the N-Mannich base as shown for dox-NGR previously herein and for ciprosaliform. The structure may be thoroughly characterized by NMR and mass spectral techniques. Ciprosaliform is similarly formulated as its hydrochloride salt.

Fluoroquinolone antibiotics may be improved by attaching a targeting group via the cleavable trigger. The goals for targeting are to locate the antibiotic on or near bacteria before the trigger fires to maximize antibiotic activity and to minimize drug side effects. Although antibiotics are specific for bacteria, most of them have some side effects. The concept of a targeted fluoroquinolone is illustrated in FIG. 23 with ciprosaliform tethered to vancomycin. The strategy is attractive when both the targeting group and the cargo are antibiotics. A further attractive design feature is for the two antibiotics to have different mechanisms. In this case the vancomycin portion should inhibit bacterial cell wall synthesis and the cipro portion should inhibit DNA processing and replication. For the construct to be effective, the MIC values for the two drugs must be matched in molar units since the two drugs are delivered 1:1. In the case of vancociproform, the two drugs are vancosalicylamide and ciproform. Although the MIC of ciproform is not known since it is a transient, it is approximated with the MIC of ciprosaliform, a prodrug of ciproform. With this approximation the MIC of vancosalicylamide is half that of ciprosaliform. The MIC of the targeted drug vancociproform is half that of vancosalicylamide; hence the data support the concept of targeting. The observation that vancosalicylamide has a lower MIC than vancomycin is consistent with the lower MIC of the chlorobiphenyl-vancomycin currently in clinical development. Chlorobiphenyl-vancomycin also bears a hydrophobic group on the vancosaminyl sugar.

A complementary design of a targeted fluoroquinolone utilizes an antimicrobial peptide (KLAKKLA)₂ SEQ ID NO: 3. Antimicrobial peptides are significantly more toxic to bacteria than to mammalian cells. Selectivity is proposed to result from negatively charged bacterial cell membranes versus neutral mammalian cell membranes. The biological activity of (KLAKKLA)₂ against several bacteria versus mammalian cells is shown in Table 10. Of particular importance to the design proposed here is the similarity of the MIC values of the antimicrobial peptide with those of ciprosaliform as shown in Table 9. Again, the mechanisms of action of the two antibiotics are distinct, the antimicrobial peptide disrupts the cell membrane and the fluoroquinolone disrupts DNA processing and replication.

TABLE 10
Biological activity of (KLAKKLA)₂.^{a}

| | | S. aureus | | |
|---|---|---|---|---|
| E. coli | P. aeruginosa | ATCC | 3T3 mouse | Human |
| ATCC 25922 | ATCC 27853 | 25723 | fibroblasts | erythrocytes |
| 6 | 3 | 6 | >272 | >750 |

^{a}Bacterial lysis is measured as MICs (μM), and 3T3 mouse fibroblast lysis and human erythrocyte lysis is measured as sub-lethal concentrations (μM).

The design of an antimicrobial peptide-targeted ciproform (ciprosaliform-KLAKKLA, Structure M, FIG. 4) is shown in FIG. 24 and its synthesis is shown in FIG. 25. The hydroxylamine ether tether may be synthesized and attached to polystyrene beads. Fmoc solid state peptide synthesis will be performed on an automated peptide synthesizer and the resulting peptide will be deprotected and released from the resin with Reagent K. After purification by HPLC, the tether may be attached to 5-formyl-ciprosaliform via an oximation reaction. 5-Formyl-ciprosaliform is the same derivative of ciprofloxacin used in the synthesis of vancociproform shown in FIG. 23 and in the synthesis of dox-ngr.

As will be readily apparent to one of skill in the art, the design in FIG. 25 is a platform design amenable to other fluoroquinolone antibiotics such as moxifloxacin and to other targeting peptides and antibiotics.
Experimental: UV-vis spectrometry was performed with a diode array spectrophotometer and workstation. Mass spectral data was collected with a Perkin Elmer™ Sciex API III, equipped with an ion-spray source and workstation, scanned at 0.2 amu resolution.

HPLC Elution Methods: The following HPLC elution gradients were employed with A=acetonitrile and B=0.15% TFA aqueous solution (pH=1.7) unless otherwise noted. Aqueous solutions for HPLC solvents were filter with a 0.45-micron nylon filter and acetonitrile for HPLC was filtered with a 0.22-micron nylon filter.

Analytical HPLC: is a liquid chromatograph equipped with a diode array UV-vis detector and workstation; chromatographies were performed with a 5 μm reverse phase C₁₈ microbore column, (2.1 mm×100 mm), eluting at 0.5 mL/min. Method EIPLC III (330 nm, 280 nm), A:B, 5:95 to 20:80 at 10 min, 50:50 at 18 min, 70:30 at 20 min, isocratic until 22 min, 5:95 at 24.8 min, isocratic until 25 min.

Preparative HPLC: is a pump with an absorbance detector and workstation; chromatographies were performed with a Vydac 214TP1022 reverse phase C₄ column (22 X 250 mm), eluting at 9.0 mL/min. Method HPLC IX (330 nm), A:B, 15:85 at 0 min, isocratic until 1 min, 25:75 at 20 min, 40:60 at 40 min, 70:30 at 45 min, isocratic until 50 min 15:85 at 55 min.

N-(2-Hydroxybenzamido-methyl)-ciprofloxacin: To a stirring solution of 3 mg (21.9 μmol) of salicylamide in 1.0 mL of DMF was added 10 μL (120 μmol) of a 37% formalin solution. The reaction was stirred in a sealed 25 mL pear flask for 25 min at 60° C., at which time 5 mg (15.1 μmol) of ciprofloxacin free-base was added to form a white suspension that was stirred at 60° C. After 25 min, a clear solution had formed and the reaction was removed from the heat. The solution was evacuated to dryness by rotary evaporation at 55° C. and 0.6 mbar to give a red film. Product was dissolved in 0.5 mL DMF and injected onto the preparative hplc using method HPLC IX with product eluting at 15.2 min (B=0.15% HCl) or 22.5 min (B=0.15% TFA). Unreacted ciprofloxacin eluted at 9.0 min (B=0.15% HCl) or 11.6 min (B=0.15% TFA). Collected sample was transfer to a 100 nL round bottom flask and evacuated to dryness by rotary evaporation at 45° C. and 0.6 mbar. Product was then put on a vacuum line at 0.1 mbar for 30 min. Purity was determined by hplc using method HPLC m, and the product was stored at −80° C. until needed. The yield was 5.3 mg (11.0 μmol, 72.8% yield) of N-(5-formyl-2-hydroxybenzamido-methyl)-ciprofloxacin at 93.0% purity (7% ciprofloxacin). The material showed an ESI-MS peak at m/z 481.0 [M+1] (calculated, 481.2) and the structure was established NMR spectroscopy.

### Example 9

Design, Synthesis and Evaluation of Doxorubicin-Formaldehyde Conjugate Targeted to Epidermal Growth Factor Receptor Tyrosine Kinase Domain of Cancer Cells and their Associated Angiogenesis

Peptide growth factors are known to modulate signaling pathways involved in cell proliferation and death in both normal and malignant cells. The epidermal growth factor receptor (EGFR) is a tyrosine kinase that is overexpressed in a wide variety of solid human cancers including non-small-cell lung, breast, head and neck, bladder, and ovarian carcinomas. EGFR has been associated in numerous studies with an advanced disease state and poor prognosis. Angiogenesis, tumor proliferation, invasion, and cell adhesion have all been linked to overexpression of EGFR. The epidermal growth factor (EGF) and transforming growth factor (TGF-α) ligands have shown potent induction of angiogenesis in vivo, which is essential to tumor metastasis. Co-expression of TGF-α and EGFR also shows a strong correlation with microvessel density in invasive breast cancer.

A large body of both experimental and clinical evidence now suggests that the EGFR is a legitimate target for cancer therapy. Two separate therapeutic strategies for inhibiting EGFR have shown promising results in clinical studies. Monoclonal antibodies targeting the extracellular domain of the EGFR block ligand binding and therefore, receptor activation. The second strategy uses small molecule inhibitors (Iressa™, Tarceva™, etc.) of the intracellular tyrosine kinase domain which prevents autophosphorylation. Iressa™ (gefitinib) has recently been approved by the Food and Drug Administration in the United States for the treatment of advanced non-small-cell lung cancer. Regardless of the mechanism of EGFR blockade, greater tumor cell inhibition is observed when the inhibiting molecule is used in combination with a cytotoxin. The EGFR monoclonal antibody C225 has demonstrated in vivo tumor cell inhibition in mice bearing prostate carcinoma xenografts, alone or in combination with doxorubicin. Cell cycle analysis of A43 1 and MDA-MB-468 breast cancer cells treated with both C225 and doxorubicin clearly indicated a greater proportion of cells in Aₒ phase, which has been shown to be a measure of apoptosis. A431 and MDA-486 xenografts displayed enhanced antitumor responses to the combination of doxorubicin and C225.

An important example of targeting the EGFR in MDA-MB-23 1, and BT-20 human breast cancer cells, was the conjugate of genistein (a soybean derived TKI) with recombinant human epidermal growth factor (EGF). This produced a mild cytotoxic agent with tyrosine kinase inhibitory activity. A 1000 fold enhancement of antitumor activity (30 nM versus 120 μM) for the EGF-genistein conjugate relative to EGF, genistein, and unconjugated EGF with genistein, demonstrated the ability of EGF to deliver a cytotoxin to tumor cells. After the incubation of cells with EGF-genistein, a fluorescein isothiocyanate (FITC) conjugated goat anti-mouse IgG for EGFR showed the binding and internalization of EGF- genistein. SCID mice bearing human breast cancer xenografts treated with the EGF-genistein conjugate (2 μg/day) exhibited tumor disappearance in 2 of 5 mice and >50% reduction in 3 of 5 mice within 10 days. Control mice and mice treated with genistein alone, experienced a >200% increase in tumor size over the same time period. This study provides good evidence that the EGFR is over-expressed in tumor cells at a reasonable level for the delivery of a cytotoxin in vivo. Using a TKI such as Tarceva™ conjugated to the preactivated doxorubicin, doxsaliforn, would produce an EGFR targeted cytotoxin where the targeting group itself is also a potent anititumor agent. This conjugate should be more effective because both Tarceva and doxsaliform are singly more potent cytotoxins than genistein (IC₅₀s of 50 and 60 nM versus 120 μM) and have different cytotoxic mechanisms.

The middle of the 1990s gave rise to the discovery of the 4-anilinoquinazolines, a series of potent and selective inhibitors of EGFR and human epidermal growth factor (HER-2) kinases. Both Tarceva™ and Iressa™ inhibit tumor growth in vivo. Tarceva™ and Iressa™ bind to the intracellular tyrosine kinase domain of EGFR with high affinity and selectivity. The expression level of EGFR is critical to the successful delivery of sufficient drug to kill the tumor cell. Calculation of the approximate concentration of drug delivered to a tumor can be done based on such expression levels. For example, MDA-MB-231 breast cancer cells are known to express EGFR at approximately 4×10⁵ per cell and based on a cell volume of about 4×10⁻¹² L (20 μm diameter), one could expect a cellular concentration of drug at approximately 160 nM assuming a K_{d} of less than 40 nM. This could be higher if each receptor bound and released more than one molecule of drug, and it could be lower if diffusion out of the cell was fast relative to the binding constant of the TKI. With an IC₅₀ of 50 nM, doxsaliform combined with a TKI should be lethal to the tumor cell. If breast tumor cells respond to treatment by increased expression of EGFR, it will work to the advantage of the drug.

The tether which links the TKI to the cytotoxin must be of an appropriate length to avoid inhibition of binding of the targeting molecule. The recent publication of the X-ray crystal structure of Tarceva™ bound to the tyrosine kinase domain of EGFR provides a wealth of information on the three dimensional topology of the binding site.

The anilino portion of Tarceva™ is bound in a hydrophobic pocket with the ether linkages directed out of the binding domain into solvent. The quinazoline N-3 nitrogen atom is not within bonding range of Thr⁷⁶⁶, but a water of hydration connects the two through a weak hydrogen bond. The N-1 nitrogen of the quinazoline hydrogen bonds with Met⁷⁶⁹, and these hydrogen bonds describe the orientation of Tarceva™ in the hydrophobic pocket. Neither of the ether tails form significant bonding interactions and are directed out of the enzyme. These ether tails present an excellent point for attachment of an ether tether to link Tarceva™ or other TKIs to doxsaliform.

To determine the appropriate tether length, a Tarceva-doxsaliform conjugate was modeled using the published crystal structure coordinates in the Pymol™ and O™ modeling programs. Using an ether tether only one carbon longer than that already on Tarceva™ attached to doxsaliform, the modeled compound fit into the EGFR binding domain in identical fashion to that of Tarceva™ itself without any detectable negative steric or electronic interactions. The tether holds doxsaliform 7 Å from the top side of EGFR and 16 Å from the nearest residue on the bottom side. The TK domain is particularly open and accessible near the ligand binding site and modeling reveals that the receptor should even accommodate two doxsaliform molecules tethered to a single TKI. This would permit the delivery of twice the concentration of cytotoxin in a single molecule which may be necessary if EGFR is expressed at a lower than expected level or EGFR is expressed at a lower level in response to treatment as a resistance mechanism.

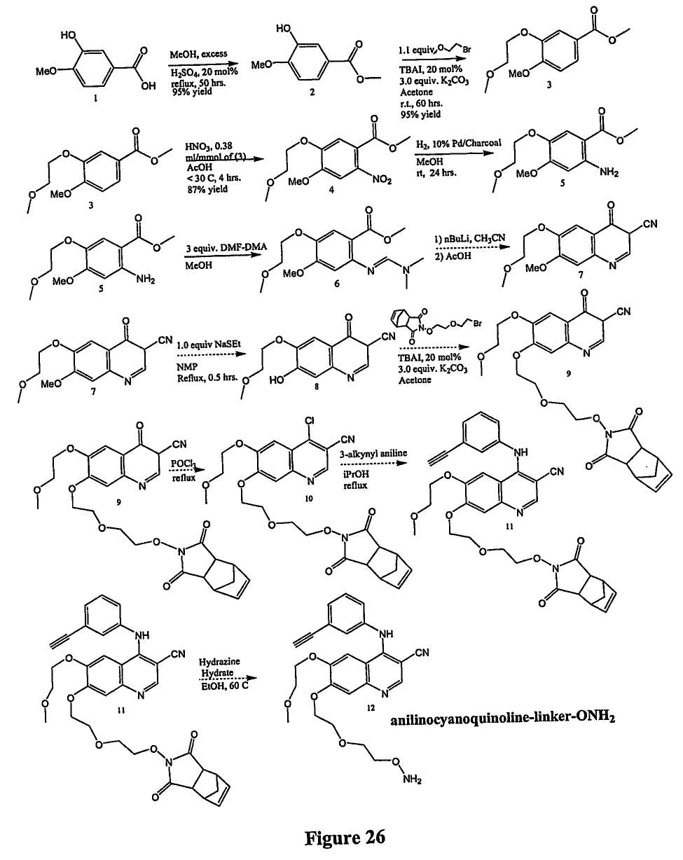

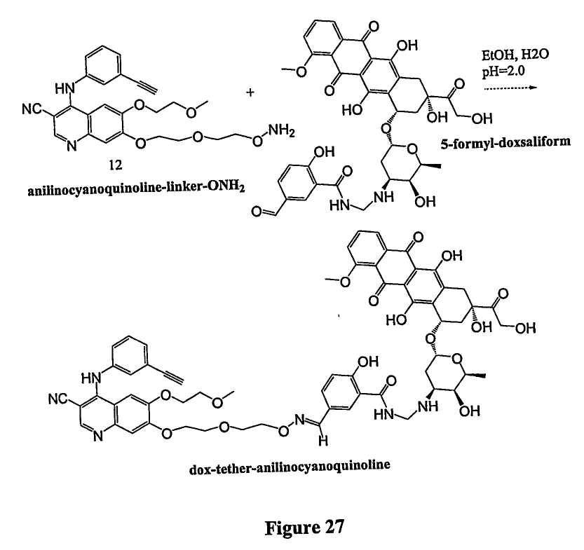

Design and Synthesis: Based upon modeling studies and binding studies an anilinocyanoquinoline was selected as the targeting group of choice for delivering doxorubicin-formaldehyde conjugate to cancer cells via the epidermal growth factor receptor tyrosine kinase domain (EGFR-TK). The proposed synthesis of the targeting group bonded to tether is shown in FIG. 25, and the coupling of the targeting group with tether to 5-formyldoxsaliform is shown in FIG. 26. Steps shown with solid arrows have been achieved and steps shown with dotted arrows are the expected synthetic reaction steps.

### Example 10

Design, Synthesis and Evaluation of Cisplatin-Formaldehyde Conjugates Targeted to Tumor Cells and their Associated Angiogenesis

The principal mechanism of action of both cisplatin and carboplatin is DNA alkylation. By forming interstrand or intrastrand covalent bonds with two guanine nucleotides of DNA, these drugs can effectively impede DNA replication. Additionally, cisplatin can crosslink proteins to DNA. The cisplatin derivatives proposed herein will be aimed towards improving cisplatin cytotoxicity in two ways. First, a targeting strategy will be employed that will allow the localization of the drugs selectively in the cancer cells. Second, the novel drugs will be structurally engineered so that they may crosslink DNA three times, or crosslink DNA and also a protein simultaneously.

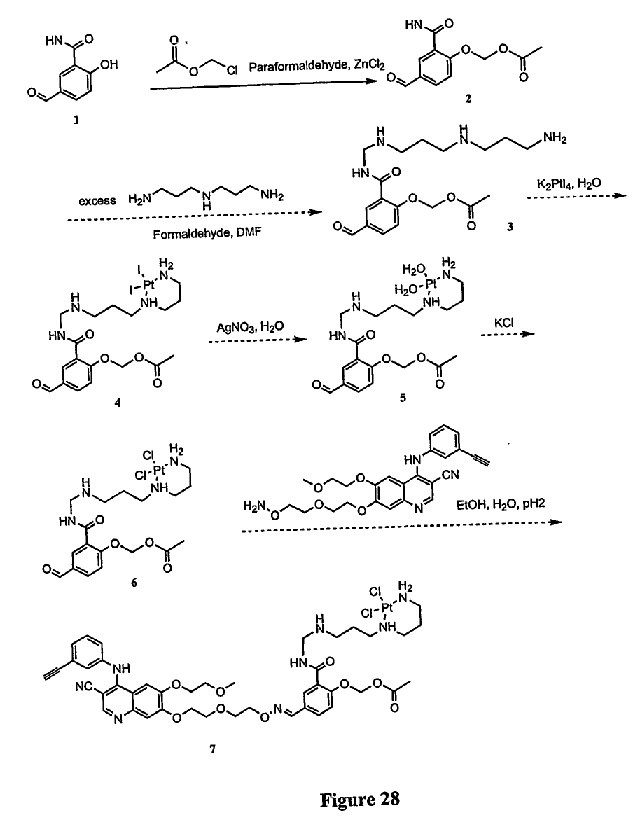

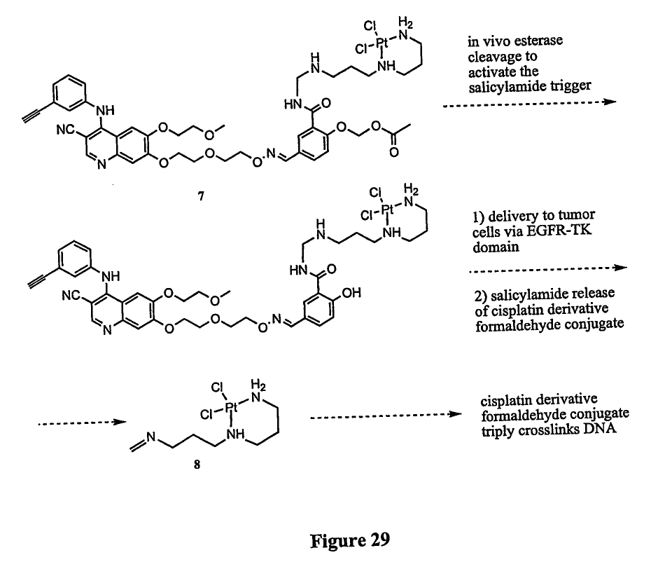

Design and synthesis: The design and proposed synthesis of the first platinum derivative (Structure B, FIG. 2) is shown in FIG. 28. The design incorporates the salicyamide trigger and anilinocyanoquinoline targeting group for the EGFR-TK domain, both previously described. The proposed mechanism of action of the prodrug conjugate incorporating the targeting domain for the EGFR-TK domain is shown in FIG. 29.

The design of the cisplatin derivative, FIG. 28, is based on molecular modeling and earlier design of targeted doxorubicin-formaldehyde conjugates. This platinum drug, once released from its targeting group via the hydrolysis of a chemical trigger, will contain a formaldehyde Schiff base that will be accessible to a nearby adenine nucleotide in the major groove of DNA. The amine group of the adenine may potentially attack the formaldehyde Schiff base of the platinum compound, resulting in a drug triply linked to the DNA as shown schematically in FIG. 29. This third covalent link will likely impart a greater toxicity to cancer cells. However, because this drug will be targeted to cancer cells selectively, heightened toxicity should not cause other systemic deficiencies.

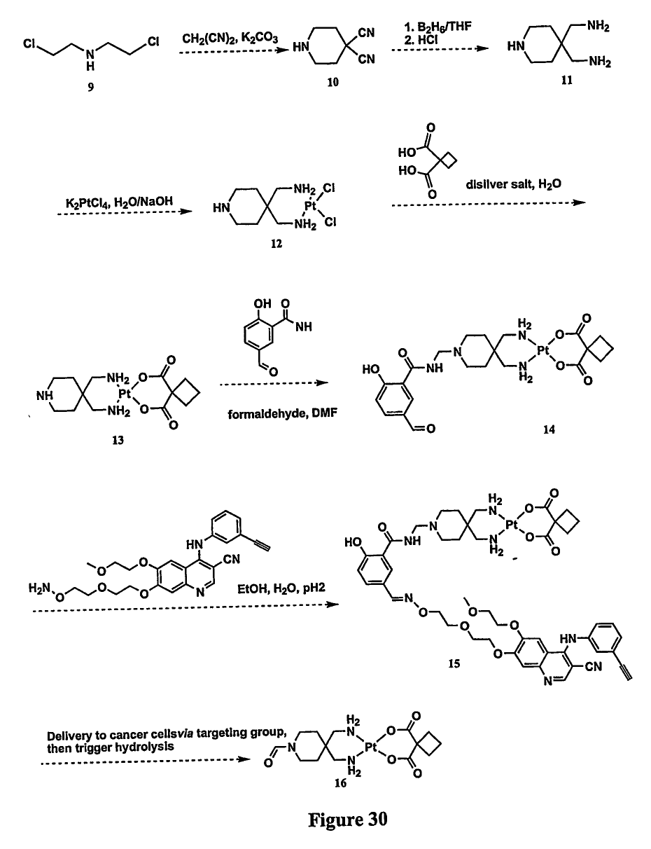

The design and synthesis of the second platinum compound (Structure B, FIG. 2) is shown in FIG. 30. It is based upon the structure of a third generation cisplatin derivative, enloplatin, which was entered into phase I clinical trials but found to be too nephrotoxic to continue on to phase II. Considering that this drug will be targeted, toxicity should not be such a substantial problem. Additionally, the structure of this molecule has been designed so that it may more easily crosslink proteins to the DNA.

The drug is extended further out of the major groove of the DNA, and will contain a formaldehyde Schiff base upon trigger firing. This Schiff base will be a target of nucleophilic attack by nucleophilic sites on the R groups of an associated protein.

Targeting of both cisplatin drugs is proposed with an anilinocyanoquinoline derivative for EGFR-TK domain, similar to strategies for targeting with homing peptides and non-steroidal antiandrogens and antiestrogens as described earlier.

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiment described hereinabove is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

What is claimed is:

1. A compound of the formula: 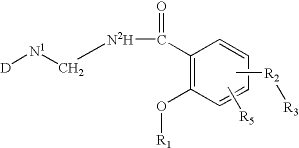
or a pharmaceutically acceptable salt thereof wherein,
D is a drug moiety comprising at least one primary or secondary amine designated N¹ selected from the group consisting of doxorubicin, daunorubicin, epidoxorubicin, ciprofloxacin, norfloxacin, gatifloxacin, levofloxacin, moxifloxacin, sparfloxacin, cisplatin, carboplatin and analogs thereof.
R₁ is H or —CH₂—O—C(O)R₄ wherein R₄ is H, linear or branched alkyl, alkenyl, alkynyl, aryl, alkoxy, aryloxy, arylalkoxy, heteroaryl, arylalkyl, heteroarylalkyl, cycloalkyl, cycloalkylalkyl, polycycloalkyl, polycycloalkylalkyl, cycloalkenyl, cycloheteroalkyl, heteroaryloxy, cycloalkenylalkyl, polycycloalkenyl, polycycloalkenylalkyl, heteroarylcarbonyl, amino, alkyl-amino, arylamino, heteroarylamino, cycloalkyloxy, or cycloalkylamino;
R₂ is absent or a bond or alkyl, alkenyl, alkynyl, allenyl, aryl, alkoxy, polyalkyloxy, aryloxy, arylalkoxy, heteroaryl, arylalkyl, heteroarylalkyl, cycloalkyl, cycloalkylalkyl, polycycloalkyl, polycycloalkylalkyl, cycloalkenyl, cycloheteroalkyl, heteroaryloxy, cycloalkenylalkyl, polycycloalkenyl, polycycloalkenylalkyl, heteroarylcarbonyl, amino, alkyl-amino, arylamino, heteroarylamino, cycloalkyloxy, or cycloalkylamino;
R₃ is absent or a targeting compound capable of selectively binding to a specific target site in a mammal selected from the group consisting of a cell, a tissue, a bodily fluid, a receptor, a ligand and a cell surface molecule; and,
R₅ can be H, cyano, acyl, nitro, alkoxycarbonyl, aminocarbonyl, hydroxyl, alkoxyl, acyloxy, or amido.

2. The compound of claim 1, wherein R₄ is C1-C20 linear or branched alkyl, alkoxy, alkenyl, alkynyl, aryl, or heteroaryl.

3. The compound of claim 1, wherein R₂ is C4-C20 linear alkyl, alkenyl, alkynyl, allenyl, or polyalkyloxy.

4. The compound of claim 1, wherein R₂ is:
—CH₂OCH₂C≡—CCH₂—,
13 CH₂OCH₂—C≡C—C≡H₂—,
—CH₂(OCH₂CH₂)ₙ—wherein n is an integer between 1 and 20,
CH═N—OCH₂CH₂)ₙ—wherein n is 1, 2 or 3,
—CH═N—OCH₂C(O)NHCH₂CH₂OCH₂CH₂—,
—CH═N—OCH₂C≡C—CH₂—,
—CH═N—OCH₂C—C≡CC≡C—CH₂—,
CH═NOCH₂CH₂OCH₂CH₂—,
—CH═N—OCH₂C(O)—, or
N,N′-disubstituted piperazine.

5. The compound of claim 1, wherein R₃ is a moiety that binds specifically to receptors overexpressed in cancer cells.

6. The compound of claim 1, wherein R₃ is a moiety that binds specifically to endothelial cells undergoing angiogenesis.

7. The compound of claim 1, wherein R₃ is a moiety that binds specifically to biological molecules unique to bacterial cells.

8. The compound defined in claim 1, which is selected from the group consisting of:
N-(2-hydroxybenzamidomethyl)-doxorubicin);
N-(5-{4-[3-(4-Cyano-3-trifluoromethyl-phenyl)-5,5-dimethyl-2,4-dioxo-imidazolidin-1-yl]-but-2-ynyloxymethyl)-2-hydroxy-benzamidomethyl)-doxorubicin; and,
E/Z-N-(2-Hydroxy-5-{[2-(2-{2-[(2-{4-[1-(4-hydroxy-phenyl)-2-phenyl-but-1-enyl]-phenoxy}-ethyl)-methyl-amino]-ethoxy}-ethoxy)-ethoxyimino]-methyl}-benzamidomethyl)-doxorubicin.

9. The compound defined in claim 1, which is selected from the group consisting of:

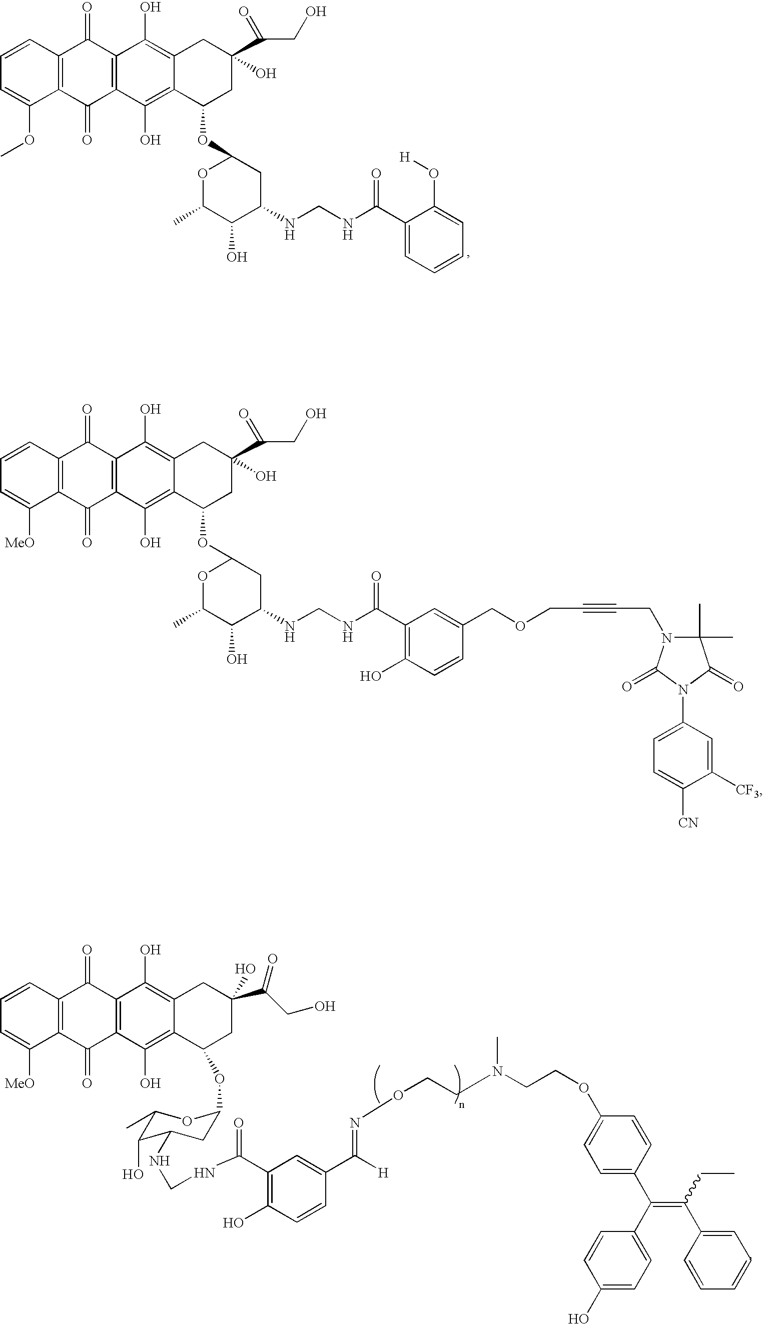

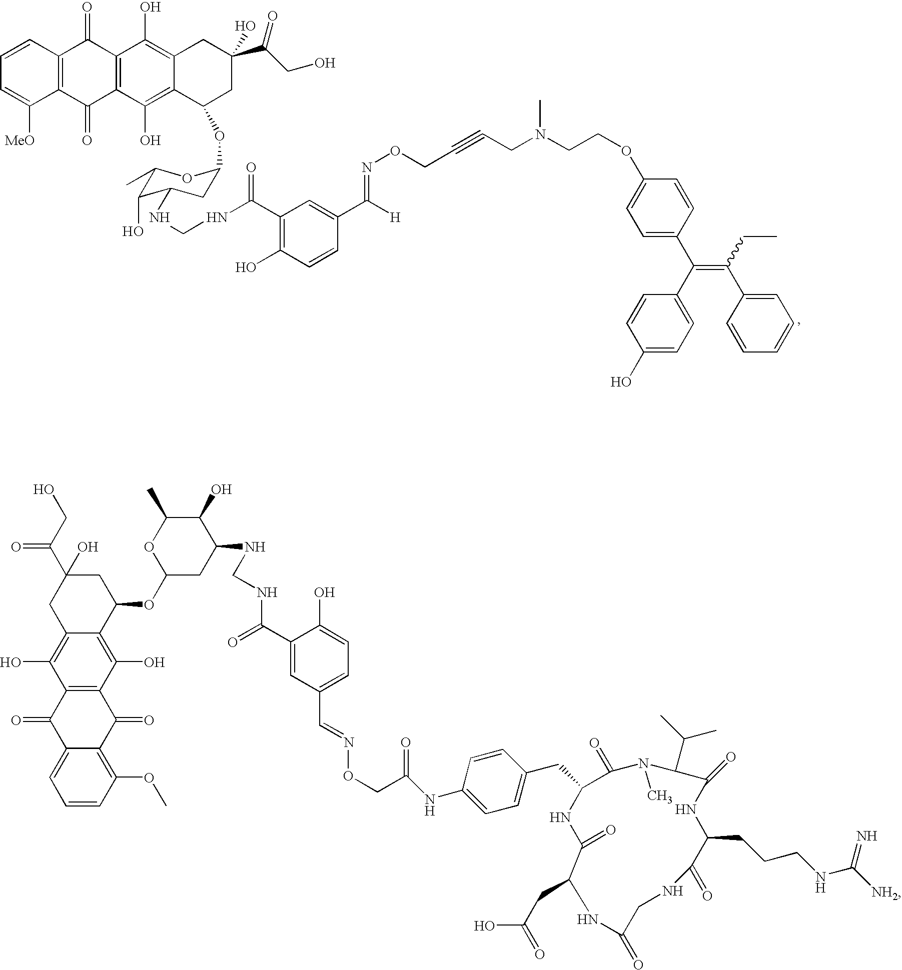

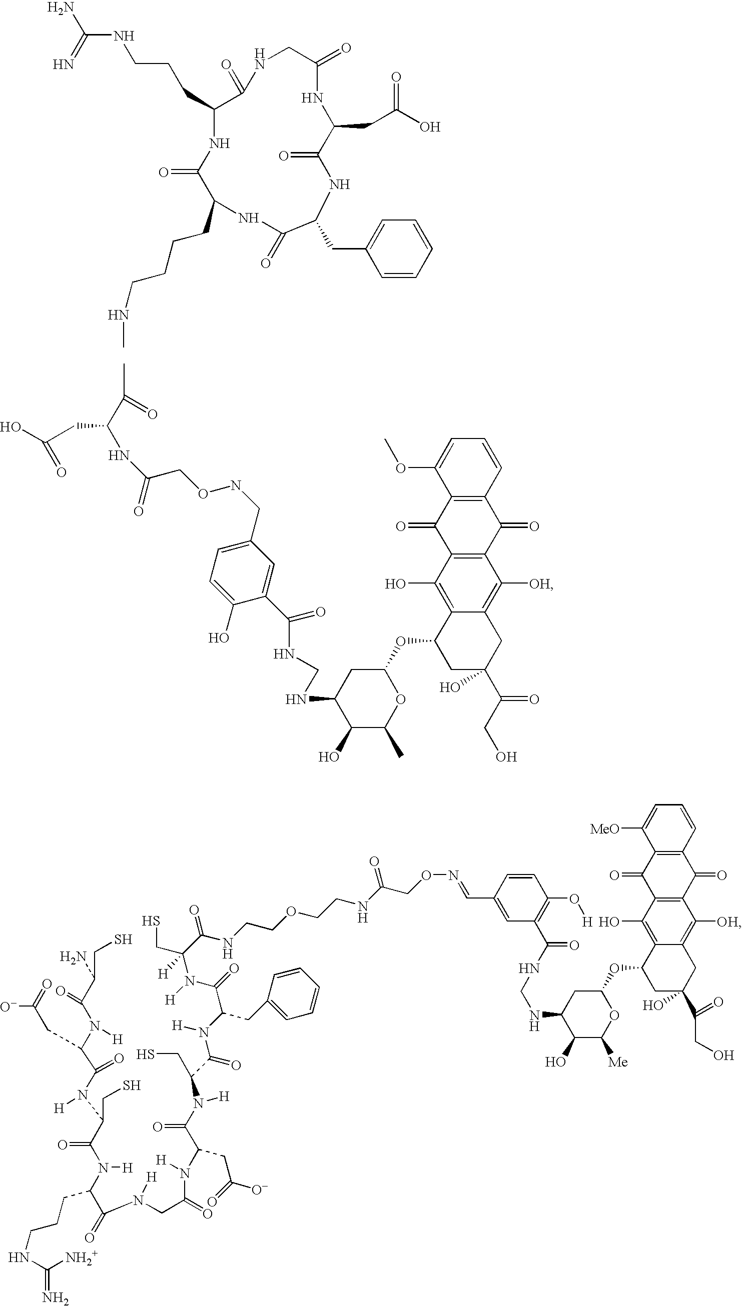

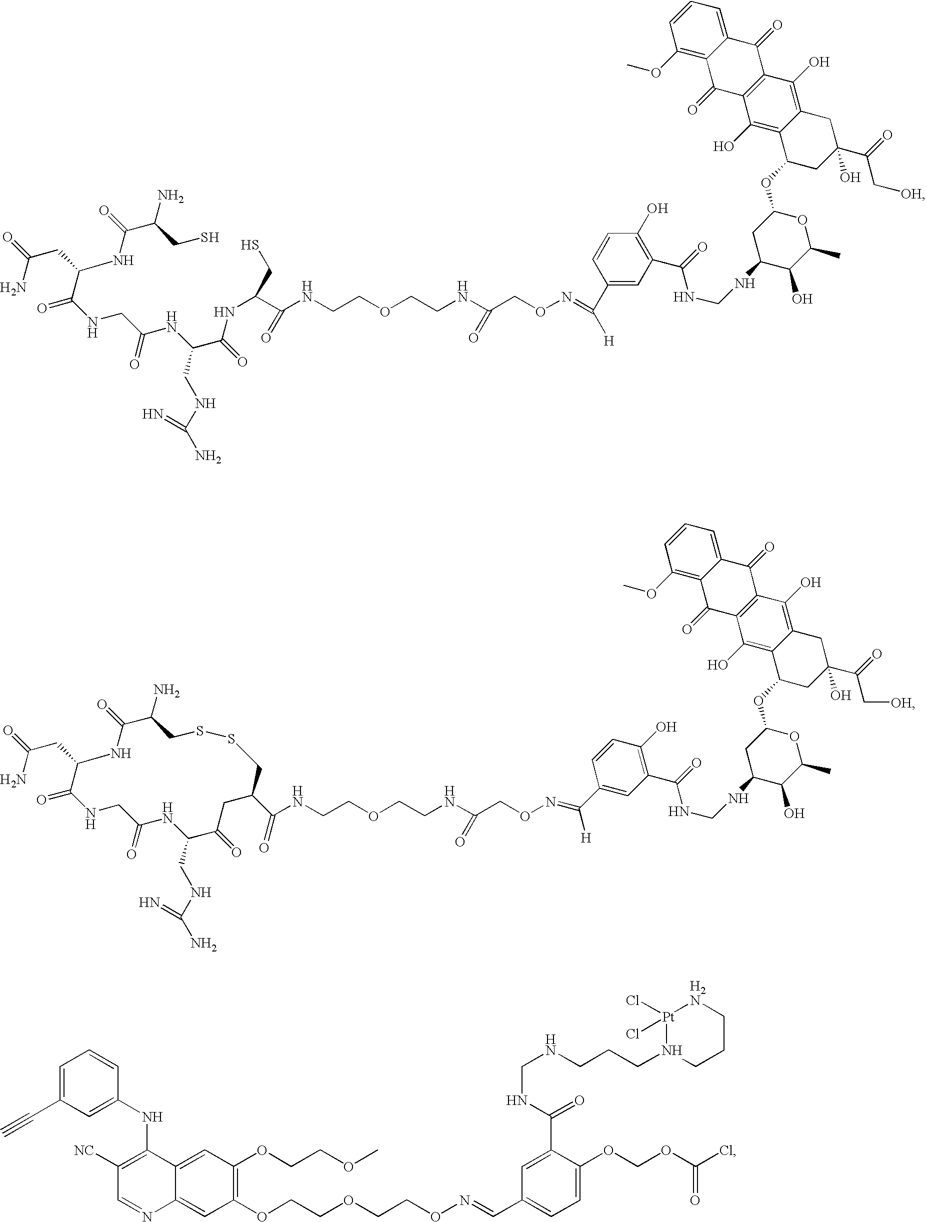

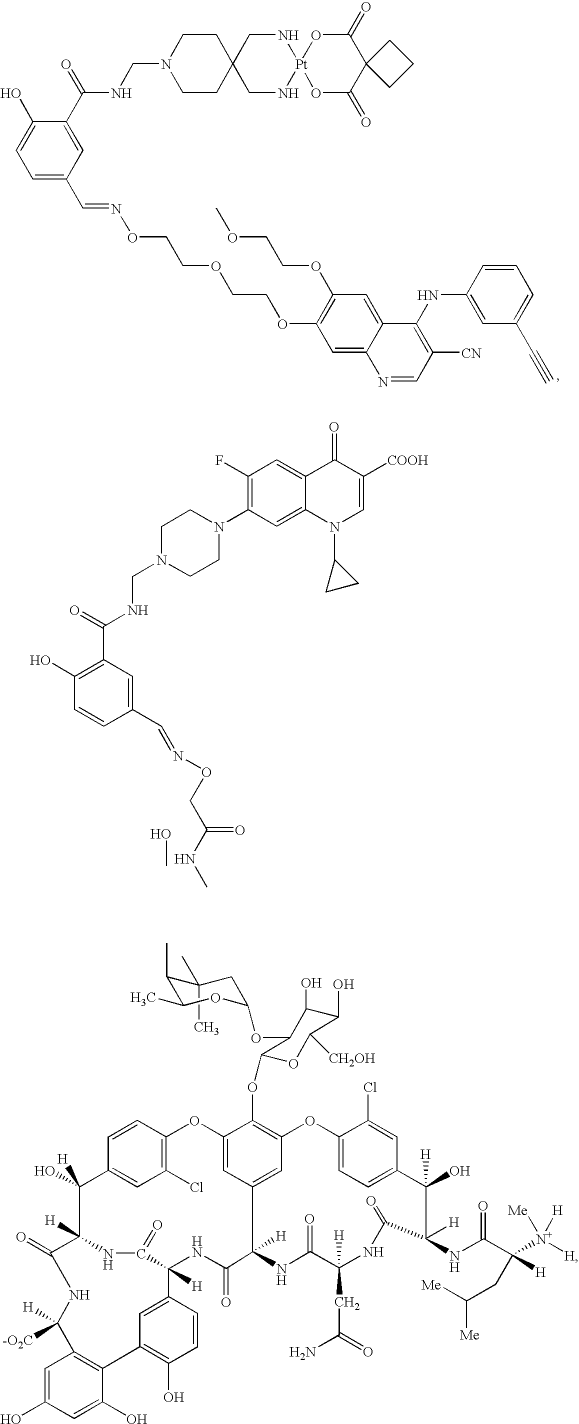

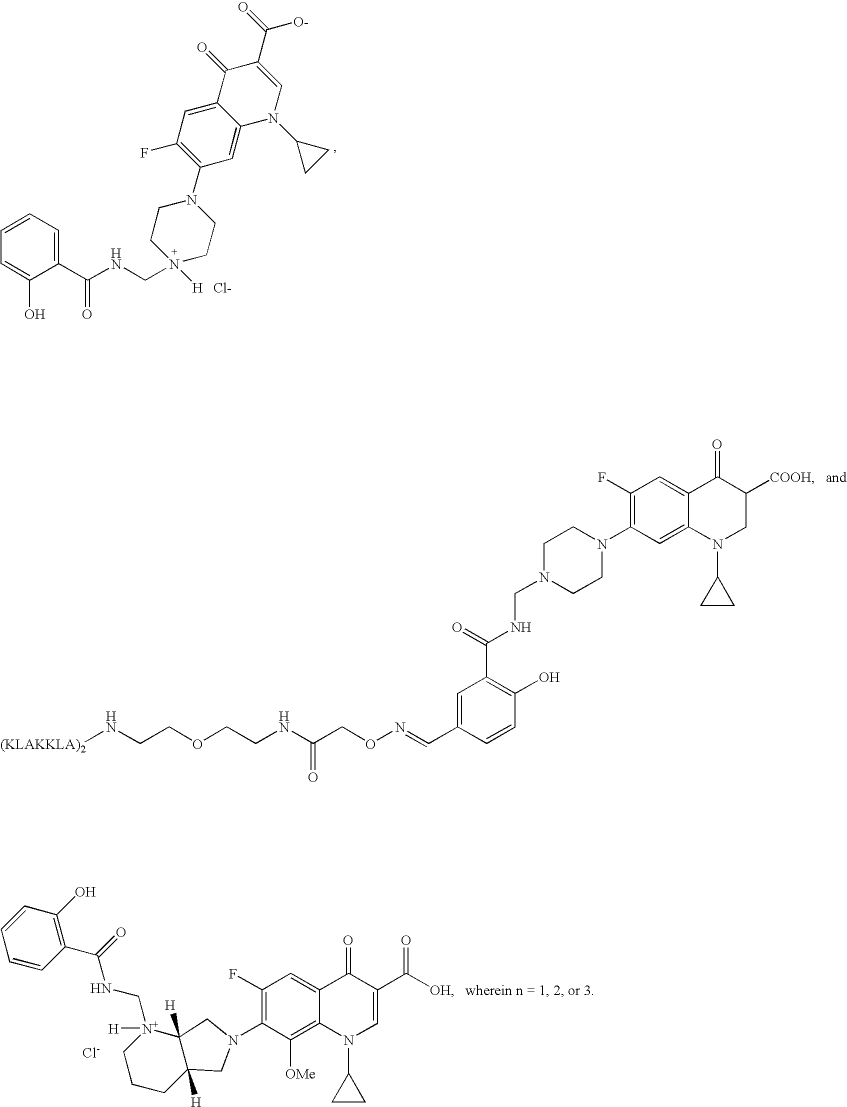

10. A pharmaceutical composition comprising a therapeutically effective amount of a compound of claim 1 and a pharmaceutically acceptable carrier.

11. A compound of the formula: 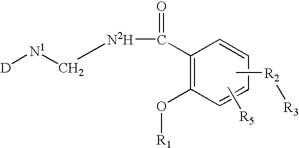
or a pharmaceutically acceptable salt thereof wherein,
D is a drug moiety comprising at least one primary or secondary amine designated N^{l};
R₁ is H or —CH₂—O—C(O)R₄ wherein R₄ is H, linear or branched alkyl, alkenyl, alkynyl, aryl, alkoxy, aryloxy, arylalkoxy, heteroaryl, arylalkyl, heteroarylalkyl, cycloalkyl, cycloalkylalkyl, polycycloalkyl, polycycloalkylalkyl, cycloalkenyl, cycloheteroalkyl, heteroaryloxy, cycloalkenylalkyl, polycycloalkenyl, polycycloalkenylalkyl, heteroarylcarbonyl, amino, alkyl-amino, arylamino, heteroarylamino, cycloalkyloxy, or cycloalkylamino;
R₂ is a bond or alkyl, alkenyl, alkynyl, allenyl, aryl, alkoxy, polyalkyloxy, aryloxy, arylalkoxy, heteroaryl, arylalkyl, heteroarylalkyl, cycloalkyl, cycloalkylalkyl, polycycloalkyl, polycycloalkylalkyl, cycloalkenyl, cycloheteroalkyl, heteroaryloxy, cycloalkenylalkyl, polycycloalkenyl, polycycloalkenylalkyl, heteroarylcarbonyl, amino, alkyl-amino, arylamino, heteroarylamino, cycloalkyloxy, or cycloalkylamino;
R₃ is a targeting compound capable of selectively binding to a specific target site in a mammal selected from the group consisting of a cell, a tissue, a bodily fluid, a receptor, a ligand and a cell surface molecule and,
R₅ is H, cyano, acyl, nitro, alkoxycarbonyl, aminocarbonyl, hydroxyl, alkoxyl, acyloxy, or amido.

12. A pharmaceutical composition comprising a therapeutically effective amount of a compound of claim 11 and a pharmaceutically acceptable carrier.

13. A method of treating cancer in a mammal comprising administering a therapeutically effective amount of a compound of claim 1 to a mammal.

14. The method of claim 13, wherein the compound of claim 1 is N-(2-hydroxybenzamidomethyl)-doxorubicin) and the cancer is selected from the group consisting of Hodgkin's disease, non-Hodgkin's lymphoma, and acute leukemia.

15. The method of claim 13, wherein the compound of claim 1 is N-(2-hydroxybenzamidomethyl)-doxorubicin) and the cancer is a solid tumor in a tissue selected from the group consisting of lung, liver, breast, and ovary.

16. The method of claim 13, wherein the compound of claim 1 is N-(5-{4-[3-(4-Cyano-3-trifluoromethyl-phenyl)-5,5-dimethyl-2,4-dioxo-imidazolidin-1-yl]-but-2-ynyloxymethyl)-2-hydroxy-benzamidomethyl)-doxorubicin and the cancer is prostate cancer.

17. The method of claim 13, wherein the compound of claim 1 is E/Z-N-(2-Hydroxy-5-{[2-(2-{2-[(2-{4-[1-(4-hydroxy-phenyl)-2-phenyl-but-1-enyl]-phenoxy}-ethyl) -methyl-amino]-ethoxy}-ethoxy)-ethoxyimino]-methyl}-benzamidomethyl)-doxorubicin and the cancer is breast cancer.

18. A method of inhibiting or causing the regression of angiogenesis in a mammal comprising administering a therapeutically effective amount of a compound of claim 1 to a mammal.

19. The method of claim 18, wherein the compound of claim 1 is selected from the group consisting of:
cyclic DOXSF-RGD-4C,
a cyclic DOXSF-RGD-4C,
cyclic-(N-Me-VRGDf-NH)DOXSF,
anilinocyanoquinoline-cisplatinSF,
anilinocyanoquinoline-DOXSF,
cyclic-DOX-NGR, and
acyclic-DOX-NGR.

20. A method of cross-linking DNA in a cell comprising contacting a cell with a compound of claim 1.

21. A method of forming DNA adducts in a cancer cell comprising administering a compound of claim 1 to a mammal containing a cancer cell.

22. A method of preventing or treating an infection in an organism comprising administering a therapeutically effective amount of a compound of claim 1 to an organism.

23. The method of claim 22, wherein the infection is produced by a gram positive or gram negative bacteria or mycobacteria and the compound is selected from the group consisting of
vancociproform,
ciprosaliform,
ciprosaliform-KLAKKLA, and
moxisaliform.

24. A method of making a compound of claim 1 comprising:
a) contacting salicylamide with formaldehyde in the presence of a drug moiety comprising at least one primary amine or cyclic secondary amine to form an N-Mannich base;
b) covalently-binding the N-Mannich base to a targeting compound capable of selectively binding to a specific target site in a mammal selected from the group consisting of a cell, a tissue, a bodily fluid, a receptor, a ligand and a cell surface molecule.

25. A method of making a targeted prodrug compound comprising:
a) contacting a salicylamide analog with formaldehyde in the presence of a drug moiety comprising at least one primary amine or cyclic secondary amine to form an N-Mannich base;
b) covalently-binding the N-Mannich base to a targeting compound capable of selectively binding to a specific target site in a mammal selected from the group consisting of a cell, a tissue, a bodily fluid, a receptor, a ligand and a cell surface molecule.

26. The method of claim 23, wherein the salicylamide analog comprises a compound of the formula: 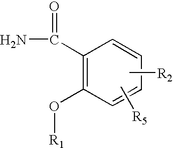
wherein
R₁ is H or —CH₂—OC(O)R₄ wherein R₄ is H, linear or branched alkyl, alkenyl, alkynyl, aryl, alkoxy, polyalkyloxy, aryloxy, arylalkoxy, heteroaryl, arylalkyl, heteroarylalkyl, cycloalkyl, cycloalkylalkyl, polycycloalkyl, polycycloalkylalkyl, cycloalkenyl, cycloheteroalkyl, heteroaryloxy, cycloalkenylalkyl, polycycloalkenyl, polycycloalkenylalkyl, heteroarylcarbonyl, amino, alkyl-amino, arylamino, heteroarylamino, cycloalkyloxy, or cycloalkylamino;
R₂ is —CH₂OCH₂C≡CCH₂—,
—CH₂OCH₂—C≡C—C≡C—CH₂—,
—CH₂(OCH₂CH₂)ₙ— wherein n is an integer between 1 and 20,
—CH═N—(OCH₂CH₂)ₙ—N(CH₃)CH₂CH₂— wherein n is 1, 2 or 3,
—CH═N—OCH₂C(O)NHCH₂CH₂OCH₂CH₂—,
—CH═N—OCH₂C≡C—CH₂—,
—CH═N—OCH₂C—C═C—C═C—CH₂—,
—CH═NOCH₂CH₂OCH₂CH₂—,
—CH═N—OCH₂C(O)—, or
N,N′-disubstituted piperazine.
R₅ is H, cyano, acyl, nitro, alkoxycarbonyl, aminocarbonyl, hydroxyl, alkoxyl, acyloxy, or amido.

27. The method of claim 23, wherein the salicylamide analog is: [chemical structure]
